(19) United States
(12) Patent Application Publication
(10) Pub. No.: US 2025/0144050 A1
(43) Pub. Date: May 8, 2025

(54) AMINOTHIOL-CONJUGATES, COMPOSITIONS, AND METHODS OF USE THEREOF

(71) Applicant: THE BURLINGTON HC RESEARCH GROUP, INC., Jericho, VT (US)

(72) Inventors: Dale M. WALKER, Jericho, VT (US); Vernon E. WALKER, Jericho, VT (US)

(21) Appl. No.: 18/686,757

(22) PCT Filed: Aug. 26, 2022
(86) PCT No.: PCT/US2022/041687

§ 371 Date: Feb. 26, 2024

Related U.S. Application Data
(60) Provisional application No. 63/237,937, filed on Aug. 27, 2021.

(51) Int. Cl. A61K 31/145; A61K 31/517; A61K 33/243; A61K 45/06; A61P 35/00

(52) U.S. Cl. CPC A61K 31/145; A61K 31/517; A61K 33/243; A61K 45/06; A61P 35/00

(57) ABSTRACT

The present disclosure relates to a method of treating a neoplastic condition in a subject in need thereof. This method involves administering to the subject a combination therapy comprising (i) an aminothiol-conjugate of Formula (I): wherein Core, Linker, R₁, R₂, R₃, m, n, and p are as described above. The present disclosure also relates to methods of treating a subject in need of antimicrobial treatment, methods of increasing sensitivity of a cell to treatment with an anti-neoplastic drug or antimicrobial drug, and combination therapeutics comprising one or more aminothiol-conjugates of Formula (I).

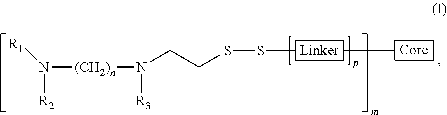

This application claims benefit of U.S. Provisional Application Ser. No. 63/237,937, filed Aug. 27, 2021, which is hereby incorporated by reference in its entirety.

### FIELD

The present disclosure relates to aminothiol-conjugates, combination therapies, and methods of their use in the treatment of subjects in need thereof.

### BACKGROUND

In the current aminothiol drug formulations referred to as the phosphorothioates, protection of the biologically-active aminothiol moiety relies upon conjugation of the aminothiol to a phosphate group. In this formulation, the phosphate group is bound to the sulfhydryl moiety of the aminothiol and it serves the purpose of protecting the active metabolite from adventitious reactivity during the process of drug delivery to target and non-target cells. In the vicinity of cell membranes, the phosphate group is removed by cell membrane-bound alkaline phosphatase. Then the active metabolite (the aminothiol) is taken into the cell by passive diffusion or, under some conditions, active transport by a plasma membrane-associated transport system.

Delivery of the phosphorothioates to normal cells is successful because many/most non-stressed/non-diseased cells produce the alkaline phosphatase isoform that is localized in the cell membrane. However, the same prodrugs are not as effective or are ineffective for the treatment of stressed or diseased cells for several reasons including (i) rapid clearance from circulation, (ii) inability of some cells, and especially stressed or diseased cells, to metabolize the phosphorothioates to their active forms, (iii) vulnerability to metabolism distal to target cells, and (iv) vulnerability to conversion to toxic byproducts (Block et al., “Commentary: the Pharmacological Antioxidant Amifostine—Implications of Recent Research for Integrative Cancer Care,” Integr. Cancer Ther. 4:329-351 (2005); Calabro-Jones et al., “The Limits to Radioprotection of Chinese Hamster V79 cells by WR-1065 under Aerobic Conditions,” Radiat. Res. 149:550-559 (1998); Meier et al., “Degradation of 2-(3-Aminopropylamino)-ethanethiol (WR-1065) by Cu-Dependent Amine Oxidases and Influence on Glutathione Status of Chinese Hamster Ovary Cells,” Biochem. Pharmacol. 50:489-496 (1995), Santini et al., “The Potential of Amifostine: From Cytoprotectant to Therapeutic Agent,” Haematologica 84 (11) 1035-1042 (1999)). Other limitations include (i) the inability to take advantage of multiple different drug absorption mechanisms, which can differ between diseased versus normal cells and between diseased cells with differing pathologies, (ii) the inability to target cell uptake or transport systems to enhance drug uptake into cells, (iii) the inability to target or exclude specific cell types, (iv) the inability to alter drug circulation or retention times, (v) the inability to target or exclude specific drug clearance mechanisms, and (vi) the poor/limited ability to incorporate the aminothiols and phosphorothioates into drug delivery modules such as nanoparticles or liposomes due to the small size and hydrophilicity of the aminothiols. New drug formulations for the aminothiols are needed to overcome these problems and limitations.

Further, new therapies are needed for the treatment of infectious diseases and neoplastic conditions. Despite certain improvements in medical treatment for such diseases (antibiotics and vaccines for infectious diseases and antineoplastic drugs for neoplastic conditions), there remain many obstacles. A primary issue is the emergence of drug-resistant pathogens and drug-resistant neoplastic conditions. For example, in spite of recent advancements in the development of new therapeutic options, the lack of anticancer agents that surmount drug resistance is the single biggest barrier to achieving improved therapy for neoplasia in general (Lim and Ma, “Emerging Insights of Tumor Heterogeneity and Drug Resistance Mechanisms in Lung Cancer Targeted Therapy,” J. Hematol. Oncol. 12 (1): 134 (2019) and Vasan et al., “A View on Drug Resistance in Cancer,” Nature 575 (7782): 299-309 (2019)). Inherent and/or acquired drug resistance poses a difficult problem because (i) aggressive cancers frequently are heterogeneous such that not all cells respond to available treatments and (ii) resistance can occur via multiple mechanisms such as mutation induction and gene expression changes that can be triggered simultaneously (Harrison and Huang, “Exploiting Vulnerabilities in Cancer Signaling Networks to Combat Targeted Therapy Resistance,” Essays Biochem. 62 (4): 583-593 (2018)). The current approach to this barrier is to use combinations of antineoplastic drugs with differing modes of action, but to date success has been limited (Vasan et al., “A View on Drug Resistance in Cancer,” Nature 575 (7782): 299-309 (2019); Rebuzzi et al., “Combination of EGFR-TKIs and Chemotherapy in Advanced EGFR Mutated NSCLC: Review of the Literature and Future Perspectives,” Crit. Rev. Oncol. Hematol. 146:102820 (2020); Westover et al., “Mechanisms of Acquired Resistance to First- and Second-Generation EGFR Tyrosine Kinase Inhibitors,” Ann. Oncol. 29 (suppl_1): 110-i19 (2018); and Herbst et al., “The Biology and Management of Non-Small Cell Lung Cancer,” Nature 553 (7689): 446-454 (2018)). The need for novel effective anticancer agents and rationally designed combination therapies that prevent and overcome drug resistance is imperative (Herbst et al., “The Biology and Management of Non-Small Cell Lung Cancer,” Nature 553 (7689): 446-454 (2018)).

Thus, there exists a need for an effective and easily administered therapy against these conditions and for the treatment of their drug resistant forms.

The present invention is directed to overcoming these and other deficiencies in the art.

### SUMMARY

A first aspect of the present disclosure relates to a method of treating a neoplastic condition in a subject in need thereof. This method involves administering to the subject a combination therapy comprising (i) an aminothiol-conjugate of Formula (I):

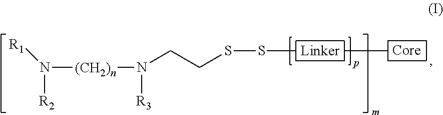

where

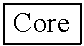

is optional and, if present, is an atom, a molecule, or a macromolecule;

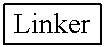

is a linker group, where the linker group is a polymer, a section of a polymer, an arm of a polymer, an arm of a copolymer, or a branch of a dendrimer;
where each

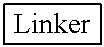

can be the same or different;
R₁, R₂, and R₃ are independently selected from hydrogen and C₁₋₆ alkyl;
m is 1 to 100,000;
n is 1 to 10; and
p is independently 0 to 2,500,
or a pharmaceutically acceptable salt, hydrate, polymorph, or solvate thereof, and (ii) an anti-neoplastic drug, where the combination therapy is administered in an amount effective to treat the neoplastic condition in the subject.

A second aspect of the present disclosure relates to a combination therapeutic comprising:
(i) an aminothiol-conjugate of Formula (I):

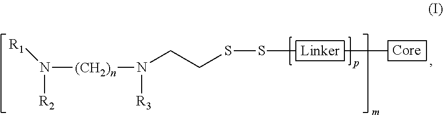

where

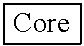

is optional and, if present, is an atom, a molecule, or a macromolecule;

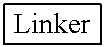

is a linker group, where the linker group is a polymer, a section of a polymer, an arm of a polymer, an arm of a copolymer, or a branch of a dendrimer;
where each

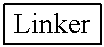

where each can be the same or different;
R₁, R₂, and R₃ are independently selected from hydrogen and C₁₋₆ alkyl;
m is 1 to 100,000;
n is 1 to 10; and
p is independently 0 to 2,500,
or a pharmaceutically acceptable salt, hydrate, polymorph, or solvates thereof, and
(ii) an anti-neoplastic drug.

A third aspect of the present disclosure relates to a method of treating a subject in need of antimicrobial treatment. This method involves administering to the subject a combination therapy comprising (i) an aminothiol-conjugate of Formula (I):

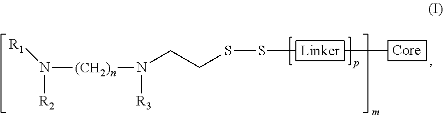

where

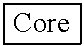

is optional and, if present, is an atom, a molecule, or a macromolecule;

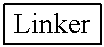

where each is a linker group, where the linker group is a polymer, a section of a polymer, an arm of a polymer, an arm of a copolymer, or a branch of a dendrimer;
where each

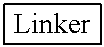

where each can be the same or different;
R₁, R₂, and R₃ are independently selected from hydrogen and C₁₋₆ alkyl;
m is 1 to 100,000;
n is 1 to 10; and
p is independently 0 to 2,500,
or a pharmaceutically acceptable salt, hydrate, polymorph, or solvate thereof, and (ii) an antimicrobial drug, where the combination therapy is administered in an amount effective to treat a microbial condition in the subject.

A fourth aspect of the present disclosure relates to a combination therapeutic comprising:
(i) an aminothiol-conjugate of Formula (I):

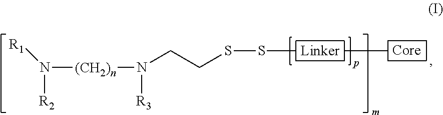

where

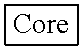

is optional and, if present, is an atom, a molecule, or a macromolecule;

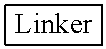

is a linker group, where the linker group is a polymer, a section of a polymer, an arm of a polymer, an arm of a copolymer, or a branch of a dendrimer;
where each

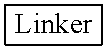

can be the same or different;
R₁, R₂, and R₃ are independently selected from hydrogen and C₁₋₆ alkyl;
m is 1 to 100,000;
n is 1 to 10; and
p is independently 0 to 2,500,
or a pharmaceutically acceptable salt, hydrate, polymorph, or solvates thereof, and (ii) an antimicrobial drug.

A fifth aspect of the present disclosure relates to a method of increasing sensitivity of a cell to treatment with an anti-neoplastic drug or antimicrobial drug. This method involves selecting a neoplastic cell, a microbial cell (e.g., a bacterial cell, a fungal cell, or a parasitic cell), or a cell infected with a microbe (e.g., a cell infected with a virus, bacterium, fungus, or parasite); and administering to the cell an aminothiol-conjugate of Formula (I):

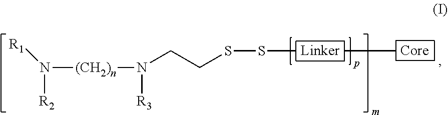

where

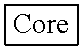

is optional and, if present, is an atom, a molecule, or a macromolecule;

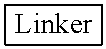

is a linker group, where the linker group is a polymer, a section of a polymer, an arm of a polymer, an arm of a copolymer, or a branch of a dendrimer;
where each

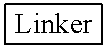

can be the same or different;
R₁, R₂, and R₃ are independently selected from hydrogen and C₁₋₆ alkyl;
m is 1 to 100,000;
n is 1 to 10; and
p is independently 0 to 2,500,
or a pharmaceutically acceptable salt, hydrate, polymorph, or solvate thereof in an amount effective to increase sensitivity of the cell to treatment with the anti-neoplastic drug or antimicrobial drug.

The disclosure relates generally to the field of drug delivery that involves the use of polymeric carrier(s) in which a carrier molecule is covalently bound to a molecule of interest (i.e., aminothiol). The drug delivery system may achieve, for example, (i) increased water solubility for the drug, (ii) increased stability of the drug against degrading enzymes or reduction of uptake by the reticulo-endothelial system, (iii) targeted delivery of drugs to specific sites, and (iv) ease of incorporation of a drug into drug delivery modules such as nanoparticles and liposomes. The disclosure also relates to reformulating aminothiol drugs for the purpose of protecting one or more active moieties and for enhancing the pharmacokinetics and pharmacodynamics of the reformulated entity for delivery to humans and other animals alone or in combination with other drugs or active agents (e.g., antineoplastic and/or antimicrobial drugs). As set forth in the Examples below, a series of experiments were conducted to evaluate whether co-treatment with aminothiol-conjugate of Formula (I) and anti-neoplastic therapeutic(s) enhances anticancer effectiveness and overcomes drug resistance. The examples show significant and unexpected drug effects for the aminothiol-conjugates described herein when used alone or in combination with anti-neoplastic drugs. These surprising effects include, but are not limited to, sensitizing cancer cells to treatment with anti-neoplastic therapeutics and reducing the dose of anti-neoplastic therapeutics needed for inhibition of cancer cell growth and/or for induction of cancer cell death.

Additional unexpected findings include: (i) reversal of drug resistance and (ii) improvement in efficacy for neoplastic drugs and aminothiol-conjugates when used in combination (synergistic effects; including reducing the level of the anticancer agent used in combination with aminothiol-conjugates according to the present disclosure from high levels that cannot be tolerated by patients, to levels that can be tolerated by patients and/or that have minimal side effects). Indeed, useful drug resistance reversal requires that cell sensitivity to a neoplastic agent be achieved at doses that can be tolerated by a patient. Further, use of the aminothiol-conjugates according to the present disclosure (e.g., 4SP65) may prevent drug resistance development. For example, use of cisplatin at drug concentrations that can be achieved in circulation in patients without induction of intolerable toxic effects, only can achieve cytostatic/growth inhibition effects. However, when combined with an aminothiol-conjugate according to the present disclosure (e.g., 4SP65), cisplatin achieves cytostatic and cytocidal effects and does so at low drug concentrations that can be achieved safely in circulation in patients. This phenomenon is important because if a therapy only achieves growth inhibition effects, then it may only slow progression of the cancer, which may allow cancer cells time to adapt to treatment and establish drug-resistant cells. A therapeutic that achieves cytostatic and cytocidal effects may induce disease resolution, which prevents the development of drug resistance, cancer recurrence, and disease progression, because dead cells and cells that cannot grow are unable to change gene expression profiles and/or establish clones and colonies of drug-resistant cells. The present disclosure therefore provides for significant and surprising advances in the use of aminothiols and treatment of cancer and infectious disease in subjects.

### BRIEF DESCRIPTION OF THE DRAWINGS

FIG. 1 shows the structure of WR1065, the active moiety of amifostine. The linear formula is NH₂(CH₂); NH(CH₂)₂SH.

FIG. 2 shows the structure of WR255591, the active moiety of phosphonol. The linear formula is CH₃NH(CH₂)₃NH(CH₂)₂SH.

FIGS. 3A-3B show two generic structures of an aminothiol (and analogues thereof), where X is selected from the group consisting of —PO₃H₂, hydrogen, sulfhydryl, sulfur, acetyl, isobutyryl, pivaloyl, and benzoyl, wherein each of R₁, R₂, and R₃ is independently selected from hydrogen and C₁₋₆ alkyl, wherein n is an integer of from 1 to 10, and (in FIG. 3B) wherein n′ is an integer of from 1 to 10. Two exemplary structures of active moieties of the generic aminothiols shown in FIGS. 3A-3B are where X is hydrogen.

FIG. 4 shows the general structure of polyethylene glycol (polyethylene oxide), wherein ‘n’ can be any integer from 1 or greater. The linear formula is H—(O—CH₂—CH₂)ₙ—OH, where in ‘n’ can be any integer, with a range of 1 to 2500 being most desirable for the applications presented here. Commonly used variants include monomethoxy PEG or dihydroxyl PEG. See, e.g., suitable monomethoxy Poly(ethylene glycol) or dihydroxyl Poly(ethylene glycol) at Sigmaaldrich.com, which is hereby incorporated by reference in its entirety.

FIGS. 5A-5B show the general structure of thiol-terminated polyethylene glycol (polyethylene oxide) wherein ‘n’ can be any integer from 1 to 2500. See, e.g., suitable Poly(ethylene glycol) dithiols at Sigmaaldrich.com, Homobifunctional PEGs, which is hereby incorporated by reference in its entirety.

FIG. 6 shows the general structure of 4-arm, thiol-terminated, star polyethylene glycol, wherein ‘n’ can be any integer, with a range from 1 to 2500 being most desirable for the applications presented here. See, e.g., suitable PEG polymers and dendrimers at Sigmaaldrich.com, PEG Dendrimers and Multi-arm PEGs, each of which is hereby incorporated by reference in its entirety.

FIG. 7 shows the general structure of a thiol-terminated polyethylene glycol conjugated via a disulfide bond to an aminothiol, wherein ‘n₁’ can be any integer from 1 to 4 and ‘n₂’ can be any integer from 1 to 4. See. e.g., suitable PEG polymers and dendrimers at Sigmaaldrich.com, PEG Dendrimers and Multi-arm PEGs, which is hereby incorporated by reference in its entirety. As described herein, the conjugate according to certain embodiments of the present disclosure is a 4-arm, thiol-terminated, star polyethylene glycol conjugated via a disulfide bond to an aminothiol and has the structure shown in FIG. 7, where ‘n₂’ is 4.

FIG. 8 shows the general structure of 6-arm-PEG. See, e.g., suitable PEG polymers and dendrimers at Sigmaaldrich.com, PEG Dendrimers and Multi-arm PEGs, which is hereby incorporated by reference in its entirety. This general design can be expanded further to create an 8-arm star PEG scaffold, for example.

FIG. 9 shows the general structure for folate (folic acid). The linear formula is C₁₉H₁₁₉N₇O₆. By altering the terminal carboxyl group to the appropriate moiety (e.g., SH) and then carrying out the addition of an aminothiol or PEG, respectively, a conjugate of folic acid with an aminothiol or PEG can be synthesized (Chen et al., “Folate-Mediated Intracellular Drug Delivery Increases the Anticancer Efficacy of Nanoparticulate Formulation of Arsenic Trioxide,” Mol. Cancer Ther. 8 (7): 1955-63 (2009); Kang et al., “Folic Acid-Tethered Pep-1 Peptide-Conjugated Liposomal Nanocarrier for Enhanced Intracellular Drug Delivery to Cancer Cells: Conformational Characterization and in vitro Cellular Uptake Evaluation,” Int. J. Nanomed. 8:1155-1165 (2013), each of which is hereby incorporated by reference in its entirety). The folic acid conjugate offers the advantage that it can interact with the folic acid receptor on the surface of cells and trigger active transport of the aminothiol-conjugates of the present disclosure into the cell cytosol.

FIG. 10 shows the general structure of spermine polymer. The linear formula is NH₂C₂H₄(NC₃H₆NHC₄H₈)ₙ—NHC₃H₆NH₂, wherein ‘n’ can be any integer equal to or greater than 1. By altering a terminal NH-group to the appropriate moiety (e.g., SH) and then carrying out the addition of an aminothiol or PEG, respectively, a conjugate of spermine with an aminothiol or PEG can be synthesized. The spermine polymer conjugate offers the advantage that it can interact with the polyamine receptor on the surface of cells and trigger active transport of the prodrug into the cell cytosol (see Zhang et al., “Short Biodegradable Polyamine for Gene Delivery and Transfection of Brain Capillary Endothelial Cells,” J. Control Release 143:359-366 (2010), which is hereby incorporated by reference in its entirety).

FIG. 11 shows a general structure of an aminothiol-conjugate as described herein. The conjugate may include, e.g., any structure as shown in FIG. 3 through FIG. 6 and FIG. 8 through FIG. 10, and can vary with the drug delivery and drug activation conditions and needs of the stress condition or disease for which therapeutic intervention is desired. In some embodiments, two conjugates can be combined; for example folic acid can be linked to a PEG-containing polymer that is then linked to an aminothiol via a disulfide bond.

FIG. 12 shows a general structure of an aminothiol-conjugate as described herein. The conjugate may include, e.g., any structure as shown in FIG. 3 through FIG. 6 and FIG. 8 through FIG. 10, and can vary with the drug delivery and drug activation conditions and needs of the stress condition or disease for which therapeutic intervention is desired. In some embodiments, two conjugates can be combined; for example folic acid can be linked to a PEG-containing polymer that is then linked to an aminothiol via a disulfide bond. In some embodiments, the length of the carbon chain between the sulfur moiety and the first nitrogen of the aminothiol can vary in length (see FIG. 3B, supra), as shown in FIG. 12.

FIG. 13 shows a MALDI-TOF spectra of 4SP65. The top panel shows the AMW (average molecular weight)=10531.950, which corresponds to the incorporation of four molecules of WR1065 onto the 10,000 MW backbone of the 4-arm PEG polymer. The bottom panel shows an expansion of the MALD-TOF spectra showing the PEG repeats in the backbone of the polymer.

FIG. 14 shows the 400 MHz 1H NMR spectra for 1LP65 in D20. The peaks at 3.88 (2H), 3.71 (18H), 3.65 (2H), 3.4 (3H), and 3.06 (2H) are from the mPEG6-SH backbone, the other resonances are from the 2-((3-aminopropyl)amino) ethanethiol moiety.

FIG. 15 shows the ESI⁺ mass spectrum for 1LP65 showing the mass of 444.74 (M+H) which corresponds with the calculated mass for this structure.

FIGS. 16A-16B are plots showing a fitted logistic curve (FIG. 16A) showing concentration parameters to measure drug effectiveness and dose-response curves (FIG. 16B) for growth of normal human mammary epithelial cells (NHMECs) treated with 4SP65 or 1LP65. In FIG. 16A, individual dose-response metrics representing the degree of effectiveness of a theoretical drug are shown on the Y-axis and defined as follows: 100 indicates no growth inhibition, demonstrating growth of sham-exposed control cells above starting cell numbers; GI₅₀ indicates drug level inducing ‘growth inhibition of 50%’ after starting cell numbers are removed, consistent with ‘slowing of progressive disease’; TGI indicates drug level inducing ‘total growth inhibition’ of starting cell numbers, consistent with ‘induction of stable disease’: LC₅₀: drug effects plotted at −50 to indicate a ‘lethal concentration of 50%’ that inhibits cell growth and reduces starting cell numbers by half, consistent with ‘induction of partial disease resolution’; LC₉₉: drug effects plotted at −99 to indicate a ‘lethal concentration of 99%’ that inhibits cell growth and reduces starting cell numbers to nearly 0, consistent with ‘near complete disease resolution’. In FIG. 16B, the curves show percentage of cell growth, represented by relative fluorescence counts/units (RFUs), in strains of NHMECs exposed to 4SP65 or 1LP65, with the dotted line at ‘0’ on the Y-axis denoting relative fluorescence of starting cell numbers at the initiation of drug treatments. Tested strains of NHMECs from three distinct sources included #M99005 (or ‘S-1’), #70043304 (or ‘S-2’), and #1669 (or ‘S-3’). Strains were plated, incubated for approximately 24 hours prior to initiation of treatment, and scored after a 48-hour exposure over a dose range of 4SP65 or 1LP65. Concentrations of 150 μM 4SP65 and 500 μM 1LP65 were the highest levels that could be tested based upon the maximum solubility of 4SP65 in aqueous medium and the need for sufficient dilution of DMSO using aqueous medium to dilute 40 mM 1LP65 in DMSO. Error bars represent SEMs of replicate experiments.

FIGS. 17A-17F are dose-response curves for growth of human cancer cell lines (HCC38, MBA-MB-231 (FIG. 17A); A549, H460 (FIG. 17B); H1437, H1975 (FIG. 17C); DU145, LNCaP, PC3 (FIG. 17D); SKOV3, TOV21G, PANC1 (FIG. 17E); HMESO1, PPMMill, and HL60 (FIG. 17F) exposed to 4SP65, 1LP65, or amifostine (AMF). Graphs in FIGS. 17A-17F show percentage of cell growth (cytostatic growth inhibitory effects above dotted lines, total growth inhibitory effects at dotted horizontal lines, and lethal effects below dotted lines) after 48-hour drug treatments, with the dotted line representing starting cell numbers. Data points represent average values across 3-7 experiments for individual drugs and cell lines (see FIG. 18); error bars represent SEs for the means of related experiments, with a minimum of four biological replicates/treatment level included in each experiment.

FIG. 18 is a table showing comparisons of dose-response metrics for 4SP65, 1LP65, and amifostine, and values reported for selected cytotoxic chemotherapeutic agents and PRIMA-1. ^{a}Cell lines were plated, incubated for approximately 24 hours prior to addition of experimental drugs, and scored after a 48-hour treatment over a dose range of 4-star PEG-S-S-WR1065 (4SP65), m-PEG6-S-S-WR1065 (1LP65), or amifostine (AMF). The dose-response metrics measured included inhibitory concentration 50% (IC₅₀), growth inhibitory 50% (GI₅₀), total growth inhibition (TGI), lethal concentration 50% and 99% (LC₅₀, LC₉₉) in cancer cells. Mean concentrations for each drug are given as μM, with standard errors provided for each experimental drug tested. ^{b} Major oncogenic gene alterations are listed in parentheses after the name of each human cancer cell line. ^{c}Relative potency, a measurement of the fold-difference in the efficacy of the drug in the denominator compared to the drug in the numerator at a given dose-response metric; e.g., 4SP65 is 67-fold more effective than amifostine in achieving an IC₅₀ in HCC38 cells. ^{d} ND, not definable for the given dose-response metric when the highest concentration of amifostine used to treat the listed human cancer cell lines was 500 μM. Values above 100 M for amifostine are listed for comparison purposes only because plasma concentrations of 100 μM amifostine and exposures of over 3 hours are not readily achievable in humans or animals due to inherent dose-limiting toxicities and drug delivery restrictions (Grdina et al., “Amifostine: Mechanisms of Action Underlying Cytoprotection and Chemoprevention,” Drug Metabol. Drug Interact. 16 (4): 237-79 (2000), which is hereby incorporated by reference in its entirety). ^{c}Metrics for cisplatin and paclitaxel are from the NCI Database of Screening Results (Monks et al., “Feasibility of a High-Flux Anticancer Drug Screen using a Diverse Panel of Cultured Human Tumor Cell Lines,” J. Natl. Cancer Inst. 83 (11): 757-766 (1991), which is hereby incorporated by reference in its entirety) and the GI₅₀ values for PRIMA-1 are from Perdrix et al., “PRIMA-1 and PRIMA-1 (Met) (APR-246): From Mutant/Wild Type p53 Reactivation to unexpected Mechanisms Underlying their Potent Anti-Tumor Effect in Combinatorial Therapies,” Cancers (Basel) 9 (12): 172 (2017), which is hereby incorporated by reference in its entirety.

FIG. 19 shows the average dose-response metrics across human cancer cell lines exposed to 4SP65, 1LP65, or amifostine. ^{a} Cancer cell lines were plated, incubated for approximately 24 hours prior to addition of experimental drugs, and scored after a 48-hour treatment over a dose range of 4-star PEG-S-S-WR1065 (4SP65), m-PEG6-S-S-WR1065 (1LP65), or amifostine. The response metrics measured included inhibitory concentration 50% (IC₅₀), growth inhibitory 50% (GI₅₀), total growth inhibition (TGI), lethal concentration 50% and 99% (LC₅₀, LC₉₉) in cancer cells and cytotoxic concentration 50% (CC₅₀) in normal mammary epithelial cells. Mean concentrations for each drug are given as μM, with standard errors provided for each experimental drug tested. Additional abbreviations include: WT, wild-type; Mut, Mutant. ^{b} Human cancer cell lines included HCC38 and MDA-MB-231 breast cancer cells; A549, H460, H1437, and H1975 non-small lung cancer cells, HMESO and PPMMill pleural mesothelioma cells; PANC1 pancreatic cancer cells; DU145, LNCaP, and PC3 prostate cancer cells: SKOV3 and TOV21G ovarian cancer cells; and HL60 acute promyelocytic leukemia cells. ^{c}Included A549, H460, PPMMill, and LNCaP cell lines. ^{d} Included HCC38, MDA-MB-231, H1975, HMESO1, PANC1, DU145, and PC3 cells. ^{e}Included HCC38, MDA-MB-231, A549, H460, H1437, H1975, DU145, LNCaP, PC3, SKOV3, and TOV21G cells. ^{f} Included A549, H460, and LNCaP cells. ^{g}& Included HCC38, MDA-MB-231, H1975, DU145, and PC3 cells. ^{h} GI50 values <100 μM amifostine in 4/14 human cancer cell lines, including HCC38, PC3, SKOV3, and TOV21G cells. ⁱ GI50 values >100 μM amifostine in 7/14 human cancer cell lines; GI50 values were not definable in 3/14 cancer cell lines. ^{j} Amifostine concentrations >100 μM generated TGI level effects in only two cell lines, HCC38 and PC3. ^{k} Not definable for the given dose-response metric when the highest concentration of amifostine used to treat the listed human cancer cell lines was 500 μM. Values above 100 μM for amifostine are listed for comparison purposes only because serum concentrations of 100 μM amifostine and exposures of over 3 hours are not readily achievable in humans or animals due to inherent dose-limiting toxicities and drug delivery restrictions (Grdina et al., “Amifostine: Mechanisms of Action Underlying Cytoprotection and Chemoprevention,” Drug Metabol. Drug Interact. 16 (4): 237-79 (2000), which is hereby incorporated by reference in its entirety).

FIG. 20 is a graph showing relative drug potency ratios based on the dose-response metrics for 4SP65, 1LP65, and amifostine across a panel of human cancer cell lines. For each dose-response metric, the values obtained after a 48-hour treatment of 11 human cancer cell lines with each individual drug (FIG. 18) were first averaged and then fold-differences in drug potency were calculated as ratios for 4SP65 compared to amifostine, 4SP65 compared to 1LP65, and 1LP65 compared to amifostine. IC₅₀, GI₅₀, and TGI values were measurable in only four or fewer cancer cell lines, depending upon metric assessed, following maximum exposures of 500 μM amifostine, and thus the solid bars represent minimal fold differences in drug potency between 4SP65 or 1LP65 compared to amifostine. Maximum exposures to 500 μM amifostine did not yield LC₅₀ or LC₉₉ values in any cancer cell line, so fold differences in drug potency for cytolytic effects between 4SP65 or 1LP65 versus amifostine can't be calculated.

FIGS. 21A-21F are dose-response curves for growth of human cancer cell lines exposed to 4SP65, cisplatin (DDP), or both drugs. The graphs in FIGS. 21A-21F show percentage of cell growth (cytostatic growth inhibitory effects above dotted lines, total growth inhibitory effects at dotted horizontal lines, and lethal effects below dotted lines) after 48-hour treatments, with the dotted line representing starting cell numbers. Cell lines evaluated include A549 (FIG. 21A), H460 (FIG. 21B), HMESO1 (FIG. 21C), PPMMill (FIG. 21D), SKOV3 (FIG. 21E), and TOV21G (FIG. 21F). Data points represent average values across 3 or 4 experiments for single or combined drug treatments of each cell line (see Table 3); error bars represent SEs for the means of related experiments, with a minimum of four biological replicates/treatment level included in each experiment.

FIGS. 22A-22B show dose-response curves for growth of human cancer cell lines exposed to 4SP65 (4SP), gefitinib (GEF), or both drugs. The graphs in FIGS. 22A-22B show percentage of cell growth (cytostatic growth inhibitory effects above dotted lines, total growth inhibitory effects at dotted horizontal lines, and lethal effects below dotted lines) after 48-hour treatments, with the dotted line representing starting cell numbers. Data points represent average values across 4 and 5 experiments for single or combined drug treatments of H1975 (FIG. 22A) and A549 cells (FIG. 22B), respectively; error bars represent SEs for the means of related experiments, with a minimum of 4 biological replicates/treatment level included in each experiment.

FIGS. 23A-23F show synergy reports for human cancer cell lines treated with 4SP65 and cisplatin in combination. SynergyFinder 2.0 (Ianevski et al. “SynergyFinder 2.0: Visual Analytics of Multi-Drug Combination Synergies,” Nucleic Acids Res. 48 (W1): W488-W493 (2020), which is hereby incorporated by reference in its entirety) was used to produce dose-response curves for 4SP65 (4SP) and cisplatin (DDP), a dose-response matrix for growth inhibition, a heat map showing clustering of degrees of synergism, a volcano plot showing the distribution of synergy strength, and to derive synergy scores following single drug and combination drug treatments of A459 cells (FIG. 23A), H460 cells (FIG. 23B), HMESO1 cells (FIG. 23C), PPM-Mill cells (FIG. 23D), SKOV3 cells (FIG. 23E), and TOV21G (FIG. 23F) cells for 48 hours.

FIGS. 24A-24B shows synergy reports for human cancer cell lines treated with 4SP65 and gefitinib in combination. SynergyFinder 2.0 (Ianevski et al. “SynergyFinder 2.0: Visual Analytics of Multi-Drug Combination Synergies,” Nucleic Acids Res. 48 (W1): W488-W493 (2020), which is hereby incorporated by reference in its entirety) was used to produce dose-response curves for 4SP65 (4SP) and gefitinib (GEF), a dose-response matrix for growth inhibition, a heat map showing clustering of degrees of synergism, a volcano plot showing the distribution of synergy strength, and to derive synergy scores following single drug and combination drug treatments of H1975 cells (FIG. 24A) and A549 cells (FIG. 24B) for 48 hours.

FIG. 25 is a schematic showing the metabolism of BRGs (aminothiol-conjugates according to the present disclosure) and redox modulation effects of WR1065, the PEG-scaffold, and combinations. Hexagon: a BRG drug diamond+triangle with disulfide bond, BRG drug, diamond: WR1065, triangle: a PEG scaffold, GSH: glutathione, Trx: thioredoxin.

FIG. 26 is a schematic showing the metabolism of amifostine and redox modulation effects of WR1065. Under ideal conditions amifostine is converted to WR1065 by membrane-bound alkaline phosphatase. This hydrolysis reaction does not perturb the redox balance. WR1065 is reported to donate hydrogen ions and, thus, can reduce oxidized species such as oxidized glutathione (GSSG), thioredoxin (with an intramolecular disulfide bond), peroxiredoxin (with an intramolecular disulfide bond), and NADP⁺ (the oxidized form). Other oxidized molecules also can be reduced by WR1065. The net effect is to increase the reducing capacity of cells; under stress condition, this WR1065-mediated effect increases the cellular ability to neutralize electrophilic species.

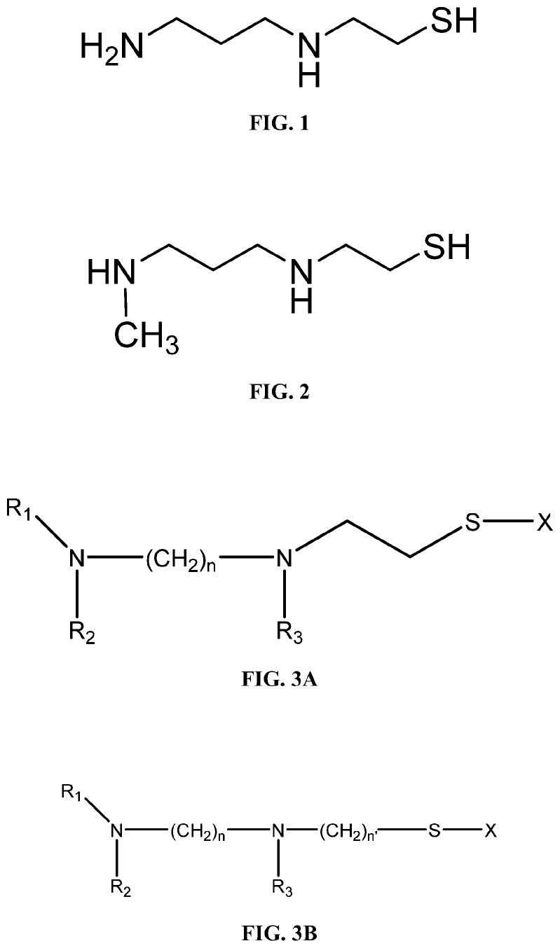

### DETAILED DESCRIPTION

The present disclosure relates to improved methods of achieving protection of the active moiety(ies) of phosphorothioate compounds (i.e., aminothiols), delivery of the protected compounds, and activation at desired sites in vivo in humans and animals. The present disclosure also relates to methods for achieving increased drug efficacy and reduced toxicity. The present disclosure relates to methods for achieving improved therapeutic efficacy and lower toxicity of aminothiols described herein through use of the aminothiol-conjugates of the disclosure administered alone or in combination with other therapeutics (e.g., anti-neoplastic or anti-microbial drugs). Such protected drugs can be delivered without the use of additional delivery methods or modules, or can be combined with drug delivery systems that achieve intracellular, intracytoplasmic, active or passive targeted cell delivery or exclusion, and/or intra-subcellular organelle delivery.

Unless otherwise indicated, the definitions and embodiments described in this and other sections are intended to be applicable to all embodiments and aspects of the present application herein described for which they are suitable as would be understood by a person skilled in the art.

Unless defined otherwise, all technical and scientific terms used in this disclosure have the same meanings as commonly understood by one of ordinary skill in the art to which this disclosure belongs.

Preferences and options for a given aspect, feature, embodiment, or parameter of the invention should, unless the context indicates otherwise, be regarded as having been disclosed in combination with any and all preferences and options for all other aspects, features, embodiments, and parameters of the disclosure.

In this specification and the appended claims, the singular forms “a,” “an,” and “the” include plural references unless the context clearly dictates otherwise.

The terms “comprising,” “comprises,” and “comprised of” as used herein are synonymous with “including,” “includes,” or “containing,” “contains,” and are inclusive or open-ended and do not exclude additional, non-recited members, elements, or method steps.

The terms “comprising,” “comprises,” and “comprised of” also encompass the term “consisting of.” The transitional term “comprising,” which is synonymous with “including,” “containing,” or “characterized by,” is inclusive or open-ended and does not exclude additional, un-recited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed subject matter. In some embodiments or claims where the term comprising is used as the transition phrase, such embodiments can also be envisioned with replacement of the term “comprising” with the terms “consisting of” or “consisting essentially of.”

Terms of degree such as “substantially,” “about,” and “approximately” and the symbol “˜” as used herein mean a reasonable amount of deviation of the modified term such that the end result is not significantly changed. These terms of degree should be construed as including a deviation of at least ±0.1% (and up to ±1%, ±5%, or ±10%) of the modified term if this deviation would not negate the meaning of the word it modifies. Unless otherwise clear from context, all numerical values provided herein are modified by the term about. All numerical values provided herein that are modified by terms of degree set forth in this paragraph (e.g., “substantially,” “about,” “approximately,” and “˜”) are also explicitly disclosed without the term of degree. For example, “about 1%” is also explicitly disclosed as “1%”.

The term “and/or” as used herein means that the listed items are present, or used, individually or in combination. In effect, this term means that “at least one of” or “one or more” of the listed items is used or present.

The recitation of numerical ranges by endpoints includes all numbers and fractions subsumed within the respective ranges, as well as the recited endpoints. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member.

The term “subject” is inclusive of the definition of the term “patient” and inclusive of the term “healthy subject” (i.e., an individual (e.g., a human)) who is entirely normal in all respects or with respect to a particular condition.

The term “patient” means a subject (e.g., a human) who has presented a clinical manifestation of a particular symptom or symptoms suggesting the need for treatment, who is treated preventatively or prophylactically for a condition, or who has been diagnosed with a condition to be treated.

The terms “treat”, “treatment of”, “treating” and the like refer to both therapeutic treatment and prophylactic or preventative measures, wherein the object is to protect against (partially or wholly) or slow down (for example, lessen or postpone the onset of) an undesired physiological condition, disorder or disease, or to obtain beneficial or desired clinical results such as partial or total restoration or inhibition in decline of a parameter, value, function or result that had or would become abnormal. For example, beneficial or desired clinical results include, but are not limited to, alleviation of symptoms; diminishment of the extent or vigor or rate of development of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether or not it translates to immediate lessening of actual clinical symptoms, or enhancement or improvement of the condition, disorder or disease. Treatment seeks to elicit a clinically significant response without excessive levels of side effects.

The term “combination therapy” means the administration of two or more therapeutic agents to treat a medical condition or disorder described in the present disclosure. Such administration encompasses co-administration of these therapeutic agents in a substantially simultaneous manner, such as in a single capsule, or dosage presentation, having a fixed ratio of active ingredients, or in multiple, separate forms, e.g., oral dosage forms, parenteral dosage forms, etc., for each active ingredient. In addition, such administration also encompasses use of each type of therapeutic agent in a sequential manner in the same patient, with delivery of the individual therapeutics separated by, e.g., 1-24 hours, 1-27 days, or 1 or more weeks. In either case, the treatment regimen will provide beneficial effects of the drug combination in treating the conditions or disorders described herein.

Suitable subjects in accordance with the methods described herein include, without limitation, a mammal, e.g., a human. In certain embodiments, the subject is an infant, a child, an adolescent, a young adult, an adult, or a geriatric adult. Additional suitable subjects include, but are not limited to, an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

The term “drug resistant” refers to the condition that occurs when cancer cells or microorganisms, such as bacteria or viruses, don't respond to a drug that is usually able to kill or weaken them. Drug resistance may be present before treatment is given or may occur during or after treatment with the drug. In cancer treatment, there may be many causes of resistance to anticancer drugs. For example, DNA changes or other genetic changes may change the way the drug enters cancer cells or the way the drug is broken down within cancer cells. Drug resistance can lead to cancer treatment being less effective or ineffective (e.g., disease progression while being treated with the cancer treatment) or can lead to cancer recurrence following treatment.

The terms “microorganisms” or “microbes” may refer to bacteria, viruses, fungi, parasites, protozoa, algae, slime molds, and the like. In some embodiments, the terms “microorganisms” or “microbes” refers to bacteria, viruses, fungi, and/or parasites.

As used herein, “active moiety” refers to reactive groups such as—SH and/or—NH and the compounds bearing these groups that make up part of the structure of the active metabolites of amifostine, phosphonol, and structurally-related compounds and analogs.

As used herein, “amifostine” refers to the name given to the phosphorothioate form of WR-1065 (also referred to as “WR1065”), WR-1065 being the biologically active moiety and physiological metabolite of amifostine.

As used herein, “aminothiol” refers to any molecule having the structure shown in FIG. 3.

As used herein, “prodrug” refers to an inactive drug derivative that is converted to an active form inside of cells and/or the body and preferably at the site of action.

As used herein, “aminothiol prodrug” refers to a therapeutically inactive prodrug that is composed in part of an aminothiol or aminothiol analog bonded to a conjugate molecule via a bioreducible disulfide bond. Under the appropriate conditions the disulfide bond is reduced, resulting in release of the aminothiol so that its therapeutic benefits can be realized. One example is the aminothiol-conjugate of formula (I) (see FIG. 11). Another example is that shown in FIG. 12. The aminothiol prodrugs of the present disclosure are sometimes referred to as “aminothiol-conjugates”, “BRG prodrugs”, or “BRG compounds”.

As used herein, “bioreducible” or “bioreducible disulfide bond” refers to a bond or disulfide bond that can be reduced by processes, enzymes, reactions, or other mechanisms that are present in vivo, in organ systems, and/or inside of cells.

As used herein, “conjugate” refers to any synthetic or naturally occurring polymer, copolymer, dendrimer, other conjugate, molecule, chemical or combination of the aforementioned that is bound to or conjugated to a therapeutically active aminothiol or aminothiol analog.

As used herein, “dendrimer” refers to any synthetic polymer with a branching, tree-like architecture.

As used herein, “PEG” is the abbreviated form of ‘polyethylene glycol’.

As used herein, “phosphonol” is the name given to the phosphorothioate WR-3789, with WR-255591 being the biologically active moiety and metabolite of phosphonol.

As used herein, “phosphorothioate” refers to the general name given to aminothiols that have a phosphate group bound to the sulfhydryl moiety.

As used herein, “polyethylene glycol” (also poly(ethylene glycol); polyethylene oxide) is the name given to molecules with the general structure of H—(O—CH₂—CH₂)ₙ—OH. PEG (see below) can have alternative groups, such as sulfhydryl moieties, which are not shown in this general formula (see also en.wikipedia.org/wiki/Polyethylene_glycol). Examples of such other alternative groups include —COOH, —OH, and NH₂.

As used herein, “4SP65” is the abbreviation used to designate the trifluoroactic acid salt of the aminothiol prodrug composed of WR-1065 conjugated by a disulfide bond to 4-arm star PEG, average molecular weight 10,000 Daltons (see, e.g., SigmaAldrich.com PEG Dendrimers and Multi-arm PEGs, which is hereby incorporated by reference in its entirety). As used herein, “WR-1065” is the name given to the active moiety of amifostine.

It is used here as representative of the active moieties of phosphorothioate drugs.

As used herein, “WR-2721” is a synonym for amifostine.

As described herein, metabolites of phosphorothioates include compounds described as aminothiols, tethered forms of the aminothiols, cysteamine, and cystamine. The aminothiols include, but are not limited to, the active metabolites of the phosphorothioates referred to as amifostine (WR-2721), phosphonol (WR-3689), WR-131527, structurally-related phosphorothioates, analogs of the aminothiols or phosphorothioates, their dephosphorylated active metabolites, and agents as described U.S. Pat. No. 6,489,312 to Stogniew, which is hereby incorporated by reference in its entirety.

The present disclosure also relates to methods for protecting the sulfhydryl moiety of these drugs during the delivery process. For example, the present disclosure relates to the use of polymers or copolymers composed entirely or in part of polyethylene glycol (PEG), other conjugates, or combinations thereof (referred to hence as ‘conjugates’). The molecular weight of these conjugates can vary as desired to optimize the drug formulation for a specific purpose, and the polymer can have any shape, including linear, multi-armed (star), or branching, tree-like (as in dendrimers) (Balogh, “Dendrimer 101” Adv. Exp. Med. Biol. 620:136-155 (2007); Mintzer et al., “Exploiting Dendrimer Multivalency to Combat Emerging and Re-Emerging Infectious Diseases,” Molecular Pharmaceutics 9:342-354 (2012), each of which is hereby incorporated by reference in its entirety), or can be of irregular shape. The conjugate also can be selected for its ability to interact with cell surface receptors and/or to enhance compound uptake by a cell-mediated active transport system. The conjugate is bound to the aminothiol through formation of a disulfide bond to the sulfhydryl moiety of the aminothiol. The disulfide bond is bioreducible in the presence of appropriate intracellular conditions, enzymes, reaction pathways, or combinations thereof.

A first aspect of the present disclosure relates to a method of treating a neoplastic condition in a subject in need thereof. This method involves administering to the subject a combination therapy comprising (i) an aminothiol-conjugate of Formula (I):

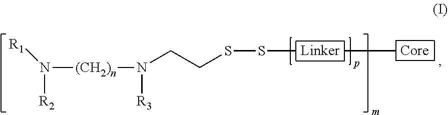

where

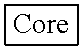

is optional and, if present, is an atom, a molecule, or a macromolecule;

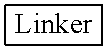

is a linker group, where the linker group is a polymer, a section of a polymer, an arm of a polymer, an arm of a copolymer, or a branch of a dendrimer;
where each

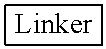

can be the same or different;
R₁, R₂, and R₃ are independently selected from hydrogen and C₁₋₆ alkyl;
m is 1 to 100,000;
n is 1 to 10; and
p is independently 0 to 2,500,
or a pharmaceutically acceptable salt, hydrate, polymorph, or solvate thereof, and
(ii) an anti-neoplastic drug, where the combination therapy is administered in an amount effective to treat the neoplastic condition in the subject.

A second aspect of the present disclosure relates to a combination therapeutic comprising:

(i) an aminothiol-conjugate of Formula (I):

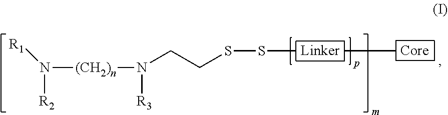

where

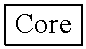

is optional and, if present, is an atom, a molecule, or a macromolecule;

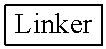

is a linker group, where the linker group is a polymer, a section of a polymer, an arm of a polymer, an arm of a copolymer, or a branch of a dendrimer;
where each

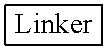

can be the same or different;
R₁, R₂, and R₃ are independently selected from hydrogen and C₁₋₆ alkyl;
m is 1 to 100,000;
n is 1 to 10; and
p is independently 0 to 2,500,
or a pharmaceutically acceptable salt, hydrate, polymorph, or solvate thereof, and
(ii) an anti-neoplastic drug.

The term “alkyl” means an aliphatic hydrocarbon group which may be straight or branched having about 1 to about 6 carbon atoms in the chain, (e.g., about 1 carbon atom, about 2 carbon atoms, about 3 carbon atoms, about 4 carbon atoms, about 5 carbon atoms, or about 6 carbon atoms). Branched means that one or more lower alkyl groups such as methyl, ethyl, or propyl are attached to a linear alkyl chain. Exemplary alkyl groups include methyl, ethyl, n-propyl, i-propyl, n-butyl, t-butyl, n-pentyl, and 3-pentyl.

In some embodiments of the methods and combination therapeutics of the present disclosure, p is independently selected from 0 to 2,500; from 0 to 1,000; from 0 to 500; from 0 to 250; 1 to 2,500; from 1 to 1,000; from 1 to 500; from 1 to 250; from 10 to 2,500; from 10 to 1,000; from 10 to 500; or from 10 to 250.

In certain embodiments of the methods and combination therapeutics of the present disclosure, the aminothiol-conjugate is that according to Formula (I), where m is 1, 2, 3, 4, 5, 6, 7, 8, 9, 10, 11, 12, 13, 14, or 15.

As mentioned above, each

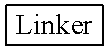

(also referred to herein as “Linker”) in the aminothiol-conjugate of Formula (I) can be the same or different. For example, where m of the aminothiol-conjugate according to Formula (I) is 3 (e.g., where the aminothiol-conjugate has 3 arms as described herein), each Linker of the three arms may be independently selected to form a symmetrical or asymmetrical molecule. For example, where m is 3, the three Linkers may each be a different Linker, the three Linkers may be the same Linker, or the three Linkers may include two Linkers that are the same and one that is different. Accordingly, in some embodiments of the methods and combination therapeutics of the present disclosure, each Linker in the aminothiol-conjugate of Formula (I) is the same. In other embodiments, each Linker in the aminothiol-conjugate of Formula (I) is different. In other embodiments, at least two Linker in the aminothiol-conjugate of Formula (I) are the same. In other embodiments, at least three Linker in the aminothiol-conjugate of Formula (I) are the same. In other embodiments, at least four Linker in the aminothiol-conjugate of Formula (I) are the same. In other embodiments, at least five Linker in the aminothiol-conjugate of Formula (I) are the same. In other embodiments, at least six Linker in the aminothiol-conjugate of Formula (I) are the same. In other embodiments, at least seven Linker in the aminothiol-conjugate of Formula (I) are the same. In other embodiments, at least eight Linker in the aminothiol-conjugate of Formula (I) are the same.

In some embodiments of the methods and combination therapeutics of the present disclosure, the molecular weight of the aminothiol-conjugate is 100,000 Daltons or less. The molecular weight of the aminothiol-conjugate may be about 100,000 Daltons; about 20,000 Daltons; about 10,000 Daltons; about 5,000 Daltons; about 3,000 Daltons; about 2,000 Daltons; about 1,000 Daltons; about 500 Daltons; or less than about 500 Daltons. In one embodiment, the molecular weight of the aminothiol-conjugate is about 10,000 Daltons. In certain embodiments, the molecular weight of the aminothiol-conjugate is about 9,000 to about 11,000 Daltons. In certain embodiments, the molecular weight of the aminothiol-conjugate is about 10,000 Daltons to about 11,000 Daltons. This includes, e.g., 4SP65, as described herein. In certain embodiments, the molecular weight of the aminothiol-conjugate is about 400 to about 500 Daltons. This includes, e.g., 1LP65, as described herein. In some embodiments of the methods and combination therapeutics of the present disclosure, the aminothiol-conjugate of Formula (I) has an average molecular weight in the range from about 0.25 kDa to about 100 kDa. In other embodiments, the aminothiol-conjugate of Formula (I) has an average molecular weight in the range from about 0.1 kDa to about 1 kDa.

In some embodiments of the methods and combination therapeutics of the present disclosure, the aminothiol-conjugate is present in a composition as a mixture of a two or more different aminothiol-conjugates of Formula (I).

In such embodiments of the methods and combination therapeutics of the present disclosure, the mixture of the two or more different aminothiol-conjugates of formula (I) is characterized by an average molecular weight. In some embodiments, each aminothiol-conjugate in the mixture of two or more different aminothiol-conjugates has an average molecular weight, independently, in the range of about 100 Da to about 10,000 Da; about 100 Daltons to about 9,000 Da; about 100 Da to about 8,000 Da; about 100 Da to about 7,000 Da; about 100 Da to about 6,000 Da; about 100 Da to about 5,000 Da; about 100 Da to about 4,000 Da; about 100 Da to about 3,000 Da; about 100 Da to about 2,000 Da; about 100 Da to about 1,000 Da; about 100 Da to about 900 Da; about 100 Da to about 800 Da; about 100 Da to about 700 Da; about 100 Da to about 600 Da; about 100 Da to about 500 Da; about 100 Da to about 400 Da; about 100 Da to about 300 Da; about 100 Da to about 200 Da; about 250 Da to about 100 kDa; about 250 Da to about 50 kDa; about 250 Da to about 10 kDa; about 250 Da to about 1 kDa; about 250 Da to about 900 Da; about 250 Da to about 800 Da; about 250 Da to about 700 Da; about 250 Da to about 600 Da; about 250 Da to about 500 Da; about 250 Da to about 400 Da; about 250 Da to about 300 Da; about 300 Da to about 100 kDa; about 400 Da to about 100 kDa; about 500 Da to about 100 kDa; about 600 Da to about 100 kDa; about 700 Da to about 100 kDa; about 800 Da to about 100 kDa; about 900 Da to about 100 kDa; about 1 kDa to about 90 kDa; about 1 kDa to about 80 Da; about 1 kDa to about 70 kDa; about 1 kDa to about 60 kDa; about 1 kDa to about 50 kDa; about 1 kDa to about 40 kDa; about 1 kDa to about 30 kDa; about 1 kDa to about 20 kDa; about 1 kDa to about 10 kDa; about 1 kDa to about 5 kDa; about 2 kDa to about 20 kDa; about 3 kDa to about 20 kDa; about 4 kDa to about 20 kDa; about 5 kDa to about 20 kDa; about 6 kDa to about 20 kDa; about 7 kDa to about 20 kDa; about 8 kDa to about 20 kDa; about 9 kDa to about 20 kDa; about 10 kDa to about 20 kDa; about 1 kDa to about 19 kDa; about 1 kDa to about 18 kDa; about 1 kDa to about 17 kDa; about 1 kDa to about 16 kDa; about 1 kDa to about 15 kDa; about 1 kDa to about 14 kDa; about 1 kDa to about 13 kDa; about 1 kDa to about 12 kDa; about 1 kDa to about 11 kDa; about 1 kDa to about 10 kDa; about 5 kDa to about 15 kDa; about 6 kDa to about 14 kDa; about 7 kDa to about 13 kDa; about 8 kDa to about 12 kDa; about 9 kDa to about 11 kDa; or any amount there between.

According to the present disclosure

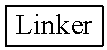

is a linker group, where the linker group is a polymer, a section of a polymer, an arm of a polymer, an arm of a copolymer, or a branch of a dendrimer, an atom, or a molecule. In certain embodiments the section of a polymer refers to a repeating unit of a polymer. In some embodiments, Linker is a polyethylene glycol (PEG) or a repeating unit of PEG.

Molecular mass, in the context of a water-soluble polymer such as PEG (or PEGylated compounds), refers to the nominal average molecular mass of a polymer, typically determined by size exclusion chromatography, light scattering techniques, or intrinsic viscosity determination in water or organic solvents. Molecular weight in the context of a polymer, such as PEG, can be expressed as either a number-average molecular weight (Mn) or a weight-average molecular weight (Mw). Unless otherwise indicated, all references to molecular weight herein refer to the number-average molecular weight. Both molecular weight determinations, number-average and weight-average, can be measured using gel permeation chromatographic techniques. Other methods for measuring molecular weight values can also be used, such as the use of end-group analysis or the measurement of colligative properties (e.g., freezing-point depression, boiling-point elevation, or osmotic pressure) to determine number-average molecular weight or the use of light scattering techniques, ultracentrifugation, or viscometry to determine weight-average molecular weight. The polymers described herein may or may not be polydisperse (i.e., number-average molecular weight and weight-average molecular weight of the polymers are not equal). Monodisperse polymers are uniform polymers in which all molecules have the same degree of polymerization or relative molecular mass. In some embodiments, the polymer may have a polydispersity index (PDI, a measure of the broadness of molecular weight distribution of a polymer) equal to 1. If polydisperse, the polymers may possess low polydispersity values such as less than about 1.2, less than about 1.15, less than about 1.10, less than about 1.05, less than about 1.04, less than about 1.03, less than about 1.02, and less than about 1.01. As used herein, references will at times be made to a single polymer having either a weight-average molecular weight or number-average molecular weight; such references will be understood to mean that the single polymer was obtained from a composition of polymers having the stated molecular weight.

Linker may be a moiety with a molecular weight of 100,000 Daltons or less; 20,000 Daltons or less; 10,000 Daltons or less; 5,000 Daltons or less; 3,000 Daltons or less; 2,000 Daltons or less; 1,000 Daltons or less; 500 Daltons or less; 400 Daltons or less; or 200 Daltons or less. Linker may be a moiety with a molecular weight of 200 Daltons to 100,000 Daltons; 200 Daltons to 20,000 Daltons; 200 Daltons to 10,000 Daltons; 200 Daltons to 5,000 Daltons; 200 Daltons to 3,000 Daltons; 200 Daltons to 2,000 Daltons; 200 Daltons to 1,000 Daltons; 200 Daltons to 500 Daltons; or 200 Daltons to 400 Daltons. Linker may be a moiety with a molecular weight of 400 Daltons to 100,000 Daltons; 400 Daltons to 20,000 Daltons; 400 Daltons to 10,000 Daltons; 400 Daltons to 5,000 Daltons; 400 Daltons to 3,000 Daltons; 400 Daltons to 2,000 Daltons; 400 Daltons to 1,000 Daltons; or 400 Daltons to 500 Daltons. Linker may be a moiety with a molecular weight of 500 Daltons to 100,000 Daltons; 500 Daltons to 20,000 Daltons; 500 Daltons to 10,000 Daltons; 500 Daltons to 5,000 Daltons; 500 Daltons to 3,000 Daltons; 500 Daltons to 2,000 Daltons; or 500 Daltons to 1,000 Daltons. Linker may be a moiety with a molecular weight of 50 Daltons to 100,000 Daltons; 50 Daltons to 20,000 Daltons; 50 Daltons to 10,000 Daltons; 50 Daltons to 5,000 Daltons; 50 Daltons to 3,000 Daltons; 50 Daltons to 2,000 Daltons; 50 Daltons to 1,000 Daltons; or 50 Daltons to 100 Daltons. Linker may be a moiety with a molecular weight of 1,000 Daltons to 100,000 Daltons; 1,000 Daltons to 20,000 Daltons; 1,000 Daltons to 10,000 Daltons; 1,000 Daltons to 5,000 Daltons; 1,000 Daltons to 3,000 Daltons; or 1,000 Daltons to 2,000 Daltons. Linker may be a moiety with a molecular weight of 2,000 Daltons to 100,000 Daltons; 2,000 Daltons to 20,000 Daltons; 2,000 Daltons to 10,000 Daltons; 2,000 Daltons to 5,000 Daltons; or 2,000 Daltons to 3,000 Daltons. Linker may be a moiety with a molecular weight of 3,000 Daltons to 100,000 Daltons; 3,000 Daltons to 20,000 Daltons; 3,000 Daltons to 10,000 Daltons; or 3,000 Daltons to 5,000 Daltons. Linker may be a moiety with a molecular weight of 5,000 Daltons to 100,000 Daltons; 5,000 Daltons to 20,000 Daltons; or 5,000 Daltons to 10,000 Daltons. Linker may be a moiety with a molecular weight of 10,000 Daltons to 100,000 Daltons; 10,000 Daltons to 20,000 Daltons. Linker may be a moiety with a molecular weight of 20,000 Daltons to 100,000 Daltons. Linker may be a moiety with a molecular weight of about 100,000 Daltons; 20,000 Daltons; 10,000 Daltons; 5,000 Daltons; 3,000 Daltons; 2,000 Daltons; 1,000 Daltons; 500 Daltons; 400 Daltons; or 200 Daltons.

In some embodiments of the methods and combination therapeutics of the present disclosure, Linker is a polymer, a section of a polymer, an arm of a polymer, or an arm of a co-polymer. Polymers as described herein include polyethylene glycol (PEG), branched PEG, polysialic acid (PSA), polysaccharides, pullulane, chitosan, hyaluronic acid, chondroitin sulfate, dermatan sulfate, starch, dextran, carboxymethyl-dextran, polyalkylene oxide (PAO), copolymers of polyalkylene oxides, polyoxamer (such as PLURONIC), polyalkylene glycol (PAG), polypropylene glycol (PPG), polyoxazoline, polyacryloylmorpholine, polyvinyl alcohol (PVA), polycarboxylate, polyvinylpyrrolidone, polyphosphazene, polyethylene-co-maleic acid anhydride, polystyrene-co-maleic acid anhydride, poly(l-hydroxymethylethylene hydroxymethylformal) (PHF), and 2-methacryloyloxy-2′-ethyltrimethylammonium phosphate (MPC), spermine polymer (Zhang and Vinogradov “Short Biodegradable Polyamine for Gene Delivery and Transfection of Brain Capillary Endothelial Cells,” J. Control Release 143:359-366 (2010), which is hereby incorporated by reference in its entirety), and other polymers.

In some embodiments of the methods and combination therapeutics of the present disclosure, Linker is polyethylene glycol (polyethylene oxide); thiol-terminated polyethylene glycol (polyethylene oxide); folic acid derivative; a conjugate of folic acid derivative with PEG; spermine; or a polymer of spermine.

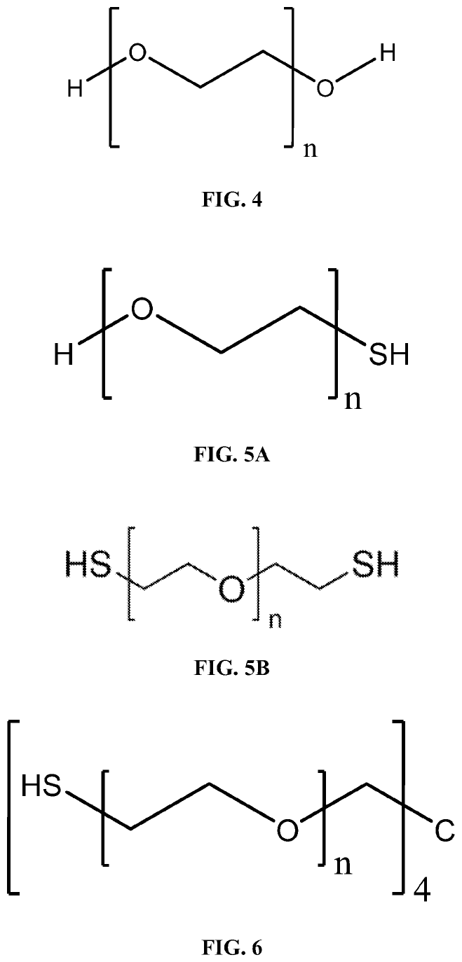

In some embodiments of the methods and combination therapeutics of the present disclosure, Linker is polyethylene glycol (polyethylene oxide) or a polyethylene glycol (polyethylene oxide) derivative (see, e.g., FIG. 4). In one embodiment, Linker is polyethylene glycol (polyethylene oxide), wherein ‘n’ can be any integer of 1 or greater. The linear formula is H—(O—CH₂—CH₂)ₙ—OH, where in ‘n’ can be any integer, with a range of 1 to 2500 being most desirable for the applications presented here. PEG may include a terminal end group. For example, PEG may terminate in a hydroxyl, a thiol, a methoxy or other alkoxyl group, a methyl or other alkyl group, an aryl group, a carboxylic acid, an amine, an amide, an acetyl group, a guanidino group, or an imidazole. Other contemplated end groups include azide, alkyne, maleimide, aldehyde, hydrazide, hydroxylamine, or alkoxyamine moieties.

A suitable Linker may also be thiol-terminated polyethylene glycol (polyethylene oxide) wherein ‘n’ can be independently any integer from 1 to 2500 (see exemplary structures shown in FIGS. 5A-5B). Exemplary suitable poly(ethylene glycol) dithiols are described at Sigmaaldrich.com, Homobifunctional PEGs, which is hereby incorporated by reference in its entirety. FIG. 4 shows a general structure of polyethylene glycol (polyethylene oxide), and FIGS. 5A-5B show the general structure of thiol-terminated polyethylene glycol (polyethylene oxide) wherein ‘n’ can be independently any integer from 1 to 2500.

In some embodiments of the methods and combination therapeutics of the present disclosure, the Linker is a polyethylene glycol (PEG) linker having an average molecular weight in the range of about 0.05 kDa to about 10 kDa, about 0.5 kDa to about 10 kDa, about 1 kDa to about 10 kDa, about 2 kDa to about 10 kDa, about 3 kDa to about 10 kDa, about 4 kDa to about 10 kDa, about 5 kDa to about 10 kDa, about 6 kDa to about 10 kDa, about 7 kDa to about 10 kDa, about 8 kDa to about 10 kDa, about 9 kDa to about 10 kDa, about 1 kDa to about 9 kDa, about 1 kDa to about 8 kDa, about 1 kDa to about 7 kDa, about 1 kDa to about 6 kDa, about 1 kDa to about 5 kDa, about 1 kDa to about 4 kDa, about 1 kDa to about 3 kDa, about 1 kDa to about 2 kDa, about 0.05 kDa to about 9 kDa, about 0.05 kDa to about 8 kDa, about 0.05 kDa to about 7 kDa, about 0.05 kDa to about 6 kDa, about 0.05 kDa to about 5 kDa, about 0.05 kDa to about 4 kDa, about 0.05 kDa to about 3 kDa, about 0.05 kDa to about 2 kDa, 0.05 to about 1 kDa, or any range there between. In some embodiments of the methods and combination therapeutics according to the present disclosure, the Linker is a PEG having an average molecular weight in the range from about 0.05 kDa to about 1 kDa.

In some embodiments of the methods and combination therapeutics of the present disclosure, the

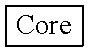

(also referred to herein as “Core”) is absent.

In other embodiments, the Core is a Linker.

In other embodiments, the Core is a carbon atom.

In further embodiments, the Core is —(O—CH₂—CH₂)ₙ—, where n is independently selected from 1 to 2500, from 1 to 1000, from 1 to 500, from 1 to 250, 1 to 100, from 1 to 50, from 1 to 25, from 1 to 10, from 1 to 5, from 10 to 2500, from 10 to 1000, from 10 to 500, or from 10 to 50.

Certain embodiments of the of the methods and combination therapeutics of the present disclosure relate to the aminothiol-conjugate of Formula (I), where

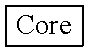

is optional and, if present, is a polymer core or

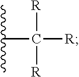

and R is independently selected from hydrogen and C₁₋₆ alkyl. In some embodiments, the aminothiol-conjugate has the formula of Formula (I), where

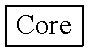

is a pentaerythritol core (e.g., that of 4SP65). In some embodiments, the Core is

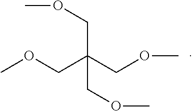

As noted above, Linker and/or Core may also be a conjugate of folic acid derivative with or without PEG. The general structure for folate (folic acid) is shown in FIG. 9. By altering the terminal carboxyl group to the appropriate moiety (e.g., SH) and then carrying out the addition of an aminothiol or PEG, respectively, a conjugate of folic acid with an aminothiol or PEG can be synthesized (Chen et al., “Folate-Mediated Intracellular Drug Delivery Increases the Anticancer Efficacy of Nanoparticulate Formulation of Arsenic Trioxide,” Mol. Cancer Ther. 8 (7): 1955-1963 (2009); Kang et al., “Folic Acid-Tethered Pep-1 Peptide-Conjugated Liposomal Nanocarrier for Enhanced Intracellular Drug Delivery to Cancer Cells: Conformational Characterization and in vitro Cellular Uptake Evaluation,” Int. J. Nanomed. 8:1155-1165 (2013), each of which is hereby incorporated by reference in its entirety). The folic acid conjugate offers the advantage that it can interact with the folic acid receptor on the surface of cells and trigger active transport of the prodrug into the cell cytosol.

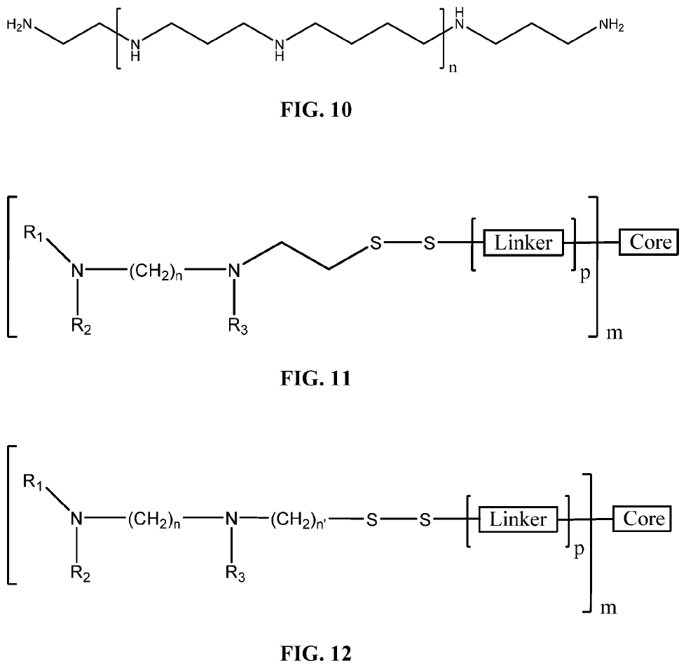

As noted above, Linker and/or Core may also be spermine or a polymer of spermine. By altering a terminal NH (2) group to the appropriate moiety (e.g., SH) and then carrying out the addition of an aminothiol or PEG, respectively, a conjugate of spermine polymer with an aminothiol or PEG can be synthesized. The general structure of spermine polymer is shown in FIG. 10. The linear formula is NH₂C₂H₄(NC₃H₆NHC₄H₈)ₙ—NHC₃H₆NH₂, wherein ‘n’ can be any integer equal to or greater than 1. The spermine polymer conjugate offers the advantage that it can interact with the polyamine receptor on the surface of cells and trigger active transport of the prodrug into the cell cytosol.

In certain embodiments of the methods and combination therapeutics of the present disclosure, Linker can be attached to the core to form thiol-terminated, polyethylene glycol (see, e.g., FIG. 6 and FIG. 8). In certain embodiments, Linker can be attached to the core to form one arm of a multi-armed thiol-terminated, polyethylene glycol (see, e.g., FIG. 6 and FIG. 8). Thus, in some embodiments of the methods and combination therapeutics of the present disclosure, the aminothiol-conjugate of Formula (I) is a multi-armed PEG-containing molecule. In accordance with such embodiments, the aminothiol-conjugate of Formula (I) may be an asymmetric multi-armed PEG-containing molecule (i.e., where at least one arm is different from the others) or a symmetric multi-armed PEG-containing molecule (i.e., where each arm is the same). In certain embodiments, the aminothiol-conjugate is a thiol-terminated, star polyethylene glycol having from 1 to 8 arms. For instance, Linker can be attached to the core to form thiol-terminated 1-arm-PEG, 2-arm-PEG, 3-arm-PEG, 4-arm-PEG, 5-arm-PEG, 6-arm-PEG, 7-arm-PEG, and 8-arm-PEG. See e.g., suitable PEG polymers and dendrimers at D'souza and Shegokar, “Polyethylene Glycol (PEG): A Versatile Polymer for Pharmaceutical Applications,” Expert Opin. Drug Deliv. 13 (9): 1257-1275 (2016); Parveen and Sahoo, “Long Circulating Chitosan/PEG Blended PLGA Nanoparticle for Tumor Drug Delivery,” Eur. J. Pharm. 670 (2-3): 372-383 (2011); Fairbanks et al., “Photoinitiated Polymerization of PEG-Diacrylate with Lithium Phenyl-2,4,6-Trimethylbenzoylphosphinate: Polymerization Rate and Cytocompatibility,” Biomaterials 35 (30): 6702-6707 (2009); www.sigmaaldrich.com/US/en/search/peg-sh?focus=products&page=1&perpage=30&sort=relevance&term=peg-sh&type=product; and www.sigmaaldrich.com/US/en/search/peg-dendrimers?focus=products&page=1&perpage=30&sort=relevance&term=peg % 20dendrimers& type=product, each of which is hereby incorporated by reference in its entirety.

As described herein above, the aminothiol-conjugate of the present disclosure may be a mixture of the two or more different aminothiol-conjugates of Formula (I). For example, compositions described herein may include a mixture of multi-armed thiol-terminated, polyethylene glycol aminothiol-conjugates described herein above, having from 1 to 8 arms. Isolation and purification of preparations of the aminothiol-conjugates and mixtures thereof described herein may be performed by usual purification methods, e.g., size exclusion chromatography.

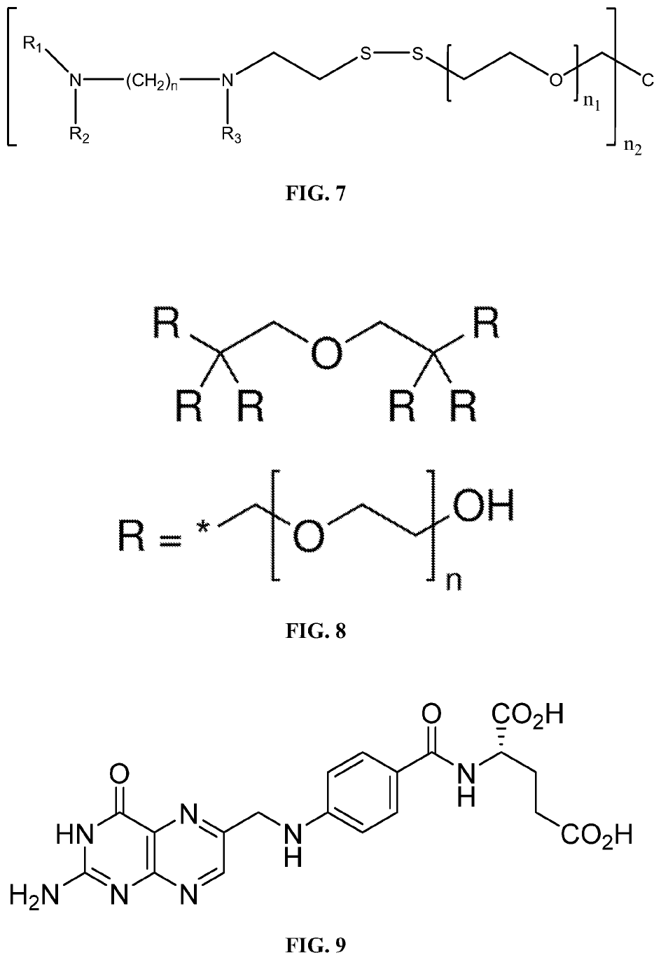

An exemplary structure of a thiol-terminated polyethylene glycol conjugated via a disulfide bond to an aminothiol is shown in FIG. 7, wherein ‘n₁’ can be any integer from 1 to 2500 and ‘n₂’ can be any number of arms that can be accommodated around a core without inducing undesirable steric hindrance or interference. In one embodiment, ‘n₁’ can be any integer from 1 to 2500 and ‘n₂’ can be any integer from 1 to 8. In certain embodiments, the thiol-terminated polyethylene glycol conjugated via a disulfide bond to an aminothiol is shown in FIG. 7, wherein ‘n₁’ can be any integer from 1 to 4 and ‘n₂’ can be any integer from 1 to 4. See, e.g., suitable PEG polymers and dendrimers at Sigmaaldrich.com, PEG Dendrimers and Multi-arm PEGs, which is hereby incorporated by reference in its entirety. In one embodiment, the aminothiol-conjugate is a 4-arm, thiol-terminated, star polyethylene glycol conjugated via a disulfide bond to an aminothiol. In this embodiment, the 4-arm, thiol-terminated, star polyethylene glycol conjugated via a disulfide bond to an aminothiol has the structure shown in FIG. 7, where ‘n₂’ is 4.

As described herein, the aminothiol portion of the aminothiol-conjugate of Formula (I) has the following formula:

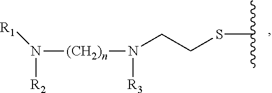

where each R₁, R₂, and R₃ is independently selected from hydrogen and C₁₋₆ alkyl, and where n is an integer of from 1 to 10. Aminothiols (and analogues thereof) that may be used to synthesize aminothiol-conjugates described herein include those of the exemplary generic structures shown in FIG. 3A and FIG. 3B. For instance, a generic structure of an aminothiol is:

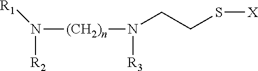

where X is selected from the group consisting of —PO₃H₂, hydrogen, sulfhydryl, sulfur, acetyl, isobutyryl, pivaloyl, and benzoyl; and each of R₁, R₂, and R₃ is independently selected from hydrogen and C₁₋₆ alkyl, and wherein n is an integer of from 1 to 10. Further, two exemplary structures of active moieties of the generic aminothiols shown in FIGS. 3A-3B that may be used to synthesize aminothiol-conjugates described herein are where X is hydrogen. In certain embodiments, the aminothiol is an active moiety of amifostine (NH₂(CH₂)₃NH(CH₂)₂SH) or phosphonol (CH₃NH(CH₂)₃NH(CH₂)₂SH).

In some embodiments of the methods and combination therapeutics of the present disclosure, the Core is a polymer core, a dendrimer core, a dendrimer core with an interior dendritic structure (i.e., branches), a therapeutic agent, or a derivative of a therapeutic agent.

Dendrimers have been extensively studied as vehicles for the delivery of therapeutics or as carriers for in vivo imaging (Lee et al., “Designing Dendrimers for Biological Applications,” Nat. Biotech. 23 (12): 1517-1526 (2005); Esfand & Tomalia, “Poly(amidoamine) (PAMAM) Dendrimers: From Biomimicry to Drug Delivery and Biomedical Applications,” Drug Discov. Today 6 (8): 427-36 (2001); Sadler & Tam, “Peptide Dendrimers: Applications and Synthesis,” Rev. Mol. Biotechnol. 90:195-229 (2002); Cloninger, “Biological Applications of Dendrimers,” Curr. Opin. Chem. Biol. 6:742-48 (2002); Niederhafner et al., “Peptide Dendrimers,” J. Peptide Sci. 11:757-88 (2005); Tekade et al., “Dendrimers in Oncology: An Expanding Horizon,” Chem. Rev. 109 (1): 49-87 (2009), each of which is hereby incorporated by reference in its entirety). Dendrimers are highly branched macromolecules with well-defined three-dimensional architectures (GEORGE R. NEWKOME ET AL., DENDRIMERS AND DENDRONS: CONCEPTS, SYNTHESIS, APPLICATIONS (2001), which is hereby incorporated by reference in its entirety). The appeal of dendrimers lies in their unique perfectly branched architectures, which affords them different properties than corresponding linear polymers of the same composition and molecular weights (Lee et al., “Designing Dendrimers for Biological Applications,” Nat. Biotech. 23 (12): 1517 26 (2005), which is hereby incorporated by reference in its entirety). As dendrimers increase in generation, they exponentially increase the number of termini, while only linearly increasing in radius; thus, the termini become more densely packed giving the entire structure a globular shape, where the termini radiate outwards from a central core. Various types of amide dendrimer cores have been described in the art. Suitable cores include those described in Tarallo et al., “Dendrimers Functionalized With Membrane-Interacting Peptides for Viral Inhibition,” Int'l J. Nanomed. 8:521-534 (2013); Carberry et al., “Dendrimer Functionalization With a Membrane-Interacting Domain of Herpes Simplex Virus Type 1: Towards Intracellular Delivery,” Chem. Eur. J. 18:13678-13685 (2012); Jung et al., “Synthesis of First- and Second-Generation Poly(amide)-Dendronized Polymers via Ring-Opening Metathesis Polymerization,” Macromolecules 44:9075-9083 (2011); Ornelas et al., “Strain-Promoted Alkyne Azide Cycloaddition for the Functionalization of Poly(amide)-Based Dendrons and Dendrimers,” J. Am. Chem. Soc. 132:3923-3931 (2010); Ornelas et al., “Construction of Well-Defined Multifunctional Dendrimers Using a Trifunctional Core,” Chem. Commun. 5710-5712 (2009); Goyal et al., “Enhanced Cooperativity in Hydrolytic Kinetic Resolution of Epoxides using Poly(styrene) Resin-Supported Dendronized Co-(Salen) Catalysts,” Adv. Synth. Catal. 350:1816-1822 (2008); and Yoon et al., “Monofunctionalization of Dendrimers with Use of Microwave-Assisted 1,3-Dipolar Cycloadditions,” Org. Lett. 9:2051-2054 (2007), each of which is hereby incorporated by reference in its entirety.

The use of any type of dendrimer is contemplated, including but not limited to poly(amidoamine) (PAMAM) dendrimers such as dense star polymers and Starburst polymers, poly(amidoamine-organosilicon) (PAMAMOS) dendrimers, (Poly(Propylene Imine)) (PPI) dendrimers, tecto dendrimers, multilingual dendrimers, chiral dendrimers, hybrid dendrimers/linear polymers, amphiphilic dendrimers, micellar dendrimers, and Fréchet-type dendrimers.

In some embodiments of the methods and combination therapeutics of the present disclosure, the molecular weight of the Core is 100,000 Daltons or less. For example, the Core may be 95,000 Daltons or less, 90,000 Daltons or less, 85,000 Daltons or less, 80,000 Daltons or less, 75,000 Daltons or less, 70,000 Daltons or less, 65,000 Daltons or less, 60,000 Daltons or less, 55,000 Daltons or less, 50,000 Daltons or less, 45,000 Daltons or less, 40,000 Daltons or less, 35,000 Daltons or less, 30,000 Daltons or less, 25,000 Daltons or less, 20,000 Daltons or less, 15,000 Daltons or less, 10,000 Daltons or less, 5,000 Daltons or less, 4,000 Daltons or less, 3,000 Daltons or less, 2,000 Daltons or less, 1,000 Daltons or less, 900 Daltons or less, 800 Daltons or less, 700 Daltons or less, 600 Daltons or less, 500 Daltons or less, 400 Daltons or less, 300 Daltons or less, 200 Daltons or less, or 100 Daltons or less.

In some embodiments of the methods and combination therapeutics of the present disclosure, the aminothiol-conjugate of Formula (I) has the following structure:

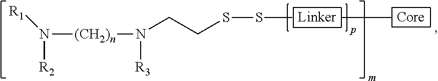

where

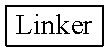

is polyethylene glycol (PEG) or a repeating unit of PEG; Core is optional and, if present, is a polymer core or

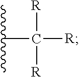

m is 1, 2, 3, 4, 5, 6, 7, or 8; R is independently selected from hydrogen and C₁₋₄ alkyl; R₁, R₂, and R₅ are H; n is 3; and each p is independently 1 to 2,500. In accordance with such embodiments, the

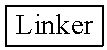

may be the same or different.

In some embodiments of the methods and combination therapeutics of the present disclosure, the aminothiol-conjugate of Formula (I) has the structure of:

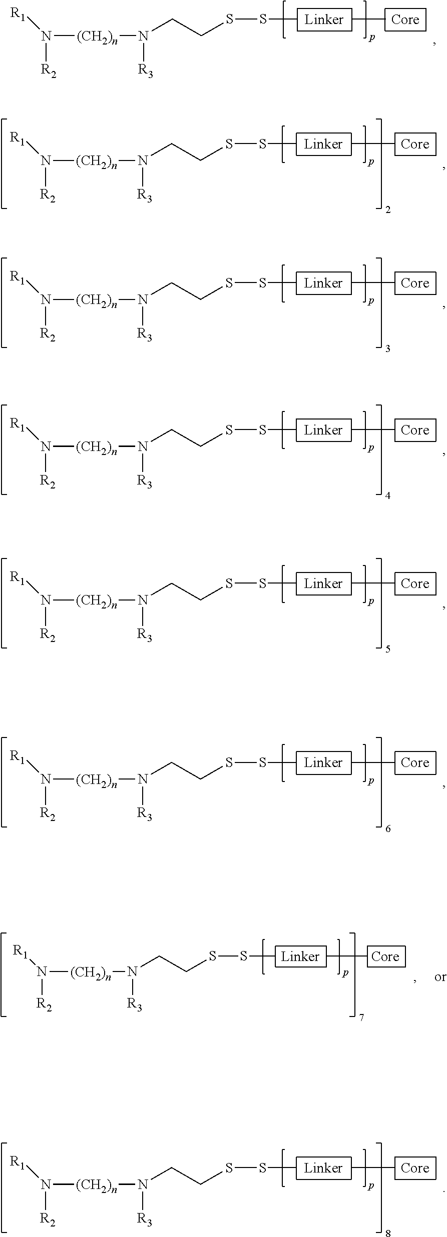

According to these embodiments, in each structure of the aminothiol-conjugate of Formula (I)

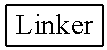

can be the same or different.

In some embodiments of the methods and combination therapeutics of the present disclosure, the aminothiol-conjugate of Formula (I) has the following structure:

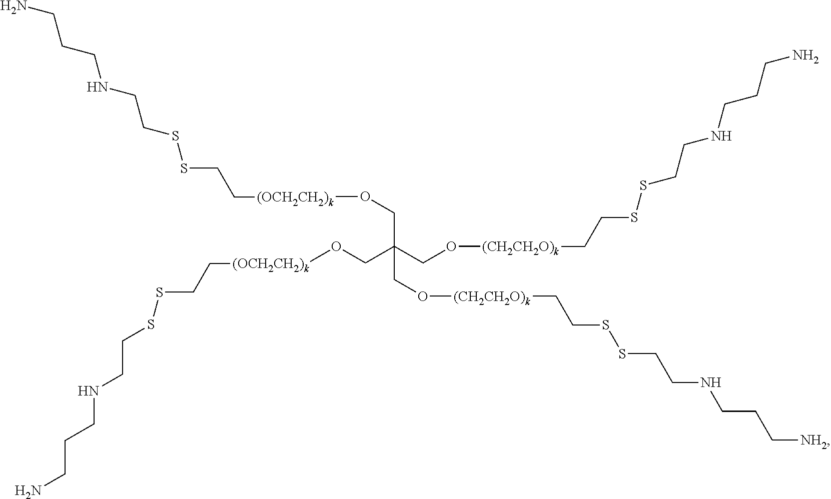

where k is independently 1 to 2,500, or a pharmaceutically acceptable salt thereof. Accordingly, k may be independently selected from 1 to 2,500; from 1 to 2,000; from 1 to 1,750; from 1 to 1,500; from 1 to 1,250; from 1 to 1,000; from 1 to 750; from 1 to 500; from 1 to 250; from 1 to 200; or from 1 to 150. In accordance with such embodiments, the aminothiol-conjugate of Formula (I) may be 4SP65.

In some embodiments of the methods and combination therapeutics of the present disclosure, the aminothiol-conjugate of Formula (I) has the following structure:

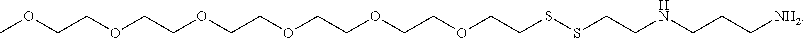 [SMILES: COCCOCCOCCOCCOCCOCCSSCCNCCCN]

In some embodiments of the methods and combination therapeutics of the present disclosure, the aminothiol-conjugate of Formula (I) has the following structure:

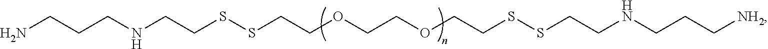

wherein n is independently 1 to 2,500. Accordingly, n may be independently selected from 1 to 2,500; from 1 to 2,000; from 1 to 1,750; from 1 to 1,500; from 1 to 1,250; from 1 to 1,000; from 1 to 750; from 1 to 500; from 1 to 250; from 1 to 200; or from 1 to 150.

In certain embodiments of the methods and combination therapeutics of the present disclosure, the aminothiol-conjugate according to the present disclosure is not a compound of Formula (II) or Formula (III) below:

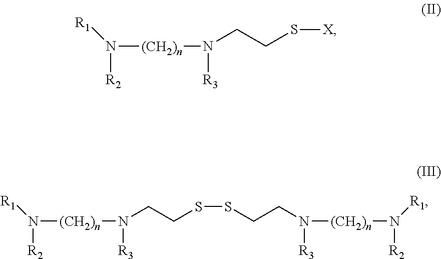

where X is selected from the group consisting of —PO₃H₂, hydrogen, acetyl, isobutyryl, pivaloyl, and benzoyl, where each of R₁, R₂, and R₃ is independently selected from the group consisting of hydrogen and C₁₋₆ alkyl, wherein n is an integer having a value of from 1 to 10. In one embodiment, the aminothiol-conjugate according to the present disclosure is not amifostine.

In certain embodiments of the methods and combination therapeutics of the present disclosure, the aminothiol-conjugate according to the present disclosure is not a compound of Formula (II) or Formula (III), where X is an intracellularly-cleavable protecting group selected from the group consisting of a peptide, a sulfur-containing amino acid, glutathione, a sulfur-containing antioxidant, an oxygen-containing antioxidant, a photoreversible thiol tag, and (R)-tert-butyl-2-[(tert-butoxycarbonyl)amino]-3-(tryitylsulfanyl) propanoate.

In certain embodiments of the methods and combination therapeutics of the present disclosure, the aminothiol-conjugate according to the present disclosure is not a compound of Formula (II) or Formula (III), where X is the thiol-protected form of the aminothiol selected from the group consisting of a homodimer of the aminothiol, a heterodimer of the aminothiol and a different aminothiol, and cysteamine.

Improved sulfhydryl protecting groups combined with intracellular drug delivery system(s) for the aminothiols, their metabolites, and/or their analogs to cells where therapeutic effects are desired should meet three conditions. First, the protecting group should have the capacity to prevent adventitious reactivity of the aminothiols during drug delivery; second, the protecting group should be removable by systems or processes available in target cells and particularly within the intracellular milieu and/or within lysosomes; and third, the protecting group should be non-toxic to animal and human cells. Other desirable conditions that can be met include (i) increasing drug circulation times, (ii) making the drug amenable to cell absorption via mechanisms that are not applicable to aminothiols alone, (iii) making the drug amenable to intracellular uptake by cell receptor transport systems (the folic acid and polyamine transport systems are two examples), (iv) altering the mechanisms by which the drug is cleared from circulation and/or the human or animal body, increasing the stability of the disulfide bond in circulation and/or optimizing its reduction intracellularly. This may require adding groups reported to stabilize the disulfide bond in circulation, such as methyl groups or ring structures (Brulisauer et al., “Disulfide-Containing Parenteral Delivery Systems and their Redox-Biological Fate,” J. Control Release 195:147-154 (2014), which is hereby incorporated by reference in its entirety). Other stabilizing modifications have been reported (Guo et al., “Advances in Redox Sensitive Drug Delivery Systems of Tumor Microenvironment,” J. Nanobiotechnology 16 (1): 74 2018) and Wang et al., “Disulfide Based Prodrugs for Cancer Therapy,” RSC Adv. 10:234397 (2020), which are hereby incorporated by reference in their entirety).

The aminothiols and their analogs react readily with a subset of proteins and nucleic acids, and thus, the active moieties need to be released at or near the sites where reactivity is desired to achieve a therapeutic effect of the drug. As thiols, aminothiols participate in the systems that maintain the redox balance of cells (Nagy, P., “Kinetics and Mechanisms of Thiol-Disulfide Exchange Covering Direct Substitution and Thiol Oxidation-Mediated Pathways,” Antioxid Redox Signal 18 (13): 1623-1641 (2013), which is hereby incorporated by reference in its entirety). WR1065 also is a reductant that can contribute significant reducing equivalents to the cell cytosol. The PEG-SH scaffold is predicted to be a nucleophile, and thus, it also can participate in nucleophile-electrophile interactions. Since these therapeutic effects occur intracellularly as opposed to extracellularly, intracellular delivery represents the optimal delivery site. Intracellular delivery will optimize opportunities for reactivity of the active drug metabolite(s) with target cellular elements as opposed to reaction with targets that are not associated with therapeutic effects, including but not limited to extracellular targets.

Conjugation of a therapeutic aminothiol to another molecule for the purpose of altering the pharmacokinetics and pharmacodynamics from that of the corresponding phosphorothioate is a method that can be used to alter or enhance aminothiol delivery to, and activation in, stressed or diseased cells. Polymers or copolymers, including dendrimers, composed entirely or partially of PEG or comparable biocompatible materials designed to alter and improve drug pharmacokinetics and pharmacodynamics and that also are amenable to cell uptake and intracellular delivery of the aminothiols can be used to meet these goals. Methods are presented below for resolving these problems by using drug formulations that consist of an aminothiol moiety bound to a conjugate as described herein. Such formulations can be used alone or can be combined with additional methods to achieve optimal intracytoplasmic drug delivery and drug efficacy.

Intracellular delivery methods and compositions have been developed by others for effecting intracellular delivery of other drug molecules. Some of those methods and compositions (e.g., those explicitly described or referenced herein) can be used to effect intracellular delivery of aminothiols. However, it is believed that no others have previously proposed to use such compositions and methods in connection with aminothiols. Thus, compositions and methods that have been described by others for protecting the sulfhydryl group of an active pharmaceutical entity can be used to facilitate intracellular delivery of aminothiol compounds, even if those compositions and methods are not among those explicitly described in this disclosure.

Amifostine, as representative of the class of drugs known as phosphorothioates, is an inactive prodrug composed of the therapeutically active aminothiol WR1065 and a phosphate group that is conjugated to the aminothiol via a bond to the aminothiol's sulfhydryl group. This prodrug has specific pharmacokinetic and pharmacodynamics characteristics that make it suitable for delivery to, and activation by, many but not all normal cells (e.g., activated and resting T lymphocytes and resting B lymphocytes) of humans and other animals. However, these characteristics are not suitable for prodrug delivery to, and activation by, most stressed or diseased cells. Thus, in order to realize the therapeutic benefits of the aminothiol, new prodrugs that contain and can release the aminothiol under physiologic conditions of stress and/or disease, and to cells that are stressed or diseased, are needed.

In the following discussion, the terms ‘amifostine’ and ‘WR-1065’ (the active moiety of amifostine) will be used as representative examples of all phosphorothioates, aminothiols, their analogs, and the active metabolites of the parent drugs (prodrugs).

Amifostine is a phosphorothioate that is metabolized in vivo to its active moiety WR-1065 (Grdina et al., “Thiol and Disulfide Metabolites of the Radiation Protector and Potential Chemopreventive Agent WR-2721 are Linked to Both its Anti-Cytotoxic and Anti-Mutagenic Mechanisms of Action,” Carcinogenesis 16:767 774 (1995); Purdie et al., “Interaction of Cultured Mammalian Cells with WR-2721 and its Thiol, WR-1065: Implications for Mechanisms of Radioprotection,” Int. J. Radiat. Biol. Relat. Stud. Phys. Chem. Med. 43:517-527 (1983); Shaw et al., “Pharmacokinetic Profile of Amifostine,” Semin. Oncol. 23:18-22 (1996), each of which is hereby incorporated by reference in its entirety). The sulfhydryl moiety of WR1065 is involved in its therapeutic effects (Grdina et al., “Amifostine: Mechanisms of Action Underlying Cytoprotection and Chemoprevention,” Drug Metabol. Drug Interact. 16:237-279 (2000); Grdina et al., “Differential Activation of Nuclear Transcription Factor Kappab, Gene Expression, and Proteins by Amifostine's Free Thiol in Human Microvascular Endothelial and Glioma Cells,” Semin. Radiat. Oncol. 12:103-111 (2002); Grdina et al., “Relationships Between Cytoprotection and Mutation Prevention by WR-1065,” Mil Med 167:51-53 (2002); Grdina et al. “Radioprotectors: Current Status and New Directions,” Radiat. Res. 163:704-705 (2002), each of which is hereby incorporated by reference in its entirety), and thus, this moiety requires protection from adventitious reactivity during drug delivery and until the drug is metabolized at the plasma membrane, and this protection in the case of amifostine, is provided by the phosphate group. The phosphate group is removed when the drug is brought into close proximity to cell plasma membranes and/or the drug is taken up into the plasma membrane. The dephosphorylation step is carried out by membrane-bound alkaline phosphatase, an enzyme that is produced by many, but not all human and animal cells. After removal of the phosphate group, the active moiety is taken up into the intracellular milieu from which it can be distributed further to subcellular organelles or to other cells, and where therapeutic effects are induced. Cellular uptake of many, but not all forms of the aminothiols occurs by passive diffusion, but some drug forms are taken up by active transport through a plasma membrane-associated transport system, and active transport of other drug forms may occur at some drug concentrations but not others (Grdina et al., “Differential Activation of Nuclear Transcription Factor Kappab, Gene Expression, and Proteins By Amifostine's Free Thiol in Human Microvascular Endothelial and Glioma Cells,” Semin. Radiat. Oncol. 12:103-111 (2002); Grdina et al., “Relationships between Cytoprotection and Mutation Prevention by WR-1065,” Mil Med 167:51-53 (2002); Grdina et al., “Radioprotectors: Current Status and New Directions,” Radiat. Res. 163:704-705 (2002), each of which is hereby incorporated by reference in its entirety). For cells that cannot take up the drug and/or cannot metabolize the drug, the active form can be delivered to these cells via cell- and tissue-distribution processes. Previously known methods for administering phosphorothioates to a human or animal include, but are not limited to, oral delivery, intraperitoneal injection, subcutaneous injection, intravenous injection, inhalation, incorporation into nanoparticles (Pamujula et al., “Oral Delivery of Spray Dried PLGA/Amifostine Nanoparticles,” J. Pharm. Pharmacol. 56:1119-1125 (2004); Pamujula et al., “Preparation and In Vitro Characterization of Amifostine Biodegradable Microcapsules,” Eur. J. Pharm. Biopharm. 57:213-218 (2004); Pamujula et al., “Radioprotection in Mice Following Oral Delivery of Amifostine Nanoparticles,” Int. J. Radiat. Biol. 81:251-257 (2005), each of which is hereby incorporated by reference in its entirety), or using other drug delivery systems (Gu et al., “Tailoring Nanocarriers for Intracellular Protein Delivery,” Chem. Soc. Rev. 40:3638-3655 (2011); Hoffman et al., “The Origins and Evolution of “Controlled” Drug Delivery Systems,” J. of Controlled Release 132:153-163 (2008); Imbuluzqueta et al., “Novel Bioactive Hydrophobic Gentamicin Carriers for the Treatment of Intracellular Bacterial Infections,” Acta. Biomater. 7:1599-1608 (2011); Leucuta et al., “Systemic and Biophase Bioavailability and Pharmacokinetics of Nanoparticulate Drug Delivery Systems,” Curr. Drug Del. 10:208-240 (2013); Patel et al., “Recent Developments in Protein and Peptide Parenteral Delivery Approaches,” Ther. Delivery 5:337-365 (2014); Patel et al., “Particle Engineering to Enhance or Lessen Particle Uptake by Alveolar Macrophages and to Influence the Therapeutic Outcome,” Eur. J. Pharm. Biopharm. 89:163-174 (2015); Sakagami, “Systemic Delivery of Biotherapeutics through the Lung: Opportunities and Challenges for Improved Lung Absorption,” Ther. Del. 4:1511-1525 (2013); Torchilin, “Recent Approaches to Intracellular Delivery of Drugs and DNA and Organelle Targeting,” Ann. Rev. Biomed. Eng. 8:343-375 (2006), each of which is hereby incorporated by reference in its entirety).

Amifostine is inactive until metabolized by cell membrane-bound alkaline phosphatase, which removes the phosphate group, thereby, releasing WR1065 with its free thiol for uptake into cells (Capizzi, “The Preclinical Basis for Broad-Spectrum Selective Cytoprotection of Normal Tissues from Cytotoxic Therapies by Amifostine (Ethyol),” Eur. J. Cancer 32A: Suppl 4: S5-16 (1996); Shaw et al., “Pharmacokinetic Profile of Amifostine,” Semin. Oncol. 23:18-22 (1996); Yu et al., “The Radioprotective Agent, Amifostine, Suppresses the Reactivity of Intralysosomal Iron,” Redox Report: Communications in Free Radical Research 8:347-355 (2003), each of which is hereby incorporated by reference in its entirety). Amifostine has little to no activity in diseased or stressed cells because many diseased cells, including pathogen-infected cells, tumor cells, and cells in the microenvironment of metastatic cells, produce little to no membrane-bound enzyme but can and often do produce significant amounts of various alkaline phosphatase isoenzymes (Guerreiro et al., “Distinct Modulation of Alkaline Phosphatase Isoenzymes by 17beta-Estradiol and Xanthohumol in Breast Cancer MCF-7 Cells,” Clin. Biochem. 40:268-273 (2007); Kato et al., “Effect of Hyperosmolality on Alkaline Phosphatase and Stress-Response Protein 27 of MCF-7 Breast Cancer Cells,” Breast Cancer Res Treat. 23:241-249 (1992); Van Hoof et al., “Interpretation and Clinical Significance of Alkaline Phosphatase Isoenzyme Patterns,” Crit. Rev. in Clin. Lab. Sci. 31:197-293 (1994); Walach et al., “Leukocyte Alkaline Phosphatase, CA15-3, CA125, and CEA in Cancer Patients,” Tumori 84:360-363, each of which is hereby incorporated by reference in its entirety) that are released into the extracellular milieu or circulation so that amifostine bioactivation is remote to target cells. Plasma-membrane bound alkaline phosphatase is a GPI-anchored protein (Marty et al., “Effect of Anti-Alkaline Phosphatase Monoclonal Antibody on B Lymphocyte Function,” Immunol. Lett. 38:87-95 (1993), which is hereby incorporated by reference in its entirety) that is expressed by some, but not all, cell types. Defects in GPI-anchor synthesis can result from mutations or epigenetic alterations in key genes essential for GPI-anchor synthesis and high rates of mutation induction and epigenetic alterations are common in cancer and have been reported to occur in critical GPI-anchor synthesis genes (Dobo et al., “Defining EMS and ENU Dose-Response Relationships using the Pig-a Mutation Assay in Rats,” Mutat. Res. 725:13-21 (2011); Dobrovolsky et al., “Detection of In Vivo Mutation in the Hprt and Pig-a Genes of Rat Lymphocytes,” Methods Mol. Biol. 1044:79-95 (2013), each of which is hereby incorporated by reference in its entirety). Alkaline phosphatase also is present intracellularly in the rough endoplasmic reticulum where it is synthesized, in the Golgi apparatus where additional processing may occur, in Golgi-derived vesicles, in some lysosomes, and around the nuclear envelope (Tokumitsu et al., “Alkaline Phosphatase Biosynthesis in the Endoplasmic Reticulum and its Transport Through the Golgi Apparatus to the Plasma Membrane: Cytochemical Evidence,” J. Histochem. Cytochem. 31:647-655 (1983), which is hereby incorporated by reference in its entirety). Its localization varies with cell cycle in activated B lymphocytes (Souvannavong et al., “Expression and Visualization During Cell Cycle Progression of Alkaline Phosphatase in B Lymphocytes from C3H/HeJ Mice,” J. Leukocyte Biol. 55:626-632 (1994), which is hereby incorporated by reference in its entirety), with synthesis occurring around the mitotic phase of the cell cycle (Tokumitsu et al., “Immunocytochemical Demonstration of Intracytoplasmic Alkaline Phosphatase in HeLa TCRC-1 Cells,” J. Histochem. Cytochem. 29:1080-1087 (1981), which is hereby incorporated by reference in its entirety). Plasma membrane-bound alkaline phosphatase is dependent upon correct microtubule organization to achieve its correct orientation in the cell membrane (Gilbert et al., “Microtubular Organization and its Involvement in the Biogenetic Pathways of Plasma Membrane Proteins in Caco-2 Intestinal Epithelial Cells,” J. Cell. Biol. 113:275-288 (1991), which is hereby incorporated by reference in its entirety), and microtubule organization can be altered in cancer cells and cells infected with viruses (Nyce, “Drug-Induced DNA Hypermethylation and Drug Resistance in Human Tumors,” Cancer Res. 49:5829-5836 (1989); Oshimura et al., “Chemically Induced Aneuploidy in Mammalian Cells: Mechanisms and Biological Significance in Cancer,” Environ. Mutagen. 8:129-159 (1986), which is hereby incorporated by reference in its entirety).

Alkaline phosphatase localization and expression is not uniform across all cell types or across all cell states or conditions, but instead is highly variable. Some cells to which drug delivery is desired do not produce membrane-bound alkaline phosphatase, or produce it only under limited conditions, or only produce it during developmental stages that are of limited duration. In some disease states, such as during inflammation, infection, or neoplastic transformation, membrane-bound alkaline phosphatase expression and localization are altered. Alkaline phosphatase is released into the extracellular milieu during some infectious conditions as a generalized response to pathogens (Murthy et al., “Alkaline Phosphatase Band-10 Fraction as a Possible Surrogate Marker for Human Immunodeficiency Virus Type 1 Infection in Children,” Arch. Path. & Lab. Med. 118:873-877 (1994), which is hereby incorporated by reference in its entirety). Activated B lymphocytes can shed alkaline phosphatase into the surrounding cellular milieu (Burg et al., “Late Events in B Cell Activation. Expression Of Membrane Alkaline Phosphatase Activity,” J. Immunol. 142:381-387 (1989), which is hereby incorporated by reference in its entirety) and alkaline phosphatase also is present in serum. Alkaline phosphatase is not expressed in quiescent B lymphocytes; it also is not expressed in active and inactive T-lymphocytes. Release of alkaline phosphatase into the extracellular milieu can result in metabolism of phosphorothioates to their active metabolites at a distance from cell membranes. This phenomenon reduces uptake by cells, increases the availability of metabolites for participation in non-therapeutic reactions, and makes the active moieties available for further metabolism to aldehydes and other compounds with cytotoxic effects.

The active form of amifostine (WR-1065) must be present inside of cells for beneficial effects to be observed. WR-2721 (amifostine), WR-1065, WR-33278, WR-1065-cysteine, and other disulfide forms of the parent compound WR-2721 did not show evidence of activity if present outside of V79 cells (Smoluk et al., “Radioprotection of Cells in Culture by WR-2721 and Derivatives: Form of the Drug Responsible for Protection,” Cancer Res. 48:3641-3647 (1988), which is hereby incorporated by reference in its entirety). In contrast, intracellular levels of WR-1065 correlated with significant protection against gamma-radiation. Results were similar for Hela cells, me-180 cells, Ovary 2008 cells, HT-29/SP-1d cells, and Colo 395 tumor cell lines (Smoluk et al., “Radioprotection of Cells in Culture by WR-2721 and Derivatives: Form of the Drug Responsible for Protection,” Cancer Res. 48:3641-3647 (1988), which is hereby incorporated by reference in its entirety). For optimal cytoprotection, sufficient and sustained intracellular levels of WR-1065, the active form of amifostine, were necessary (Souid et al., “Determination of the Cytoprotective Agent WR-2721 (Amifostine, Ethyol) and its Metabolites in Human Blood using Monobromobimane Fluorescent Labeling and High-Performance Liquid Chromatography,” Cancer Chemother. Pharmacol. 42:400-406 (1998), which is hereby incorporated by reference in its entirety). If the cells were transferred to drug-free medium for 4 hours before exposure to radiation, the intracellular levels of WR-1065 and WR-33278 decreased markedly along with cytoprotection from radiation damage (Grdina et al., “Thiol and Disulfide Metabolites of the Radiation Protector and Potential Chemopreventive Agent WR-2721 are Linked to Both its Anti-Cytotoxic and Anti-Mutagenic Mechanisms of Action,” Carcinogenesis 16:767-774 (1995), which is hereby incorporated by reference in its entirety). In vivo tissue levels of WR-1065 were similar in monkeys and in humans and tissue levels of drug were informative for cytoprotective effects (Cassatt et al., “Preclinical Modeling of Improved Amifostine (Ethyol) use in Radiation Therapy,” Semin. Radiat. Oncol. 12:97-102 (2002); Shaw et al., “Metabolic pathways of WR-2721 (ethyol, amifostine) in the BALB/c mouse,” Drug Metab Dispos. 22:895-902 (1994), each of which is hereby incorporated by reference in its entirety).

In summary, reliance upon the drug formulations known as the phosphorothioates for delivery of their therapeutically active metabolites, the aminothiols, is associated with several significant problems including (1) inability to metabolize the drug to its active form by some cell types, including but not limited to stressed or diseased cells, (2) inability to activate/metabolize the drug under some physiological or disease conditions, (3) activation of the drug in milieus where its activity is not desired, (4) activation of the drug at a distance from the optimal cellular or subcellular milieu, (5) activation in milieus where the products are vulnerable to metabolism to toxins, (6) lack of ability to achieve targeted cell delivery or targeted cell exclusion, and (7) lack of adequate drug uptake into tumor cells because of the relative acidic milieu of tumor tissue compared to the neutral pH of normal tissue (Santini et al., “The Potential of Amifostine: from Cytoprotectant to Therapeutic Agent,” Haematologica 84:1035-1042 (1999) and Schuchter, “Guidelines for the Administration of Amifostine,” Semin Oncol 23:40-43 (1996), each of which is hereby incorporated by reference in its entirety). These problems adversely affect the ability to obtain a therapeutic effect in stressed or diseased cells.

Taken together, these findings support the conclusion that reliance upon a phosphate group for protection of the sulfhydryl moiety of an aminothiol during delivery, and reliance upon alkaline phosphatase for metabolism of the parent drug to its active moiety have significant disadvantages that can affect drug efficacy adversely. The above considerations demonstrate the need for new drug formulations. Methods for achieving these results are described herein.

Three criteria should be satisfied to address the above described problems. Sulfhydryl groups are highly reactive moieties that will form covalent bonds with a variety of moieties present in the bodies and cells of living organisms. Thus, therapeutic drugs that contain one or more sulfhydryl groups that have roles in the pharmacological effects of those drugs require protection of the sulfhydryl moiety during delivery to prevent reactivity with neighboring molecules not related to the drug's desired therapeutic effects. To achieve this protection, any molecular group can be used if it meets the requirements that (i) it achieves the desired protective effect during delivery, (ii) it is amenable to cellular uptake into the cytosol, (iii) it can be removed intracellularly, (iv) it is not toxic to cells (either before or after removal from the active aminothiol moiety), and (v) it achieves intracellular therapeutic aminothiol levels in a time frame that can be achieved given the half-life of the prodrug in circulation (i.e. within an acceptable time frame).

Any method that achieves intracellular drug delivery at therapeutic intracellular levels within an acceptable time frame, including but not limited to delivery into intracellular organelles, will serve the purpose of delivering aminothiol drugs to a milieu where their activity is desired and where they will have a beneficial effect. That is, the observations made in this disclosure relate importantly to realization that intracellular delivery of an intracellularly-cleavable aminothiol-protecting-moiety conjugate beneficially affects administration of aminothiols. The observations made in this disclosure also relate to realization that intracellular delivery-however achieved-of an aminothiol compound having a reactive active moiety is advantageous relative to extracellular delivery of the corresponding phosphorothioate of the aminothiol compound.

Targeted cell delivery and/or targeted cell exclusion is desirable because of the recognized toxicity of aminothiols. For delivery by certain methods, such as oral delivery or inhalation delivery, the delivery method or system should be one that has the capacity to protect the drug from degradation by, and/or reactivity with, enzymes found in the lumen of organs through which the drug will pass. Thus, for oral delivery the methods must achieve protection from luminal enzymes and factors of the gastrointestinal tract, and for inhalation delivery, the methods must protect against degradation by respiratory tract exudates/secretions.

Targeted drug delivery can be either passive or active (Banerjee et al., “Poly(ethylene glycol)-Prodrug Conjugates: Concept, Design, and Applications,” J. Drug Deliv. 2012:1-17 (2012), which is hereby incorporated by reference in its entirety). The enhanced permeability and retention (EPR) effect achieves passive drug targeting by releasing, or causing the accumulation of, drug outside the target site, and it relies upon altered environmental conditions. The EPR effect takes advantage of the hyperpermeable vasculature and reduced lymphatic drainage of tumors and inflamed areas to increase drug accumulation in these areas, thereby, providing passive targeting. Active targeting is based upon taking advantage of potential interactions between a ligand-receptor, antigen-antibody, enzyme substrate (biological pairs). Targeting agents are attached to the surface of the prodrug by conjugation chemistries. Examples of common targeting moieties include peptide ligands, sugar residues, antibodies, or aptamers that have as their biological pair receptors, selectins, antigens, or mR.VAs expressed by cells or organs. For example, luteinizing hormone-releasing hormone peptide is used to target receptors overexpressed by several cancer cells. Added groups can be ones that serve as ligands for receptors and/or that trigger receptor-mediated endocytosis.

Finally, to achieve drug activation, any group used to protect the sulfhydryl group of the aminothiol must be one that can be released or removed once the drug has been successfully delivered into the cytoplasm of target and/or non-target cells.

As described herein, the active form of the drug is protected during delivery and it is desirable to obtain release of the aminothiol once delivery has been completed. In general, any compositions or method(s) that provide protection of the sulfhydryl group of the aminothiols during delivery, that result in intracellular release of the active form of the drug following delivery to the desired site(s), and that result in therapeutic intracellular drug levels can be used. Protection of the sulfhydryl moiety of the aminothiols prior to intracellular delivery is essential for obtaining therapeutic benefits of these drugs. Because protection systems should have the characteristic of being able to release the active moiety of the drug once intracytoplasmic delivery has been achieved, systems that address both protection during delivery and release after delivery are discussed together.

For the conjugates described herein, common characteristics include the following. The aminothiol is bound to the conjugate via a bioreducible disulfide bond between the sulfhydryl group of the aminothiol and a sulfhydryl group on the conjugate, or at the end of one or more arms, for multi-arm polymers/copolymers, or at the end of one or more branches, for branching dendrimers. The disulfide bonds are reducible by oxidation-reduction and thiol-disulfide exchange reactions that function primarily in the cytosol and in the cytosolic conditions of target cells, but not extracellularly or in circulation conditions (Navath et al., “Stimuli-Responsive Star Poly(Ethylene Glycol) Drug Conjugates for Improved Intracellular Delivery of the Drug in Neuroinflammation,” J. Controlled Release 142:447-456 (2010) and Brulisauer et al., “Disulfide-Containing Parenteral Delivery Systems and their Redox-Biological Fate,” J. Control Release 195:147-154 (2014), which are hereby incorporated by reference in their entirety). Bonds to sulfhydryl groups that link the aminothiol to the conjugate and that are reducible by cellular processes, reactions, enzymes, or other elements can be used. Reduction of the disulfide bond or other linking bonds results in the release of the aminothiol so that its therapeutic effects can be realized. The conjugate can have a linear, branched or dendrimeric architecture and the molecular weight of the conjugate can vary from low to high, based upon the number of repeating units in the polymer/copolymer and/or the number of branches and repeating units in the dendrimer. Conjugates may or may not have biologic activity.

Conjugates that meet these conditions include the following:
(i) A conjugate that is composed, entirely or in part, of polyethylene oxide (PEG) (Bondar et al., “Lipid-Like Trifunctional Block Copolymers of Ethylene Oxide and Propylene Oxide: Effective and Cytocompatible Modulators of Intracellular Drug Delivery.” Int. J. Pharm. 461:97-104 (2014); Khorsand et al., “Intracellular Drug Delivery Nanocarriers of Glutathione-Responsive Degradable Block Copolymers Having Pendant Disulfide Linkages,” Biomacromolecules 14:2103-2111 (2013), each of which is hereby incorporated by reference in its entirety). Other characteristics as described above apply.
(ii) A conjugate composed entirely or in part of folic acid.
(iii) A conjugate composed entirely or in part of spermine or a polymer of spermine.
(iv) A biocompatible moiety that contains a sulfhydryl moiety that can be conjugated via a reducible disulfide bond to the sulfhydryl group of the aminothiol. It should be noted that the number of differing moieties that can be conjugated to the sulfhydryl moiety of an aminothiol and that can meet the above conditions and requirements potentially is very large, and can continue to expand in the future as a result of new research. From this large group, moieties with the following characteristics can serve as protecting groups for conjugation to a therapeutic aminothiol: (a) moieties with a molecular weight of 100,000 Daltons or less, (b) moieties composed of biocompatible, non-toxic materials, (c) moieties amenable to cellular uptake at a rate that achieves intracellular levels of aminothiol in the range of 1 micromole or less per 10⁶ cells within the circulating half-life of the prodrug, and (d) moieties that are not amenable to conversion to toxins or that have low toxicity at dose levels that result in therapeutic effects of the aminothiol.
(v) It should be noted that the above listed drug delivery systems can be used in combination with each other. They also can be engineered further to provide targeted cell or tissue type delivery or targeted cell/tissue-type exclusion. In addition, new nanoscopic delivery systems are being developed frequently, and a variety of materials for use in the formation of nanoscopic drug delivery vehicles is expanding rapidly.

Methods for synthesis of a prodrug composed of an aminothiol or aminothiol analog and a PEG polymer, PEG-containing copolymer, or a dendrimer are described below.

In general, the following steps must be completed to bond a conjugate to the sulfhydryl group of an aminothiol or aminothiol analog. First, it is necessary to protect the amine groups of the aminothiol from reactivity; this process is referred to as ‘bocing’ and can be carried out using a variety of different protecting groups. The one condition that must be met is that the protecting groups must be removable as the last step of the synthesis by mechanisms that do not damage the polymer, copolymer or dendrimer or the aminothiol components of the prodrug. In the second step the sulfhydryl group of the aminothiol is bound to an intermediate via a disulfide bond between the sulfhydryl group of the conjugate and the sulfhydryl group of the aminothiol. In the third step this disulfide is reacted with a polymer, copolymer, or dendrimer. These conjugates must have at least one sulfhydryl group at one end of the molecule (for a linear polymer or copolymer) or at the ends of one or more arms (for a multi-arm polymer or copolymer) or at the end of the branches of a dendrimer. In the last step, the amine protecting groups must be removed using methods that do not damage the structure of the newly synthesized prodrug.

Aminothiol-conjugates of the present disclosure can be produced according to known methods. For example, aminothiol-conjugates of formula (I) can be prepared according to Schemes 1-2 outlined below.

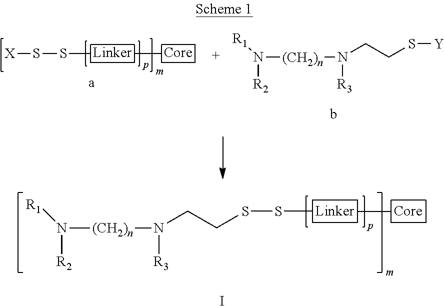

X and Y are each H, a suitable leaving group, or suitable activating group. X and Y can be the same or different.

Reaction of the disulfide (a) with compound (b) leads to formation of the aminothiol-conjugate of Formula (I). The reaction can be carried out in a variety of solvents, for example in water, buffer, methanol (MeOH), ethanol (EtOH), dimethylformamide (DMF), or other such solvents or in the mixture of such solvents. The reaction can be carried out at a temperature of 0° C. to 100° C., at a temperature of 0° C. to 40° C., or at a temperature of 0° C. to 25° C. During the reaction process, amino groups in the compound (b) can be protected by a suitable protecting group which can be selectively removed at a later time if desired. A detailed description of these groups and their selection and chemistry is contained in “The Peptides, Vol. 3”, Gross and Meinenhofer, Eds., Academic Press, New York, 1981, which is hereby incorporated by reference in its entirety. Thus, useful protective groups for the amino group are benzyloxycarbonyl (Cbz), t-butyloxycarbonyl (Boc), 2,2,2-trichloroethoxycarbonyl (Troc), t-amyloxycarbonyl, 4-methoxybenzyloxycarbonyl, 2-(trichlorosilyl) ethoxycarbonyl, 9-fluorenylmethoxycarbonyl (Fmoc), phthaloyl, acetyl (Ac), formyl, trifluoroacetyl, and the like. Any suitable commercially available disulfide (a) can be used according to the present disclosure. Alternatively, disulfide (a) can be prepared according to known methods.

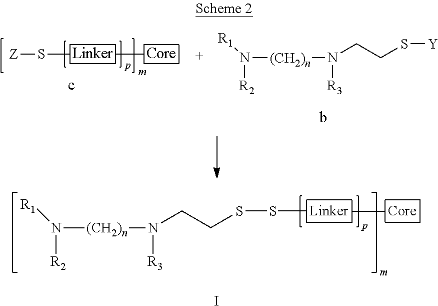

Y and Z are H, a suitable leaving group, or suitable activating group. Y and Z can be the same or different.

Alternatively, the aminothiol-conjugate of Formula (I) can be prepared by reacting the thiol (c) with compound (b). The reaction can be carried out in a variety of solvents, for example in water, buffer, methanol (MeOH), ethanol (EtOH), dimethylformamide (DMF), or other such solvents or in the mixture of such solvents. The reaction can be carried out at a temperature of 0° C. to 100° C., at a temperature of 0° C. to 40° C., or at a temperature of 0° C. to 25° C. During the reaction process, amino groups in the compound (b) can be protected by a suitable protecting group which can be selectively removed at a later time if desired. A detailed description of these groups and their selection and chemistry is contained in “The Peptides, Vol. 3”, Gross and Meinenhofer, Eds., Academic Press, New York, 1981, which is hereby incorporated by reference in its entirety. Thus, useful protective groups for the amino group are benzyloxycarbonyl (Cbz), t-butyloxycarbonyl (Boc), 2,2,2-trichloroethoxycarbonyl (Troc), t-amyloxycarbonyl, 4-methoxybenzyloxycarbonyl, 2-(trichlorosilyl) ethoxycarbonyl, 9-fluorenylmethoxycarbonyl (Fmoc), phthaloyl, acetyl (Ac), formyl, trifluoroacetyl, and the like. Any suitable commercially available thiol (c) can be used according to the present disclosure. Alternatively, thiol (c) can be prepared according to known methods.

The PEG-(SZ)ₘ and PEG-(S-SX)ₘ molecules that can be used for the preparation of the aminothiol-conjugates of Formula (I) are usually prepared using a polymerization reaction and are present as a mixture of PEG polymers having different lengths and molecular weights. Thus, the aminothiol-conjugates of formula (I) prepared from the PEG-(SZ)ₘ or PEG-(S-SX)ₘ molecules can also exist as a mixture of polymers, each having different molecular weight, where each arm may have a different length.

Some PEG-(SZ)ₘ and PEG-(S-SX)ₘ molecules can be prepared as PEG polymers having the same arm(s) length and molecular weight. Thus, the aminothiol-conjugates of formula (I) prepared from such PEG-(SZ)ₘ or PEG-(S-SX)ₘ molecules can exist as polymers having the same arm(s) length.

The PEG-(SZ)ₘ and PEG-(S-SX)ₘ polymers that can be used as a scaffold for conjugation with compound (b) can be linear PEG polymers of the same or different arm length (e.g., 1 or 2 arm) and molecular weight or a multi-arm polymer with differing numbers of arms (e.g., 3, 4, 5, 6, 7, 8, 9, 10, 11, or 12 arms) and molecular weight.

Schemes 3-7 describe the synthesis of an exemplary aminothiol-conjugate of Formula (I). Synthesis described in Schemes 3-7 can be modified to prepare aminothiol-conjugates of Formula (I) where

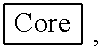

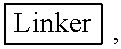

R₁, R₂, R₃, m, n, and p are different from the ones exemplified in the schemes below.

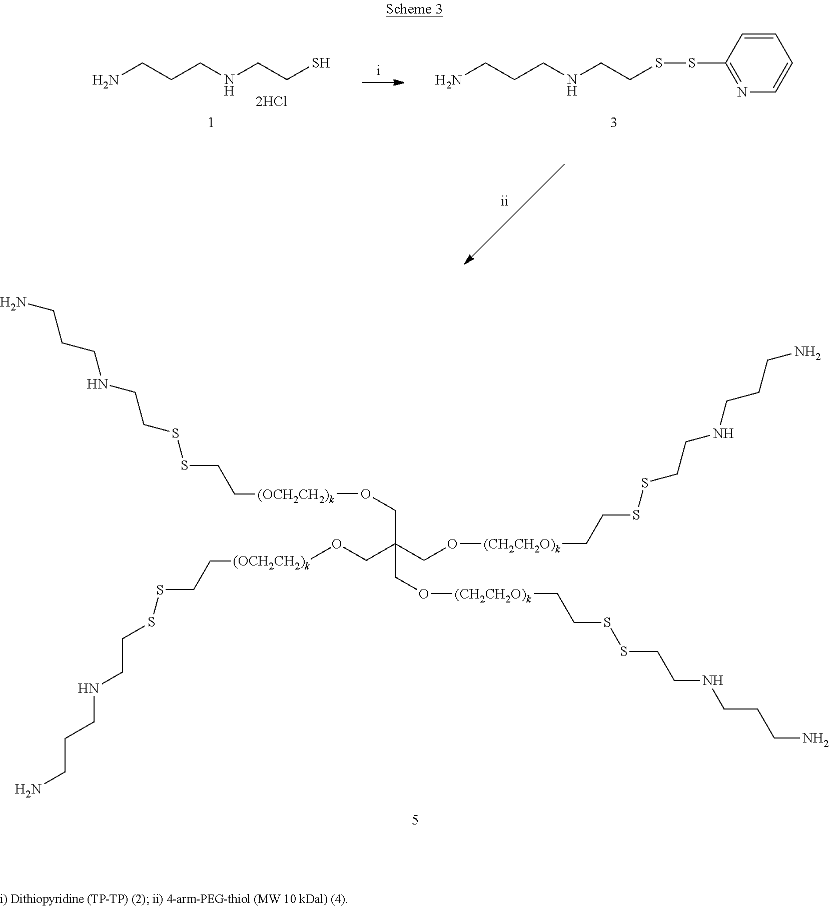

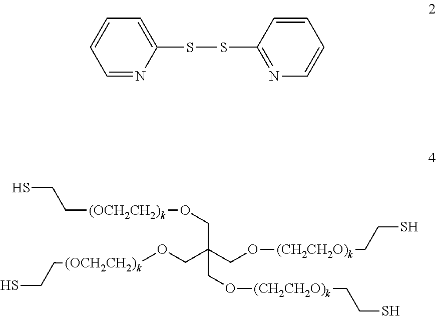

Synthesis of 4-star-PEG-S-S-WR1065 conjugate (5) is shown in Scheme 3. WR1065 dihydrochloride (1) was reacted with dithiopyridine (TP-TP) (2) to form WR1065-S-TP at room temperature. The intermediate (3) was reacted with 4-arm-PEG-thiol (MW 10 kDal) (4) to form the 4-star-PEG-S-S-WR1065 conjugate (5) of average Mw 10.536 kDal. The above scheme does not show steps to protect and then deprotect the nitrogens in WR1065 during the synthesis of 4-star-PEG-S-S-WR1065 conjugate (5). The nitrogens on WR1065 (1) had to be protected and in the last step the protecting groups had to be removed (Schemes 4-7).

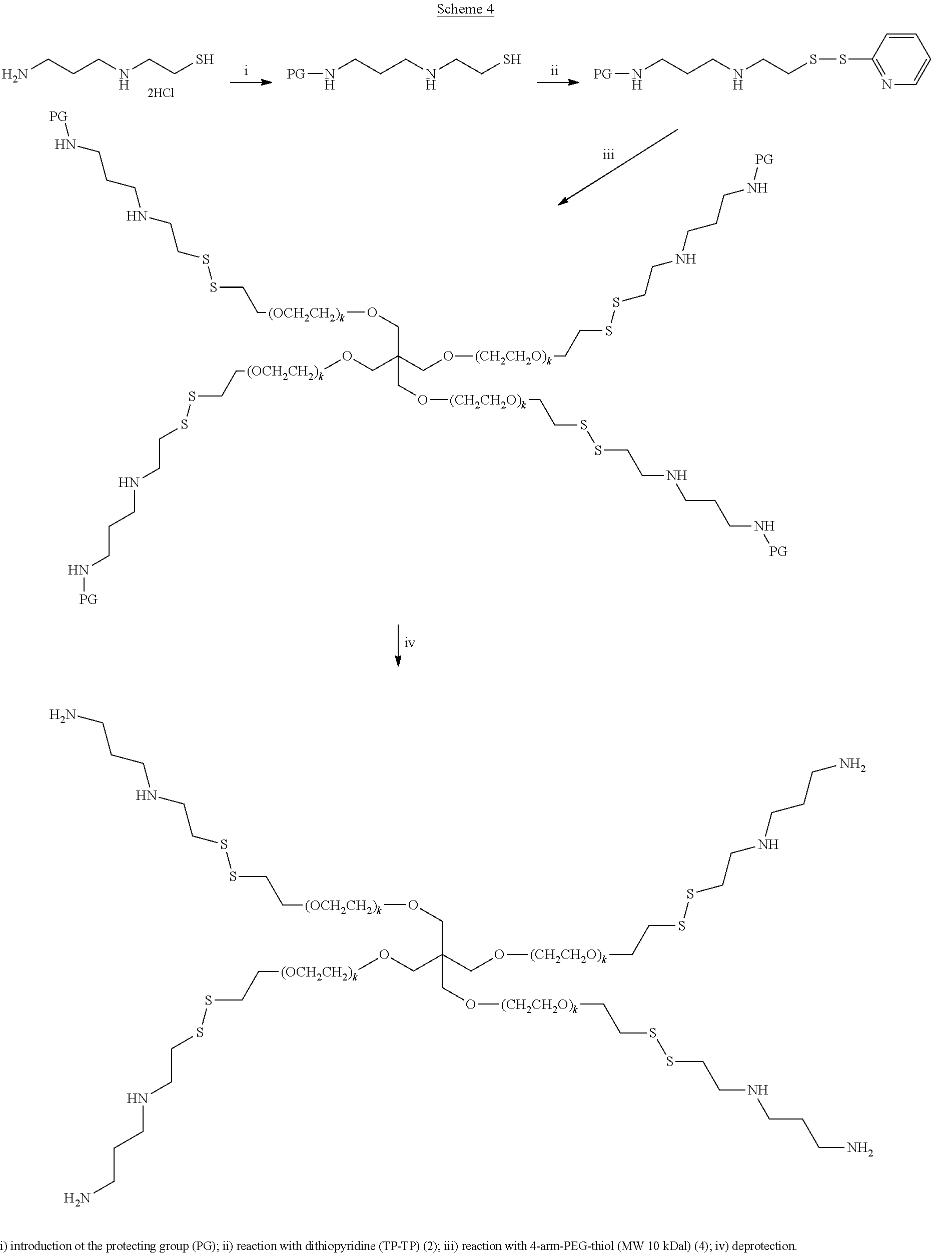

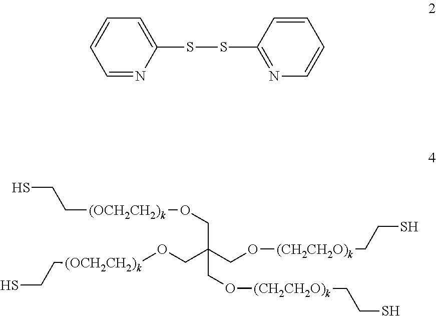

PG is any suitable protecting group.

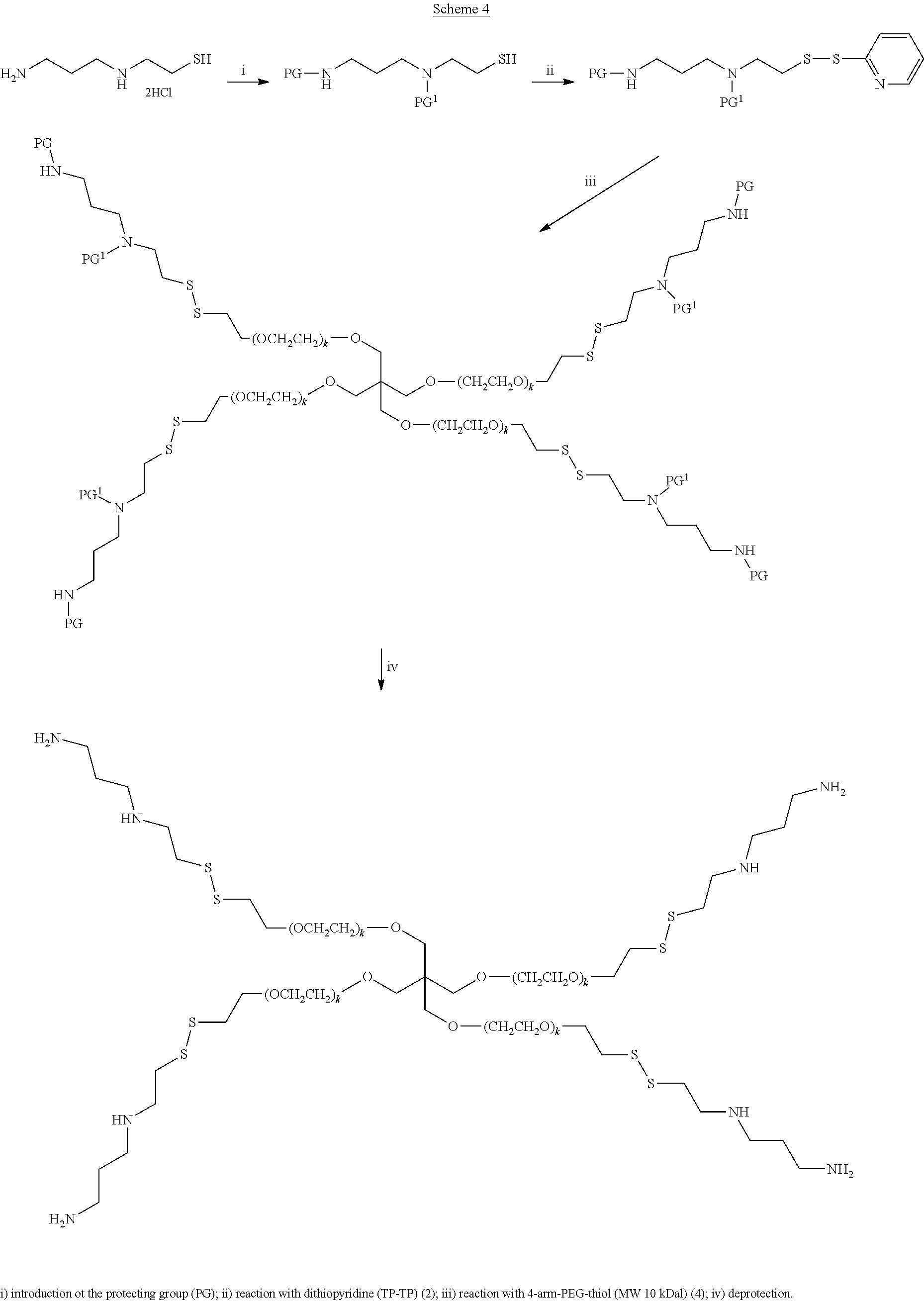

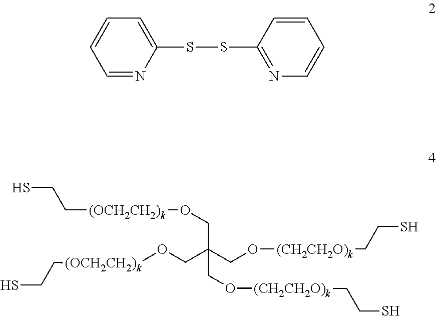

PG and PG¹ are each a suitable protecting group. PG and PG¹ can be the same or different.

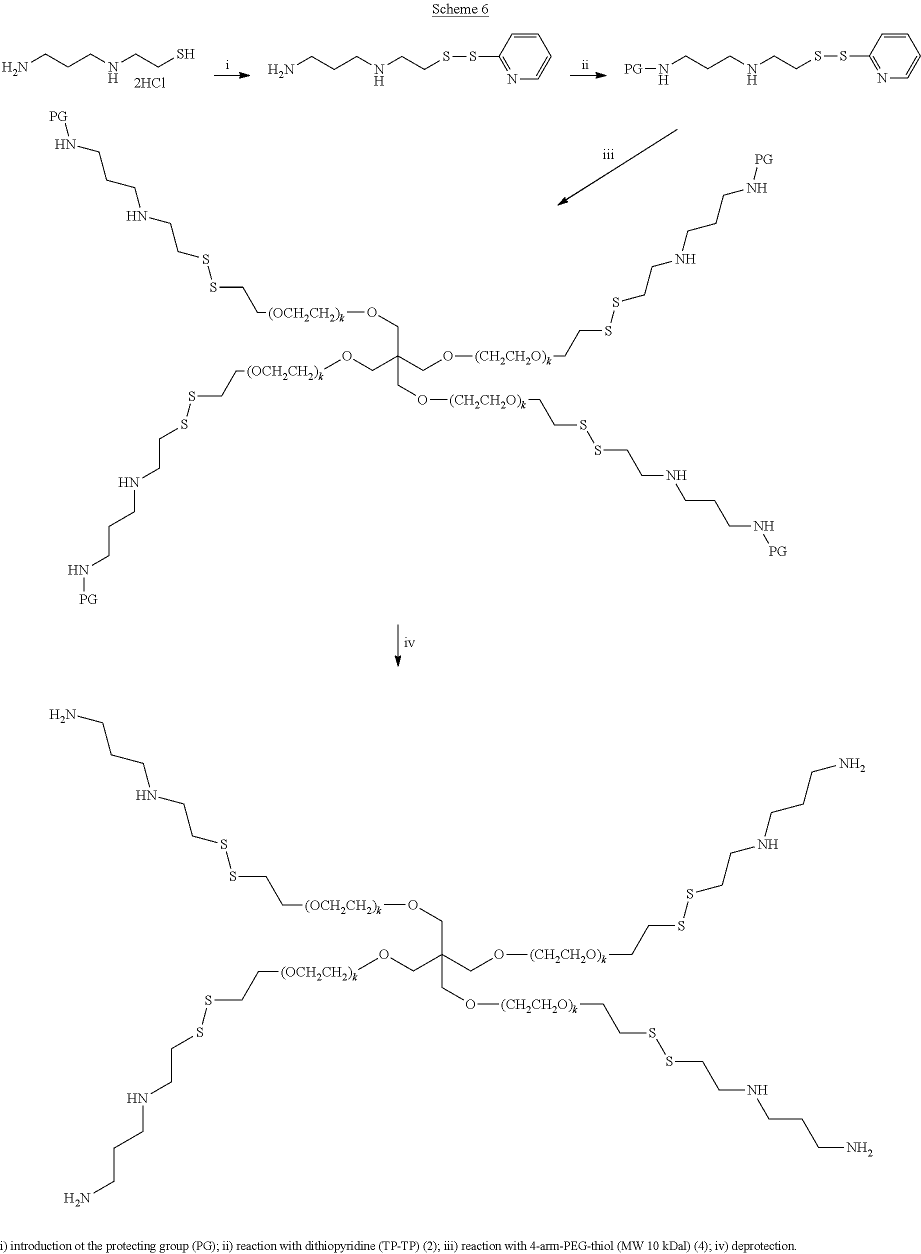

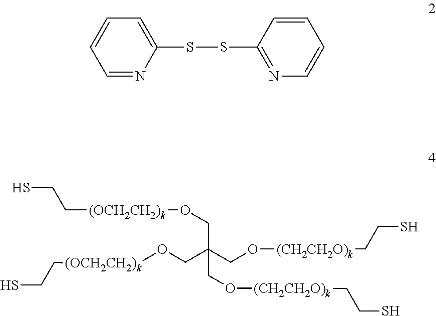

PG is any suitable protecting group.

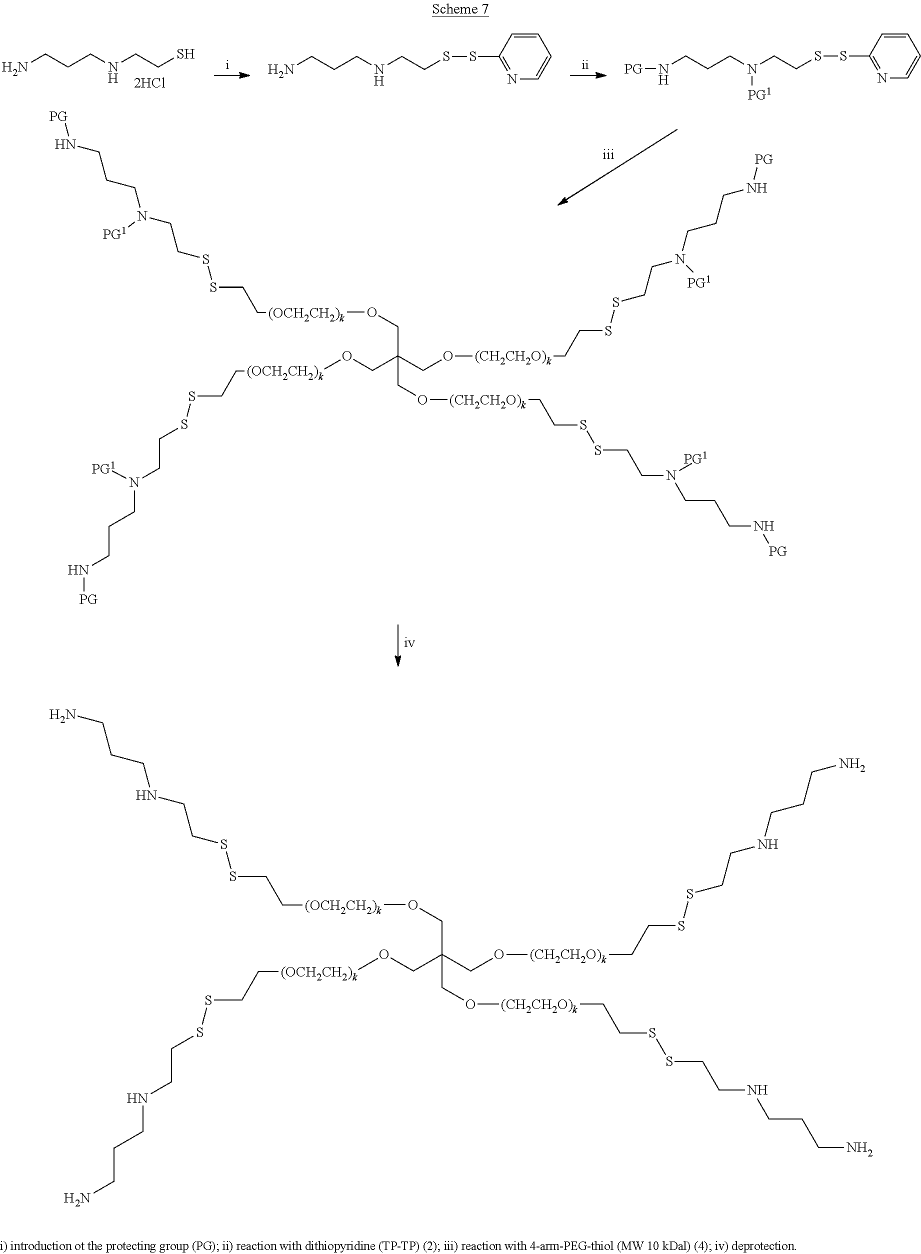

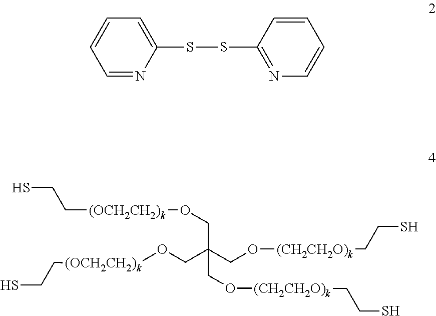

PG and PG¹ are each a suitable protecting group. PG and PG¹ can be the same or different.

The PEG-SH molecule used as a scaffold for conjugation of WR1065 can be a linear PEG polymer of differing length and molecular weight or a multi-arm polymer with differing numbers of arms (e.g., 4 arms as shown above (4) or 6, 8, etc. arms) and molecular weight.

In some embodiments of the methods and combination therapeutics of the present disclosure, the PEG-(SH) m molecules are used as a mixture of polymers, each having different molecular weight, where each arm may have a different length. In alternative embodiments, PEG-(SH) m molecules are present as a polymer having a particular molecular weight or a narrow range of molecular weights.

The aminothiol-conjugate of Formula (I) may be delivered intracellularly or intracytoplasmically to cells (e.g., target cells). In general, any method described in the literature or developed in the future that has characteristics that allow the release of the aminothiol by any biologic or cellular mechanism can be used. Thus, to achieve enhanced drug delivery, the aminothiol-conjugate of Formula (I) can be delivered in combination with other drug delivery modules as presented below. Targeted drug delivery and targeted drug exclusion are desirable but not necessary.

A variety of particulate carriers for intracellular drug delivery have been developed and/or described. Nanoparticles also are referred to as nanovesicles, nanocarriers, or nanocapsules and include lysosomes, micelles, capsules, polymersomes, nanogels, dendritic and macromolecular drug conjugates, and nanosized nucleic acid complexes. A summary of categories into which nanoparticles are sometimes divided includes the following items (1)-(18).

(1) Cell penetrating agents such as amphiphilic polyproline helix P11LRR (such as those described in Li et al., “Cationic Amphiphilic Polyproline Helix P11LRR Targets Intracellular Mitochondria,” J. Controlled Release 142:259-266 (2010), which is hereby incorporated by reference in its entirety) or peptide-functionalized quantum dots, such as those described in (Liu et al., “Cell-Penetrating Peptide-Functionalized Quantum Dots for Intracellular Delivery,” J. Nanosci. Nanotechnol. 10:7897-7905 (2010), which is hereby incorporated by reference in its entirety).

(2) Carriers responsive to pH, such as carbonate apatite (Hossain et al., “Carbonate Apatite-Facilitated Intracellularly Delivered siRNA for Efficient Knockdown of Functional Genes,” J. Controlled Release 147:101-108 (2010), which is hereby incorporated by reference in its entirety).

(3) C2-streptavidin delivery systems, which have been used to facilitate drug delivery to macrophages and T-leukemia cells (such as those described in Fahrer et al., “The C2-Streptavidin Delivery System Promotes the Uptake of Biotinylated Molecules in Macrophages and T-leukemia cells,” Biol. Chem. 391, 1315-1325 (2010), which is hereby incorporated by reference in its entirety).

(4) CH (3)-TDDS drug delivery systems.

(5) Hydrophobic bioactive carriers (such as those described in Imbuluzqueta et al., “Novel Bioactive Hydrophobic Gentamicin Carriers for the Treatment of Intracellular Bacterial Infections,” Acta. Biomater. 7:1599-1608 (2011), which is hereby incorporated by reference in its entirety).

(6) Exosomes (such as those described in Lakhal et al., “Intranasal Exosomes for Treatment of Neuroinflammation? Prospects and Limitations,” Mol. Ther. 19:1754-1756 (2011); Zhang et al., “Newly Developed Strategies for Multifunctional Mitochondria-Targeted Agents In Cancer Therapy,” Drug Discovery Today 16:140-146 (2011), each of which is hereby incorporated by reference in its entirety).

(7) Lipid-based delivery systems (such as those described in Bildstein et al., “Transmembrane Diffusion of Gemcitabine by a Nanoparticulate Squalenoyl Prodrug: An Original Drug Delivery Pathway,” J. Controlled Release 147:163-170 (2010); Foged, “siRNA Delivery with Lipid-Based Systems: Promises and Pitfalls,” Curr. Top. Med. Chem. 12:97-107 (2012); Holpuch et al., “Nanoparticles for Local Drug Delivery to the Oral Mucosa: Proof of Principle Studies,” Pharm. Res. 27:1224-1236 (2010); Kapoor et al., “Physicochemical Characterization Techniques for Lipid Based Delivery Systems for siRNA,” Int. J. of Pharm. 427:35-57 (2012), each of which is hereby incorporated by reference in its entirety), including microtubules, such as those described in (Kolachala et al., “The Use of Lipid Microtubes as a Novel Slow-Release Delivery System for Laryngeal Injection,” The Laryngoscope 121:1237-1243 (2011), which is hereby incorporated by reference in its entirety).

(8) Liposome or liposome-based delivery systems.

(9) Micelles, including disulfide cross-linked micelles, such as those described in (Li et al., “Delivery of Intracellular-Acting Biologics in Pro-Apoptotic Therapies,” Curr. Pharm. Des. 17:293-319 (2011), which is hereby incorporated by reference in its entirety). Carriers with disulfide bonds can be formulated so that one or more disulfide bonds link to the aminothiol. A variety of micelles have been described, such as phospholipid-polyaspartamide micelles for pulmonary delivery.

(10) Microparticles, such as those described in (Ateh et al., “The Intracellular Uptake of CD95 Modified Paclitaxel-Loaded Poly(Lactic-Co-Glycolic Acid) Microparticles,” Biomater. 32:8538-8547 (2011), which is hereby incorporated by reference in its entirety).

(11) Molecular carriers, such as those described in (Hettiarachchi et al., “Toxicology and Drug Delivery by Cucurbit[n]uril Type Molecular Containers,” PloS One 5: e10514 (2010), which is hereby incorporated by reference in its entirety).

(12) Nanoparticles referred to as ‘nanocarriers’, such as those described in (Gu et al., “Tailoring Nanocarriers for Intracellular Protein Delivery,” Chem. Soc. Rev. 40:3638-3655 (2011), which is hereby incorporated by reference in its entirety), some of which have been formulated for delivery of agents to HIV infected cells, such as those described in (Gunaseelan et al., “Surface Modifications of Nanocarriers for Effective Intracellular Delivery of Anti-HIV Drugs,” Adv. Drug Delivery Rev. 62:518-531 (2010), which is hereby incorporated by reference in its entirety).

(13) Nanoscopic multi-variant carriers.

(14) Nanogels (such as those described in Zhan et al., “Acid-Activatable Prodrug Nanogels for Efficient Intracellular Doxorubicin Release,” Biomacromolecules 12:3612-3620 (2011) and Zhang et al., “Folate-Mediated poly(3-hydroxybutyrate-co-3-hydroxyoctanoate) Nanoparticles for Targeting Drug Delivery,” Eur. J. Pharm. Biopharm. 76:10-16 (2010), each of which is hereby incorporated by reference in its entirety).

(15) Hybrid nanocarrier systems, which consist of components of two or more particulate delivery systems (such as those described in Pittella et al., “Enhanced Endosomal Escape of siRNA-Incorporating Hybrid Nanoparticles from Calcium Phosphate and PEG-Block Charge-Conversional Polymer for Efficient Gene Knockdown With Negligible Cytotoxicity,” Biomater. 32:3106-3114 (2011), which is hereby incorporated by reference in its entirety). Copolymeric micelle nanocarrier (such as those described in Chen et al., “pH and Reduction Dual-Sensitive Copolymeric Micelles for Intracellular Doxorubicin Delivery,” Biomacromolecules 12:3601-3611 (2011), which is hereby incorporated by reference in its entirety); liposomal nanocarriers, (such as those described in (Kang et al., “Design of a Pep-1 Peptide-Modified Liposomal Nanocarrier System for Intracellular Drug Delivery: Conformational Characterization and Cellular Uptake Evaluation,” J. of Drug Targeting 19:497-505 (2011), which is hereby incorporated by reference in its entirety).

(16) Nanoparticles can be constructed with a variety of nanomaterials (such as those described in Adeli et al., “Synthesis of New Hybrid Nanomaterials: Promising Systems for Cancer Therapy,” Nanomed. Nanotechnol. Biol. Med. 7:806-817 (2011); Al-Jamal et al., “Enhanced Cellular Internalization and Gene Silencing with a Series of Cationic Dendron-Multiwalled Carbon Nanotube: siRNA Complexes,” FASEB J 24:4354-4365 (2010); Bulut et al., “Slow Release and Delivery of Antisense Oligonucleotide Drug by Self-Assembled Peptide Amphiphile Nanofibers,” Biomacromolecules 12:3007-3014 (2011), each of which is hereby incorporated by reference in its entirety).

(17) Peptide-based drug delivery systems, which include a variety of cell penetrating peptides and including (but not limited to) TAT-based delivery systems (such as those described in Johnson et al., “Therapeutic Applications of Cell-Penetrating Peptides,” Methods Mol. Biol. 683:535-551 (2011), which is hereby incorporated by reference in its entirety).

(18) Polymers or copolymer-based delivery systems, such as those described in Edinger et al., “Bioresponsive Polymers for the Delivery of Therapeutic Nucleic Acids,” Wiley Interdiscip. Rev. Nanomed. and Nanobiotechnol. 3:33-46 (2011), which is hereby incorporated by reference in its entirety.

Additional intracellular drug delivery systems that may be considered to fall into the category of nanoparticles include the following items (a)-(u).

(a) Aptamers such as those described in Orava et al., “Delivering Cargoes into Cancer Cells Using DNA Aptamers Targeting Internalized Surface Portals,” Biochim. Biophys. Acta. 1798:2190-2200 (2010), which is hereby incorporated by reference in its entirety.

(b) Bacterial drug delivery systems (such as those described in Pontes et al., “Lactococcus Lactis as a Live Vector: Heterologous Protein Production and DNA Delivery Systems,” Protein Expression Purif. 79:165-175 (2011), which is hereby incorporated by reference in its entirety).

(c) Protein-based, self-assembling intracellular bacterial organelles (bacterial shells) (such as those described in Corchero et al., “Self-Assembling, Protein-Based Intracellular Bacterial Organelles: Emerging Vehicles for Encapsulating, Targeting And Delivering Therapeutical Cargoes,” Microb. Cell Factories 10:92 (2011), which is hereby incorporated by reference in its entirety).

(d) Blended systems (such as those described in Lee et al., “Lipo-Oligoarginines as Effective Delivery Vectors to Promote Cellular Uptake,” Mol. Biosyst. 6:2049-2055 (2010), which is hereby incorporated by reference in its entirety).

(e) Covalently modified proteins (such as those described in Muller, “Oral Delivery of Protein Drugs: Driver for Personalized Medicine,” Curr. Molec. Bio. 13:13-24 (2011), which is hereby incorporated by reference in its entirety).

(f) Drug-loaded irradiated tumor cells (such as those described in Kim, et al., “Delivery of Chemotherapeutic Agents Using Drug-Loaded Irradiated Tumor Cells to Treat Murine Ovarian Tumors,” J. Biomed. Sci. 17:61 (2010), which is hereby incorporated by reference in its entirety).

(g) Dual loading using micellplexes (such as those described in Yu et al., “Overcoming Endosomal Barrier by Amphotericin B-Loaded Dual pH-Responsive PDMA-b-PDPA Micelleplexes for siRNA Delivery,” ACS Nano 5:9246-9255 (2011), which is hereby incorporated by reference in its entirety).

(h) Ethosomes (such as those described in Godin et al., “Ethosomes: New Prospects in Transdermal Delivery,” Crit. Rev. Ther. Drug Carrier Syst. 20:63-102 (2003), which is hereby incorporated by reference in its entirety).

(i) Inhalation-based delivery systems (such as those described in Patton et al., “The Particle Has Landed—Characterizing the Fate of Inhaled Pharmaceuticals,” J. of Aerosol Medicine and Pul. Drug Del. 23: Suppl 2: S71-87 (2010), which is hereby incorporated by reference in its entirety).

(j) Irradiated tumor cell-based delivery system (such as those described in Kim, et al., “Delivery of Chemotherapeutic Agents Using Drug-Loaded Irradiated Tumor Cells to Treat Murine Ovarian Tumors,” J. Biomed. Sci. 17:61 (2010), which is hereby incorporated by reference in its entirety).

(k) Lipid-based carriers.

(l) Lipospheres, such as acoustically active lipospheres.

(m) Microencapsulated drug delivery (such as those described in Oettinger and D'Souza, “Microencapsulated Drug Delivery: A New Approach to Pro-Inflammatory Cytokine Inhibition,” J. Microencapsul. 29 (5): 455-462 (2012), which is hereby incorporated by reference in its entirety).

(n) A delivery system referred to as molecular umbrellas (such as those described in Cline et al., “A Molecular Umbrella Approach to the Intracellular Delivery of Small Interfering RNA,” Bioconjug. Chem. 22 (11): 2210-2216 (2011), which is hereby incorporated by reference in its entirety).

(o) Niosomes (non-ionic surfactant-based liposomes).

(p) Photo-activatable drug delivery systems.

(q) Polymeric microcapsule (such as those described in Pavlov et al., “Neuron Cells Uptake of Polymeric Microcapsules and Subsequent Intracellular Release,” Maccromol. Biosci. 11 (6): 848-854 (2011), which is hereby incorporated by reference in its entirety).

(r) Self-emulsifying drug delivery system (such as those described in Lei et al., “Development of a Novel Self-Microemulsifying Drug Delivery System for Reducing HIV Protease Inhibitor-Induced Intestinal Epithelial Barrier Dysfunction,” Mol. Pharm. 7 (3): 844-853 (2010), which is hereby incorporated by reference in its entirety).

(s) Trojan horse delivery systems.

(t) Vesicles including but not limited to reduction sensitive vesicles (such as those described in Park et al., “Reduction-Sensitive, Robust Vesicles with a Non-Covalently Modifiable Surface as a Multifunctional Drug-Delivery Platform,” Small 6 (13): 1430-1441 (2010), which is hereby incorporated by reference in its entirety).

(u) Viral vectors and viral-like systems (such as those described in Bacman et al., “Organ-Specific Shifts in mtDNA Heteroplasmy Following Systemic Delivery of a Mitochondria-Targeted Restriction Endonuclease,” Gene Ther. 17 (6): 713-720 (2010); Chailertvanitkul et al., “Adenovirus: a Blueprint for Non-Viral Gene Delivery,” Curr. Opin. Biotechnol. 21 (15): 627-632 (2010), each of which is hereby incorporated by reference in its entirety).

It should be noted that the above listed drug delivery systems can be used in combination with each other. They also can be engineered further to provide target cell or tissue type delivery or targeted cell/tissue-type exclusion. In addition, new nanoscopic delivery systems are being developed frequently, and a variety of materials for use in the formation of nanoscopic drug delivery vehicles is expanding rapidly.

The above delivery systems can be used in combination with enhanced delivery techniques. Examples of such techniques include the following items (I)-(XV).

(I) Amphotercin B-mediated drug delivery enhancement.

(II) Ultrasound-mediated techniques (such as those described in Grimaldi et al., “Ultrasound-Mediated Structural Changes in Cells Revealed by FTIR Spectroscopy: a Contribution to the Optimization of Gene and Drug Delivery,” Spectrochim. Acta Part A 84:74-85 (2011); Yudina et al., “Ultrasound-Mediated Intracellular Drug Delivery Using Microbubbles and Temperature-Sensitive Liposomes,” J. Controlled Release 155:442-448 (2011), each of which is hereby incorporated by reference in its entirety).

(III) Temperature-sensitive delivery and/or release systems.

(IV) pH-sensitive delivery and/or release systems.

(V) Redox-responsive delivery systems, such as those described in (Zhao et al., “A Novel Human Derived Cell-Penetrating Peptide in Drug Delivery,” Mol. Biol. Rep. 38:2649-2656 (2011), which is hereby incorporated by reference in its entirety).

(VI) Bioreducible delivery systems (such as those described in Liu et al., “Bioreducible Micelles Self-Assembled from Amphiphilic Hyperbranched Multiarm Copolymer for Glutathione-Mediated Intracellular Drug Delivery,” Biomacromolecules 12:1567-1577 (2011), which is hereby incorporated by reference in its entirety).

(VII) Methods to enhance endolysomal escape (such as those described in Paillard et al., “The Importance of Endo-Lysosomal Escape with Lipid Nanocapsules for Drug Subcellular Bioavailability,” Biomaterials 31:7542-7554 (2010), which is hereby incorporated by reference in its entirety).

(VIII) Inhalation methods (such as those described in Zhuang et al., “Treatment of Brain Inflammatory Diseases by Delivering Exosome Encapsulated Anti-Inflammatory Drugs from the Nasal Region to the Brain,” Mol. Ther. 19:1769-1779 (2011), which is hereby incorporated by reference in its entirety).

(IX) Methods to enhance oral delivery (such as those described in Muller, “Oral Delivery of Protein Drugs: Driver for Personalized Medicine,” Curr. Molec. Bio. 13:13-24 (2011), which is hereby incorporated by reference in its entirety).

(X) Targeted cell delivery systems, some of which have been developed for use in the delivery of anti-HIV drugs (such as those described in Bronshtein et al., “Cell Derived Liposomes Expressing CCR5 as a New Targeted Drug-Delivery System for HIV Infected Cells,” J. Controlled Release 151:139-148 (2011); Gunaseelan et al., “Surface Modifications of Nanocarriers for Effective Intracellular Delivery of Anti-HIV Drugs,” Adv. Drug Delivery Rev. 62:518-531 (2010); Kelly et al., “Targeted Liposomal Drug Delivery to Monocytes and Macrophages.,” J. Drug Deliv. 2011:727241 (2011), each of which is hereby incorporated by reference in its entirety).

(XI) Slow or on-demand release systems (such as those described in Hu et al., “Multifunctional Nanocapsules for Simultaneous Encapsulation of Hydrophilic and Hydrophobic Compounds and On-Demand Release,” ACS Nano 6:2558-2565 (2012), which is hereby incorporated by reference in its entirety).

(XII) Targeted delivery to one or more receptors (such as those described in Ming, “Cellular Delivery of siRNA and Antisense Oligonucleotides via Receptor-Mediated Endocytosis,” Expert Opin. on Drug Delivery 8:435-449 (2011), which is hereby incorporated by reference in its entirety).

(XIII) Targeted delivery to one or more different subcellular organelles (such as those described in Paulo et al., “Nanoparticles for Intracellular-Targeted Drug Delivery,” Nanotechnol. 22:494002 (2011); Zhang et al., “Newly Developed Strategies for Multifunctional Mitochondria-Targeted Agents In Cancer Therapy,” Drug Discovery Today 16:140-146 (2011), which is hereby incorporated by reference in its entirety).

(XIV) Methods to improve or to regulate drug uptake (such as those described in Lorenz, S et al., “The Softer and More Hydrophobic the Better: Influence of the Side Chain Of Polymethacrylate Nanoparticles for Cellular Uptake,” Macromol. Bioscience 10:1034-1042 (2010); Ma et al., “Distinct Transduction Modes of Arginine-Rich Cell-Penetrating Peptides for Cargo Delivery into Tumor Cells,” Int. J. Pharm. 419:200-208 (2011), each of which is hereby incorporated by reference in its entirety).

(XV) Methods that use erythrocytes as drug carriers as described in, e.g., Millan et al., “Drug, Enzyme and Peptide Delivery using Erythrocytes As Carriers,” J. Control Release 95:27-49 (2004), which is hereby incorporated by reference in its entirety.

Although delivery of amifostine (the phosphorothioate) using nanoparticles has been reported previously (Pamujula et al., “Oral Delivery of Spray Dried PLGA/Amifostine Nanoparticles,” J. Pharm. Pharmacol. 56:1119-1125 (2004); Pamujula et al., “Preparation and In Vitro Characterization of Amifostine Biodegradable Microcapsules,” Eur. J. Pharm. Biopharm. 57:213-218 (2004); Pamujula et al., “Radioprotection in Mice Following Oral Delivery of Amifostine Nanoparticles,” Int. J. Radiat. Biol. 81:251-257 (2005); Pamujula et al., “Radioprotection of mice following oral administration of WR-1065/PLGA nanoparticles,” Int. J. Radiat. Biol. 84:900-908 (2008), each of which is hereby incorporated by reference in its entirety), this delivery system was different than the aminothiol-conjugates and compositions described herein and does not resolve the problems associated with dependence upon alkaline phosphatase for drug activation. Unlike aminothiol-conjugates described herein, such delivery systems do not resolve the problems of adventitious drug reactivity in circulation or drug release distal to target cells. This previous attempt also fails to address the potential toxicity problems associated with activation of the drug outside of cells.

Other methods that can be used to alter or improve drug delivery and/or uptake include the use of surfactants as described in U.S. Pat. No. 6,489,312 to Stogniew, which is hereby incorporated by reference in its entirety.

Other methods that can be used to alter or improve drug delivery include Tween 20, Tween 80, and other similar surfactants. The purpose of using surfactants is to reduce disulfide bond reduction and other potential forms of drug metabolism during delivery and before the drug reaches target cells.

Other methods that can be used to alter or improve drug delivery include the use of agents to enhance drug uptake into cells and/or the activity of plasma membrane transporters/systems. Examples include α-difluoromethylornithine (Eflornithine or DFMO), amino acids (Uemura et al., “Polyamine Transport is Mediated by Both Endocytic and Solute Carrier Transport Mechanisms in the Gastrointestinal Tract,” Am. J. Physiol. Gastrointest. Liver Physiol. 299: G517-G522 (2010), which is hereby incorporated by reference in its entirety).

Other methods that can be used to alter or improve drug delivery, stability during circulation in plasma, and/or uptake into target cells include the use of shielding with macromolecules (Brulisauer et al., “Disulfide-Containing Parenteral Delivery Systems and their Redox-Biological Fate,” J. Control Release 195:147-154 (2014), which is hereby incorporated by reference in its entirety).

Other methods that can be used to alter or improve drug delivery, stability during circulation in plasma, and/or uptake into target cells include shielding with macromolecules or coating with something like N-(2-hydroxypropyl) methacrylamide (HPMA)/PHPMA. It may be necessary to adjust the structural properties of the drug carrier so that the rate of cleavage in the different redox-biological milieus can be modulated (Brulisauer et al., “Disulfide-Containing Parenteral Delivery Systems and their Redox-Biological Fate,” J. Control Release 195:147 154 (2014), which is hereby incorporated by reference in its entirety).

In certain embodiments, the active form of the aminothiol (or analogue thereof) is released intracytoplasmically to achieve therapeutic effects. In general any drug delivery system and/or drug protection method that includes the capacity to release the active form of the drug following intracytoplasmic delivery can be used. The key to the selection of one or more of the protection and delivery systems described above is to recognize that once the drug has been delivered into the cytoplasm of target cells, the delivery/protection method must allow for release of the aminothiol. Thus, binding of the conjugate to the aminothiol must be carried out so as to result in a reducible (bioreducible) disulfide bond (Benham et al., “Disulfide Bonding Patterns and Protein Topologies,” Protein Sci. 2:41-54 (1993); Liu et al., “Disulfide Bond Structures of IgG Molecules: Structural Variations, Chemical Modifications and Possible Impacts to Stability and Biological Function,” mAbs 4:17-23 (2012), each of which is hereby incorporated by reference in its entirety).

As used herein, treatment means any manner in which one or more of the symptoms of a disease or disorder are ameliorated or otherwise beneficially altered. A therapeutically effective amount of aminothiol-conjugates as described herein can be, e.g., an amount sufficient to prevent the onset of a disease state or to shorten the duration of a disease state, or to decrease the severity of one or more symptoms. Treatment includes inhibition and attenuation of, e.g., viruses or pathogenic microorganisms in the subject.

Amifostine, phosphonol, and structurally-related phosphorothioates and analogs have been shown to have therapeutic efficacy when used as chemoprotectants, cytoprotectants, radioprotectants, anti-fibrotic agents, anti-tumor agents with anti-metastatic, anti-invasive, and anti-mutagenic effects, antioxidants, free radical scavengers, anti-viral agents, and as agents that prevent tumor induction, slow tumor cell growth, have antitumor/anticancer effects and/or enhance the efficacy of anticancer agents (Grdina et al., “Differential Activation of Nuclear Transcription Factor Kappab, Gene Expression, and Proteins By Amifostine's Free Thiol in Human Microvascular Endothelial and Glioma Cells,” Semin. Radiat. Oncol. 12:103-111 (2002); Grdina et al., “Relationships between Cytoprotection and Mutation Prevention by WR-1065,” Mil Med 167:51-53 (2002); Grdina et al., “Radioprotectors: Current Status and New Directions,” Radiat. Res. 163:704-705 (2002); Poirier et al., “Antiretroviral Activity of the Aminothiol WR1065 Against Human Immunodeficiency Virus (HIV-1) in Vitro and Simian Immunodeficiency Virus (SIV) Ex Vivo,” AIDS Res. Ther. 6:24 (2009); Walker et al., “WR1065 Mitigates AZT-ddI-Induced Mutagenesis and Inhibits Viral Replication,” Environ. Mol. Mutagen. 50:460 472 (2009), each of which is hereby incorporated by reference in its entirety). Experimental results have shown that WR-1065, the active metabolite of amifostine, exhibited antiviral efficacy against HIV, influenza virus A and B, and three species of adenovirus. Later studies also demonstrated efficacy against SIV (Poirier et al., “Antiretroviral Activity of the Aminothiol WR1065 Against Human Immunodeficiency Virus (HIV-1) in Vitro and Simian Immunodeficiency Virus (SIV) Ex Vivo,” AIDS Res. Ther. 6:24 (2009), which is hereby incorporated by reference in its entirety) and a NIAID/DMID contract laboratory demonstrated efficacy against Ebola virus.

In certain embodiments of the methods according to the present disclosure, the subject is one in need of treatment with an antiviral agent, a chemoprotectant, a cytoprotectant, a radioprotectant, an anti-fibrotic agent, an anti-tumor agent, an antioxidant, or a combination thereof.

As noted above, one aspect of the present disclosure relates to a method of treating a neoplastic condition in a subject in need thereof by administering to said subject a combination therapeutic comprising an aminothiol-conjugate as described herein in an amount effective to treat the neoplastic condition in the subject. Another aspect of the present disclosure relates to a method of treating a subject at risk of developing a neoplastic condition by administering to said subject a combination therapeutic comprising an aminothiol-conjugate as described herein under conditions effective to reduce the risk of developing the neoplastic condition. Such a subject at risk of developing a neoplastic condition includes, e.g., a subject receiving repeated diagnostic radiation exposures.

For instance, sensitive tumor types identified through in vitro studies include: breast cancer, ovarian cancer, malignant melanoma (Brenner et al., “Variable Cytotoxicity of Amifostine in Malignant and Non-Malignant Cell Lines,” Oncol. Rep. 10 (5): 1609-1613 (2003), which is hereby incorporated by reference in its entirety); ovarian cancer (Calabro-Jones et al., “The Limits to Radioprotection of Chinese Hamster V79 Cells by WR-1065 Under Aerobic Conditions,” Radiat. Res. 149:550-559 (1998) (“Calabro-Jones”), which is hereby incorporated by reference in its entirety); cervical carcinoma cells (HeLa cells and Me-180-VCII) (see Calabro-Jones); colon carcinoma (see Calabro-Jones); lung cancer (A549 cells and H1299): (verbal communication from Dr. A. Kajon; Pataer et al., “Induction of Apoptosis in Human Lung Cancer Cells Following Treatment With Amifostine and an Adenoviral Vector Containing Wild-Type p53,” Cancer Gene Ther. 13 (8): 806-14 (2006), each of which is hereby incorporated by reference in its entirety); and myelodysplastic syndrome (Ribizzi et al., “Amifostine Cytotoxicity and Induction of Apoptosis in a Human Myelodysplastic Cell Line,” Leuk. Res. 24 (6): 519 525 (2000), which is hereby incorporated by reference in its entirety). Sensitive tumor types identified through in vivo studies include, for example, metastatic melanoma (Glover et al., “WR-2721 and High-Dose Cisplatin: An Active Combination in the Treatment of Metastatic Melanoma,” J. Clin. Oncol. 5 (4): 574-578 (1987), which is hereby incorporated by reference in its entirety); radiation-induced tumor types (Grdina et al., “Protection Against Late Effects of Radiation by S-2-(3-aminopropylamino)-ethylphosphorothioic Acid,” Cancer Res. 51 (16): 4125-4130 (1991), which is hereby incorporated by reference in its entirety) (reporting that amifostine reduced the occurrence of all tumors representing 160 tumor classification codes for a wide range of radiation-induced tumor types in mice); lymphoreticular tumors (e.g., fibrosarcoma-lymph node, histiocytic leukemia, histiocytic lymphoma, lymphocytic-lymphoblastic lymphoma, myelogenous leukemia, plasma cell tumors, undifferentiated leukemia, undifferentiated lymphoma, unclassified lymphoma, mixed histiocytic-lymphocytic leukemia, and mixed histiocytic-lymphocytic lymphoma) (Grdina et al., “Protection Against Late Effects of Radiation by S-2-(3-aminopropylamino)-ethylphosphorothioic Acid,” Cancer Res. 51 (16): 4125-30 (1991), which is hereby incorporated by reference in its entirety); radiation-induced mammary tumors (Inano et al., “Inhibitory Effects of WR-2721 and Cysteamine on Tumor Initiation in Mammary Glands of Pregnant Rats by Radiation,” Radiat Res. 153 (1): 68-74 (2000), which is hereby incorporated by reference in its entirety); radiation-induced sarcomas (Milas et al. “Inhibition of Radiation Carcinogenesis in Mice by S-2-(3-Aminopropylamino)-ethylphosphorothioic Acid,” Cancer Res. 44 (12 Pt 1): 5567-5569 (1984), which is hereby incorporated by reference in its entirety); neutron-induced tumorigenicity (Grdina et al., “Protection by WR-151327 Against Late-Effect Damage From Fission-Spectrum Neutrons,” Radiat. Res. 128 (1 Suppl): S124-7 (1991) (reporting that the WR1065 analog WR151327 reduced fission-spectrum neutron-induced tumor induction in male and female mice when administered 30 min prior to irradiation) and Carnes et al., “In Vivo Protection by the Aminothiol WR-2721 Against Neutron-Induced Carcinogenesis,” Int. J. Radiat. Biol. 61 (5): 567-576 (1992) (reporting that WR2721 protected against neutron-induced tumor induction in male and female B6C3F1 mice), each of which is hereby incorporated by reference in its entirety); myelodysplastic syndrome (Mathew et al., “A Phase II Study of Amifostine in Children With Myelodysplastic Syndrome: A Report From the Children's Oncology Group Study (AAML0121),” Pediatr. Blood Cancer 57 (7): 1230-1232 (2011), which is hereby incorporated by reference in its entirety); and secondary tumors induced by radiation or chemotherapy in animal models (Grdina et al., “Protection Against Late Effects of Radiation by S-2-(3-Aminopropylamino)-ethylphosphorothioic Acid,” Cancer Res. 51 (16): 4125-4130 (1991); Grdina et al., “Radioprotectants: Current Status and new Directions,” Oncology 63 (Suppl. 2): 2 10 (2002); Grdina et al., “Radioprotectors in Treatment Therapy to Reduce Risk in Secondary Tumor Induction,” Pharmacol. Ther. 39 (1-3): 21-25 (1988), each of which is hereby incorporated by reference in its entirety). Tumor types demonstrated to be sensitive to the anti-neoplastic effects of WR1065 (delivered as an aminothiol-conjugate according to the present disclosure) also include prostate cancer and pancreatic cancer.

Further, the anti-neoplastic (or anti-cancer) effects of aminothiols (e.g., WR-1065 delivered as amifostine (WR2721) and WR1065)) have been well-established. Exemplary anti-neoplastic (or anti-cancer) effects include anti-neoplastic transformation, anti-mutagenesis in normal cells, anti-angiogenesis, inhibition or reduction of tumor cell growth, inhibition or reduction of tumor cell invasion, and inhibition or reduction of tumor cell metastasis. Exemplary anti-neoplastic (or anti-cancer) effects identified through in vitro or in vivo studies are summarized below.

Anti-neoplastic transformation: In in vitro experiments, V79 cells were irradiated with gamma rays and exposed to 1 milliM WR1065 simultaneously and the incidence of neoplastic transformation was assessed (Hill et al., “2-[(Aminopropyl)amino]ethanethiol (WR1065) is Anti-Neoplastic and Anti-Mutagenic When Given During 60Co Gamma-Ray Irradiation,” Carcinogenesis 7 (4): 665-668 (1986), which is hereby incorporated by reference in its entirety). Neoplastic transformation was reduced significantly even though cell viability was not changed. In in vitro experiments, WR1065 and WR151326, at 1 milliM each, were shown to protect C3H/10T1/2 cells from neoplastic transformation induced by exposure to fission neutrons, and this effect was observed for two different radiation exposure protocols (Balcer-Kubiczek et al, “Effects of WR-1065 and WR-151326 on Survival and Neoplastic Transformation in C3H/10T1/2 Cells Exposed to TRIGA or JANUS Fission Neutrons,” Int. J. Radiat. Biol. 63 (1): 37-46 (1993), which is hereby incorporated by reference in its entirety). The cells were exposed to WR1065 or WR151326 before, during, and after the radiation exposure. The protection factor for WR1065 was 3.23, while for WR151326 it was 1.8. In in vivo experiments, WR2721 administered at 100 micrograms/g body weight protected young rats from the formation of radiation-induced hepatic foci; this effect was more pronounced in female rats, the gender most susceptible to hepatocellular focus formation (Grdina et al, “Protective Effect of S-2-(3-Aminopropylamino) ethylphosphorothioic Acid Against Induction of Altered Hepatocyte Foci in Rats Treated Once With Gamma-Radiation Within one day After Birth,” Cancer Res. 45 (11 Pt 1): 5379-5381 (1985), which is hereby incorporated by reference in its entirety). In in vivo experiments, WR2721 exposure inhibited radiation-induced cell transformation in a mouse model, with 26% of mice receiving WR2721 plus radiation developing tumors, compared to 87% of mice receiving radiation alone (Milas et al. “Inhibition of Radiation Carcinogenesis in Mice by S-2-(3-Aminopropylamino)-ethylphosphorothioic Acid,” Cancer Res. 44 (12 Pt 1): 5567-5569 (1984), which is hereby incorporated by reference in its entirety). In vivo studies were conducted to determine if WR2721 could protect immune system cells from the damaging effects (lung colonization and increased tumor take/seeding capacity using a fibrosarcoma) of whole body irradiation plus chemotherapy with cyclophosphamide in a mouse model (Milas et al., “Protection by S-2-(3-Aminopropylamino) ethylphosphorothioic Acid Against Radiation- and Cyclophosphamide-Induced Attenuation in Antitumor Resistance,” Cancer Res. 44 (6): 2382-2386 (1984), which is hereby incorporated by reference in its entirety). The authors found that WR2721 almost completely eliminated the tumor-take enhancing effects of whole body irradiation in C3Hf/Kam mice. In in vivo experiments, female C57/BL/6JANL×BALB/cJANLF1 mice were exposed to 0, 206 cGy gamma rays, 417 cGy gamma rays, or the same doses of radiation with 400 mg/kg WR2721; animals were held for life (Grdina et al., “Protection Against Late Effects of Radiation by S-2-(3-Aminopropylamino)-ethylphosphorothioic Acid,” Cancer Res. 51 (16): 4125-30 (1991), which is hereby incorporated by reference in its entirety). 90% of the irradiated animals died of tumors; significant protection was seen for WR2721 treated mice that were irradiated with 206 cGy. Lymphoreticular tumors were particularly sensitive to the protective effect; total life expectancy was extended 65 days. In in vivo experiments, Amifostine has been shown to reduce radiation-induced mammary tumors in pregnant rats (Grdina et al., “Amifostine: Mechanisms of Action Underlying Cytoprotection and Chemoprevention,” Drug Metabol. Drug Interact. 16 (4): 237-279 (2000), which is hereby incorporated by reference in its entirety).

Anti-Mutagenesis In Normal Cells: In in vitro experiments, using WR1065 at 4 milliM and simultaneous gamma ray irradiation of V79 cells, HPRT mutations were reduced significantly and cell viability was increased (Hill et al., “2-[(Aminopropyl)amino]ethanethiol (WR1065) is Anti-Neoplastic and Anti-Mutagenic When Given During 60Co Gamma-Ray Irradiation,” Carcinogenesis 7 (4): 665-668 (1986), which is hereby incorporated by reference in its entirety). In in vitro experiments, a dose of 4 milliM resulted in significant increases in cellular glutathione levels and cysteine levels, and these were associated with significant cytoprotection and anti-mutagenesis against 60Co gamma-photon and neutron radiation (Grdina et al., “Thiol and Disulfide Metabolites of the Radiation Protector and Potential Chemopreventive Agent WR-2721 are Linked to Both its Anti-Cytotoxic and Anti-Mutagenic Mechanisms of Action,” Carcinogenesis 16 (4): 767 774 (1995), which is hereby incorporated by reference in its entirety). In in vitro experiments, WR1065 protected GO T-lymphocytes from mutation induction due to ionizing radiation, showing protection in a non-cycling cell (Clark et al., “Hprt Mutations in Human T-Lymphocytes Reflect Radioprotective Effects of the Aminothiol, WR-1065,” Carcinogenesis 17 (12): 2647-2653 (1996), which is hereby incorporated by reference in its entirety). In in vitro experiments in GO T-lymphocytes, WR1065 reduced the induction of mutations indicative of gross structural alterations (Clark et al., “The Aminothiol WR-1065 Protects T Lymphocytes From Ionizing Radiation-Induced Deletions of the HPRT Gene,” Cancer Epidemiol. Biomarkers. Prev. 6 (12): 1033-1037 (1997), which is hereby incorporated by reference in its entirety). In in vitro experiments, amifostine reduced cyclophosphamide-induced mutations in the HPRT gene 8-fold in mouse splenocytes (Grdina et al., “Chemopreventive Doses of Amifostine Confer no Cytoprotection to Tumor Nodules Growing in the Lungs of Mice Treated With Cyclophosphamide,” Semin. Oncol. 26 (2 Suppl 7): 22-27 (1999), which is hereby incorporated by reference in its entirety). In in vitro experiments using a mouse model injected IV with fibrosarcoma cells intended to colonize the lung, the ability of WR1065 to prevent HPRT mutations due to cyclophosphamide exposure was evaluated (Kataoka et al., “Antimutagenic Effects of Amifostine: Clinical Implications,” Semin. Oncol. 23 (4 Suppl 8): 53-57 (1996), which is hereby incorporated by reference in its entirety). At 100 mg/kg, WR1065 did not reduce the anticancer effectiveness of cyclophosphamide, but did reduce significantly HPRT mutation frequencies induced by this chemotherapeutic agent. In in vitro experiments, it was found that WR1065, at a concentration of 4 milliM, provided significant protection against induction of mutations in the HPRT gene due to exposure to the chemotherapeutic agent cis-DDP (Nagy et al., “Protection Against cis-diamminedichloroplatinum Cytotoxicity and Mutagenicity in V79 Cells by 2-[(Aminopropyl)amino]ethanethiol,” Cancer Res. 46 (3): 1132-1135 (1986), which is hereby incorporated by reference in its entirety). In in vitro experiments, the ability of WR1065, at 4 milliM, to protect against mutation induction in the HPRT gene, induction of single strand breaks, and cell killing by bleomycin, nitrogen mustard, cis-DDP, or x-ray radiation was assessed (Nagy et al., “Protective Effects of 2-[(Aminopropyl)amino] Ethanethiol Against Bleomycin and Nitrogen Mustard-Induced Mutagenicity in V79 Cells,” Int. J. Radiat. Oncol. 12 (8): 1475-1478, (1986.), which is hereby incorporated by reference in its entirety). WR1065 protected against all of these effects for each agent, but the degree of protection varied with the agent. In in vitro experiments, WR1065 and WR151326 were tested for their ability to prevent mutation induction at the HPRT gene due to exposure to fission-spectrum neutrons (Grdina et al., “Protection by WR1065 and WR151326 Against Fission-Neutron-Induced Mutations at the HGPRT Locus in V79 Cells,” Radiat. Res. 117 (3): 500-510 (1989), which is hereby incorporated by reference in its entirety). Both agents protected against mutation induction, with WR1065 being more effective than WR151326 at preventing mutations. In in vivo experiments using B6C3F1 male mice, the ability of WR2721, at a dose of 400 mg/kg, to protect against mutation induction by JANUS fission-spectrum neutrons was assessed (Grdina et al., “The Radioprotector WR-2721 Reduces Neutron-Induced Mutations at the hypoxanthine-guanine Phosphoribosyl Transferase Locus in Mouse Splenocytes When Administered Prior to or Following Irradiation,” Carcinogenesis 13 (5): 811-814 (1992), which is hereby incorporated by reference in its entirety). WR1065 reduced the mutant frequency when administered before, during, or after irradiation. However, the highest reduction factor was obtained when the dose administered was 50 mg/kg instead of 400 mg/kg.

Anti-Angiogenesis: Amifostine reduced the mRNA levels of VEGF isoforms VEGF (165) and VEGF (190) and angiogenesis in chicken embryo chorioallantoic membranes at doses not associated with signs of toxicity (Giannopoulou et al., “Amifostine has Antiangiogenic Properties in Vitro by Changing the Redox Status of Human Endothelial Cells,” Free Radic. Res. 37 (11): 1191-1199 (2003), which is hereby incorporated by reference in its entirety). WR2721 also reduced the mRNA levels of inducible nitric oxide synthase, and also reduced laminin and collagen deposition amounts in the same model “without affecting the expression of the corresponding genes.” See id. MMP-2 protein levels were not affected, but gene expression was reduced. Last, plasmin activity was increased by amifostine. The authors concluded that these effects showed evidence that WR1065 inhibits angiogenesis. In another study, amifostine was shown to increase serum angiostatin levels 4-fold (Grdina et al., “Inhibition of Spontaneous Metastases Formation by Amifostine,” Int. J. Cancer 97 (2): 135-141 (2002), which is hereby incorporated by reference in its entirety). Using the same in vivo mouse model system that was used in Grdina et al. (Grdina et al., “Inhibition of Spontaneous Metastases Formation by Amifostine,” Int. J. Cancer 97 (2): 135-141 (2002), which is hereby incorporated by reference in its entirety), the authors found that doses of WR2721 of 200 mg/ml (instead of 50 mg/ml) did not change angiostatin levels (Grdina et al., “Antimetastatic Effectiveness of Amifostine Therapy Following Surgical Removal of Sa-NH Tumors in Mice,” Semin. Oncol. 29 (6 Suppl 19): 22-28 (2002), which is hereby incorporated by reference in its entirety). The authors concluded that the mechanism for these effects was a redox driven process.

Inhibition or Reduction of Tumor Cell Growth: In a study relating to radiation-induced sarcomas, one half of mice were exposed to amifostine and 30 mins later the right hind legs of all mice (controls and amifostine-treated) were exposed to 3400 to 5700 rads (Milas et al. “Inhibition of Radiation Carcinogenesis in Mice by S-2-(3-Aminopropylamino)-ethylphosphorothioic Acid,” Cancer Res. 44 (12 Pt 1): 5567-5569 (1984), which is hereby incorporated by reference in its entirety). Tumor cell growth rate was decreased in WR-2721 plus radiation-exposed mice when compared to mice exposed to radiation alone. In a study relating to Sa-NH sarcoma cells, C3Hf/Kam mice were injected with Sa-NH sarcoma cells and treated with WR2721 at 50 mg/kg every other day for 6 days while the tumors grew; then the tumors were removed by limb amputation and WR2721 was administered immediately after surgery and again 2 days later (Grdina et al., “Inhibition of Spontaneous Metastases Formation by Amifostine,” Int. J. Cancer 97 (2): 135-141 (2002), which is hereby incorporated by reference in its entirety). Results to this point showed that amifostine was able to induce a slight delay in tumor growth, from 12 to 13 days for tumors to reach ideal size for amputation. In a study using Chinese hamster ovarian cells (CHO cells), WR1065 when administered at 4 milliM to Chinese hamster ovarian cells resulted in cell cycle delay at the G2/M phase (Grdina et al., “Inhibition of Topoisomerase II Alpha Activity in CHO K1 Cells by 2-[(Aminopropyl)amino]ethanethiol (WR-1065),” Radiat. Res. 138 (1): 44-52 (1994), which is hereby incorporated by reference in its entirety.) In a further study relating to CHO cells, WR1065 exposure in the range of 4 microM to 4 millimolar for 30 min resulted in cell accumulation in G2 (Murley et al., “WR-1065, An Active Metabolite of the Cytoprotector Amifostine, Affects Phosphorylation of Topoisomerase II Alpha Leading to Changes in Enzyme Activity and Cell Cycle Progression in CHO AA8 Cells,” Cell Prolif. 30 (6-7): 283-294 (1997), which is hereby incorporated by reference in its entirety). Further, it has been shown that WR1065-induced inhibition of topoisomerase II-alpha can result in alteration in cell population distribution throughout the cell cycle (Kataoka et al., “Activation of the Nuclear Transcription Factor kappaB (NFkappaB) and Differential Gene Expression in U87 Glioma Cells After Exposure to the Cytoprotector Amifostine,” Int. J. Radiat. 53 (1): 180-189 (2002), which is hereby incorporated by reference in its entirety).

Inhibition or Reduction of Tumor Cell Invasion: In in vitro experiments in a model using chicken embryo chorioallantoic membranes, WR1065, at doses not associated with signs of toxicity, reduced gene expression of MMP-2 (an enzyme associated with tumor cell invasion), but protein levels were not affected (Giannopoulou et al., “Amifostine has Antiangiogenic Properties in Vitro by Changing the Redox Status of Human Endothelial Cells,” Free Radic. Res. 37 (11): 1191-9 (2003), which is hereby incorporated by reference in its entirety). In in vitro experiments, WR2721 decreased the activity of matrix metalloproteinases (MMPs) −2 and −9 by 30 to 40%. WR2721 also inhibited the migration of Sa-NH cells through Matrigel in a dose dependent manner (Grdina et al., “Inhibition of Spontaneous Metastases Formation by Amifostine,” Int. J. Cancer 97 (2): 135-41 (2002), which is hereby incorporated by reference in its entirety). In in vivo experiments, C3Hf/Kam mice were injected with Sa-NH sarcoma cells and treated with WR2721 at 50 mg/kg every other day for 6 days while the tumors grew; then the tumors were removed by limb amputation and WR2721 was administered immediately after surgery and again 2 days later (Grdina et al., “Inhibition of Spontaneous Metastases Formation by Amifostine,” Int. J. Cancer 97 (2): 135-41 (2002), which is hereby incorporated by reference in its entirety).

Inhibition or Reduction of Tumor Cell Metastasis: For the purpose of investigating the anti-metastatic effects of WR1065, C3Hf/Kam mice were injected with Sa-NH sarcoma cells and treated with WR2721 at 50 mg/kg every other day for 6 days while the tumors grew; then the tumors were removed by limb amputation and WR2721 was administered immediately after surgery and again 2 days later (Grdina et al., “Inhibition of Spontaneous Metastases Formation by Amifostine,” Int. J. Cancer 97 (2): 135-41 (2002), which is hereby incorporated by reference in its entirety). Amifostine reduced the number of animals with metastases and the number of metastases per animal. In another study, Amifostine was shown to have paradoxical effects; pulmonary metastases were reduced significantly in animals administered 50 mg/kg. The dose of 100 mg/kg was less effective and 200 mg/kg had no effect on metastases in this study (Grdina et al., “Antimetastatic Effectiveness of Amifostine Therapy Following Surgical Removal of Sa-NH Tumors in Mice,” Semin. Oncol. 29 (6 Suppl 19): 22-8 (2002), which is hereby incorporated by reference in its entirety). In a further study, it was found that WR2721 almost completely eliminated the tumor-take enhancing effects of whole body irradiation in C3Hf/Kam mice, and significantly reduced lung nodule formation in mice that received WBI (whole body irradiation) with or without cyclophosphamide 5 days earlier (Milas et al., “Protection by S-2-(3-Aminopropylamino)ethylphosphorothioic Acid Against Radiation- and Cyclophosphamide-Induced Attenuation in Antitumor Resistance,” Cancer Res. 44 (6): 2382-6 (1984), which is hereby incorporated by reference in its entirety). Further, of the partial responses observed in patients with metastatic melanoma, 53% occurred in patients who had received prior chemotherapy, and metastatic sites that responded included subcutaneous sites, lymph nodes, lung, and liver (Glover et al., “WR-2721 and High-Dose Cisplatin: An Active Combination in the Treatment of Metastatic Melanoma,” J. Clin. Oncol. 5 (4): 574-8 (1987), which is hereby incorporated by reference in its entirety). The mean response time was 4.5 months.

In addition to the anti-cancer effects described above, aminothiols (e.g., amifostine (WR2721) and WR1065) have been shown to have effects on other anti-cancer therapies. Exemplary effects on other anti-cancer therapies include enhancement of other anti-cancer therapies (e.g., enhanced cytotoxicity of chemotherapeutic agents, enhanced cytotoxic effects of radiation therapy, improved response to chemotherapy, selective radioprotective effect on non-cancerous cells). Exemplary effects on other anti-cancer therapies identified through in vitro or in vivo studies are summarized below.

Enhancement of anti-cancer therapies: In in vitro experiments, WR1065 enhanced the cytotoxicity of the chemotherapeutic agent bleomycin in human lymphocytes at the GO stage of the cell cycle (Hoffmann et al., “Structure-Activity Analysis of the Potentiation by Aminothiols of the Chromosome-Damaging Effect of Bleomycin in GO Human Lymphocytes,” Environ. Mol. Mutagen. 37 (2): 117-27 (2001), which is hereby incorporated by reference in its entirety). In in vitro experiments, WR2721 combined with mafosfamide resulted in survival of normal myeloid and erythroid progenitor cells while increasing the degree of cell death of leukemic cells (List, “Use of Amifostine in Hematologic Malignancies, Myelodysplastic Syndrome, and Acute Leukemia,” Semin. Oncol. 26 (2 Suppl 7): 61-5 (1999), which is hereby incorporated by reference in its entirety). In in vivo experiments, the combination of WR2721 and cisplatin resulted in improved partial responses compared to cisplatin alone (53% partial response versus 10%, respectively) in patients with advanced malignant melanoma (Glover et al., “WR-2721 and High-Dose Cisplatin: An Active Combination in the Treatment of Metastatic Melanoma,” J. Clin. Oncol. 5 (4): 574-8 (1987), which is hereby incorporated by reference in its entirety. Minor responses were observed in an additional 3 out of 36 patients (8%). In in vivo experiments in the Canine Sarcoma Study, evidence was found that WR2721 enhanced the cytotoxic effects of radiation therapy for a subset of tumors, and did not affect the cytotoxicity of radiation in the remaining tumors (Koukourakis, “Amifostine: Is There Evidence of Tumor Protection?” Semin. Oncol. 30 (6 Suppl. 18): 18-30 (2003), which is hereby incorporated by reference in its entirety). In in vivo experiments, WR2721 had synergistic cytotoxicity when administered to mice in combination with oxygen radical-generating chemotherapeutic agents. In mice treated with WR2721, the glutathione synthesis pathway appeared to be inactivated. In addition, WR33278 was found to have strong inhibitory effects upon gamma-glutamylcysteine synthetase, which is the rate limiting enzyme in glutathione synthesis. Similar results were obtained for cysteamine and for oxygen radicals. Oxygen radicals increased the rate at which WR1065 was oxidized to WR33278 (Schor, “Mechanisms of Synergistic Toxicity of the Radioprotective Agent, WR2721, and 6-hydroxydopamine,” Biochem. Pharmacol. 37 (9): 1751-62 (1988), which is hereby incorporated by reference in its entirety). In in vivo experiments, WR2721 given 30 minutes before whole body irradiation significantly increased the local radiocurability of 8 mm diameter fibrosarcoma tumors (Milas et al., “Protection by S-2-(3-Aminopropylamino)ethylphosphorothioic Acid Against Radiation- and Cyclophosphamide-Induced Attenuation in Antitumor Resistance,” Cancer Res. 44 (6): 2382-6 (1984), which is hereby incorporated by reference in its entirety). In in vivo experiments in mice bearing subcutaneous human ovarian carcinoma xenografts OVCAR-3, WR2721 enhanced the anti-tumor efficacy of carboplatin (Treskes et al., “Effects of the Modulating Agent WR2721 on Myelotoxicity and Antitumour Activity in Carboplatin-Treated Mice,” Eur. J. Cancer. 30A (2): 183-7 (1994), which is hereby incorporated by reference in its entirety). In in vivo experiments in a mouse model of two different tumor types, when amifostine was combined with MISO, additive toxicity effects were observed (Rojas et al., “Interaction of Misonidazole and WR-2721-II. Modification of Tumour Radiosensitization,” Br. J. Cancer 47 (1): 65-72 (1983), which is hereby incorporated by reference in its entirety). Effects of the drugs appeared to be related to the oxygen status of the tumors and MISO can act as an oxygen-mimetic to reduce the radioprotection of WR2721. In in vivo experiments, amifostine was shown to enhance the cytotoxic effects of some chemotherapeutic agents such as cisplatin, carboplatin, and paclitaxel (Kurbacher et al., “Chemoprotection in Anticancer Therapy: The Emerging Role of Amifostine (WR-2721),” Anticancer Res. 18 (3C): 2203-10 (1998), which is hereby incorporated by reference in its entirety).

Further, it has been shown that anticancer effects occur at drug doses that lack or have minimal cytotoxic effects in normal cells, including bovine arterial endothelial cells (see Brenner et al., “Variable Cytotoxicity of Amifostine in Malignant and Non-Malignant Cell Lines,” Oncol. Rep. 10 (5): 1609-13 (2003), which is hereby incorporated by reference in its entirety); liver (in vivo) (Shaw et al., “Metabolic Pathways of WR-2721 (ethyol, amifostine) in the BALB/c Mouse,” Drug Metab. Dispos. 22 (6): 895-902 (1994) (no observable cytotoxicity at >7400 picomol/10 (6) cells); kidney (in vivo) (Shaw et al., “Metabolic Pathways of WR-2721 (ethyol, amifostine) in the BALB/c Mouse,” Drug Metab. Dispos. 22 (6): 895-902 (1994) (no observable cytotoxicity at >17,000 picomol/10 (6) cells); small intestine (in vivo) (Shaw et al., “Metabolic Pathways of WR-2721 (ethyol, amifostine) in the BALB/c Mouse,” Drug Metab. Dispos. 22 (6): 895-902 (1994) (no observable cytotoxicity at >3000 picomol/10 (6) cells). Each of the above-cited references is hereby incorporated by reference in its entirety.

The combination of the aminothiol-conjugate and the therapy (e.g., anti-neoplastic drug or the antimicrobial) described herein may have a synergistic effect.

The term synergistic refers to a therapeutic combination that is more effective than the additive effect of the two or more individual agents of the combination. Determining the synergistic interaction between the aminothiol-conjugates described herein and one or more therapeutic agents (e.g., anti-neoplastic drug or the antimicrobial) may be based on the results of the tests described herein. For example, drug combination responses may be calculated based on the highest single agent (HSA) model (Yadav et al., “Searching for Drug Synergy in Complex Dose-Response Landscapes Using an Interaction Potency Model,” Comput. Struct. Biotechnol. J. 13:504-513 (2015), which is hereby incorporated by reference in its entirety). For example, certain combinations described herein have been evaluated in several analytical systems and the obtained data can be processed using a standard program for the quantitative determination of synergism, additive effect, and antagonism with antitumor agents. Using the HSA model, synergy scores <−10 indicate the interaction between two drugs is antagonistic; scores from −10 to 10 indicate the interaction between two drugs is additive; and scores >10 indicate the interaction between two drugs is synergistic. Accordingly, in certain embodiments, the combination of the aminothiol-conjugate and the therapy achieves an HSA synergy score of >10, >15, >20, >25, >30, >35, >40, >45, >50, or more. In certain embodiments, the combination of the aminothiol-conjugate and the therapy achieves an HSA synergy score as described herein, see e.g., Table 3. The synergistic effects may be achieved in connection with the cell types and subjects described herein.

The combination of the aminothiol-conjugate and the therapy (e.g., anti-neoplastic drug or the antimicrobial) described herein can provide “synergy” and be “synergistic” when the effect achieved with the combination is greater as compared to the sum of the effects achieved when using the compounds separately. A synergistic effect can be achieved when the active ingredients are administered in combination as described herein.

Accordingly, the subject according to the present disclosure may be one that is suffering from a neoplastic condition and the combination therapeutic is administered under conditions effective to treat the neoplastic condition. Treatment may include any anti-cancer effect in the subject, as described herein (e.g., anti-neoplastic transformation, anti-mutagenesis in normal cells, anti-angiogenesis, inhibition or reduction of tumor cell growth, inhibition or reduction of tumor cell invasion, and inhibition or reduction of tumor cell metastasis).

As used herein, the terms “neoplasm”, “neoplastic disorder”, “neoplasia”, “cancer”, “tumor”, and “proliferative disorder” refer to cells having the capacity for autonomous growth, i.e., an abnormal state of condition characterized by rapidly proliferating cell growth which generally forms a distinct mass that show partial or total lack of structural organization and functional coordination with normal tissue. The terms are meant to encompass hematopoietic neoplasms (e.g., lymphomas or leukemias) as well as solid neoplasms (e.g., sarcomas or carcinomas), including all types of pre-cancerous and cancerous growths, or oncogenic processes, metastatic tissues or malignantly transformed cells, tissues, or organs, irrespective of histopathologic type or stage of invasiveness. Hematopoietic neoplasms are malignant tumors affecting hematopoietic structures (structures pertaining to the formation of blood cells) and components of the immune system, including leukemias (related to leukocytes (white blood cells) and their precursors in the blood and bone marrow) arising from myeloid, lymphoid or erythroid lineages, and lymphomas (relates to lymphocytes). Solid neoplasms include sarcomas, which are malignant neoplasms that originate from connective tissues such as muscle, cartilage, blood vessels, fibrous tissue, fat or bone. Solid neoplasms also include carcinomas, which are malignant neoplasms arising from epithelial structures (including external epithelia (e.g., skin and linings of the gastrointestinal tract, lungs, and cervix), and internal epithelia that line various glands (e.g., breast, pancreas, thyroid). Examples of neoplasms that are particularly susceptible to treatment by the methods of the disclosure include leukemia, breast cancers, prostate cancers, lung cancers, ovarian and pancreatic cancer; neoplasms that are expected to be susceptible to treatment by the methods of the disclosure include hepatocellular cancers, sarcoma, vascular endothelial cancers, central nervous system cancers (e.g., astrocytoma, gliosarcoma, neuroblastoma, oligodendroglioma, and glioblastoma), bronchus cancers, larynx cancers, esophagus cancers, colon cancers, colorectal cancers, gastro-intestinal cancers, melanomas, endometrial cancer, renal and bladder cancer, liver cancer, and endocrine cancer (e.g., thyroid).

In some embodiments, the neoplastic condition is selected from the group consisting of breast cancer (e.g., a breast cancer associated with TP53 and PIK3CA mutations, a breast cancer associated with TP53, BRAF, and KRAS mutations), non-small cell lung cancer (e.g., a lung cancer associated with TP53 wild-type; (DKN2A, KRAS, and STK1/mutations; and/or over-expression of EGFR protein, a lung cancer associated with TP53 wild-type; and/or (DKN2A, KRAS, MAPK, MYC and PIK3CA, and/or STK11 mutations, a lung cancer associated with TP53, JAK, MAPK, and NRTK3 mutations, and lung cancer associated with EGFR L858R and T790M mutations plus TP53 and PIK3CA mutations), a malignant pleural mesothelioma (e.g., a malignant pleural mesothelioma associated with a TP53 mutation), a pancreatic cancer (e.g., a pancreatic cancer associated with TP53 null, KRAS, and (DKN2A/p16 mutations), a prostate cancer (e.g., a prostate cancer associated with TP53, BRAF, BRCA2, EGFR, JAK2, MAPK, NRTK3, PIK3CA, and STK11 mutations; a prostate cancer associated with TP53 wild-type; ABL1/2, AKT2, ALK, BRCA1 2, EGFR, ERBB2/3/4, FLT3 4, VEGE, JAK, KIT, MAPK, MET, mTOR, MYC, NTRK3, ROS mutations; a prostate cancer associated with TP53 mutation, chromosome 17p deletion, and PTEN LOH), an ovarian cancer (e.g., an ovarian cancer associated with TP53 null, HRAS, and PIK3CA mutations, an ovarian cancer associated with TP53 wild-type; and/or hypermutated with BRCA2, KRAS, and PIK3CA mutations), and a leukemia (e.g., a leukemia associated with biallelic deletion of TP53, NRAS mutation, and C-MYC amplification).

In some embodiments of the methods and combination therapeutics of the present disclosure, the neoplastic condition is selected from the group consisting of lung cancer (e.g., non-small lung cancer), breast cancer, ovarian cancer, cervical cancer, colon cancer, skin cancer (e.g., melanoma or malignant melanoma), lymphoreticular tumors, prostate cancer, colorectal cancer, bladder cancer, lymphoma (e.g., Hodgkin lymphoma or non-Hodgkin lymphoma), endometrial cancer, leukemia, kidney cancer, pancreatic cancer, thyroid cancer, liver cancer, sarcomas, myelodysplastic condition, undifferentiated tumors, and combinations thereof.

As used herein, anti-neoplastic drug refers to agents (e.g., chemotherapeutic agents) toxic to neoplastic or cancer cells, which drugs are known in the art (see, e.g., U.S. Patent Appl. Pub. Nos. 2019/0323089 and 2021/0093715, each of which is hereby incorporated by reference in its entirety). A chemotherapeutic agent is a chemical compound useful in the treatment of cancer. Examples of chemotherapeutic agents include: alkylating agents (e.g., thiotepa and cyclosphosphamide (CYTOXAN®); alkyl sulfonates (e.g., busulfan, improsulfan, and piposulfan); aziridines (e.g., benzodopa, carboquone, meturedopa, and uredopa); ethylenimines and methylamelamines (e.g., altretamine, triethylenemelamine, trietylenephosphoramide, triethiylenethiophosphoramide, and trimethylolomelamine); acetogenins (e.g., bullatacin and bullatacinone); delta-9-tetrahydrocannabinol (e.g., dronabinol, MARINOL®); beta-lapachone; lapachol; colchicines; betulinic acid; camptothecins (e.g., topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin, and bizelesin synthetic analogues); podophyllotoxin; podophyllinic acid; teniposide; cryptophycins (e.g., cryptophycin 1 and cryptophycin 8); dolastatin; duocarmycin (including, e.g., KW-2189 and CB1-TM1); eleutherobin; pancratistatin; sarcodictyin; spongistatin; nitrogen mustards (e.g., chlorambucil, cyclophosphamide, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard); nitrosureas (e.g., carmustine, chlorozotocin, fotemustine, lomustine, nimustine, and ranimnustine); antibiotics such as the enediyne antibiotics (e.g., calicheamicins such as calicheamicin gammall and calicheamicin omegall); dynemicin, (including, e.g., dynemicin A); esperamicin; as well as neocarzinostatin chromophore and related chromoprotein enediyne antiobiotic chromophores, aclacinomy sins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites (e.g., methotrexate, cytarabine, gemcitabine (GEMZAR®), tegafur (UFTORAL®), capecitabine (XELODA®), an epothilone, and 5-fluorouracil (5-FL)); folic acid analogues (e.g., denopterin, methotrexate, pteropterin, and trimetrexate); purine analogs (e.g., fludarabine, 6-mercaptopurine, thiamiprine, and thioguanine); pyrimidine analogs (e.g., ancitabine, azacitidine, 6-azauridine, carmofur, cytarabine, dideoxyuridine, doxifluridine, enocitabine, and floxuridine): anti-adrenals (e.g., aminoglutethimide, mitotane, and trilostane); folic acid replenisher (e.g., frolinic acid); aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; etoglucid; gallium nitrate; hydroxyurea; lentinan; lonidainine: maytansinoids (e.g., maytansine and ansamitocins); mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran, spirogermanium; tenuazonic acid; triaziquone; 2,2′,2″-trichlorotriethylamine; trichothecenes (e.g., T-2 toxin, verracurin A, roridin A and anguidine): urethan; vindesine (ELDISINE®, FILDESIN®); dacarbazine; mannomustine; mitobronitol; mitolactol; pipobroman; gacytosine; arabinoside (“Ara-C”); thiotepa; taxoid (e.g., paclitaxel (TAXOL®), albumin-engineered nanoparticle formulation of paclitaxel (ABRAXANE™), and docetaxel (TAXOTERE®)); chloranbucil; 6-thioguanine; mercaptopurine: methotrexate; platinum analogs such as cisplatin and carboplatin; vinblastine (VELBAN®); platinum; etoposide (VP-16); ifosfamide; mitoxantrone; vincristine (ONCOVIN®); oxaliplatin: leucovovin; vinorelbine (NAVELBINE®); novantrone; edatrexate; daunomycin; aminopterin; ibandronate; topoisomerase inhibitor RFS 2000; difluorometlhylornithine (DMFO); retinoids such as retinoic acid; bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine: topoisomerase 1 inhibitor (e.g., LURTOTECAN®); rmRH (e.g., ABARELIX®); sorafenib (Bayer); SU-11248 (Pfizer); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

In some embodiments of the methods and combination therapeutics of the present disclosure, the anti-neoplastic drug is a protein kinase inhibitor. Suitable protein kinase inhibitors include, without limitation, imatinib, nilotinib, dasatinib, bosutinib, ponatinib, gefitinib, erlotinib, afatinib, osimertinib, lapatinib, neratinib, sorafenib, sunitinib, pazopanib, axitinib, lenvatinib, cabozatinib, vandetanib, regorafenib, vemurafenib, dabrafenib, trametinib, cobimetinib, crizotinib, certinib, alectinib, brigatinib, lorlatinib, ibrutinib, acalibrutinib, midostaurin, ruxolitinib, idelalisib, copanlisib, palbociclib, ribociclib, and abemaciclib (see, e.g., Kannaiyan and Mahadevan, “A Comprehensive Review of Protein Kinase Inhibitors for Cancer Therapy,” Expert. Rev. Anticancer Ther. 18 (12): 1249-1270 (2018), which is hereby incorporated by reference in its entirety).

In some embodiments of the methods and combination therapeutics of the present disclosure, the anti-neoplastic drug is a chemotherapeutic agent that is a platinum (e.g., cisplatin and carboplatin), taxane (e.g., paclitaxel), anthracycline (e.g., doxorubicin), antimetabolite (e.g., 5-fluorouracil), or tyrosine kinase inhibitor (e.g., imatinib). In some embodiments, the anti-neoplastic drug is selected from the group consisting of cisplatin, erlotinib, gefitinib, afatinib, osimertinib, abemaciclib, tetraplatin, ipraplatin, abemaciclib, carboplatin, oxaliplatin, nedaplatin, lobaplatin, and/or derivatives of any thereof. In some embodiments of the methods and combination therapeutics of the present disclosure, the anti-neoplastic drug is cisplatin. In other embodiments, the anti-neoplastic drug is gefitinib.

In some embodiments of the methods and combination therapeutics of the present disclosure, the combination therapeutic according to the present disclosure can inhibit tumor cells and reverse cells to a normal state; improve blood circulation in order to suppress the growth and metastasis of tumor; increase the sensitivity to radiotherapy and chemotherapy and reduce the toxic and adverse effects of radiotherapy and chemotherapy; enhance the anti-tumor ability of the organism; and/or prevent the relapse of tumors.

In some embodiments, the subject is one that receives radiation therapy, chemotherapy, or a combination thereof and the aminothiol-conjugate or pharmaceutical composition comprising aminothiol-conjugate is administered under conditions effective to reduce or decrease the adverse or undesirable side-effects of the radiation therapy, chemotherapy, or combination thereof.

In one embodiment, the subject is one that receives a cancer therapy (e.g., radiation therapy, chemotherapy, or a combination thereof) and the aminothiol-conjugate or pharmaceutical composition comprising aminothiol-conjugate is administered under conditions effective to enhance the efficacy of the cancer therapy, as compared to treatment with the cancer therapy alone.

In some embodiments, the neoplastic condition being treated is resistant to a treatment with the anti-neoplastic drug. In accordance with such embodiments, the combination therapeutic comprising an aminothiol-conjugate of Formula (I) and the neoplastic drug is administered to sensitize the cells and thus treat the neoplastic condition, where the condition is not responsive to treatment with the anti-neoplastic drug alone (due to resistance to that anti-neoplastic drug).

Sensitized cancer cells respond better to cancer therapy (are inhibited or killed faster or more often) than non-sensitized cells, as described herein below.

In methods described herein, the subject may be a mammal e.g., a human, a non-human primate, a dog, a cat, a horse, a cow, or a rodent.

In some embodiments, the mammal is a human. In some embodiments, the mammal is a non-human animal.

According to the present disclosure, the aminothiol-conjugate of Formula (I) and the anti-neoplastic drug can be formulated together in a single pharmaceutical composition or in separate pharmaceutical compositions. Suitable pharmaceutical compositions are described infra.

In some embodiments of the present disclosure, a dosage unit of the combination therapeutic comprises about 0.001 mg to about 1,000 mg of the aminothiol-conjugate of Formula (I); about 0.01 mg to about 500 mg of the aminothiol-conjugate of Formula (I); or about 0.001 mg to about 500 mg of the aminothiol-conjugate of Formula (I).

In some embodiments of the present disclosure a dosage unit of the combination therapeutic comprises about 0.001 mg to about 2,000 mg; about 0.001 mg to about 1,500 mg; about 0.001 mg to about 1,000 mg; about 0.001 mg to about 900 mg; about 0.001 mg to about 800 mg: about 0.001 mg to about 700 mg; about 0.001 mg to about 600 mg; about 0.001 mg to about 500 mg; about 0.001 mg to about 400 mg; about 0.001 mg to about 300 mg; about 0.001 mg to about 200 mg; about 0.001 mg to about 100 mg; about 0.001 mg to about 90 mg; about 0.001 mg to about 80 mg; about 0.001 mg to about 70 mg; about 0.001 mg to about 60 mg; about 0.001 mg to about 50 mg; about 0.001 mg to about 40 mg; about 0.001 mg to about 30 mg; about 0.001 mg to about 20 mg; about 0.001 mg to about 10 mg; about 0.001 mg to about 9 mg; about 0.001 mg to about 8 mg; about 0.001 mg to about 7 mg; about 0.001 mg to about 6 mg; about 0.001 mg to about 5 mg: about 0.001 mg to about 4 mg; about 0.001 mg to about 3 mg; about 0.001 mg to about 2 mg; about 0.001 mg to about 1 mg; about 0.001 mg to about 0.9 mg; about 0.001 mg to about 0.8 mg; about 0.001 mg to about 0.7 mg; about 0.001 mg to about 0.6 mg; about 0.001 mg to about 0.5 mg; about 0.001 mg to about 0.4 mg; about 0.001 mg to about 0.3 mg; about 0.001 mg to about 0.2 mg; about 0.001 mg to about 0.1 mg; about 0.001 mg to about 0.09 mg; about 0.001 mg to about 0.08 mg; about 0.001 mg to about 0.07 mg; about 0.001 mg to about 0.06 mg; about 0.001 mg to about 0.05 mg; about 0.01 mg to about 0.03 mg; about 0.001 mg to about 0.02 mg; about 0.001 mg to about 0.01 mg; about 0.001 mg to about 0.009 mg; about 0.001 mg to about 0.008 mg; about 0.001 mg to about 0.007 mg; about 0.001 mg to about 0.006 mg; about 0.001 mg to about 0.005 mg; about 0.001 mg to about 0.004 mg; about 0.001 mg to about 0.003 mg; or about 0.001 mg to about 0.002 mg of the aminothiol-conjugate of Formula (I).

In some embodiments of the present disclosure a dosage unit of the combination therapeutic comprises about 0.01 mg to about 500 mg; about 0.01 mg to about 400 mg; about 0.01 mg to about 300 mg; about 0.01 mg to about 200 mg; about 0.01 mg to about 100 mg; about 0.01 mg to about 90 mg; about 0.01 mg to about 80 mg; about 0.01 mg to about 70 mg; about 0.01 mg to about 60 mg; about 0.01 mg to about 50 mg; about 0.01 mg to about 40 mg; about 0.01 mg to about 30 mg; about 0.01 mg to about 20 mg; about 0.01 mg to about 10 mg; about 0.01 mg to about 9 mg; about 0.01 mg to about 8 mg; about 0.01 mg to about 7 mg; about 0.01 mg to about 6 mg; about 0.01 mg to about 5 mg; about 0.01 mg to about 4 mg; about 0.01 mg to about 3 mg; about 0.01 mg to about 2 mg; about 0.01 mg to about 1 mg; about 0.01 mg to about 0.9 mg; about 0.01 mg to about 0.8 mg; about 0.01 mg to about 0.7 mg; about 0.01 mg to about 0.6 mg; about 0.01 mg to about 0.5 mg; about 0.01 mg to about 0.4 mg; about 0.01 mg to about 0.3 mg; about 0.01 mg to about 0.2 mg; about 0.01 mg to about 0.1 mg; about 0.01 mg to about 0.09 mg; about 0.01 mg to about 0.08 mg: about 0.01 mg to about 0.07 mg; about 0.01 mg to about 0.06 mg; about 0.01 mg to about 0.05 mg; about 0.01 mg to about 0.04 mg; about 0.01 mg to about 0.03 mg; or about 0.01 mg to about 0.02 mg of the aminothiol-conjugate of Formula (I).

In some embodiments of the present disclosure a dosage unit of the combination therapeutic comprises about 0.001 mg to about 1,000 mg of the anti-neoplastic drug.

In some embodiments of the present disclosure a dosage unit of the combination therapeutic comprises about 0.001 mg to about 2,000 mg; about 0.001 mg to about 1,500 mg; about 0.001 mg to about 1,000 mg; about 0.001 mg to about 900 mg; about 0.001 mg to about 800 mg: about 0.001 mg to about 700 mg; about 0.001 mg to about 600 mg; about 0.001 mg to about 500 mg; about 0.001 mg to about 400 mg; about 0.001 mg to about 300 mg; about 0.001 mg to about 200 mg; about 0.001 mg to about 100 mg; about 0.001 mg to about 90 mg; about 0.001 mg to about 80 mg; about 0.001 mg to about 70 mg; about 0.001 mg to about 60 mg; about 0.001 mg to about 50 mg; about 0.001 mg to about 40 mg; about 0.001 mg to about 30 mg; about 0.001 mg to about 20 mg; about 0.001 mg to about 10 mg; about 0.001 mg to about 9 mg; about 0.001 mg to about 8 mg; about 0.001 mg to about 7 mg; about 0.001 mg to about 6 mg; about 0.001 mg to about 5 mg: about 0.001 mg to about 4 mg; about 0.001 mg to about 3 mg; about 0.001 mg to about 2 mg: about 0.001 mg to about 1 mg; about 0.001 mg to about 0.9 mg; about 0.001 mg to about 0.8 mg; about 0.001 mg to about 0.7 mg; about 0.001 mg to about 0.6 mg; about 0.001 mg to about 0.5 mg; about 0.001 mg to about 0.4 mg; about 0.001 mg to about 0.3 mg; about 0.001 mg to about 0.2 mg; about 0.001 mg to about 0.1 mg; about 0.001 mg to about 0.09 mg; about 0.001 mg to about 0.08 mg; about 0.001 mg to about 0.07 mg; about 0.001 mg to about 0.06 mg; about 0.001 mg to about 0.05 mg; about 0.01 mg to about 0.03 mg; about 0.001 mg to about 0.02 mg; about 0.001 mg to about 0.01 mg; about 0.001 mg to about 0.009 mg; about 0.001 mg to about 0.008 mg; about 0.001 mg to about 0.007 mg; about 0.001 mg to about 0.006 mg; about 0.001 mg to about 0.005 mg; about 0.001 mg to about 0.004 mg: about 0.001 mg to about 0.003 mg; or about 0.001 mg to about 0.002 mg of the anti-neoplastic drug.

A third aspect of the present disclosure relates to a method of treating a subject in need of antimicrobial treatment. This method involves administering to the subject a combination therapy comprising (i) an aminothiol-conjugate of Formula (I):

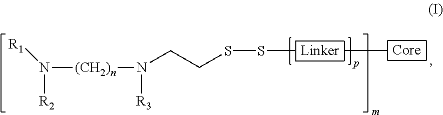

where

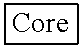

is optional and, if present, is an atom, a molecule, or a macromolecule;

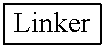

is a linker group, where the linker group is a polymer, a section of a polymer, an arm of a polymer, an arm of a copolymer, or a branch of a dendrimer;
where each

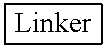

can be the same or different;
R₁, R₂, and R₃ are independently selected from hydrogen and C₁₋₆ alkyl;
m is 1 to 100,000;
n is 1 to 10; and
p is independently 0 to 2,500, or a pharmaceutically acceptable salt, hydrate, polymorph, or solvate thereof, and
(ii) an antimicrobial drug, where the combination therapy is administered in an amount effective to treat a microbial condition in the subject.

A fourth aspect of the present disclosure relates to a combination therapeutic comprising:
(i) an aminothiol-conjugate of Formula (I):

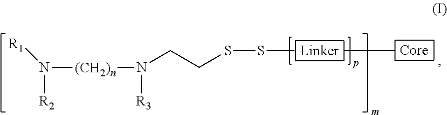

where

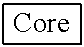

is optional and, if present, is an atom, a molecule, or a macromolecule;

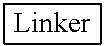

is a linker group, where the linker group is a polymer, a section of a polymer, an arm of a polymer, an arm of a copolymer, or a branch of a dendrimer;
where each

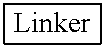

can be the same or different;
R₁, R₂, and R₅ are independently selected from hydrogen and C₁₋₆ alkyl;
m is 1 to 100,000;
n is 1 to 10; and
p is independently 0 to 2,500, or a pharmaceutically acceptable salt, hydrate, polymorph, or solvate thereof, and
(ii) an antimicrobial drug.

Suitable aminothiol-conjugates of Formula (I) for use in the methods and combination therapeutics of the present disclosure are described in detail supra. Thus, in some embodiments, the aminothiol-conjugate of Formula (I) has the following structure:

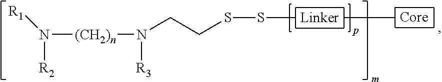

where

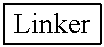

is polyethylene glycol (PEG) or a repeating unit of PEG;

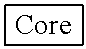

is optional and, if present, is a polymer core or

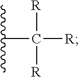

m is 1, 2, 3, 4, 5, 6, 7, or 8; R is independently selected from hydrogen and C₁₋₆ alkyl; R₁, R₂, and R₃ are H; n is 3; and each p is independently 1 to 2,500. In accordance with such embodiments, the

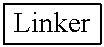

may be the same or different

In some embodiments of the methods and combination therapeutics of the present disclosure, the aminothiol-conjugate of Formula (I) has the structure of:

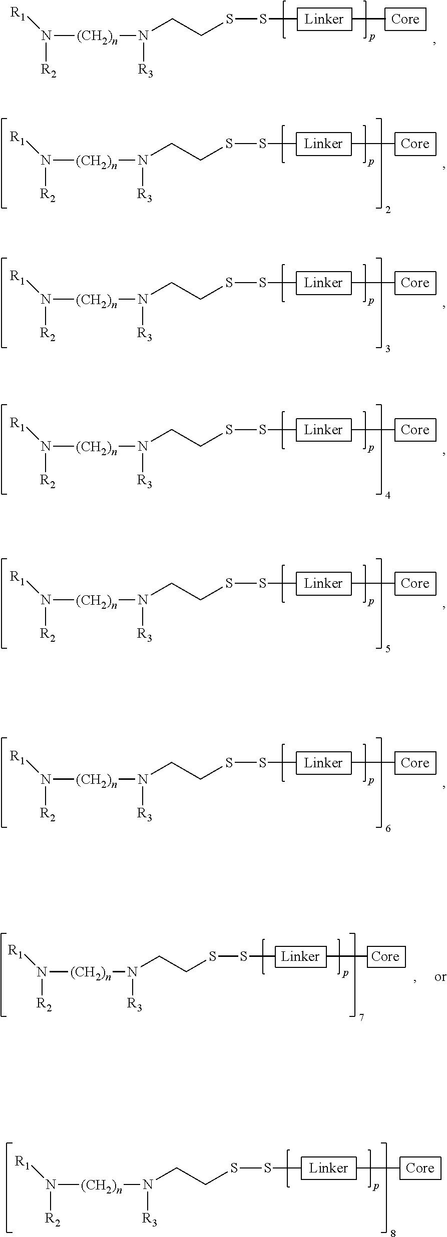

According to these embodiments, in each structure of the aminothiol-conjugate of Formula (I)

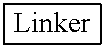

can be the same or different.

In some embodiments of the methods and combination therapeutics of the present disclosure, the aminothiol-conjugate of Formula (I) has the following structure:

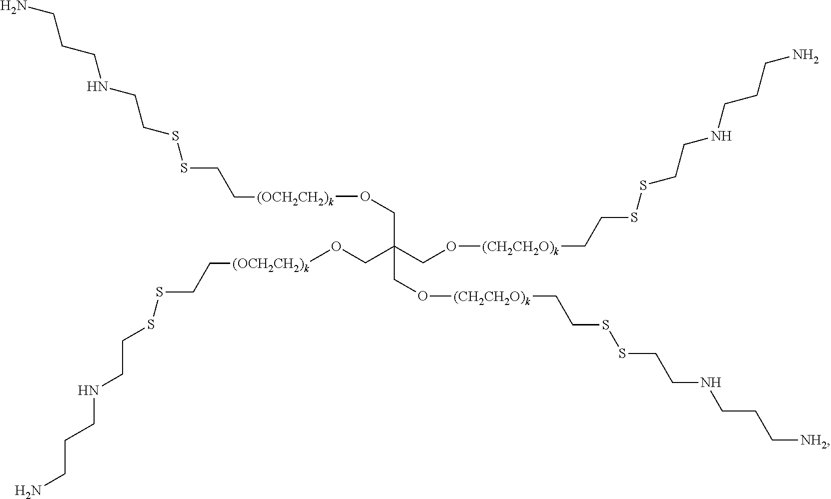

where k is independently 1 to 2,500, or a pharmaceutically acceptable salt thereof. Accordingly, k may be independently selected from 1 to 2,500; from 1 to 2,000; from 1 to 1,750; from 1 to 1,500; from 1 to 1,250; from 1 to 1,000; from 1 to 750; from 1 to 500; from 1 to 250; from 1 to 200; or from 1 to 150. In accordance with such embodiments, the aminothiol-conjugate of Formula (I) may be 4SP65.

In some embodiments of the methods and combination therapeutics of the present disclosure, the aminothiol-conjugate of Formula (I) has the following structure:

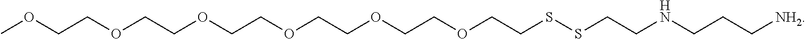 [SMILES: COCCOCCOCCOCCOCCOCCSSCCNCCCN]

In some embodiments of the methods and combination therapeutics of the present disclosure, the aminothiol-conjugate of Formula (I) has the following structure:

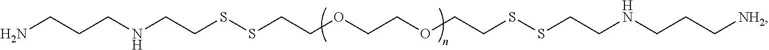

wherein n is independently 1 to 2,500. Accordingly, n may be independently selected from 1 to 2,500; from 1 to 2,000; from 1 to 1,750; from 1 to 1,500; from 1 to 1,250; from 1 to 1,000; from 1 to 750; from 1 to 500; from 1 to 250; from 1 to 200; or from 1 to 150.

According to the present disclosure, antimicrobial drug is selected from the group consisting of an antibiotic drug, an antiviral drug, an antifungal drug, an antiparasitic drug, or a derivative thereof.

In some embodiments of the methods and combination therapeutics of the present disclosure, the antimicrobial drug is selected from the group consisting of (i) eukaryotic and prokaryotic protein kinase inhibitors, (ii) nucleoside analogs, (iii) bacterial cell wall or cell envelope breakdown agents, (iv) antifungal drugs, (v) antiparasitic drugs, (vi) antiviral drugs, or derivatives thereof.

In some embodiments of the methods and combination therapeutics of the present disclosure, the antimicrobial drug is an antiviral drug selected from the group consisting of (i) eukaryotic and prokaryotic protein kinase inhibitors and (ii) nucleoside analogs or derivatives thereof.

Suitable antimicrobial drugs include, but are not limited to, Anti-HIV therapeutics include Abacavir, Amprenavir (Agenerase) (Fosamprenavir is the prodrug formulation), Atazanavir, Cidofovir, Cobicistat (Tybost), Darunavir, Delavirdine, Didanosine, Dolutegravir, Doravirine (Pifeltro), Efavirenz, Elvitegravir, Emtricitabine, Enfuvirtide, Entecavir, Etravirine (Intelence), Fomivirsen, Ibalizumab (Trogarzo), Indinavir, Lamivudine, Lopinavir, Loviride (also a broad-spectrum antiviral agent), Maraviroc, Nevirapine, Raltegravir, Rilpivirine (Edurant), Ritonavir, Saquinavir, Stavudine, Tenofovir disoproxil (also used for Hepatitis B), Tipranavir, Trizivir, Truvada, Valganciclovir (Valcyte), Vicriviroc, Zalcitabine, Zidovudine. Combination therapies for HIV/AIDS include Atripla (Efavirenz/emtricitabine/tenofovir), Biktarvy (Bictegravir/emtricitabine/tenofovir alafenamide), Combivir (Lamivudine/Zidovudine). COVID-19 therapies include Ensitrelvir, Lagevrio (molnupiravir), Remdesivir (Veklury), Olumiant (baricitinib), Paxlovid, (monoclonal antibodies for emergency use). Influenza therapeutics include Amantadine, Ampligen (avian influenza), Baloxavir marboxil (Xofluza) (influenza A/B), Oseltamivir (Tamiflu), Rimantadine, Moroxydine, Oseltamivir (Tamiflu), Rimantadine (influenza A), Peramivir (Rapivab), Umifenovir, Zanamivir (Relenza) (influenza A/B). Hepatitis therapeutics include (Hepatitis B): Adefovir, Descovy (Emtricitabine/tenofovir alafenamide), Telbivudine (Tyzeka), Tenofovir alafenamide, (Hepatitis C): Ribavirin, Sofosbuvir, Boceprevir, Daclatasvir (Daklinza), Simeprevir (Olysio), Taribavirin (Viramidine) (syndromes in which Ribavirin is active), Telaprevir, (Hepatitis D/B): Bulevirtide. Herpes virus therapeutics include Acyclovir (Aciclovir) (also for chickpox, varicella zoster virus), Docosanol, Edoxudine, Famciclovir, Foscarnet, Ibacitabine, Idoxuridine, Inosine pranobex, Penciclovir, Trifluridine, Tromantadine, Valaciclovir (Valtrex), Vidarabine (varicella zoster). Therapeutics for miscellaneous viruses include Ganciclovir (Cytovene) (cytomegalovirus), Imiquimod, (genital warts), Letermovir (Prevymis) (cytomegalovirus), Methisazone (small pox), Pleconaril (pocornavirus), Podophyllotoxin (genital warts), salicylic acid (warts).

A list of antibiotics by class includes the following. Aminoglycosides: amikacin, gentamicin, kanamycin, neomycin, netilmicin, tobramycin, paromomycin, streptomycin; Ansamycins: geldanamycin, herbimycin, rifaximin; Carbacephem: lorcarbef; Carbapnems: ertapenem, doripenem, imipenem/cilastatin, meropenem; Cephalosporins (1st generation): cefadroxil, cefazolin, cephradine, cehaparin, cefalexin; Cephalosporins (2nd generation): cefaclor, cefoxitin, cefotetan, cefamandole, cefametazole, cefonicid, loracarbef, cefprozil, cefuroxime; Cephalosporins (3rd generation): cefixime, cefdinir, cefditoren, cefoperazone, cefotaxime, cefpodoxime, ceftazidime, ceftibuten, ceftizoxime, moxalactam, ceftriaxone, moxalactam, ceftriaxone; Cephalosporins (4th generation): cefepime; Cephalosporins (5th generation): ceftaroline fosamil, ceftobiprole, Glycopeptides: teicoplanin, vancomycin, telavancin, dalbavancin, oritavancin; Lincosamides; clindamycin, lincomycin; Lipopeptide: daptomycin; Macrolides: azithromycin, clarithromycin, erythromycin, roxithromycin, telithromycin, spiramycin, fidaxomicin; Monobactams: aztreomam, Nitrofurans: furazolidone, nitrofurantoin, Oxazolidinones: linezolid, posizolid, radezolid, torezolid; Penicillins: amoxicillin, ampicillin, azlocillin, dicloxacillin, flucloxacillin, mexlocillin, methicillin, oxacillin, pencillin G, penicillin V, piperacillin, temocillin, ticarcillin; Polypeptides: bacitracin, colistin, polymyxin B; Quinolones/Fluoroquinolones: ciprofloxacin, enoxacin gatifloxain, gemifloxacin, levofloxacin, lomefloxacin, moxifloxacin, nadifloxacin, nalidixic acid, norfloxacin, ofloxacin, torovafloxacin, grepafloxacin, sparfloxacin, temfloxacin; Sulfonamides: mafenie, sulfacetamide, sulfadiazine, silver sulfadiazine, sulfamethizole, fulfamethoxaxole, sulffanilmide, sulfasalaxine, trimethoprim-sulfamethoxazole, sulfoamidochrysoidine; Tetracyclines: demeclocycline, doxycycline, metacycline, minocycline, oxytetracycline, tetracycline; Drugs against mycobacteria: clofazimine, dapsone, capreomycin, cycloserine, ethambutol, ethioamide, isoniazid, pyrazinamide, rifampicin, rifabutin, rifapentine, streptomycin; Others: arsphenamine, chloramphenicol, fosfomycin, fusidic acid, metronidazole, mupirocin, platensimycin, quinupristin/dalfopristin, thiamphenicol, tigecycline, tinidazole, trimetheoprim.

Suitable subjects are described in detail supra. In some embodiments, the subject is a mammal, e.g., a human.

In some embodiments of the methods of treating a subject in need of antimicrobial treatment according to the present disclosure, the subject has a condition resistant to treatment with the antimicrobial drug.

In some embodiments of the methods of treating a subject in need of antimicrobial treatment according to the present disclosure, the subject has a condition resistant to a treatment with the antimicrobial drug, such as a bacterial infection (e.g., a Methicillin-resistant Staphylococcus aureus (MRSA) infection, a vancomycin-resistant Enterococcus (VRE) infection, a multi-drug-resistant Mycobacterium tuberculosis (MDR-TB) infection, or a carbapenem-resistant Enterobacteriaceae (CRE) gut bacterial infection), a parasitic infection (e.g, an infection with a Plasmodium strain that causes malaria), a fungal infection (e.g., tinea corporis, tinea pedis, tinea cruris, tinea capitis, tinea unguium, cutaneous candidiasis), or a viral infection (e.g, an infection with an immunodeficiency virus that cause AIDS or simian AIDS (SIV)).

In some embodiments of the methods of treating a subject in need of antimicrobial treatment according to the present disclosure, the subject has a condition resistant to treatment with an antiviral drug.

In some embodiments of the methods of treating a subject in need of antimicrobial treatment according to the present disclosure, the subject is in need of antiviral treatment for HIV.

In certain embodiments of the methods of treating a subject in need of antimicrobial treatment according to the present disclosure, the subject is in need of anti-microbial therapy and the combination therapeutic comprising aminothiol-conjugate is administered under conditions effective to kill one or more pathogenic microorganisms in the subject. The microorganism may be, for example, a bacterium, a yeast, a fungus, or a parasite. The parasite may be intracellular parasite or an extracellular parasite.

In one embodiment, the subject is infected with a virus and the aminothiol-conjugate (or pharmaceutical composition including the aminothiol-conjugate) is administered under conditions effective to treat the virus. In certain embodiments, a therapeutically effective amount of the aminothiol-conjugate described herein is an amount sufficient to reduce the viral load of the target virus in the subject.

The viral infection may be any infection caused by or associated with a double-stranded DNA (dsDNA), single-stranded DNA (ssDNA), double-stranded genomic RNA (dsRNA), single-strand positive RNA, and single-strand negative RNA virus.

For example, the subject may be one that has a chronic viral infection (e.g., is infected with HIV, hepatitis, Epstein-Barr virus, or any combination thereof). The subject may have an acute viral infection (e.g., is infected with orthomyxovirus, influenza virus, adenovirus, coronavirus, parvovirus, enterovirus, variola virus, rotavirus, flavivirus, West Nile virus, yellow fever virus, Zika virus or any combination thereof).

In certain embodiments, the subject is not infected with HIV, hepatitis, and/or Epstein-Barr virus.

In one embodiment, the subject is one infected with adenoviridae, alphaviruses, flaviviridae (e.g., hepatitis C virus), herpes viruses, lentiviridae, picornaviridae, or influenza virus.

In some embodiments, the subject is infected with a virus selected from the group consisting of an adenovirus, an alphavirus (the sole genus in the Togaviridae family), a flavivirus, a herpes virus, a lentivirus (a genus in the Retroviridae family), a picornavirus, and an influenza virus. Accordingly, the subject may be infected with an adenovirus, alphaviruses (the sole genus in the Togaviridae family), flaviviruses, herpes viruses, lentiviruses (a genus in the Retroviridae family), picornaviruses, or influenza virus.

In some embodiments, the subject is infected with an alphavirus (e.g., Rubella virus, Chikungunya virus, O'Nyong-Nyong virus, Ross River virus, Semliki Forest virus, Sindbis virus, Eastern equine encephalitis virus, Venezuelan equine encephalitis virus, and Western equine encephalitis virus), an adenovirus (human adenoviruses types 1 to 57 in seven species, A to G), (e.g., Mastaadenovirus, Aviadenovirus, Atadenovirus, Siadenovirus, and Ichoadenovirus), a coronavirus (e.g., SAR coronavirus including SARS-COV-1, SARS-COV-2, and MERS coronavirus), a filovirus (e.g., Ebola virus), a flavivirus (e.g., Dengue virus, yellow fever virus, West Nile virus, Murray Valley encephalitis virus, Kyasanur encephalitis virus, Japanese encephalitis virus, tick-borne encephalitis virus, Zika virus, St. Louis encephalitis virus, and Hepatitis C virus, and GB virus-C), a herpesvirus (e.g., herpes simplex virus 1, herpes simplex virus 2, varicella zoster virus, human herpes virus 6, human herpes virus 7, cytomegalovirus, Epstein-Barr virus, human herpes virus 8, and herpes simian B virus), a lentivirus (e.g., human immunodeficiency virus type 1 and 2, simian immuno-deficiency virus), an orthomyxovirus (e.g., influenza A and B viruses), a picornavirus (e.g., Genus Enterovirus (including poliovirus, rhinoviruses, coxsackievirus, echo virus, Aichi virus, Apthovirus, Cardiovirus (encephalomyocarditis virus), Thieler's virus, Hepatovirus (hepatitis A virus), salivirus, Seneca Valley virus, or another human enteroviruses).

In one embodiment, the subject is one infected with influenza virus (e.g., H1N1, H5N1, or H3N2).

In one embodiment, the subject is one infected with adenovirus. The adenovirus may be of the species B, C, or E.

In one embodiment, the subject is one infected with Ebola virus.

In one embodiment, the subject is infected with a coronavirus (e.g., SARS-COV, SARS-COV-2, MERS-COV).

In one embodiment, the subject is infected with a virus that encodes a reverse transcriptase (including HIV and SIV), orthomyxovirus, RNA viruses (including influenza A and B, and Ebola virus), or DNA viruses (including adenovirus species).

The aminothiol-conjugate of Formula (I) and the antimicrobial drug according to the present disclosure may be formulated together in a single pharmaceutical composition or as separate pharmaceutical compositions.

As described herein supra, a dosage unit of the combination therapeutic may comprise about 0.001 mg to about 1,000 mg of the aminothiol-conjugate of Formula (I); about 0.01 mg to about 500 mg of the aminothiol-conjugate of Formula (I); or about 0.001 mg to about 500 mg of the aminothiol-conjugate of Formula (I).

In some embodiments of the present disclosure a dosage unit of the combination therapeutic comprises about 0.001 mg to about 2,000 mg; about 0.001 mg to about 1,500 mg; about 0.001 mg to about 1,000 mg; about 0.001 mg to about 900 mg; about 0.001 mg to about 800 mg; about 0.001 mg to about 700 mg; about 0.001 mg to about 600 mg; about 0.001 mg to about 500 mg; about 0.001 mg to about 400 mg; about 0.001 mg to about 300 mg; about 0.001 mg to about 200 mg; about 0.001 mg to about 100 mg; about 0.001 mg to about 90 mg; about 0.001 mg to about 80 mg; about 0.001 mg to about 70 mg; about 0.001 mg to about 60 mg; about 0.001 mg to about 50 mg; about 0.001 mg to about 40 mg; about 0.001 mg to about 30 mg; about 0.001 mg to about 20 mg; about 0.001 mg to about 10 mg; about 0.001 mg to about 9 mg; about 0.001 mg to about 8 mg; about 0.001 mg to about 7 mg; about 0.001 mg to about 6 mg; about 0.001 mg to about 5 mg; about 0.001 mg to about 4 mg; about 0.001 mg to about 3 mg; about 0.001 mg to about 2 mg; about 0.001 mg to about 1 mg; about 0.001 mg to about 0.9 mg; about 0.001 mg to about 0.8 mg; about 0.001 mg to about 0.7 mg; about 0.001 mg to about 0.6 mg; about 0.001 mg to about 0.5 mg; about 0.001 mg to about 0.4 mg; about 0.001 mg to about 0.3 mg; about 0.001 mg to about 0.2 mg; about 0.001 mg to about 0.1 mg; about 0.001 mg to about 0.09 mg; about 0.001 mg to about 0.08 mg; about 0.001 mg to about 0.07 mg; about 0.001 mg to about 0.06 mg; about 0.001 mg to about 0.05 mg; about 0.01 mg to about 0.03 mg; about 0.001 mg to about 0.02 mg; about 0.001 mg to about 0.01 mg; about 0.001 mg to about 0.009 mg; about 0.001 mg to about 0.008 mg; about 0.001 mg to about 0.007 mg; about 0.001 mg to about 0.006 mg; about 0.001 mg to about 0.005 mg; about 0.001 mg to about 0.004 mg: about 0.001 mg to about 0.003 mg; or about 0.001 mg to about 0.002 mg of the antimicrobial drug.

The combination therapeutic according to the present disclosure may be for administration to a mammal. In some embodiments of the present disclosure, the combination therapeutic is for administration to a human.

The present disclosure provides methods for overcoming the resistance of neoplastic cells (e.g., cancer cells) and microbial cells (e.g., bacterial cells, fungal cells, parasitic cells) against anti-neoplastic or antimicrobial drugs, respectively. Accordingly, in some embodiments of the methods disclosed herein, contacting a neoplastic cell or microbial cell with an aminothiol-conjugate according to the present disclosure is sufficient to sensitize the neoplastic cell or microbial cell to treatment with an anti-neoplastic drug or anti-microbial drug, respectively. One consequence of said sensitization may be that the neoplastic or microbial cell is rendered more susceptible to treatment with the anti-neoplastic drug or anti-microbial drug, respectively. Another consequence of said sensitization may be that the neoplastic or microbial cell is prevented from becoming resistant to treatment with the anti-neoplastic drug or anti-microbial drug, respectively.

Another consequence is that the neoplastic cell or microbial cell will be sensitive to the anti-neoplastic drug or anti-microbial drug at drug concentrations that can be achieved safely in patients. In some instances, the minimal amount of anti-neoplastic drug or anti-microbial drug that is needed to achieve a desired effect in a patient is higher than the amount that can be safely administered to that patient. In these situations, an aminothiol-conjugate according to the present disclosure can be used to sensitize the neoplastic cell or microbial cell, so that the amount of the anti-neoplastic drug or anti-microbial drug that is needed to achieve the desired effect in the patient is lowered. In some embodiments, the amount of the anti-neoplastic drug or anti-microbial drug that is needed to achieve the desired effect in a patient, undergoing treatment with aminothiol-conjugate according to the present disclosure, is lowered to the amount that can be safely administered to the patient.

In some embodiments, contacting a neoplastic cell or microbial cell with an aminothiol-conjugate according to the present disclosure is carried out under conditions effective to deliver the aminothiol-conjugate into the neoplastic or microbial cell.

Accordingly, a fifth aspect of the present disclosure relates to a method of increasing sensitivity of a cell to treatment with an anti-neoplastic drug or antimicrobial drug. This method involves selecting a neoplastic cell, a microbial cell (e.g., a bacterial cell, a fungal cell, or a parasitic cell), or a cell infected with a microbe (e.g., a cell infected with a virus, bacterium, fungus, or parasite); and administering to the cell (or contacting the cell with) an aminothiol-conjugate of Formula (I):

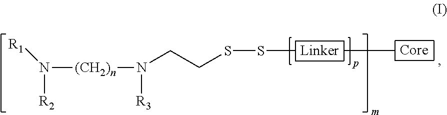

where

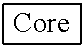

is optional and, if present, is an atom, a molecule, or a macromolecule;

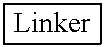

is a linker group, where the linker group is a polymer, a section of a polymer, an arm of a polymer, an arm of a copolymer, or a branch of a dendrimer;
where each

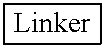

can be the same or different;
R₁, R₂, and R₃ are independently selected from hydrogen and C₁₋₆ alkyl;
m is 1 to 100,000;
n is 1 to 10; and
p is independently 0 to 2,500,
or a pharmaceutically acceptable salt, hydrate, polymorph, or solvate thereof in an amount effective to increase sensitivity of the cell to treatment with the anti-neoplastic drug or antimicrobial drug.

The term “sensitivity” as used herein is a relative term which refers to an increase in the degree of effectiveness of a therapy (e.g., involving an anti-neoplastic drug or antimicrobial drug) in, e.g., inhibiting the growth and/or viability of a neoplastic or microbial cell. The term “growth” as used herein encompasses any aspect of the growth, proliferation, and progression of a neoplastic or microbial cell, including, e.g., cell division (i.e., mitosis), cell growth (e.g., increase in cell number), an increase in genetic material (e.g., prior to cell division), increase in invasiveness, and metastasis. Reduction, inhibition, and/or suppression of cell growth includes, but is not limited to, inhibition of cell growth as compared to the growth of untreated or mock treated cells, inhibition of proliferation, induction of cell death, and reduction of cell number. An increase in sensitivity to a therapy may be measured by, e.g., using cell proliferation assays and/or cell cycle analysis assays. When a cell is sensitized, often a lower dose of a therapy (e.g., antineoplastic drug or antimicrobial drug) can be used to achieve the same therapeutic effect as that achieved by a higher dose in a cell that has not been sensitized.

In some embodiments, when the cell is a neoplastic cell, reduction, inhibition, and/or suppression of cell growth includes, but is not limited to, inhibition of metastases, inhibition of invasiveness, induction of cell senescence, induction of cell death, and reduction of cell number. An increase in sensitivity to a therapy may be measured by, e.g., using cell proliferation or viability assays and/or cell cycle analysis assays.

Accordingly, in some embodiments, the sensitivity of the neoplastic cell(s) or the microbial cell(s) to treatment with an anti-neoplastic drug in combination with the aminothiol-conjugate of Formula (I) or an antimicrobial drug in combination with the aminothiol-conjugate of Formula (I), respectively, is increased by at least ˜1%, at least ˜2%, at least ˜3%, at least ˜4%, at least ˜5%, at least ˜6%, at least ˜7%, at least ˜8%, at least ˜9%, at least ˜10%, at least ˜20%, at least ˜30%, at least ˜40%, at least ˜50%, at least ˜60%, at least ˜70%, at least ˜80%, at least ˜90%, at least ˜95%, at least ˜99%, at least ˜100%, at least ˜150%, at least ˜200%, at least ˜250%, at least ˜300%, at least ˜350%, at least ˜400%, at least ˜500%, ˜1%, ˜2%, ˜3%, ˜4%, ˜5%, ˜6%, ˜7%, ˜8%, ˜9%, ˜10%, ˜20%, ˜30%, ˜40%, ˜50%, ˜60%, ˜70%, ˜80%, ˜90%, ˜95%, ˜99%, ˜100%, ˜150%, ˜200%, ˜250%, ˜300%, ˜350%, ˜400%, ˜500% (i.e., ˜5-fold), ˜600%, ˜700%, ˜800%, ˜900%, or ˜1,000%, as compared to when the aminothiol-conjugate of Formula (I) is not administered to the selected neoplastic or microbial cell (e.g., as compared to just prior to administration of said aminothiol-conjugate of Formula (I) or as compared to administration of the antineoplastic or antimicrobial drug alone). In some embodiments, the sensitivity is increased within a range having a lower limit selected from ˜1%, ˜2%, ˜3%, ˜4%, ˜5%, ˜6%, ˜7%, ˜8%, ˜9%, ˜10%, ˜20%, ˜30%, ˜40%, ˜50%, ˜60%, ˜70%, ˜80%, ˜90%, ˜95%, ˜99%, ˜100%, ˜150%, ˜200%, ˜250%, ˜300%, ˜350%, ˜400%, ˜500%, ˜600%, ˜700%, ˜800%, and ˜900% and an upper limit selected from ˜2%, ˜3%, ˜4%, ˜5%, ˜6%, ˜7%, ˜8%, ˜9%, ˜10%, ˜20%, ˜30%, ˜40%, ˜50%, ˜60%, ˜70%, ˜80%, ˜90%, ˜95%, ˜99%, ˜100%, ˜150%, ˜200%, ˜250%, ˜300%, ˜350%, ˜400%, ˜500%, ˜600%, ˜700%, ˜800%, ˜900%, and ˜1,000%, as compared to when the aminothiol-conjugate of Formula (I) is not administered to the selected neoplastic or microbial cell (e.g., as compared to just prior to administration of said aminothiol-conjugate of Formula (I) or as compared to administration of the antineoplastic or antimicrobial drug alone).

In some embodiments, the sensitivity of the neoplastic cell(s) or the microbial cell(s) to treatment with an anti-neoplastic drug in combination with the aminothiol-conjugate of Formula (I) or an antimicrobial drug in combination with the aminothiol-conjugate of Formula (I), respectively, is increased by at least 1-fold, at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 60-fold, at least 70-fold, at least 80-fold, at least 90-fold, at least 95-fold, at least 99-fold, at least 100-fold, at least 150-fold, at least 200-fold, at least 250-fold, at least 300-fold, at least 350-fold, at least 400-fold, at least 500-fold, at least 600-fold, at least 700-fold, at least 800-fold, at least 900-fold, at least 1,000-fold, at least 2,000-fold, at least 3,000-fold, at least 4,000-fold, at least 5,000-fold, at least 6,000-fold, at least 7,000-fold, at least 8,000-fold, at least 9,000-fold, at least 10,000-fold, or any amount there between, as compared to when the aminothiol-conjugate of Formula (I) is not administered to the selected neoplastic or microbial cell (e.g., as compared to just prior to administration of said aminothiol-conjugate of Formula (I) or as compared to administration of the antineoplastic or antimicrobial drug alone). In some embodiments, the sensitivity is increased within a range having a lower limit selected from 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 95-fold, 100-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, 1,000-fold, 2,000-fold, 3,000-fold, 4,000-fold, 5,000-fold, 6,000-fold, 7,000-fold, 7,500-fold, 8,000-fold, 8,500-fold, 9,000-fold, 9,500-fold and an upper limit selected from 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 95-fold, 99-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, 1,000-fold, 2,000-fold, 3,000-fold, 4,000-fold, 5,000-fold, 6,000-fold, 7,000-fold, 7,500-fold, 8,000-fold, 8,500-fold, 9,000-fold, 9,500-fold, and 10,000-fold, as compared to when the aminothiol-conjugate of Formula (I) is not administered to the selected neoplastic or microbial cell (e.g., as compared to just prior to administration of said aminothiol-conjugate of Formula (I) or as compared to administration of the antineoplastic or antimicrobial drug alone).

In some embodiments, the cell is a neoplastic cell.

In some embodiments, the neoplastic cell is selected from the group consisting of a non-small lung cancer cell, breast cancer cell, ovarian cancer cell, cervical cancer cell, colon cancer cell, lung cancer cell, skin cancer cell (malignant melanoma cell), lymphoreticular tumor cell, lung cancer cell, prostate cancer cell, colorectal cancer cell, melanoma cell, bladder cancer cell, lymphoma cell, Hodgkin lymphoma cell, non-hodgkin lymphoma cell, endometrial cancer cell, leukemia cell, kidney cancer cell, pancreatic cancer cell, thyroid cancer cell, liver cancer cell, sarcoma cell, a cell from a myelodysplastic condition, a cell from an undifferentiated tumor, and combinations thereof.

Suitable antineoplastic drugs for use in the methods of the present disclosure are described in detail supra. In some embodiments, the anti-neoplastic drug is a protein kinase inhibitor.

For example, as described herein supra, the anti-neoplastic drug may be selected from the group consisting of cisplatin, erlotinib, gefitinib, afatinib, osimertinib, abemaciclib, carboplatin, oxaliplatin, nedaplatin, and lobaplatin or derivatives thereof. In some embodiments, the anti-neoplastic drug is cisplatin. In other embodiments, the anti-neoplastic drug is gefitinib.

In some embodiments of the methods of increasing sensitivity of a cell to treatment with an anti-neoplastic drug or antimicrobial drug according to the present disclosure, the antimicrobial drug is selected from the group consisting of an antibiotic drug, an antiviral drug, an antifungal drug (e.g., azoles, amphotericin B, imidazoles, triazoles such as fluconazole, terbinafine, voriconazole, and echinocandin B), and antiparasitic drug (e.g., chloroquine, amodiaquine, atovaquone-proguanil, primaquine, mefloquine, atovaquone-proguanil, doxycycline, azithromycin, clindamycin, quinine, furazolidone, albendazole, eflornithine, pentamidine, amphotericin B, sulfonamides such as sulfamethazine and sulfamerazine, praziquantel, niclosamide, albendazole, permethrin, ivermectin, melathion), and an antibacterial drug (e.g., aminoglycosides, carbapenems, cephalosporins, fluoroquinolones, glycopeptides and lipoglycopeptides such as vancomycin, macrolides such as erythromycin and azithromycin, monobactams such as aztreonam, oxazolidinones such as linezolid and tedizolid, penicillins, polypeptides, rifamycins, sulfonamides, streptogramins such as quinupristin and dalfoprisin, and tetracyclines).

In some embodiments of the methods of increasing sensitivity of a cell to treatment with an anti-neoplastic drug or antimicrobial drug according to the present disclosure, the antiviral drug is selected from the group consisting of nucleoside analogue reverse transcriptase inhibitors (NRTIs), nonnucleoside reverse transcriptase inhibitors (NNRTIs), protease inhibitors (e.g., darunavir, atazanavir, ritonavir), fusion inhibitors, chemokine coreceptor antagonists (e.g., CCR5 antagonists and CXCR4 antagonists), neuraminidase inhibitors (e.g., zanaamivir, oseltamivir, peramivir), attachment inhibitors, entry inhibitors, integrase inhibitors (e.g., raltegravir), and viral polymerase inhibitors (e.g., acyclovir, valacyclovir, valganciclovir, tenofovir).

In some embodiments of the methods of increasing sensitivity of a cell to treatment with an anti-neoplastic drug or antimicrobial drug according to the present disclosure, the method further involves administering to the neoplastic cell, the microbial cell, or the cell infected with a microbe, the anti-neoplastic drug or antimicrobial drug (e.g., antibiotic or antiviral drug) together with or after said administering the aminothiol-conjugate of Formula (I). Such sequential administration may be carried out as described herein.

As described herein, the methods of increasing sensitivity of a cell to treatment with an anti-neoplastic drug or antimicrobial drug according to the present disclosure may be carried out in vitro or in vivo. When carried out in vivo, suitable subjects may be those described herein according to any aspect of the present disclosure.

Suitable aminothiol-conjugates of Formula (I) for use in the methods of increasing sensitivity of a cell to treatment with an anti-neoplastic drug or antimicrobial drug according to the present disclosure are described in detail supra. Thus, in some embodiments, the aminothiol-conjugate of Formula (I) has the following structure:

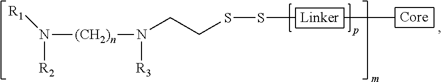

where

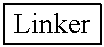

is polyethylene glycol (PEG) or a repeating unit of PEG;

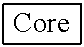

is optional and, if present, is a polymer core or

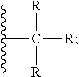

m is 1, 2, 3, 4, 5, 6, 7, or 8; R is independently selected from hydrogen and C₁₋₆ alkyl; R₁, R₂, and R₃ are H; n is 3; and each p is independently 1 to 2,500. In accordance with such embodiments, the

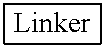

may be the same or different

In some embodiments of the methods of increasing sensitivity of a cell to treatment with an anti-neoplastic drug or antimicrobial drug according to the present disclosure, the aminothiol-conjugate of Formula (I) has the structure of:

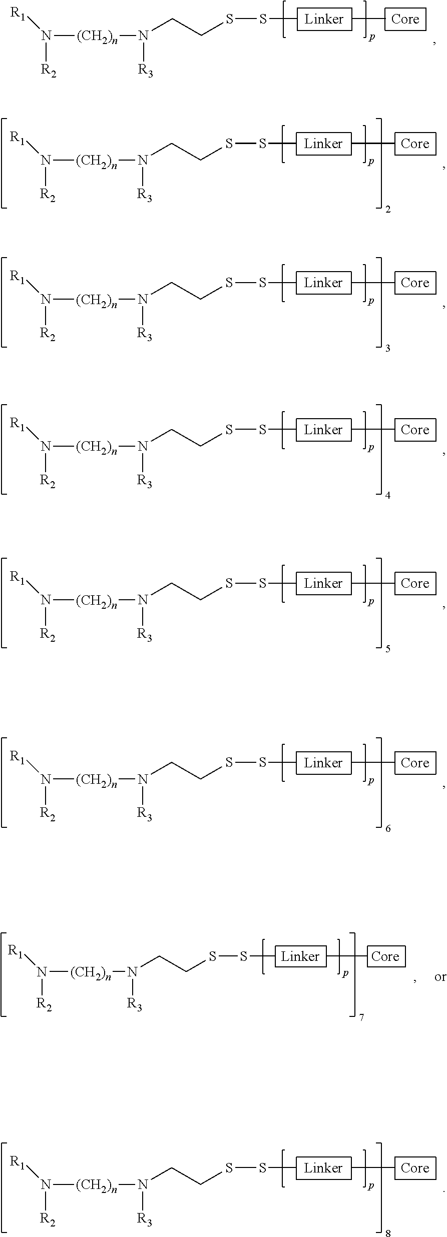

According to these embodiments, in each structure of the aminothiol-conjugate of Formula (I)

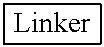

can be the same or different.

In some embodiments of the methods of increasing sensitivity of a cell to treatment with an anti-neoplastic drug or antimicrobial drug according to the present disclosure, the aminothiol-conjugate of Formula (I) has the following structure:

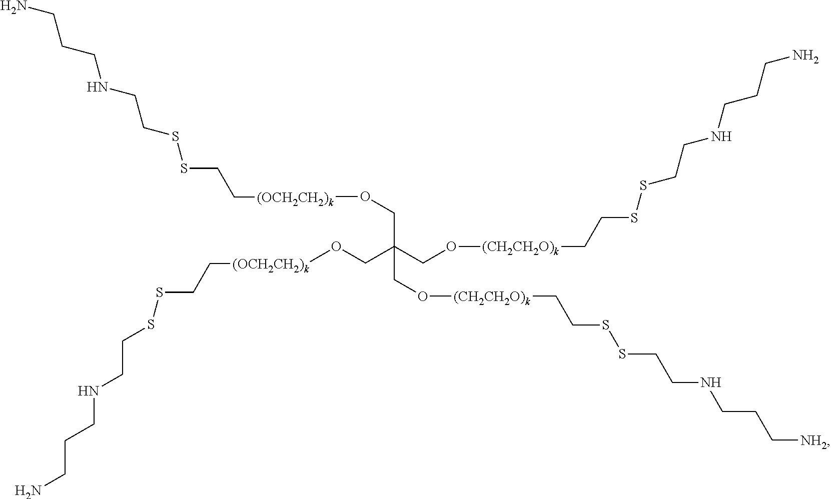

where k is independently 1 to 2,500, or a pharmaceutically acceptable salt thereof. Accordingly, k may be independently selected from 1 to 2,500; from 1 to 2,000; from 1 to 1,750; from 1 to 1,500; from 1 to 1,250; from 1 to 1,000; from 1 to 750; from 1 to 500; from 1 to 250; from 1 to 200; or from 1 to 150. In accordance with such embodiments, the aminothiol-conjugate of Formula (I) may be 4SP65.

In some embodiments of the methods of increasing sensitivity of a cell to treatment with an anti-neoplastic drug or antimicrobial drug according to the present disclosure, the aminothiol-conjugate of Formula (I) has the following structure:

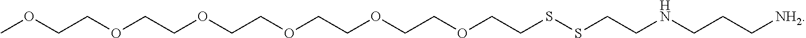 [SMILES: COCCOCCOCCOCCOCCOCCSSCCNCCCN]

In some embodiments of the methods of increasing sensitivity of a cell to treatment with an anti-neoplastic drug or antimicrobial drug according to the present disclosure, the aminothiol-conjugate of Formula (I) has the following structure:

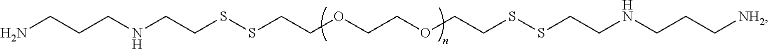

wherein n is independently 1 to 2,500. Accordingly, n may be independently selected from 1 to 2,500; from 1 to 2,000; from 1 to 1,750; from 1 to 1,500; from 1 to 1,250; from 1 to 1,000; from 1 to 750; from 1 to 500; from 1 to 250; from 1 to 200; or from 1 to 150.

In some embodiments of the methods and combination therapeutics according to the present disclosure, the aminothiol-conjugate of Formula (I) has an average molecular weight in the range from about 0.25 kDa to about 100 kDa or from about 0.1 kDa to about 1 kDa.

As described in the examples of the present application, the aminothiol-conjugate of Formula (I) can act as an antiviral agent, a chemoprotectant, a cytoprotectant, a radioprotectant, an anti-fibrotic agent, an anti-tumor agent, an antioxidant, or an antimicrobial or antiparasitic agent. Therefore, the method of using an aminothiol-conjugate of Formula (I) to treat a subject in need of aminothiol therapy (e.g., those in need of treatment with an antiviral agent, a chemoprotectant, a cytoprotectant, a radioprotectant, an anti-fibrotic agent, an anti-tumor agent, an antioxidant, or an antimicrobial or antiparasitic agent) is contemplated. Such methods involve administering to the subject the aminothiol-conjugate of Formula (I):

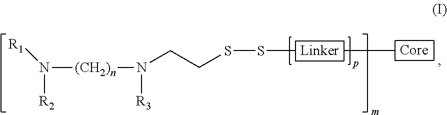

wherein

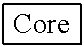

is optional and, if present, is an atom, a molecule, or a macromolecule;

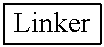

is a linker group, wherein the linker group is a polymer, a section of a polymer, an arm of a polymer, an arm of a copolymer, or a branch of a dendrimer;
wherein each

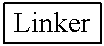

can be the same or different;
R₁, R₂, and R₃ are independently selected from hydrogen and C₁₋₆ alkyl;
m is 1 to 100,000;
n is 1 to 10; and
p is independently 0 to 2,500,
or a pharmaceutically acceptable salt, hydrate, polymorph, or solvates thereof.

Furthermore, pharmaceutical compositions including an aminothiol-conjugate of Formula (I):

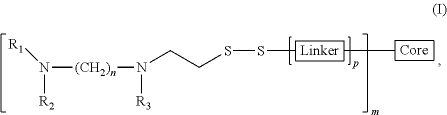

wherein

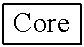

is optional and, if present, is an atom, a molecule, or a macromolecule;

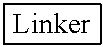

is a linker group, wherein the linker group is a polymer, a section of a polymer, an arm of a polymer, an arm of a copolymer, or a branch of a dendrimer;
wherein each

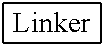

can be the same or different;
R₁, R₂, and R₃ are independently selected from hydrogen and C₁₋₆ alkyl;
m is 1 to 100,000;
n is 1 to 10; and
p is independently 0 to 2,500,
or a pharmaceutically acceptable salt, hydrate, polymorph, or solvates thereof are also contemplated by the present application.

An aminothiol-conjugate of Formula (I) is a disulfide. As described in the examples of the present application, when the cell is contacted with an aminothiol-conjugate of Formula (I), aminothiol (e.g., WR1065) and PEG-SH scaffold are released.

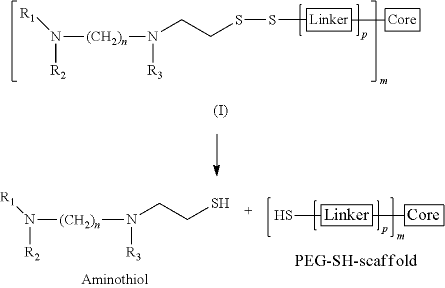

Both aminothiol and PEG-SH can be used to treat a subject in need of aminothiol therapy (e.g., those in need of treatment with an antiviral agent, a chemoprotectant, a cytoprotectant, a radioprotectant, an anti-fibrotic agent, an anti-tumor agent, an antioxidant, or an antimicrobial or antiparasitic agent). According to some embodiments of the present application, aminothiol and PEG-SH act synergistically.

Another aspect of the present disclosure relates to a method of treating a subject in need of aminothiol therapy. The method involves administering to the subject an aminothiol-conjugate of Formula (I):

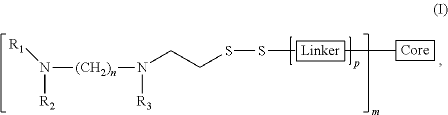

wherein

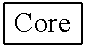

is optional and, if present, is an atom, a molecule, or a macromolecule;

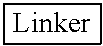

is a linker group, wherein the linker group is a polymer, a section of a polymer, an arm of a polymer, an arm of a copolymer, or a branch of a dendrimer;
wherein each

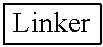

can be the same or different;
R₁, R₂, and R₃ are independently selected from hydrogen and C₁₋₆ alkyl;
m is 1 to 100,000;
n is 1 to 10; and
p is independently 0 to 2,500,
or a pharmaceutically acceptable salt, hydrate, polymorph, or solvates thereof, wherein both aminothiol and PEG-SH are used to treat the subject.

According to some embodiments of the present disclosure, the use of an aminothiol-conjugate of Formula (I) is deemed to provide a synergistic effect (aminothiol and PEG-SH act synergistically).

In accordance with the present disclosure, the use of an aminothiol-conjugate of Formula (I) is deemed to provide a synergistic effect when both aminothiol and PEG-SH scaffold exhibit increased activity when used in combination as compared to the activity they exhibit when used separately from each other.

Without being bound by theory, the synergistic activity of aminothiol-conjugate of Formula (I) is greater than the combined activity of aminothiol and PEG-SH scaffold used separately:

activity (synergistic)>activity (aminothiol)+activity (PEG-SH scaffold),
where activity (synergistic) is the activity of aminothiol and PEG-SH scaffold when used in combination; activity (aminothiol) is activity of aminothiol when used alone; and activity (PEG-SH scaffold) is activity of PEG-SH scaffold when used alone. Accordingly, the cytotoxic and/or cytolytic activity of the aminothiol-conjugate of the present disclosure on cancer cells may be greater than the additive cytotoxicity and/or cytolytic activity of aminothiol and the PEG-SH scaffold on cancer cells.

The aminothiol-conjugates and other drugs or agents described herein can be administered in combination or separately using any appropriate drug administration method(s) known or described in the future, including but not limited to intravenously, subcutaneously, orally, intraperitoneally, intranasally, intrarectally, topically, by inhalation and/or transdermal patch. The drugs can be encapsulated in any delivery module that achieves drug delivery to the desired target cell(s) including by encapsulation or incorporation into nanoparticles, micelles, liposomes, nanogels, or others (see above).

Drug dosing levels should be based upon the level of aminothiol (or analogue thereof) that is being delivered. Thus, the following discussion considers the dose of aminothiol being administered as opposed to the total amount of prodrug being administered. The total amount of prodrug will vary depending upon the nature of the prodrug being administered. The active moiety (the aminothiol) can be administered at dosages selected to provide the equivalent of 910 mg/m2 or less for a 60 kg BW adult human. This dosage is equivalent to 24.3 mg/kg BW for a 60 kg BW adult human being (or a total dose of 1456 mg for a 60 kg BW adult).

It can be desirable to administer the aminothiol at doses that are lower than this level when repeated dosing is needed or desired. In addition, it can be desirable to administer the compound using an initial high dose (a bolus dose) and then tapering down to lower doses to be repeated multiple times a week or administered as often as once a day. A dose of 740 mg/m2 aminothiol or aminothiol equivalent is associated with fewer side effects (List et al., “Stimulation of Hematopoiesis by Amifostine in Patients with Myelodysplastic Syndrome,” Blood 90:3364-3369 (1997), which is hereby incorporated by reference in its entirety), and is thus generally preferred. For daily dosing, 200-340 mg/m2 of amifostine (544 mg total dose for a 60 kg BW adult) is generally preferred (Santini et al., “The Potential of Amifostine: from Cytoprotectant to Therapeutic Agent,” Haematologica 84:1035-1042 (1999); Schuchter, “Guidelines for the Administration of Amifostine,” Semin Oncol 23:40-43 (1996), each of which is hereby incorporated by reference in its entirety). WR1065, given by injection at 500-910 mg/m², has a plasma T_{1/2} of ˜10 minutes and has a peak plasma level of ˜100 μM.

Rodent studies suggest the use of higher dosages. For example, the maximally tolerated dose (MTD) for WR-1065 (in the form of amifostine) in mice was 432 mg/kg BW administered i.p. and 720 mg/kg BW administered p.o., and the 100% effective radioprotective dose was about one half of the MTD. For aminothiol delivered in the form of phosphonol, the MTD was 893 mg/kg BW administered i.p. and 1488 mg/kg BW administered p.o., and the 100% effective radioprotective dose was about one half of the MTD. All of the aminothiols have MTDs in rodents of greater than 400 mg/kg BW.

Aminothiols including WR-1065 can be efficacious at very low concentrations, for example, down to 0.4 micromolar concentrations in some in vitro studies.

While it is generally preferred to formulate aminothiol-conjugate drugs for oral administration, the drugs can be formulated so as to allow them to be administered by other routes. It can be desirable in certain embodiments to formulate the drug for intravenous administration in order to maximize efficacy. Because of the structural similarities between WR-1065 and WR-255591, especially the similarities in the sulfhydryl ends of the molecules, WR-255591 is expected to behave in a manner similar to WR-1065.

An unusual feature of the aminothiols, and especially WR1065, is that intracellular levels of the aminothiol can be determined (Bai et al., “New Liquid Chromatographic Assay with Electrochemical Detection for the Measurement of Amifostine and WR1065,” J. Chromatogr. B. Analyt. Technol. Biomed. Life. Sci. 772:257-265 (2002); Elas et al., “Oral Administration is as Effective as Intraperitoneal Administration of Amifostine in Decreasing Nitroxide EPR Signal Decay In Vivo,” Biochim. Biophys. Acta. 1637:151-155 (2003); Shaw et al., “Pharmacokinetic Profile of Amifostine,” Semin. Oncol. 23:18-22 (1996), each of which is hereby incorporated by reference in its entirety). This makes it possible to use intracellular aminothiol levels as a guide to drug administration. For anticancer effects, prodrug administration at levels that result in 30 to 100 nanomoles aminothiol per 10⁶ cells are recommended. For some tumor types, lower intracellular levels are equally effective and can be used. For breast cancers that fall into the same classification as MDA-MB-468 cells and/or that have the same or similar genetic, epigenetic and gene expression changes, intracellular levels in the range of 0.001 to 30 nanomoles aminothiol per 10⁶ cells are effective. For antiviral effects, administering the prodrug at dose levels that result in intracellular levels in the range of 0.001 to 30 nanomoles aminothiol per 10⁶ cells also is recommended.

To obtain the optimal therapeutic effects of the aminothiols, it can be desirable to administer the prodrug more than once. For multi-day or multi-week dosing, administration of the prodrug at dose levels that result in 0.000001 to 30 nanomoles aminothiol per 10⁶ cells is recommended. For all other therapeutic effects, including radioprotective and cytoprotective effects, the prodrug can be administered at dose levels that result in intracellular levels in that range from 1 to 100 nanomoles aminothiol per 10⁶ cells.

It should be noted that the levels of prodrug that are administered will vary considerably based upon the structure of the prodrug and the nature of the target cells for which therapeutic effects are sought. For target cells that express the polyamine, folic acid, and/or amino acid transport system, prodrugs designed to take advantage of these active transport systems generally can be administered at lower amounts that the amounts needed to obtain a comparable intracellular level using a prodrug that is not actively transported into the cell. In addition, the levels of expression of active transport systems can vary between diseased or stressed cells, and can be affected by prodrug treatment, with the result that lower prodrug doses may be sufficient to obtain a therapeutic effect when multiple doses are being administered over time.

Also contemplated are combination therapies including the aminothiol-conjugate prodrug described herein and one or more other agents. The prodrugs described herein can be administered in combination with other agents employed to obtain the therapeutic benefits of the aminothiols. One of the benefits of such combination therapies is that lower doses of the therapeutic agents can be administered (as compared to typical dosage of the drug(s) alone) and/or greater therapeutic effects can be achieved. Such lower dosages can be particularly advantageous for antiretroviral drugs known to have genotoxicity and mitochondrial toxicity, and for cytotoxic chemotherapeutic agents.

The respective therapeutic agents in the combination therapies and methods described herein may be administered simultaneously (i.e., in the same drug), in parallel (i.e., in any order, in separate drugs in which one is administered immediately after the other) or in any order. The combination of therapeutics described herein may also be administered sequentially. The therapeutic agents in the combination may be in different dosage forms (e.g., one agent is a tablet or capsule and another tablet is a sterile liquid) and/or different dosing schedules (e.g., the aminothiol-conjugate described herein being administered once weekly and a chemotherapeutic administered once daily). For example, in some embodiments, the respective therapeutics are administered sequentially with the first being administered at least or at least about or about or 5 minutes, 15 minutes, 30 minutes, 1 hour, 2 hours, 3 hours, 4 hours, 6 hours, 8 hours, 10 hours, 12 hours, 16 hours, 18 hours, 20 hours, 22 hours, 24 hours, 30 hours, 36 hours, 40 hours, 48 hours, or 72 hours prior to administration of a second therapeutic in the combination. In some examples, the aminothiol-conjugate described herein is administered at least or at least about or about or 5 minutes, 15 minutes, 30 minutes, 1 hour, 2 hours, 3 hours, 4 hours, 6 hours, 8 hours, 10 hours, 12 hours, 16 hours, 18 hours, 20 hours, 22 hours, 24 hours, 30 hours, 36 hours, 40 hours, 48 hours, or 72 hours prior to administration of a second therapeutic (e.g., chemotherapeutic, antimicrobial or antibiotic drug, antiviral drug, etc). In another example, the aminothiol-conjugate as described herein is administered simultaneously or near simultaneously (e.g., within less than about 5 minutes) with a second therapeutic. In another example, the aminothiol-conjugate is administered once every 4 days, 5 days, 6 days, or 7 days.

The aminothiol-conjugates described herein (including derivatives, isomers, metabolites, or pharmaceutically acceptable esters, salts, and solvates thereof) can be incorporated into a pharmaceutically acceptable carrier, including incorporation into nanoparticles, for administration to an individual in need of the therapeutic effects of an aminothiol.

One aspect of the present disclosure relates to a pharmaceutical composition comprising an aminothiol-conjugate as described herein. In one embodiment, the aminothiol-conjugate has Formula (I), as described herein. The pharmaceutical composition may also include mixtures of different aminothiol-conjugates of Formula (I), as described herein.

The pharmaceutical composition may further include an intracellular delivery system. The intracellular delivery system may be selected from the group consisting of: (a) systems comprising a cell penetrating agent, (b) pH-responsive carriers, (c) C₂-streptavidin delivery systems, (d) CH (3)-TDDS drug delivery systems, (e) hydrophobic bioactive carriers, (f) exosomes, (g) lipid-based delivery systems, (h) liposome-based delivery systems, (i) micellar delivery systems, (j) microparticles, (k) molecular carriers, (l) nanocarriers, (m) nanoscopic multi-variant carriers, (n) nanogels, (o) hybrid nanocarrier systems consisting of components of two or more particulate delivery systems, (p) nanoparticles, (q) peptide-based drug delivery systems, and (r) polymer- or copolymer-based delivery systems. In certain embodiments, the intracellular delivery system is a nanoparticle.

In certain embodiments, the pharmaceutical composition does not include a nanoparticle delivery system.

The pharmaceutical composition may also include a surfactant.

The pharmaceutical composition may also include a reducing agent.

Another aspect of the present disclosure relates to a composition comprising one or more different aminothiol-conjugates as described herein. For example, each of the one or more different aminothiol-conjugates as described herein may be independently selected from any of the average molecular weights described herein for aminothiol-conjugates. For example, in certain embodiments, one or more of the different aminothiol-conjugates has an average molecular weight of 100,000 Daltons or less. In certain embodiments, one or more of the different aminothiol-conjugates has an independently selected average molecular weight about 100,000 Daltons; about 20,000 Daltons; about 10,000 Daltons; about 5,000 Daltons; about 3,000 Daltons; about 2,000 Daltons; or about 1,000 Daltons. In certain embodiments, one or more of the different aminothiol-conjugates has an average molecular weight of about 9,000 to about 11,000 Daltons. In certain embodiments, one or more of the different aminothiol-conjugates has an average molecular weight of about 9,000 to about 10,000 Daltons. In one embodiment, one or more of the different aminothiol-conjugates has an average molecular weight of about 10,000 Daltons. In one embodiment, one of the different aminothiol-conjugates has an average molecular weight of about 10,500 Daltons. In certain embodiments, one or more of the different aminothiol-conjugates has an average molecular weight of less than about 500 Daltons. In certain embodiments, one or more of the different aminothiol-conjugates has an average molecular weight about 400 to about 500 Daltons.

Another aspect of the present disclosure relates to a kit comprising one or more different aminothiol-conjugates as described herein.

Yet another aspect of the present disclosure relates to the kit, further comprising one or more additional therapeutic agents.

Because some of the aminothiol-conjugate prodrugs may be sensitive to oxidation, it can be desirable to administer the prodrugs in combination with reducing agents including, but not limited to, vitamin C and vitamin E. Other reducing agents include organic aldehydes, hydroxyl-containing aldehydes, and reducing sugars such as glucose, mannose, galactose, xylose, ribose, and arabinose. Other reducing sugars containing hemiacetal or keto groupings can be employed, for example, maltose, sucrose, lactose, fructose, and sorbose. Other reducing agents include alcohols, preferably polyhydric alcohols, such as glycerol, sorbitol, glycols, especially ethylene glycol and propylene glycol, and polyglycols such as polyethylene and polypropylene glycols.

The aminothiol-conjugates and portions thereof described herein also include the pharmaceutically acceptable salts thereof. The terms “pharmaceutically acceptable salts” and “a pharmaceutically acceptable salt thereof” as used herein are broad terms, and are to be given their ordinary and customary meaning to a person of ordinary skill in the art (and are not to be limited to a special or customized meaning), and refer without limitation to salts prepared from pharmaceutically acceptable, non-toxic acids or bases. Suitable pharmaceutically acceptable salts include metallic salts, e.g., salts of aluminum, zinc, alkali metal salts such as lithium, sodium, and potassium salts, alkaline earth metal salts such as calcium and magnesium salts; organic salts, e.g., salts of lysine, N,N′-dibenzylethylenediamine, chloroprocaine, choline, diethanolamine, ethylenediamine, meglumine (N-methylglucamine), procaine, and tris; salts of free acids and bases; a salt of trifluoroacetic acid; inorganic salts, e.g., sulfate, hydrochloride, and hydrobromide; and other salts which are currently in widespread pharmaceutical use and are listed in sources well known to those of skill in the art, such as, for example, The Merck Index. Any suitable constituent can be selected to make a salt of the therapeutic agents discussed herein, provided that it is non-toxic and does not substantially interfere with the desired activity. In addition to salts, pharmaceutically acceptable precursors and derivatives of the compounds can be employed. Pharmaceutically acceptable amides, lower alkyl esters, and protected derivatives can also be suitable for use in compositions. While it may be possible to administer the compounds of the preferred embodiments in the form of pharmaceutically acceptable salts, it is generally preferred to administer the compounds in neutral form.

It is generally preferred to administer the compounds of preferred embodiments orally; however, other routes of administration are contemplated as described herein. The prodrugs can be formulated into liquid preparations for, e.g., oral administration. Suitable forms include suspensions, syrups, elixirs, and the like. Particularly preferred unit dosage forms for oral administration include tablets and capsules.

The pharmaceutical compositions of the aminothiol-conjugate prodrugs are preferably isotonic with the blood or other body fluid of the recipient. The isotonicity of the compositions can be attained using sodium tartrate, propylene glycol or other inorganic or organic solutes. Sodium chloride is particularly preferred. Buffering agents can be employed, such as acetic acid and salts, citric acid and salts, boric acid and salts, and phosphoric acid and salts. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. In certain embodiments it can be desirable to maintain the active compound in the reduced state. Accordingly, it can be desirable to include a reducing agent, such as vitamin C, vitamin E, or other reducing agents as are known in the pharmaceutical arts, in the formulation.

Viscosity of the pharmaceutical compositions can be maintained at the selected level using a pharmaceutically acceptable thickening agent. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the thickening agent selected. An amount is preferably used that will achieve the selected viscosity. Viscous compositions are normally prepared from solutions by the addition of such thickening agents.

A pharmaceutically acceptable preservative can be employed to increase the shelf life of the pharmaceutical compositions. Benzyl alcohol can be suitable, although a variety of preservatives including, for example, parabens, thimerosal, chlorobutanol, or benzalkonium chloride can also be employed. A suitable concentration of the preservative is typically from about 0.02% to about 2% based on the total weight of the composition, although larger or smaller amounts can be desirable depending upon the agent selected. Reducing agents, as described above, can be advantageously used to maintain good shelf life of the formulation.

The aminothiol prodrugs can be in admixture with a suitable carrier, diluent, or excipient such as sterile water, physiological saline, glucose, or the like, and can contain auxiliary substances such as wetting or emulsifying agents, pH buffering agents, gelling or viscosity enhancing additives, preservatives, flavoring agents, colors, and the like, depending upon the route of administration and the preparation desired. See, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively). Such preparations can include complexing agents, metal ions, polymeric compounds such as polyacetic acid, polyglycolic acid, hydrogels, dextran, and the like, liposomes, microemulsions, micelles, unilamellar or multilamellar vesicles, erythrocyte ghosts or spheroblasts. Suitable lipids for liposomal formulation include, without limitation, monoglycerides, diglycerides, sulfatides, lysolecithin, phospholipids, saponin, bile acids, and the like. The presence of such additional components can influence the physical state, solubility, stability, rate of in vivo release, and rate of in vivo clearance, and are thus chosen according to the intended application, such that the characteristics of the carrier are tailored to the selected route of administration.

For oral administration, the pharmaceutical compositions can be provided as a tablet, aqueous or oil suspension, dispersible powder or granule, emulsion, hard or soft capsule, syrup or elixir. Compositions intended for oral use can be prepared according to any method known in the art for the manufacture of pharmaceutical compositions and can include one or more of the following agents: sweeteners, flavoring agents, coloring agents and preservatives. Aqueous suspensions can contain the active ingredient in admixture with excipients suitable for the manufacture of aqueous suspensions.

Formulations for oral use can also be provided as hard gelatin capsules, wherein the active ingredient(s) are mixed with an inert solid diluent, such as calcium carbonate, calcium phosphate, or kaolin, or as soft gelatin capsules. In soft capsules, the active compounds can be dissolved or suspended in suitable liquids, such as water or an oil medium, such as peanut oil, olive oil, fatty oils, liquid paraffin, or liquid polyethylene glycols. Stabilizers and microspheres formulated for oral administration can also be used. Capsules can include push-fit capsules made of gelatin, as well as soft, sealed capsules made of gelatin and a plasticizer, such as glycerol or sorbitol. The push-fit capsules can contain the active ingredient in admixture with fillers such as lactose, binders such as starches, and/or lubricants such as talc or magnesium stearate and, optionally, stabilizers. In instances where it is desirable to maintain a compound of a preferred embodiment in a reduced form (in the case of certain active metabolites), it can be desirable to include a reducing agent in the capsule or other dosage form.

Tablets can be uncoated or coated by known methods to delay disintegration and absorption in the gastrointestinal tract and thereby provide a sustained action over a longer period of time. For example, a time delay material such as glyceryl monostearate can be used. When administered in solid form, such as tablet form, the solid form typically comprises from about 0.001 wt. % or less to about 50 wt. % or more of active ingredient(s), preferably from about 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1 wt. % to about 2, 3, 4, 5, 6, 7, 8, 9, 10, 15, 20, 25, 30, 35, 40, or 45 wt. %.

Tablets can contain the active ingredients in admixture with non-toxic pharmaceutically acceptable excipients including inert materials. For example, a tablet can be prepared by compression or molding, optionally, with one or more additional ingredients. Compressed tablets can be prepared by compressing in a suitable machine the active ingredients in a free-flowing form such as powder or granules, optionally mixed with a binder, lubricant, inert diluent, surface active or dispersing agent. Molded tablets can be made by molding, in a suitable machine, a mixture of the powdered compound moistened with an inert liquid diluent.

Each tablet or capsule contains from about 10 mg or less to about 1,000 mg or more of the prodrug of choice, e.g., from about 20, 30, 40, 50, 60, 70, 80, 90, or 100 mg to about 150, 200, 250, 300, 350, 400, 450, 500, 550, 600, 650, 700, 750, 800, or 900 mg. Tablets or capsules may be provided in a range of dosages to permit divided dosages to be administered. A dosage appropriate to the patient and the number of doses to be administered daily can thus be conveniently selected. For certain applications, it can be preferred to incorporate two or more of the aminothiol-conjugate prodrugs to be administered into a single tablet or other dosage form (e.g., in a combination therapy); however, for other applications it can be preferred to provide the therapeutic agents in separate dosage forms.

Suitable inert materials include diluents, such as carbohydrates, mannitol, lactose, anhydrous lactose, cellulose, sucrose, modified dextrans, starch, and the like, or inorganic salts such as calcium triphosphate, calcium phosphate, sodium phosphate, calcium carbonate, sodium carbonate, magnesium carbonate, and sodium chloride. Disintegrants or granulating agents can be included in the formulation, for example, starches such as corn starch, alginic acid, sodium starch glycolate, Amberlite, sodium carboxymethylcellulose, ultramylopectin, sodium alginate, gelatin, orange peel, acid carboxymethyl cellulose, natural sponge and bentonite, insoluble cationic exchange resins, powdered gums such as agar, karaya or tragacanth, or alginic acid or salts thereof.

Binders can be used to form a hard tablet. Binders include materials from natural products such as acacia, tragacanth, starch and gelatin, methyl cellulose, ethyl cellulose, carboxymethyl cellulose, polyvinyl pyrrolidone, hydroxypropylmethyl cellulose, and the like.

Lubricants, such as stearic acid or magnesium or calcium salts thereof, polytetrafluoroethylene, liquid paraffin, vegetable oils and waxes, sodium lauryl sulfate, magnesium lauryl sulfate, polyethylene glycol, starch, talc, pyrogenic silica, hydrated silicoaluminate, and the like, can be included in tablet formulations.

Surfactants can also be employed, for example, anionic detergents such as sodium lauryl sulfate, dioctyl sodium sulfosuccinate and dioctyl sodium sulfonate, cationic such as benzalkonium chloride or benzethonium chloride, or nonionic detergents such as polyoxyethylene hydrogenated castor oil, glycerol monostearate, polysorbates, sucrose fatty acid ester, methyl cellulose, or carboxymethyl cellulose. Surfactants as described by U.S. Pat. No. 6,489,312 to Stogniew (which is hereby incorporated by reference in its entirety) may also be used.

Controlled release formulations can be employed wherein the amifostine or analog(s) thereof is incorporated into an inert matrix that permits release by either diffusion or leaching mechanisms. Slowly degenerating matrices can also be incorporated into the formulation. Other delivery systems can include timed release, delayed release, or sustained release delivery systems.

Coatings can be used, for example, nonenteric materials such as methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, providone and the polyethylene glycols, or enteric materials such as phthalic acid esters. Dyestuffs or pigments can be added for identification or to characterize different combinations of active compound doses

When administered orally in liquid form, a liquid carrier such as water, petroleum, oils of animal or plant origin such as peanut oil, mineral oil, soybean oil, or sesame oil, or synthetic oils can be added to the active ingredient(s). Physiological saline solution, dextrose, or other saccharide solution, or glycols such as ethylene glycol, propylene glycol, or polyethylene glycol are also suitable liquid carriers. The pharmaceutical compositions can also be in the form of oil-in-water emulsions. The oily phase can be a vegetable oil, such as olive or arachis oil, a mineral oil such as liquid paraffin, or a mixture thereof. Suitable emulsifying agents include naturally-occurring gums such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsions can also contain sweetening and flavoring agents.

When a selected aminothiol-conjugate prodrug is administered by intravenous, parenteral, or other injection, it is preferably in the form of a pyrogen-free, parenterally acceptable aqueous solution or oleaginous suspension. Suspensions can be formulated according to methods well known in the art using suitable dispersing or wetting agents and suspending agents. The preparation of acceptable aqueous solutions with suitable pH, isotonicity, stability, and the like, is within the skill in the art. A preferred pharmaceutical composition for injection preferably contains an isotonic vehicle such as 1,3-butanediol, water, isotonic sodium chloride solution, Ringer's solution, dextrose solution, dextrose and sodium chloride solution, lactated Ringer's solution, or other vehicles as are known in the art. In addition, sterile fixed oils can be employed conventionally as a solvent or suspending medium. For this purpose, any bland fixed oil can be employed including synthetic mono or diglycerides. In addition, fatty acids such as oleic acid can likewise be used in the formation of injectable preparations. The pharmaceutical compositions can also contain stabilizers, preservatives, buffers, antioxidants, or other additives known to those of skill in the art.

The duration of the injection can be adjusted depending upon various factors, and can comprise a single injection administered over the course of a few seconds or less, to 0.5, 0.1, 0.25, 0.5, 0.75, 1, 2, 3, 4, 5, 6, 7, 8, 9, 10, 11, 12, 13, 14, 15, 16, 17, 18, 19, 20, 21, 22, 23, or 24 hours or more of continuous intravenous administration.

It may be desirable to administer the drug conjugate using an osmotic pump or mini-pump or similar device intended to control and/or extend delivery of drug to target cell populations (Keraliya et al., “Osmotic Drug Delivery System as A Part of Modified Release Dosage Form,” ISRN Pharm. 528079 (2012), which is hereby incorporated by reference in its entirety).

The pharmaceutical compositions composed of one or more selected aminothiol-conjugate prodrugs can additionally employ adjunct components conventionally found in pharmaceutical compositions in their art-established fashion and at their art-established levels. Thus, for example, the compositions can contain additional compatible pharmaceutically active materials for combination therapy (such as supplementary antimicrobials, antipruritics, astringents, local anesthetics, anti-inflammatory agents, reducing agents, and the like), or can contain materials useful in physically formulating various dosage forms of the preferred embodiments, such as excipients, dyes, thickening agents, stabilizers, preservatives or antioxidants.

The aminothiol-conjugate prodrugs can be provided to an administering physician or other health care professional in the form of a kit. The kit is a package which houses a container which contains the compound(s) in a suitable pharmaceutical composition, and instructions for administering the pharmaceutical composition to a subject. The kit can optionally also contain one or more additional therapeutic agents. For example, a kit containing one or more compositions comprising one or more prodrugs in combination with one or more additional therapeutic agent (antimicrobials, antipruritics, astringents, local anesthetics, anti-inflammatory agents, reducing agents, and the like) can be provided, or separate pharmaceutical compositions containing one or more selected prodrugs and additional therapeutic agents can be provided. The kit can also contain separate doses of prodrug for serial or sequential administration. The kit can optionally contain one or more diagnostic tools and instructions for use. The kit can contain suitable delivery devices, e.g., syringes, and the like, along with instructions for administering the compound(s) and any other therapeutic agent. The kit can optionally contain instructions for storage, reconstitution (if applicable), and administration of any or all therapeutic agents included. The kits can include a plurality of containers reflecting the number of administrations to be given to a subject.

The aminothiol-conjugate prodrugs can be administered prophylactically for the prevention of induction of a stress state or disease state in cells of an individual in need of such therapy. Alternatively, therapy is preferably initiated as early as possible following the onset of signs and symptoms of a stress state or disease state. The administration route, amount administered, and frequency of administration will vary depending on the age of the patient, the severity of the infection, and any associated conditions. Contemplated amounts, dosages, and routes of administration for the prodrugs for treatment of disease states such as cancer or infection with a microbial pathogen are similar to those established for conventional anticancer and antiviral agents. Detailed information relating to administration and dosages of conventional antiretroviral agents can be found in the Physician's Desk Reference, 47th edition, which is hereby incorporated by reference in its entirety. This information can be adapted in designing treatment regimens utilizing the prodrugs.

Contemplated amounts of the aminothiol-conjugate prodrugs for oral administration to treat cancer, pathogen/microbial infections, or for cytoprotection range from about 10 mg or less to about 2000 mg or more administered from about every 24 hours or less to about every 6 hours or more (or from about 1 time daily to about 6 times daily) for about 5 days or less to about 10 days or more (40 mg/day or less to about 15,000 mg/day or more) or until there is a significant improvement in the condition. For suppressive therapy to inhibit the onset of cancer or infection in susceptible individuals, doses of from about 10 mg or less to about 1000 mg or more are orally administered once, twice, or multiple times a day, typically for up to about 12 months, or, in certain circumstances, indefinitely (from about 10 mg/day to about 1,000 mg/day). When treatment is long term, it can be desirable to vary the dosage, employing a higher dosage early in the treatment, and a lower dosage later in the treatment.

The single highest dose of amifostine administered to an adult human as documented in the literature was 1330 mg/m2. Children have been administered single doses of amifostine of up to 2700 mg/m2 with no untoward effects. The literature indicates that multiple doses (up to three times the recommended single dose of 740 to 910 mg/m2) have been safely administered within a 24-hour period. Repeated administration of amifostine at two and four hours after the initial dose does not appear to result in an increase in side effects, especially nausea, vomiting, or hypotension. It appears that the most significant deleterious side effect from the administration of amifostine is hypotension.

Contemplated amounts of the compounds of the preferred embodiments, methods of administration, and treatment schedules for individuals with AIDS are generally similar to those described above for treatment of HIV.

Known side effects of amifostine include decrease in systolic blood pressure, nausea, and vomiting. If such side effects are observed for the particular thiophosphate administered, it is generally preferred to administer an antiemetic medication prior to, or in conjunction with the thiophosphate. Suitable antiemetic medications include antihistamines (e.g., buclizine, cyclizine, dimenhydrinate, diphenhydramine, meclizine), anticholinergic agents (e.g., scopolamine), dopamine antagonists (e.g., chlorpromazine, droperidol, metoclopramide, prochlorperazine, promethazine), serotonin antagonists (e.g., dolasetron, granisetron, ondansetron), or other agents (e.g., dexamethasone, methylprednisolone, trimethobenzamide).

The therapeutic agents and combinations for use in the methods described herein can be formulated into a pharmaceutical composition as any one or more of the active compounds described herein and a physiologically acceptable carrier (also referred to as a pharmaceutically acceptable carrier or solution or diluent). Such carriers and solutions include pharmaceutically acceptable salts and solvates of compounds used in the methods described herein, and mixtures comprising two or more of such compounds, pharmaceutically acceptable salts of the compounds and pharmaceutically acceptable solvates of the compounds. Such compositions are prepared in accordance with acceptable pharmaceutical procedures such as described in Remington: The Science and Practice of Pharmacy, 20th edition, ed. Alfonso R. Gennaro (2000), which is hereby incorporated by reference in its entirety.

The term “pharmaceutically acceptable carrier” refers to a carrier that does not cause an allergic reaction or other untoward effect in patients to whom it is administered and are compatible with the other ingredients in the formulation. Pharmaceutically acceptable carriers include, for example, pharmaceutical diluents, excipients or carriers suitably selected with respect to the intended form of administration, and consistent with conventional pharmaceutical practices. For example, solid carriers/diluents include, but are not limited to, a gum, a starch (e.g., corn starch, pregelatinized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof. Pharmaceutically acceptable carriers may further comprise minor amounts of auxiliary substances such as wetting or emulsifying agents, preservatives or buffers, which enhance the shelf life or effectiveness of the therapeutic agent.

Reference to therapeutic agents described herein includes any analog, derivative, isomer, metabolite, pharmaceutically acceptable salt, pharmaceutical product, hydrate, N-oxide, crystal, polymorph, prodrug or any combination thereof.

The therapeutic agents in a free form can be converted into a salt, if need be, by conventional methods. The term “salt” used herein is not limited as long as the salt is pharmacologically acceptable; preferred examples of salts include a hydrohalide salt (for instance, hydrochloride, hydrobromide, hydroiodide and the like), an inorganic acid salt (for instance, sulfate, nitrate, perchlorate, phosphate, carbonate, bicarbonate and the like), an organic carboxylate salt (for instance, acetate salt, maleate salt, tartrate salt, fumarate salt, citrate salt and the like), an organic sulfonate salt (for instance, methanesulfonate salt, ethanesulfonate salt, benzenesulfonate salt, toluenesulfonate salt, camphorsulfonate salt and the like), an amino acid salt (for instance, aspartate salt, glutamate salt and the like), a quaternary ammonium salt, an alkaline metal salt (for instance, sodium salt, potassium salt and the like), an alkaline earth metal salt (magnesium salt, calcium salt and the like) and the like. In addition, hydrochloride salt, sulfate salt, methanesulfonate salt, acetate salt and the like are preferred as “pharmacologically acceptable salt” of the compounds disclosed herein.

Preferences and options for a given aspect, feature, embodiment, or parameter of the technology described herein should, unless the context indicates otherwise, be regarded as having been disclosed in combination with any and all preferences and options for all other aspects, features, embodiments, and parameters of the technology.

### Modes of Action

Without being bound by theory, the modes of action for the compounds of Formula (I) described herein and active moieties thereof (e.g., WR1065) are described herein below. Accordingly, contemplated herein are methods of administering compositions described herein to achieve antimicrobial (e.g., antibiotic, antiviral, antifungal, antiparasitic) and/or antineoplastic effects through the modes of action described herein.

WR1065 is a reactive nucleophile that may have the ability to toggle between weak and strong nucleophilic states. Thus, WR1065 can participate in nucleophile-electrophile reactions. WR1065 has been demonstrated to bind to nucleic acids and proteins, with the ability to alter the conformation and activity of the p53 protein (North et al., “Restoration of Wild-Type Conformation and Activity of a Temperature-Sensitive Mutant of p53 (p53 (V272M)) by the Cytoprotective Aminothiol WR1065 in the Esophageal Cancer Cell Line TE-1,” Mol. Carcinog. 33 (3): 181-188 (2002) and Pluquet et al., “Activation of p53 by the Cytoprotective Aminothiol WR1065: DNA-Damage-Independent Pathway and Redox-Dependent Modulation of p53 DNA-Binding Activity,” Biochem. Pharmacol. 65 (7): 1129-1137 (2003), which are hereby incorporated by reference in their entirety). It is postulated that this binding occurs via reaction with cysteine residues. WR1065 also is reported to bind to other transcription factors. The exact binding sites and reactions involved have not been reported.

WR1065 is reported to have reductant capacity equal to or exceeding that of dithiothreitol (DTT) (Pluquet et al., “Activation of p53 by the Cytoprotective Aminothiol WR1065: DNA-Damage-Independent Pathway and Redox-Dependent Modulation of p53 DNA-Binding Activity,” Biochem. Pharmacol. 65 (7): 1129-1137 (2003), which is hereby incorporated by reference in its entirety). As a thiol, it participates in thiol oxidation/reduction reactions and is a thiol-disulfide exchange reaction modulator (Treskes et al., “The Reversal of Cisplatin-Protein Interactions by the Modulating Agent WR2721 and its Metabolites WR1065 and WR33278,” Cancer Chemother. Pharmacol. 29 (6): 467-470 (1992) and Nagy et al., “Protection Against cis-diamminedichloroplatinum Cytotoxicity and Mutagenicity in V79 Cells by 2-[(aminopropyl)amino]ethanethiol,” Cancer Res. 46 (3): 1132-1135 (1986), each of which is hereby incorporated by reference in its entirety).

WR1065 is reported to be able to replace some residues in disulfide bonds, and to compete with others in disulfide bond formation and/or exchange. Data reported for the effects of WR1065 on thiol oxidation/reduction reactions suggest that it participates in some reactions and not others. These data suggest that it cannot reduce all disulfide bonds, but only some (Treskes et al., “The Reversal of Cisplatin-Protein Interactions by the Modulating Agent WR2721 and its Metabolites WR1065 and WR33278,” Cancer Chemother. Pharmacol. 29 (6): 467-470 (1992), which is hereby incorporated by reference in its entirety). Data suggest that the activity of WR1065 is regulated by the redox balance of the environment, even as it participates in modulating that environment.

WR1065 is reported to be a hydrogen donor, and less often, to be a hydrogen acceptor. It can alter the electrostatic characteristics of solutes and/or local microenvironments around molecules and subcomponents thereof. It also is a polyamine analog. WR1065 most closely resembles putrescine, but also has similarities to spermidine and spermine. Reported data show that WR1065 can induce some polyamine-like effects.

WR1065 is reported most often to function as an antioxidant and redox status modulator, with the ability to scavenge reactive oxygen, nitrogen, and electrophilic species. However, the antioxidant effects are reported to occur at high doses well above the levels at which antiviral and anticancer effects have been reported.

Since the PEG scaffolds of the two aminothiol-conjugates exemplified herein also are hydrophilic nucleophiles, it is probable that they have some effects similar to those of WR1065 and/or that they synergize with and/or augment the activities of WR1065. Indeed, the anticancer study results described herein support synergism between the two molecules when delivered together.

Antiviral Effects Based Upon Nucleic Acids: WR1065 and some analogs such as cysteamine and/or cystamine bind to DNA and other nucleic acids and stabilize and/or alter their conformation. This ability is considered to reflect the polyamine-like nature of WR1065 and its analogs. as polyamines also bind to nucleic acids with similar effects. There is some evidence that aminothiols (WR1065, cysteamine, cystamine, others) affect polymerases, including the viral polymerase of HIV-1 (Ho et al., “Cystamine Inhibits HIV Type 1 Replication in Cells of Monocyte/Macrophage and T Cell Lineages,” AIDS Res. Hum. Retroviruses 11 (4): 451-459 (1995) and Kalebic and Schein, “Organic Thiophosphate WR-151327 Suppresses Expression of HIV in Chronically Infected Cells,” AIDS Res. Hum. Retroviruses 10 (6): 727-733 (1994), which are hereby incorporated by reference in their entirety). Adverse effects upon nucleic acid conformation and/or polymerase activity can limit the ability of viral and/or host polymerases to bind to the viral genome and can prevent transcription. This effect will result in a general antiviral effect and is not limited to the DNA/RNA/polymerases of just one virus or one group of similar viruses.

Antiviral Effects Based Upon Modes of Action as a Nucleophile—Protease Inhibition: Many/most viruses rely upon proteases to carry out key steps involved in the viral life cycle. These proteases can be divided into three groups: cysteine proteases, serine proteases, and aspartyl proteases. These viral proteases are described as working by one of two general types of catalytic mechanisms (1-step catalysis or 2-step catalysis) to carry out similar functions needed by viruses to complete their life cycles. One important feature of viral proteases (and apparently proteases in general) is that they are promiscuous. This opens up the opportunity for WR1065 and/or a thiolated PEG scaffold (also a nucleophile) to react in these nucleophile-electrophile and hydrogen donation reactions, thereby preventing the protease from completing its virus-life cycle associated tasks (see, e.g., Sharma and Gupta, “Virus Proteases and their Inhibitors Chapter 1-Fundamentals of Viruses and their Proteases. 2017:1-24 (PMCID: PMC7150265), which is hereby incorporated by reference in its entirety).

Because of the role of proteases in viral replication, they are targets of many antiviral agents. Viruses with proteases that frequently are targeted by antiviral agents include alphaviruses, hepatitis C virus, picornaviruses, herpesviruses, adenoviruses, and flaviviruses. Lists of viruses in these families are presented below along with viruses against which WR1065 has been shown to have antiviral activity.

Adenoviridae contain five genera of viruses: Mastaadenovirus, Aviadenovirus, Atadenovirus, Siadenovirus, and Ichoadenovirus. There are 51 immunologically different human adenovirus serotypes in 6 species. These viruses all express cysteine proteases that have a catalytic triad at the active site composed of cysteine-histidine-glutamine. There is structural conservation of the active sites of these proteases between members of this virus family. WR1065 is reported to work against three species of adenovirus (Walker et al., “WR1065 Mitigates AZT-ddI-Induced Mutagenesis and Inhibits Viral Replication,” Environ. Mol. Mutagen. 50 (6): 460-472 (2009), which is hereby incorporated by reference in its entirety).

Alphaviruses are part of the Togaviridae virus family and include Chikungunya virus, O'Nyong-Nyong virus, Ross River virus, Semliki Forest virus, Sindbis virus, Eastern equine encephalitis virus, Venezuelan equine encephalitis virus, and Western equine encephalitis virus. Alphaviruses have a cysteine protease and also use host cell proteases to complete life cycle phases. The cysteine proteases of these viruses are similar to that of papain-like cysteine proteases and have a catalytic dyad at the active site composed of cysteine-histidine.

The flaviviridae family contains over 70 enveloped RNA viruses. Members of this virus family include Dengue virus, yellow fever virus, West Nile virus, Japanese encephalitis virus, Zika virus, and St. Louis encephalitis virus. Flaviviruses release RNA into host cells; this encodes a polyprotein that is processed by host cell enzymes and the viral protease to form structural and nonstructural proteins. The flavivirus protease is a chymotrypsin-like serine protease. This protease interacts with other viral proteins and viral RNA to complete its activities. Studies have shown that this protease is essential for viral replication.

Hepatitis C virus is a member of the Flaviviridae family. Two proteases essential for viral replication are serine proteases (these proteases are named NS3-4A and NS3). NS3 is a chymotrypsin-like serine protease with a catalytic triad at its active site composed of serine-histidine-aspartame. The structure of this enzyme is stabilized by zinc ion and three cysteines and a histidine. (As described infra WR1065 can bind metals, including zinc, and can interact with cysteine and histidine to alter protein conformation and function.)

Herpes viruses are DNA viruses in the Herpesviridae family. There are three subfamilies, alpha-, beta-, and gamma-Herpesviridae. Alpha-herpes family members include herpes simplex virus 1, herpes simplex virus 2, and varicella zoster virus. Beta-herpes virus members include human herpes virus 6, human herpes virus 7 and cytomegalovirus. Gamma-herpes family members include Epstein-Barr virus and human herpes virus 8. The herpes virus protease (a serine protease) is essential for viral replication.

HIV-1, human immunodeficiency virus type 1, and SIV, simian immuno-deficiency virus, are members of the Lentiviridae family. The HIV-1 protease is an aspartate protease with a catalytic triad composed of aspartate-threonine-glycine. The hydrophilicity and reliance upon hydrogen bonding provides sites and functions in which WR1065 can participate, and thus, disrupt protease function. WR1065 is reported to work against HIV-1 and SIV (Poirier et al., “Antiretroviral Activity of the Aminothiol WR1065 against Human Immunodeficiency virus (HIV-1) In Vitro and Simian Immunodeficiency virus (SIV) Ex Vivo,” AIDS Res Ther. 6:24 (2009), which is hereby incorporated by reference in its entirety).

Picornaviruses are members of the Picornaviridae family, which has 46 species and 26 genera. Enteroviruses belong in this family and include poliovirus, rhinovirus, coxsackievirus, and echo virus. Other family members include Apthovirus, Cardiovirus (encephalomyocarditis virus), Thieler's virus, and Hepatovirus. There are three picornavirus proteases, named 3C, 2A, and leader proteases; 2A and leader proteases occur in only some picornaviruses. The 3C and 2A proteases are cysteine proteases with a catalytic site composed of glycine-X-cysteine-glycine, a composition considered to be similar to that of chymotrypsin-like serine proteases (suggesting that they work in similar ways to carry out their functions). Other viruses with similar protease structures include hepatitis A virus and human rhinovirus 2 and 14, which encode papain-like cysteine proteases. The leader protease also is a cysteine protease.

Influenza viruses are members of the Orthomyxoviridae family. Influenza viruses are enveloped viruses that contains the protein hemagglutinin; this protein is critical for completion of the virus life cycle. Influenza virus relies upon cleavage of hemagglutinin to carry out its life cycle; this occurs via the activity of several host proteases including furin and proprotein convertase 5/6. Furin is located in host cell plasma membranes and also in the cytoplasm and also has roles in the activation of viral glycoproteins from many viruses, including measles virus, respiratory syncytial virus, yellow fever virus, HIV-1, and Ebola virus. Influenza hemagglutinin is activated by cleavage and this appears to occur at the plasma membrane by bound proteases and also in the cytosol. These proteases are named human airway trypsin-like protease (HAT/TMPRSS11D) and transmembrane protease serine S1 member 2 (TMPRSS2). These are serine proteases, with catalytic domains of the chymotrypsin S1 type. The linkage of these proteases to the plasma membrane is through a disulfide bond, and thus, WR1065 potentially could reduce this bond, thus, preventing enzyme localization in the plasma membrane. The human airway trypsin-like protease (HAT/TMPRSS11D) has been targeted by anti-influenza agents and has been shown to inhibit the virus. These proteases have been reported to have roles in the activation of both influenza A and B, SARS-COV, and human metapneumovirus (Bottcher-Friebertshauser et al., “Activation of Influenza Viruses by Proteases from Host Cells and Bacteria in the Human Airway Epithelium,” Pathog Dis. 69 (2): 87-100 (2013), which is hereby incorporated by reference in its entirety). WR1065 is reported to work against influenza strains representative of influenza A and influenza B (Walker et al., “WR1065 Mitigates AZT-ddI-Induced Mutagenesis and Inhibits Viral Replication,” Environ. Mol. Mutagen. 50 (6): 460-472 (2009), which is hereby incorporated by reference in its entirety).

There also are other proteases that can activate some influenza substrains, including proteases produced by bacteria. Importantly, studies have shown that inhibition of transmembrane protease serine S1 member 2 (TMPRSS2) had potent inhibitory activity against influenza virus replication in human airway epithelium and did not affect cell viability. Investigators have postulated that inhibition of host proteases that are involved in viral life cycles stages is not toxic to human cells because human cells have alternative enzymes and/or pathways that they use to resolve or bypass a blockage, while viruses lack these types of options. Combinations of a protease inhibitor and another antiviral agent have been reported to be highly synergistic.

Filoviridae: Ebola virus is a member of the Filoviridae family. Another member is Marburg virus. On its surface Ebola virus has a heterodimeric glycoprotein that is cleaved into two subunits by furin, a host cell protease. The subunits are further processed by the cysteine proteases cathepsin B and cathepsin L; these proteases are active in the acidic environment of the late endosome/lysosome. Without the activity of these proteases, Ebola virus cannot escape the lysosome and complete its life cycle. (The nitrosothiol form of WR1065 is reported to inhibit the cysteine protease cathepsin H (Whiteside et al., “Properties of Selected S-Nitrosothiols Compared to Nitrosylated WR-1065,” Radiat. Res. 157 (5): 578-588 (2002), which is here by incorporated by reference in its entirety). Blocking the activity of the cathepsin proteases has been used successfully to inhibit Ebola virus replication. However, blockage of just one protease may not be sufficient to inhibit Ebola virus replication, and there may be other unidentified proteases involved in the life cycle, which provides an opportunity for WR1065.

Another consideration: RNA viruses, including influenza viruses and filoviruses (Ebola) carry out one or more life cycle stages inside of endosomes located in the cell cytoplasm. WR1065 is reported to undergo localization in lysosomes due to its charge distribution, and endosomes can mature into lysosomes. This localization will have the effect of increasing or targeting WR1065 molecules towards proteases in lysosomes, while reducing drug concentration in the remaining cytosol.

Antiviral Effects Based Upon Modes of Action as a Thiol Oxidation Reduction Modulator: Example—Protease Inhibition: All viruses are adapted to the oxidant/reductant conditions of their host cells, but all or most also modulate these conditions so that the conditions are optimal for completing different life cycle stages. Disulfide-thiol equilibrium has roles in viral entry into cells, viral reactivity, and viral fusion (Suhail et al., “Role of Oxidative Stress on SARS-COV (SARS) and SARS-COV-2 (COVID-19) Infection: A Review.,” Protein J. 39 (6): 644-656 (2020), which is hereby incorporated by reference in its entirety), which are essential steps in viral life cycle completion. Many viruses control their environment by inducing the production of reactive oxygen and nitrogen species; this has been reported for influenza A, hepatitis C, Sendai, respiratory syncytial viruses, and SARS-COV-1 and -2 (Davis et al., “Regulation of the Dimerization and Activity of SARS-COV-2 Main Protease through Reversible Glutathionylation of Cysteine 300,” mBio 12 (4): e0209421 (2021), which is hereby incorporated by reference in its entirety). As examples, retroviral proteases, as shown by studies of HIV-1, HIV-2, and human T-cell leukemia virus 1, have proteases that are regulated by reversible oxidation of a cysteine or methionine amino acid that is located in the dimerization domain of the two subunits that make up the active protease. Davis et al. (Davis et al., “Regulation of the Dimerization and Activity of SARS-COV-2 Main Protease through Reversible Glutathionylation of Cysteine 300,” mBio. 12 (4): e0209421 (2021), which is hereby incorporated by reference in its entirety) hypothesized that cysteine moieties, especially those that are exposed on the surface of proteases such as the SARS-Cov-1 and -2 protease subunits, are susceptible to oxidation in a host intracellular oxidative environment, and that the oxidation state of these cysteines has a key role in the regulation of protein activity and virus life cycle completion (Davis et al., “Regulation of the Dimerization and Activity of SARS-COV-2 Main Protease through Reversible Glutathionylation of Cysteine 300,” mBio. 12 (4): e0209421 (2021), which is hereby incorporated by reference in its entirety). In addition to affecting enzyme conformation and activity at the catalytic site, the molecular conformation of the substrate binding site and of the substrate molecule also must be in the appropriate forms for the enzyme to carry out its function. These considerations show that there are multiple sites/opportunities for WR1065 to alter the activity of key enzymes required for viral replication.

Disulfide bonds are a type of bond structure that contributes to protein conformation and that is vulnerable to modulation by WR1065. Examples of viruses that have disulfide bonds in key proteins involved in life cycle stages are presented below.

Adenoviridae. The protease of adenovirus forms a complex with a cofactor peptide calls pVIc and without which the protease is inactive. This cofactor contains disulfide bonds that induce correct folding of the protein so that amino acids in the active site of the protease are properly positioned (Mangel and San Martin, “Structure, Function and Dynamics in Adenovirus Maturation,” Viruses 6 (11): 4536-4570 (2014), which is hereby incorporated by reference in its entirety). Contact between the adenovirus protease and the pVIc cofactor also involves 34 hydrogen bonds and a disulfide bond-all of these bonds provide sites where WR1065 or PEG-SH scaffold can interact and prevent complex formation that is required for activity. For more information about the role of disulfide bonds and thiol-disulfide exchange reactions involved in adenovirus protease activity (see, e.g., Mangel and San Martin, “Structure, Function and Dynamics in Adenovirus Maturation,” Viruses 6 (11): 4536-4570 (2014), which is hereby incorporated by reference in its entirety).

Alphaviruses. Glycoproteins in the spikes that are required for viral entry contain disulfide bonds that, if disrupted, attenuate or eliminate viral infectivity (Parrott et al., “Role of Conserved Cysteines in the Alphavirus E3 Protein,” J. Virol. 83 (6): 2584-2591 (2009), which is hereby incorporated by reference in its entirety).

Flaviviridae (e.g., hepatitis C virus). The envelop glycoprotein of hepatitis C virus affects viral assembly and entry functions and contains a disulfide bond (Wahid et al., “Disulfide Bonds in Hepatitis C Virus Glycoprotein E1 Control the Assembly and Entry Functions of E2 Glycoprotein,” J. Virol. 87 (3): 1605 1617 (2013), which is hereby incorporated by reference in its entirety).

Herpes viruses. Disulfide bonds in capsid proteins stabilize capsid structure of herpes simplex virus. These covalent cross-links are susceptible to reduction by dithiothreitol (DTT) (Szczepaniak et al., “Disulfide Bond Formation Contributes to Herpes Simplex Virus Capsid Stability and Retention of Pentons,” J. Virol. 85 (17): 8625-8634 (2011), which is hereby incorporated by reference in its entirety). As stated above, WR1065 is reported to be a more potent reducing agent than DTT (Pluquet et al., “Activation of p53 by the Cytoprotective Aminothiol WR1065: DNA-Damage-Independent Pathway and Redox-Dependent Modulation of p53 DNA-Binding Activity,” Biochem. Pharmacol. 65 (7): 1129-1137 (2003), which is hereby incorporated by reference in its entirety). Loss of disulfide bonds results in aberrant formation of viral particles (Szczepaniak et al., “Disulfide Bond Formation Contributes to Herpes Simplex Virus Capsid Stability and Retention of Pentons,” J. Virol. 85 (17): 8625-8634 (2011), which is hereby incorporated by reference in its entirety).

Lentiviridae. HIV-1 envelop protein contains disulfide bonds that contribute to its stability (Go et al., “Analysis of the Disulfide Bond Arrangement of the HIV-1 Envelope Protein CON-S gp140 DeltaCFI shows Variability in the V1 and V2 Regions,” J. Proteome Res. 10 (2): 578-591 (2011), which is hereby incorporated by reference in its entirety). Retroviral proteases, as shown by studies of HIV-1, HIV-2, human T-cell leukemia virus 1, have proteases that are regulated by reversible oxidation of a cysteine or methionine amino acid that is located in the dimerization domain of the two subunits that make up the active protease (Davis et al., “Regulation of the Dimerization and Activity of SARS-COV-2 Main Protease through Reversible Glutathionylation of Cysteine 300,” mBio. 12 (4): e0209421 (2021), which is hereby incorporated by reference in its entirety).

Papillomaviridae. Human and bovine papillomaviruses express a protein called L1 that is involved in capsid assembly and which contains a disulfide bond. Disruption of this bond was shown to prevent papillomavirus capsid assembly and disassembly (Li et al., “Intercapsomeric Disulfide Bonds in Papillomavirus Assembly and Disassembly,” J. Virol. 72 (3): 2160-2167 (1998), which is hereby incorporated by reference in its entirety).

Influenza virus. Influenza virus is an enveloped virus that contains the protein hemagglutinin; it exists in the viral envelop as a homodimer that depends upon a disulfide bond that links hemagglutinin-1 subunit to hemagglutinin-2 subunit. Hemagglutinin homodimer is formed from subunits during viral maturation, and thus, this is a site at which WR1065 can block its formation (Segal et al., “Disulfide Bond Formation During the Folding of Influenza Virus Hemagglutinin,” J. Cell Biol. 118 (2): 227 244 (1992), which is hereby incorporated by reference in its entirety).

Ebola virus. Ebola virus is a member of the Filoviridae family. Ebola virus has a heterodimer glycoprotein on its surface; the two subunits are held together by a disulfide bond (Nishimura and Yamaya, “A Synthetic Serine Protease Inhibitor, Nafamostat Mesilate, Is a Drug Potentially Applicable to the Treatment of Ebola Virus Disease,” Tohoku J. Exp. Med. 237 (1): 45-50 (2015), which is hereby incorporated by reference in its entirety).

Specific Example—SARS-Cov-2 Protease Inhibition: SARS-Cov-2 relies upon two proteases, named the main protease (M(pro) or 3CL (pro)), and papain-like protease (PL(pro)) to complete essential steps in its lifecycle. M(pro) is composed of two subcomponent monomers that carry out tasks needed for completion of initial phases of viral replication when they form a dimer (Davis et al., “Regulation of the Dimerization and Activity of SARS-COV-2 Main Protease through Reversible Glutathionylation of Cysteine 300,” mBio. 12 (4): e0209421 (2021), which is hereby incorporated by reference in its entirety). Dimer formation is required for activity.

The M(pro) proteases of SARS-Cov-2 contain multiple cysteines. The details of the steps involved in the initial autocatalytic processing of M(pro) from the two polyprotein subunits that make up the active dimer are not known, but in the case of similar proteins of other viruses, such as HIV, it is recognized that the reduction/oxidation status of these cysteines plays a key role in the activity of the protein. Thus, inappropriate oxidation or reduction of one or more cysteines has the potential to render these proteases nonfunctional.

There are multiple ways that one or both active ingredients in a BRG prodrug (i.e., an aminothiol-conjugates according to the present disclosure) can modify key cysteines and/or other amino acids so that the viral protease becomes nonfunctional. Discussion of potential inhibitory effects at enzyme catalytic sites and/or substrate binding sites are presented above.

The aminothiol-conjugates according to the present disclosure can bind to key amino acids and/or other amino acids that are required for dimer formation. Aminothiol-conjugates according to the present disclosure can bind to sufficient cysteines, or other amino acids, on polyprotein monomers to alter their conformation so that the microenvironmental conditions and distances between amino acids are altered. As an example, alterations in the distances between key amino acids in the catalytic binding site of the M(pro) protease of SARS-CoV-2 result in the inability to achieve binding between amino acids that is necessary for protein activity. Such alterations also can affect reactions in the catalytic site and/or can inhibit dimer formation, effects described for the drug ebselen (Sies and Parnham, “Potential Therapeutic Use of ebselen for COVID-19 and other Respiratory Viral Infections,” Free Radic Biol. Med. 156:107-112 (2020), which is hereby incorporated by reference in its entirety). Alterations in distances between residues have been reported for mutant versions of adenoviral proteases, where these changes resulted in loss of enzyme activity (Webster et al., “The Adenovirus Protease is Activated by A Virus-Coded Disulphide-Linked Peptide,” Cell. 72 (1): 97-104 (1993), which is hereby incorporated by reference in its entirety). At least one of the BRG active ingredients, WR1065, is recognized to have the ability to alter electrostatic interactions. Alterations in the electrostatic characteristics around key amino acids can change their reactivity, thereby, altering their reaction energies and adversely affecting protein activity. Current evidence suggests that at certain points in the SARS-Cov-2 lifecycle it is necessary for the virus to inactivate M(pro). This is achieved through oxidation by glutathionylation of cysteine 300 (see, e.g., Davis et al., “Regulation of the Dimerization and Activity of SARS-COV-2 Main Protease through Reversible Glutathionylation of Cysteine 300,” mBio. 12 (4): e0209421 (2021), which is hereby incorporated by reference in its entirety). As a potent reducing agent, WR1065, has the ability to prevent or reverse this oxidation step, and thereby, affect completion of SARS-Cov-2 later lifecycle stages. The finding that WR1065 is localized to lysosomes also can enhance its antiviral activity against at least some viruses that carry out critical steps in late endosomes/lysosomes, as discussed above.

All of these mechanisms can result in failure of the M(pro) protease to be able to function, and this alone is sufficient to result in a potent anti-SARS-Cov-2 effect. However, it also should be noted that similar processes, that is molecular conformations, interactions between amino acids on proteins, and electrostatic interactions in microenvironments around and between key interacting protein components, have roles in multiple stages of the life cycles of multiple viruses, and thus, provide multiple sites at which BRG active ingredients can induce antiviral effects. It also should be noted that the diversity of mechanisms that WR1065 can use to bring about antiviral effects will create a barrier to resistance development that will be difficult for viruses to overcome.

Antiviral activity of the aminothiol-conjugates according to the present disclosure is not dependent upon amino acid sequences in proteins. Many antiviral agents and some vaccines target specific amino acid sequences that have been identified as specific to viral proteins and not mammalian/human proteins. The disadvantage to this approach is that if the virus is able to undergo mutations that do not affect its viability but do affect target sites for antiviral agents and vaccines, then the agents can lose activity or become completely ineffective. This is not an issue for BRG active ingredients because they are not dependent upon specific amino acid sequences, but rather target overall phenomena related to protein conformation, microenvironmental characteristics, and types of molecular interactions upon which viruses depend. Since M(pro) bears a resemblance to proteases of other viruses (e.g., proteases of HIV-1, HIV-2, human T-cell leukemia virus (HTLV), picornavirus and Mason Pfizer monkey virus), the phenomena described for SARS-Cov-2 may be applicable to other viruses as well, and may be applicable to all viruses.

Safety for Normal Cells: Current evidence shows that aminothiol-conjugates of the present disclosure (e.g., 4SP65 and 1LP65) are safe for normal cells. This may be because mammalian/human cells do not depend to such a significant degree upon the oxidation-reduction status of a few limited amino acids, they do not depend upon specific/single nucleophile-electrophile interactions to activate single proteins necessary for completion of cell replication, and they are not heavily dependent upon electrostatic characteristics of micro-environmental conditions around one or just a few amino acids. In general, human and other mammalian cells depend upon complex series of post-translational modifications to amino acids, and have multiple redundant systems to ensure that key replication steps are achieved correctly so that cell growth and cell maintenance can occur.

The present technology may be further illustrated by reference to the following examples.

### EXAMPLES

The following examples are provided to illustrate embodiments of the present technology but are by no means intended to limit its scope.

### Example 1—Cytotoxic Effects of 4-Arm-Star-PEG-WR1065 (4-Arm-PEG Conjugated to WR1065) in Six Tumor Cell Lines

To evaluate the anticancer activity of 4-arm star polyethylene glycol conjugated to WR-1065 (4SP65), the anticancer efficacy of 4SP65 was determined in some of the same cell lines used by NIH/NCI, and the methodology used was the same as is used by the NCI to evaluate chemotherapeutic agents currently in use (O'Connor et al., “Characterization of the p53 Tumor Suppressor Pathway in Cell Lines of the National Cancer Institute Anticancer Drug Screen and Correlations With the Growth-Inhibitory Potency of 123 Anticancer Agents,” Cancer Res. 57 (19): 4285-300 (1997), which is hereby incorporated by reference in its entirety). Testing of 6 cancer types was completed: (i) breast cancer (MDA-MB-231 cells), (ii) lung cancer (A549), (iii) prostate cancer cell line (LNCaP), (iv) myelogenous leukemia (HL60 cells), (v) ovarian cancer (SK-OV-3), and (vi) prostate cancer (DU-145). The growth inhibitory dose 50% for each cell line is presented in Table 1.

For comparison purposes, the growth inhibitory does of 4SP65 required to reduce the growth of normal human mammary epithelial cells by 50% was above 300 micromolar. The exact value has not been determined as of yet due to the fact that 4SP65 forms a hydrogel in medium when present at a concentration that exceeds 300 micromolar.

TABLE 1
Average Growth Inhibitory Doses Required to Reduce in Vitro Cell
Growth by 50% following a 48 hour exposure to 4SP65

| | Average Growth Inhibitory Dose |
|---|---|
| Tumor Type | 50% [EC₅₀] (micromolar) |
| Breast CA (MDA-MB-231) | 4.5 |
| Lung CA (A549) | 15 |
| Prostate cancer cell line (LNCaP) | 9.5 |
| Myelogenous Leukemia (HL60) | 3 |
| Ovarian CA | 5 |
| (SK-OV-3) | |
| Prostate CA (DU-145) | 6.7 |

The methods used to obtain the growth inhibitory concentrations of 50% (EC50 values presented in Table 1) were as follows. Each cell line was grown in medium as recommended by the ATCC or as presented in the literature for that cell line. All cells were cultured in a water jacket incubator at 36-37° C. and in the presence of 5% CO₂. To ensure optimal growth and viability, all cells were grown on plates coated with FNC (In Vitrogen). Cells were refed with growth medium twice weekly until they had reached 60 to 70% confluence. At this point, the medium was replaced with growth medium supplemented with 4SP65 at doses ranging from 0 to up to 300 micromolar. Cells were allowed to grow in the presence of this supplemented medium for 48 hours, and then they were removed by trypsinization, stained with Trypan Blue and counted in a hemocytometer. Three to four replicates for each dose group per experiment were performed. The percentage cell death was determined by comparing the average number of surviving cells exposed to 4SP65 versus the average number of surviving sham exposed cells. The average EC₅₀, in micromoles, for each cell line tested are presented in Table 1. It should be noted that the methodology used does not distinguish well between cell killing versus growth arrest of cells, and thus, the EC₅₀ represents the dose of drug required to induce one or both effects. As note below, the EC₅₀ does not consider the initial cell population at the start of treatment, or time ‘zero’, leading the National Cancer Institute to develop three new special concentration parameters to improve the measurement of drug effectiveness; these extra dose-response metrics were determined in subsequent drug efficacy and cell viability assays for presented below.

### Example 2—Antiviral Effects of 4-Arm-PEG-WR1065 in Cells Infected with Mouse Coxsackie B Virus (Prophetic)

Mouse cardiomyocytes will be plated at 70 to 80% confluence in growth medium and allowed to plate down and enter the growth cycle for 24 hours. Then, the growth medium will be removed and the cells will be exposed to medium containing dilutions of mouse coxsackie B virus for 30 mins. At the end of this time period, the virus-containing medium will be removed and the cells will be fed with 4-arm-PEG-WR1065-supplemented medium, where the dose of 4-arm-PEG-WR1065 ranges from 0.5 to 20 microM. Plates of control cells will be exposed to medium containing dilutions of coxsackie B virus and then will be refed at 6 hours with unsupplemented growth medium. All plates will be refed with their respective media every three days. At 72 hours, and every three days thereafter, medium will be removed and assayed by RT-PCR for viral replication. Compared to control, virus-infected cells, viral replication is predicted to be reduced by 90% to 99% by 6 days post-exposure. The degree of viral replication is expected to continue to decline for up to 10 days post-exposure. In comparable experiments, 1LP65 will be used in place of 4-arm-PEG-WR1065.

### Example 3—Cytotoxic Effect of 4-Arm-PEG-WR1065 (4SP65) Against Bacteria, Yeast, and Fungi (Prophetic)

The antimicrobial activity of 4SP65 will be tested against the bacteria, yeast, and fungi described in U.S. Appl. Pub. No. 2008/0027030 to Stogniew and Bourthis (“Stogniew and Bourthis”), which is hereby incorporated by reference in its entirety. Experiments as described herein will be performed in which the antimicrobial agent to be tested will be 4SP65 instead of amifostine. The growth inhibitory activity of 4SP65 will be tested alone and also in combination with other drugs. The antimicrobial effects of 4SP65 are predicted to be at least 8- to 12-fold greater than those described for amifostine in Stogniew and Bourthis. In comparable experiments, 1LP65 will be used in place of 4-arm-PEG-WR1065.

### Example 4—Cytotoxic Effects of 4-Arm-PEG-WR1065 on Normal Human Mammary Epithelial Cells (M99005)

Normal human mammary epithelial cells were grown as described in Example 1, with the exception that the growth medium was as MEBM (purchased from American Type Tissue Culture Collection). When the cells had reach 50 to 60%, the growth medium was removed and medium supplemented with 0 to 300 microM 4SP65 was added to each well containing cells. At 48 hours, the cells were removed by trypsinization stained with Trypan Blue, and counted using a hemocytometer. No inhibition of cell growth was observed at any drug concentration except at the 100 microM exposure level. Cell growth was inhibited by approximately 22% to 40%. Above 100 microM and up to 300 microM no evidence of cell growth inhibition was observed. Thus, the results showed a biphasic curve that did not reach 50% growth inhibition. The finding of a biphasic growth inhibition curve for WR-1065 has been reported previously (Calabro-Jones et al. “The limits to radioprotection of Chinese hamster V79 cells by WR-1065 under aerobic conditions.” Radiat Res. 149:550-559 (1998), which is hereby incorporated by reference in its entirety). Additional experiments now have been completed and are described infra).

### Example 5—Antiviral Effects of 4-Arm-PEG-WR-1065 Against Zika Virus and/or Other Positive Strand RNA Viruses (Prophetic)

Vero cells or other cells permissive for infection by Zika virus will be grown as described above (see Example 1) until 50 to 70% confluent. Then the cells will be treated with 4SP65 for up to 48 hours at drug levels that range from 0 to 100 microM. At the end of this exposure period, growth medium supplemented with 4SP65 will be removed and replaced with growth medium containing differing infectious units of Zika virus. Evidence of virus-induced cytotoxic effects will be assessed at multiple time points post-virus exposure to determine the ability of 4SP65 to reduce or prevent viral infection. In a similar experiment, cells will be infected with the virus for 30 minutes, and then exposed to 4SP65 at doses ranging from 0 to 100 microM and for time periods that range from 0 to 48 hours. The antiviral therapeutic efficacy of 4SP65 will be determined and is expected to fall within the range of 0.1 to 13 microM. The same experimental design will be used to test the antiviral efficacy of 4SP65 against other viral pathogens of concern to humans or animals. Antiviral efficacy in the range of 0.1 to 13 microM is expected to be observed for all experiments.

### Example 6—Preparation of Compound 7

### Step 1. Boc Protection

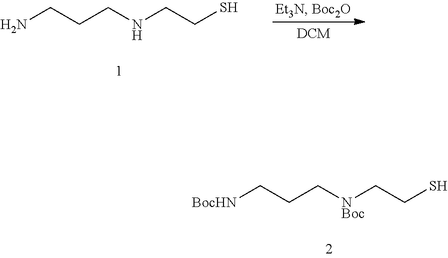 [SMILES: NCCCNCCS ; CC(C)(C)OC(=O)NCCCN(CCS)C(=O)OC(C)(C)C]

Substrate 1 (as dihydrochloride salt, 1.21 mmol) was dissolved in anhydrous dichloromethane (5 ml). Triethyl amine (6 eq.) and boc anhydride (2.1 eq) were added and the reaction was stirred at ambient temperature overnight under a positive nitrogen atmosphere. The next day, the reaction was diluted with dichloromethane and washed with brine. The organic layer was dried over magnesium sulfate and concentrated in vacuo to give compound 2 as a clear oil in 85% yield. The compound was used in the next step without purification.

Step 2. Coupling with Disulfide

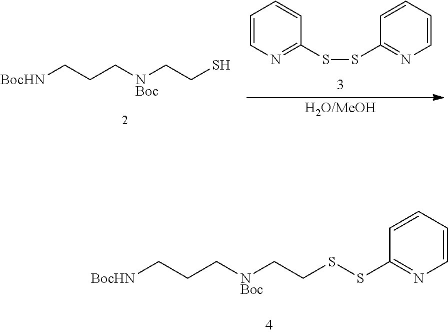

Substrate 2 (1.03 mmol) was dissolved in 1/1 water/methanol (10 ml) and disulfide 3 (2 eq.) was added. The reaction was stirred at ambient temperature under nitrogen overnight. Next day the reaction was concentrated in vacuo and diluted with dichloromethane. It was washed with brine and dried over magnesium sulfate. The product 4 was purified by column chromatography with silica gel and hexane/ethyl acetate gradient. The product was isolated in 46% yield.

Step 3. Coupling with Star Polymer

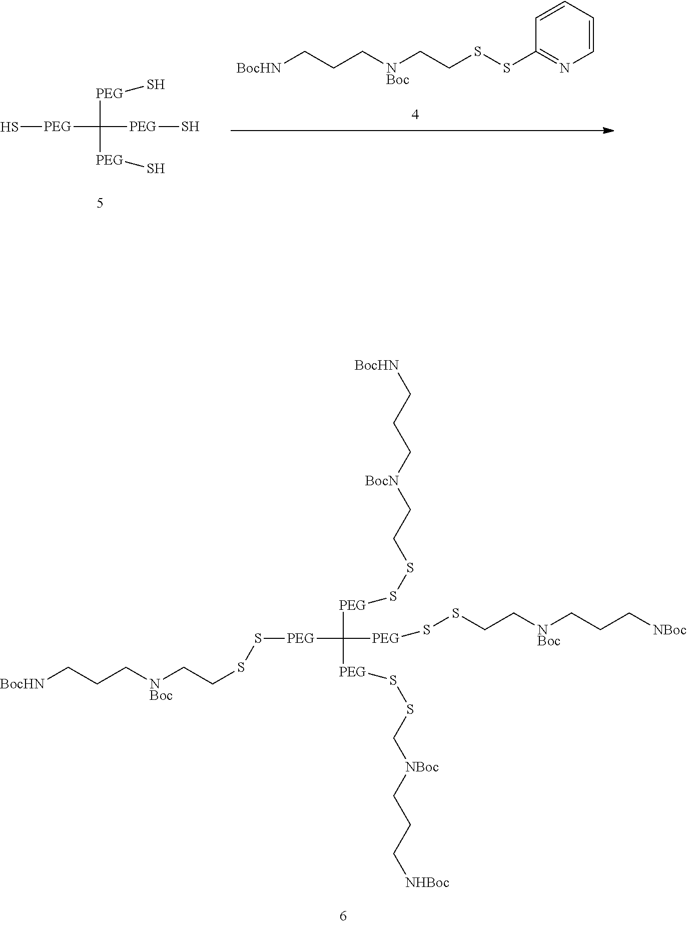

To a solution of star polymer 5 (0.75 g, average molecular weight 10,000 Daltons) in PBS (8 ml, pH 7.4) was added a solution of disulfide 5 (0.45 mmol) in ethanol (2 ml). The reaction was stirred for 4 hours at ambient temperature and then lyophilized overnight. The crude was dissolved in water (4 ml) and DMSO (2 ml) and was dialyzed against water for 48 hours with four water changes. Afterwards, the solution was lyophilized and 814 mg of conjugate 6 was isolated.

### Step 4. Deprotection to Give Conjugate 7

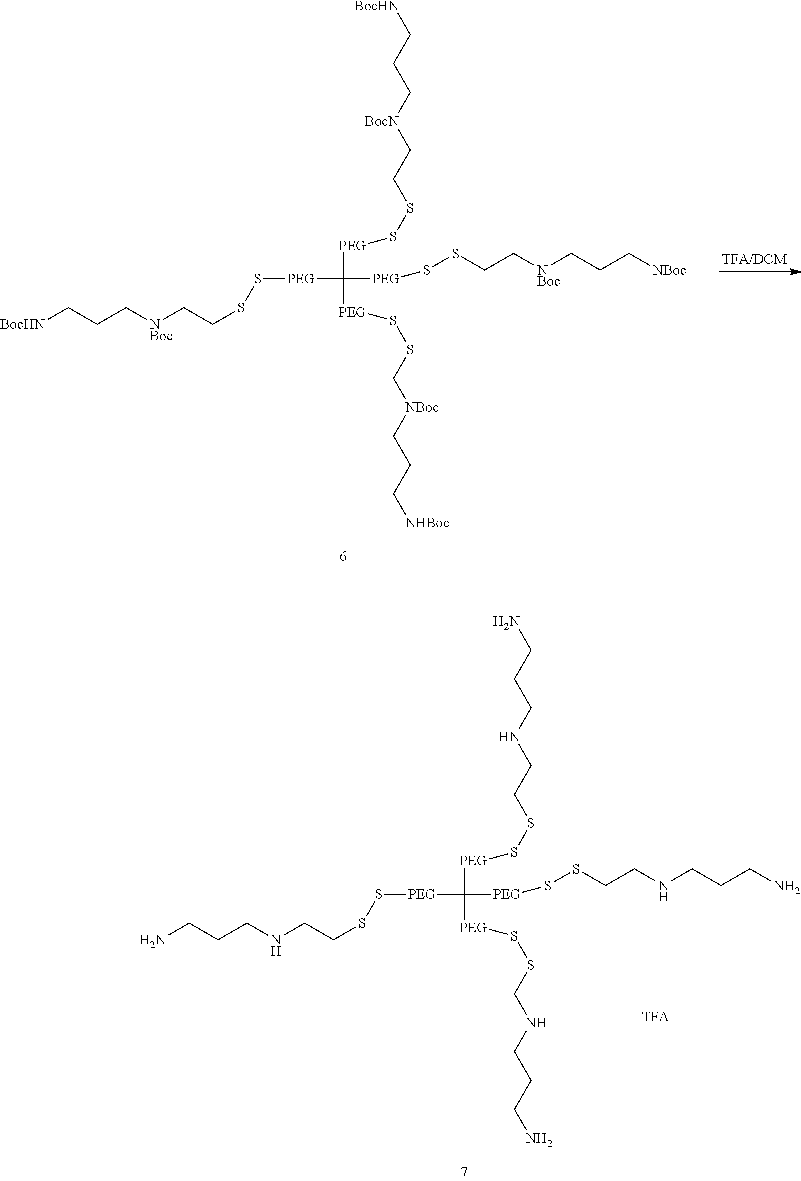

Conjugate 6 (814 mg) was treated with 1/1 TFA/dichloromethane (5 ml) for 30 min. The solvent was removed in vacuo and the residue was dried on a vacuum pump overnight. Next day, the residue was washed with ethyl ether (twice) and further dried on a vacuum pump overnight. 750 mg of conjugate 7 (“4SP65”) was obtained. MALDI analysis indicated an average mass of 10,531.95 Da, which suggested incorporation of four WR1065 units on average.

### Materials and Methods for Examples 7-12

### Chemicals, Pharmaceuticals, and Reagents

WR1065 dihydrochloride [2-(3-aminopropyl)aminoethanethiol dihydrochloride, empirical formula C₅H₁₄N₂S·2HCI, CAS #14653-77-1, 207.16 Da; catalog #W2020] and 4-arm-PEG-SH [pentaerythritol core, formula C (CH₂O(CH₂CH₂O)ₙCH₂CH₂SH) 4, 10,000 Da average; catalog #JKA7008] were purchased from Sigma-Aldrich (St. Louis, MO, USA). m-PEG6-thiol (formula C13H28O6S, CAS #441771-60-4, 312.4 Da, catalog #BP-22084) was obtained from BroadPharm (San Diego, CA, USA). Additional high-purity chemicals for synthesizing 4SP65/1LP65 were obtained by MedChem Partners. Amifostine was purchased from Moravek Biochemicals (Brea, CA, USA). Other reagents including Trypan Blue and CyQUANTR NF Cell Proliferation Assay Kits were obtained from ThermoFisher (Waltham, MA, USA).

### Cell Lines and Culture Conditions

Normal human mammary epithelial cell (NHMEC) strains used to assess potential cytotoxicity of 4SP65/1LP65 were established from reduction mammoplasty tissue from a healthy female donor (#M99005 or ‘Strain-1’) (Keshava et al., “Induction of CYP1A1 and CYP1B1 and Formation of Carcinogen-DNA Adducts in Normal Human Mammary Epithelial Cells Treated with Benzo[a]pyrene,” Cancer Lett. 221 (2): 213-24 (2005), which is hereby incorporated by reference in its entirety) or purchased from ATCC (Manassas, VA, USA; batch #70043304 or ‘Strain-2’) or Cell Applications (San Diego, CA, USA; lot #1669 or ‘Strain-3’). Human cancer cell lines including HCC38 (Cat #CRL2314™), MDA-MB-231 (Cat #HTB-26™), A549/ATCC (Cat #CL-185™), National Cancer Institute (NCI)-H460 (Cat #HTB177™), NCI-H1437 (Cat #CRL-5872™), NCI-H1975 (Cat #CRL-5908™), DU145 (Cat #HTB-81™), LNCaP (Cat #CRL-1740™), PC3 (Cat #CRL-1435™), PANC1 (Cat #CRL-1469™), SKOV3 (Cat #HTB-75™), and HL60 (Cat #CCL-240™), which were free of mycoplasma, were purchased from ATCC. HMESO1 (CLS Cell Lines Service GmbH, Cat #BHC11100791), PPMMill, and TOV21G cells (ATTC Cat #CRL-11730), obtained from Dr. Brian Cunniff, were verified to be Mycoplasma negative using the LookOutR Mycoplasma PCR Detection Kit (Sigma-Aldrich, Cat #MP0035) and to match previously annotated DNA fingerprints by the Vermont Integrative Genomics Resource DNA Analysis Facility.

Cell culture medium components from various vendors were obtained from ThermoFisher Scientific. Normal mammary epithelial cells were cultured in Mammary Epithelium Growth Medium (Lonza, Lexington, MA, USA), containing BulletKit™ growth supplements, at 37° C. in a humidified incubator with 5% CO₂. Mesothelioma and ovarian cancer cell lines were maintained in 50:50 DMEM (Corning, Manassas, VA, USA)/F12 medium (Lonza) supplemented with 10% (vol/vol) fetal bovine serum (FBS; Corning) and 100 U/mL penicillin-streptomycin (Corning). H1975 cells were cultured in an ATCC modified RPMI 1640 medium (Gibco, Waltham, MA, USA) with low L-glutamine, 10% FBS, and Pen-Strep. All other human cancer cell lines were grown in RPMI 1640 medium (Corning) supplemented with FBS and penicillin-streptomycin.

### Cell Viability Assays

To test drug effectiveness in individual cell lines, the CyQUANT® Proliferation Assay was used according to the manufacturer's instructions for quantifying viable cells with a Tecan Infinite 200 PRO plate reader (San Jose, CA, USA) to generate standard curves for cell numbers for each cell line and then to measure the starting cell numbers at drug treatment initiation. After drug treatments, fluorescence readings from 96-well plates were obtained using the CyQUANT dye and compared to standard curves for each cell line to determine cell viability. DNA dyes are a highly reliable method for determining the anticancer effects on cell proliferation (Eastman A., “Improving Anticancer Drug Development begins with Cell Culture: Misinformation Perpetrated by the Misuse of Cytotoxicity Assays,” Oncotarget. 8 (5): 8854-8866 (2017), which is hereby incorporated by reference in its entirety). Trypan blue exclusion was used to estimate cell numbers in a few pilot experiments and was thereafter applied to confirm cell death.

To assess the effects of 4SP65 on the growth of normal epithelium, experiments were performed using NHMEC strains from three donors (Keshava et al., “Induction of CYP1A1 and CYP1B1 and Formation of Carcinogen-DNA Adducts in Normal Human Mammary Epithelial Cells Treated with Benzo[a]pyrene,” Cancer Lett. 221 (2): 213-24 (2005), which is hereby incorporated by reference in its entirety). Strain-1 NHMECs were plated at 5,000 cells/well in 24-well dishes and allowed to reach 50% confluence before treatment with 0-300 μM 4SP65 for 48 hours. Cell viability was determined using a hemocytometer and trypan blue exclusion assay. Two other NHMEC strains, exposed to 0-150 μM 4SP65 or 0-500 μM 1LP65, were handled in the same fashion except that viability of cells in 96-well plates was determined using CyQUANT dye.

A series of experiments was conducted to assess the relative efficacy of 4SP65, 1LP65, and amifostine as single agents in the same human cancer cell lines. First, the effectiveness of 4SP65 was tested against 15 cancer cell lines derived from seven tissues. For short-term cell viability assays, 2000-5000 cells/well were plated in 96-well dishes and incubated for 24 hours to allow cells to enter log-phase growth. Then starting cell numbers were determined and the remaining cells were treated with 0 to ≤50 μM 4SP65 or 0 to ≤200 μM 1LP65 to characterize drug effectiveness following a single 48-hour exposure. The effectiveness of 0 to ≤500 μM amifostine was tested in sets of cancer cell lines reported to have either medium, high, or no expression of alkaline phosphatase.

### Data Analysis and Statistical Testing

The experimental design followed guidelines for comparisons between drugs used by the NCI (Monks et al., “Feasibility of a High-Flux Anticancer Drug Screen using a Diverse Panel of Cultured Human Tumor Cell Lines,” J. Natl. Cancer Inst. 83 (11): 757-66 (1991), which is hereby incorporated by reference in its entirety) along with modifications recommended by Eastman (Eastman A., “Improving Anticancer Drug Development begins with Cell Culture: Misinformation Perpetrated by the Misuse of Cytotoxicity Assays,” Oncotarget. 8 (5): 8854-8866 (2017), which is hereby incorporated by reference in its entirety), while data analyses used NCI methodology (Monks et al., “Feasibility of a High-Flux Anticancer Drug Screen using a Diverse Panel of Cultured Human Tumor Cell Lines,” J. Natl. Cancer Inst. 83 (11): 757-66 (1991), which is hereby incorporated by reference in its entirety) along with those of Brooks et al. (Brooks et al., “Applicability of Drug Response Metrics for Cancer Studies using Biomaterials,” Philos. Trans. R Soc. Lond. B Biol. Sci. 374 (1779): 20180226 (2019), which is hereby incorporated by reference in its entirety) to characterize concentration parameters that measure drug effectiveness following a single antagonist cancer drug treatment (Tong, F. P., Dissertation, Statistical Methods for Dose-Response Assays, University of California, Berkeley (2010), which is hereby incorporated by reference in its entirety). Five metrics were calculated and used to characterize the shape of the dose-response curves following exposure to a single test agent. Using cell numbers based on fluorescence measurements and standard curves [time zero, (Tz), control growth, (C), and test growth in the presence of drug at five or more concentration levels (Ti)], the percentage growth was calculated at each drug concentration level. The percentage of growth inhibition was calculated as follows:

$$[ ( Ti - Tz ) / ( C - T z ) ] \times 100 \\ for concentrations for which Ti \text{>} /= Tz \qquad Eq 1$$

$$[ ( Ti - Tz ) / Tz ] \times 100 \\ for concentrations for which Ti < T z \qquad Eq 2$$

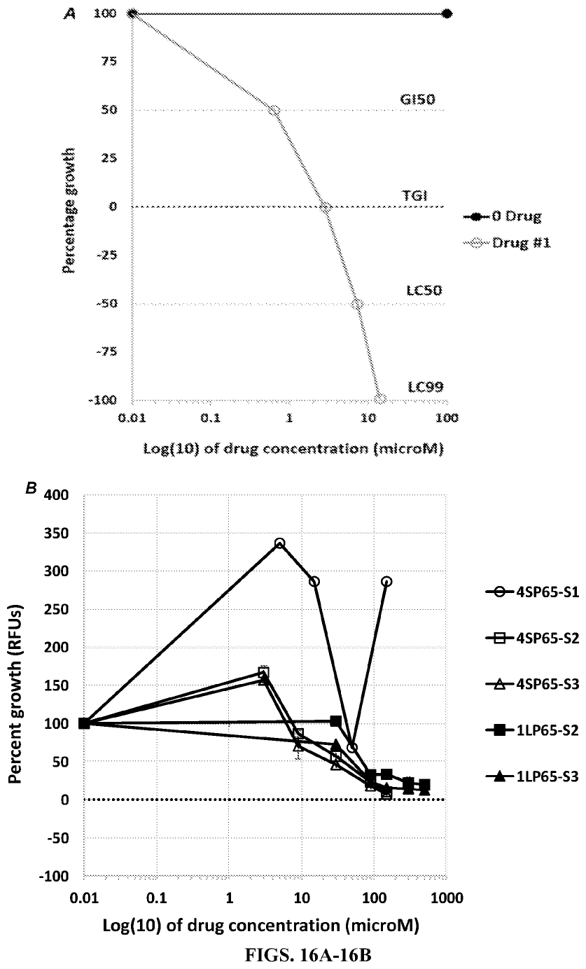

The inhibitory concentration 50% (IC₅₀) was calculated as the concentration of the drug that reduced the growth of treated cells by 50% compared to control cells. However, IC₅₀ does not consider the initial cell population at time zero, leading to the development of three new special concentration parameters to improve the measurement of drug effectiveness (FIG. 16A). The growth inhibition of 50% (GI50) was calculated as [(Ti−Tz)/(C−Tz)]×100=50. The drug concentration resulting in total growth inhibition (TGI) was calculated using Equation 1=0. The LC50/99 was calculated using Equation 2=−50 and Eq. 2=−99, respectively. To determine dose-response metrics in cancer cell lines, relationships between drug exposure levels and cell counts were used to calculate GI values for each exposure level, plotted on semi-log curves, and modelled using best fit regression curves and trend line formulas generated by Excel. For NHMECs, the relationships between drug exposure levels and fluorescence units were used to define dose-response metrics. The selectivity index (SI) values for individual drug treatments were calculated as IC₅₀ levels in NHMECs divided by the IC₅₀ values in cancer cell lines. Statistical comparisons of differences in individual dose-response metrics between drug treatments and various cell lines were conducted using Student's t-test, with P-values <0.05 considered significant.

### Example 7—Synthesis of 4SP65 and 1LP65

A novel prodrug was designed to deliver WR1065 to both normal and diseased cells to extend its use to new disease areas. The resulting family of prodrugs (referred to at times herein as BRG prodrugs which are exemplary aminothiol-conjugates of the present disclosure) deliver WR1065 conjugated to assorted PEG-thiol scaffolds, yielding conjugates stable extracellularly with single/multiple active moieties released intracellularly from the PEG scaffold non-enzymatically or enzymatically by essential thiol reduction/oxidation and thiol-disulfide exchange reaction components (Nagy, P., “Kinetics and Mechanisms of Thiol-Disulfide Exchange Covering Direct Substitution and Thiol Oxidation-Mediated Pathways,” Antioxid Redox Signal 18 (13): 1623-1641 (2013), which is hereby incorporated by reference in its entirety). A novel large-molecule therapeutic composed of WR1065 conjugated via bioreducible disulfide bonds to each thiol-terminated arm of a 4-arm PEG-SH scaffold to yield 4-‘star’ PEG-S-S-WR1065 (4SP65) was synthesized. Preclinical studies of 4SP65 as described herein above were conducted to define the relationships between 4SP65 exposures and anticancer effects such as cell growth inhibition and cell death in a panel of diverse human cancer cell lines with differing TP53 gene mutation statuses. Another small molecule therapeutic m-PEG6-S-S-WR1065 (1LP65), composed of WR1065 conjugated to one end of a small-molecule ‘linear’ PEG-thiol scaffold, was synthesized to compare of the relative cytostatic/cytocidal activities of 4SP65 (delivering four WR1065/molecule intracellularly), 1LP65 (delivering one WR1065/molecule intracellularly), and amifostine (delivering one WR1065/molecule extracellularly). These preclinical drug effectiveness studies demonstrated that the aminothiol-conjugates of the present disclosure have dose-related growth inhibitory, cytolytic, and other effects as described herein, in all human cancer cell lines tested, far exceeding the narrow growth inhibitory effects of amifostine in limited cell lines.

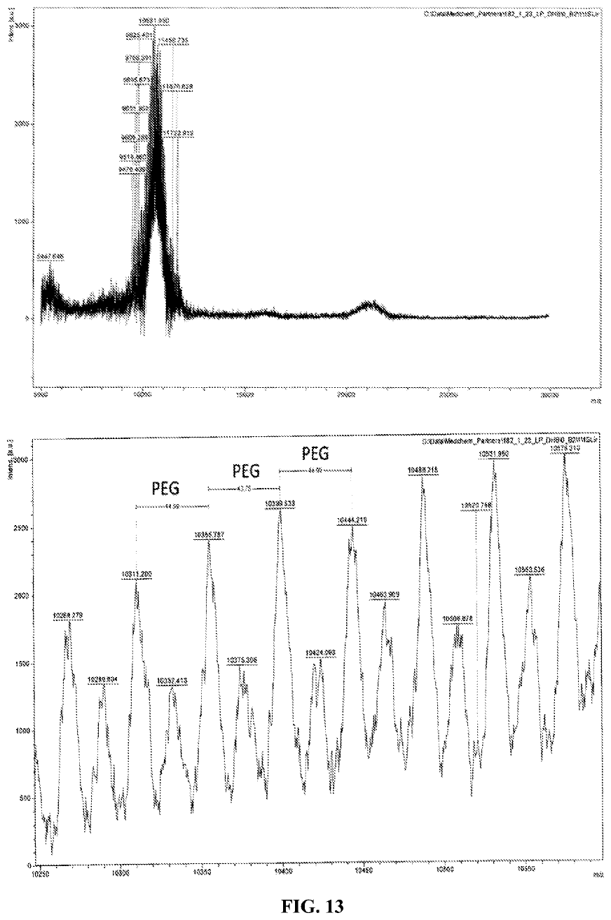

The 4SP65 aminothiol-conjugate was synthesized as described in Example 8 above. The spectrum from MALDI of conjugate 7 showed a peak corresponding to 4SP65 at an average MW of 10,531.95 Da (FIG. 13). The boc deprotected aminothiol conjugated to 4arm-PEG-SH (10,000 Da) was 146 Da so that if all four arms were reacted the result would be a polymer of 10,584 Da, indicating that the 4SP65 molecules used in reported experiments had four units of WR1065 incorporated on average.

The 1LP65 aminothiol-conjugate was prepared using the same synthetic process as described in Example 8 above except that in Step 3 of the synthesis, m-PEG6-thiol:

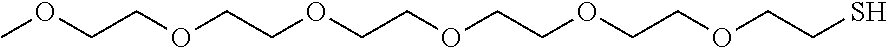 [SMILES: COCCOCCOCCOCCOCCOCCS]

was used instead of polymer 5. Following the deprotection step, carried analogously to Step 4 of Example 8, 1LP65:

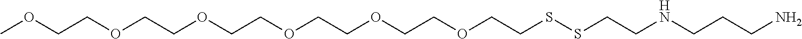 [SMILES: COCCOCCOCCOCCOCCOCCSSCCNCCCN]

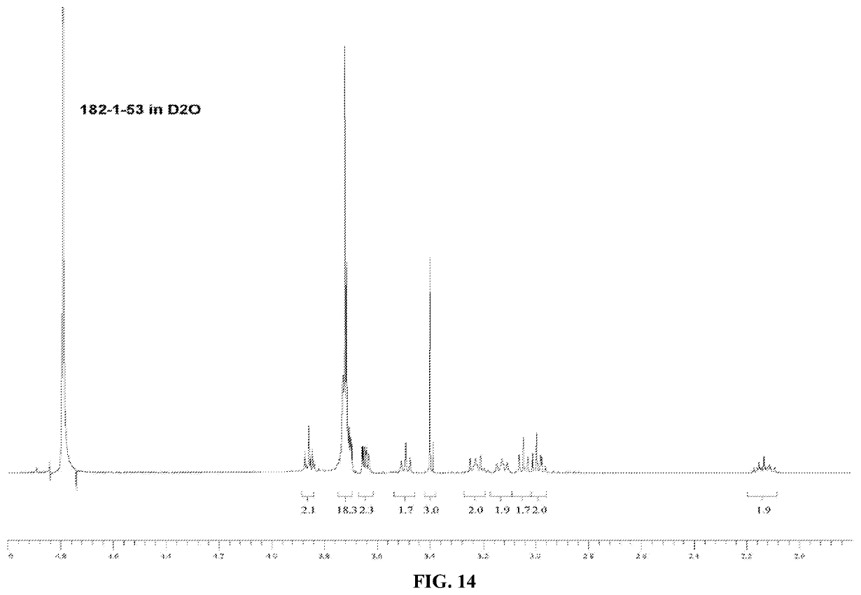

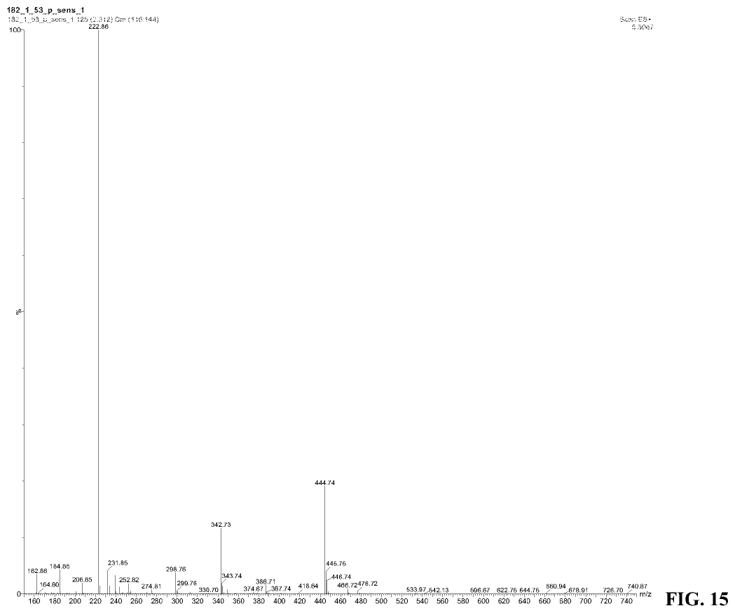

was prepared as di-trifluoroacetic acid salt (1LP65 is the abbreviation for the di-trifluoroacetic acid salt of the above prodrug). The molecular weight of the dissolved 1LP65 was 483 Da. The experimental findings related to the solubility, storage, and stability of 1LP65 and 4SP65 are presented in FIGS. 13-15.

### Example 8—Solubility, Storage, and Stability of 4SP65 and 1LP65

The solubility of 4SP65 was tested in de-ionized water, PBS, DMSO, and RPMI or DMEM base medium with and without FBS. While the drug was partially soluble in all media evaluated except DMSO, a portion of the drug absorbed liquid to form a gel that was consistent across all aliquots of drug prepared for use and that was not altered by the solvent used. heating up to 37° C. for up to 3 days, and shaking or rocking for up to 3 days at RT or 4° C. For use in experiments, an aliquot of drug was first mixed in medium, the solvent was removed and saved, and gel pieces were recovered, dried on a vacuum pump, and weighed to determine the mass of drug that did not dissolve. The difference in the starting weight of drug placed into solvent minus the weight of the residue after drying on a vacuum pump was considered to represent the amount of drug that went into solution. The amount of drug that was dissolved at a given time ranged from 40 to 61%, averaging 49.7±8.1% (SD). The weight of the 4SP65 in solution was then used for diluting the drug to concentrations of 200 or 400 μM in supplemented RPMI or DMEM/F12 medium for routine treatment of cells. The dried residue was not used in the in vitro drug efficacy studies. The formation of a gel in the aqueous solvents tested was unexpected since the 4SP65 molecule was found to have nearly four units of WR1065 incorporated on average, leaving few free thiols in polymer arms of the PEG scaffold for cross-linking reactions to occur. However, unintended topical entanglements between polymer chains of multi-arm PEG-thiol molecules without disulfide formation have been reported (Tang and Olsen, “Controlling Topological Entanglement in Engineered Protein Hydrogels with a Variety of Thiol Coupling Chemistries,” Front. Chem. 2:23 (2014), which is hereby incorporated by reference in its entirety). Aliquots of 4SP65 dissolved in FBS enriched medium has remained stable for >6 months while remnants of the original viscous polymer have maintained stability without any apparent loss of activity when stored for 60 months at −20° C.

The boc deprotected aminothiol conjugated to m-PEG6-thiol was dried on a vacuum pump overnight to yield a viscous oil that on analysis gave rise to spectra corresponding to 1LP65 as a di-trifluroacetic acid salt at a MW of 672.87 Da (FIG. 15), or a MW of 483 once dissolved in solution. As such, 1LP65 is soluble 100% DMSO or aqueous solution. However, the bulk of the product was dissolved at 40 mM in DMSO and stored in aliquots at −80° C. for future use once by further dilution in DMSO and then final dilution in medium for treatment of normal human epithelium and human cancer cell lines at working solutions of 4 mM or 400 μM drug. 1LP65 has maintained stability for six months in frozen aliquots but should be stable in DMSO at −80° C. for two to three years based upon recommended storage conditions for cystamine dihydrochloride (Selleck Chemical product information), an analogous 225 Da compound with a disulfide bond and amine groups like 1LP65.

### Example 9-4SP65 and 1LP65 have Only Growth Enhancement or Inhibitory Effects in NHMECS

Initial evaluations of the safety of 4SP65 and 1LP65 were focused on their effects on the growth of NHMECs from different donors. In a trial experiment, Strain-1 cells were exposed for 48 hours to 0, 5, 15, 50, or 150 μM 4SP65 (FIG. 16B). Treatments with 5 and 15 μM 4SP65 greatly enhanced the growth of Strain-1 cells by 336% and 286%, respectively, compared to sham-exposed control cells. In contrast, the growth of Strain-1 cells exposed to an intermediate concentration of 50 μM 4SP65 was 68% of the control cell growth. Remarkably, treatment of Strain-1 cells with a higher concentration of 150 μM 4SP65 re-established a predominant pattern of growth enhancement by the drug. In subsequent repetitive experiments, 48-hour treatments of Strain-2 and Strain-3 cells with 4SP65 induced dose-response curves, with growth enhancement of 162%±7% at 3 μM drug and dose-related decreases in growth relative to control cells of 78%±12%, 52%±8%, 21%±4%, and 8.7%±2% after respective exposures to 9, 30, 50, and 150 μM drug (FIG. 16B). Notably, the average effect per unit dose of 4SP65 (% growth÷exposure concentration) for 9, 30, 90, and 150 μM drug exposures was 8.7, 1.70, 0.23, and 0.058, respectively, suggesting that a stable level of growth inhibition was achieved, consistent with the induction of p53-mediated cell-cycle arrest in normal cells (Bitsue and Fekede, “The Effect of Amifostine, in Chemotherapy, Radiotherapy, as Potential Cytoprotectant and Immunomodulatory, in Cancer and Autoimmunity Treatment and Prevention,” International Journal of Scientific & Engineering Research 8 (9): 52-72 (2017) and Shen et al., “Binding of the Aminothiol WR-1065 to Transcription Factors Influences Cellular Response to Anticancer Drugs,” J. Pharmacol. Exp. Ther. 297 (3): 1067-1073 (2001), which are hereby incorporated by reference in their entirety). Lastly, while IC₅₀ values could not be calculated or predicted for Strain-1 and Strain-2 NHMECs exposed to 4SP65, an IC₅₀ of 76±14 μM was calculated for Strain-3 cells (FIG. 16B).

Treatment of Strain-2 and Strain-3 cells for 48 hours with 0-500 μM 1LP65 also induced dose-dependent decreases in cell growth, reaching 19.5% and 12.5% of control cell values, respectively, at the highest drug level (FIG. 16B). Following exposures to 90, 150, 300, or 500 μM 1LP65, an average effect per unit dose of 2.9, 0.31, 0.06, and 0.03, respectively, indicated that a stable level of growth inhibition was achieved. However, the responses to 1LP65 exposure in the two strains of NHMECs were sufficiently different, and the average IC₅₀ values were calculated to be >500 μM in Strain-2 cells and 210 μM in Strain-3 cells, averaging >355 μM (FIG. 16B).

### Example 10—4SP65 has Broad-Spectrum In Vitro Anticancer Effectiveness

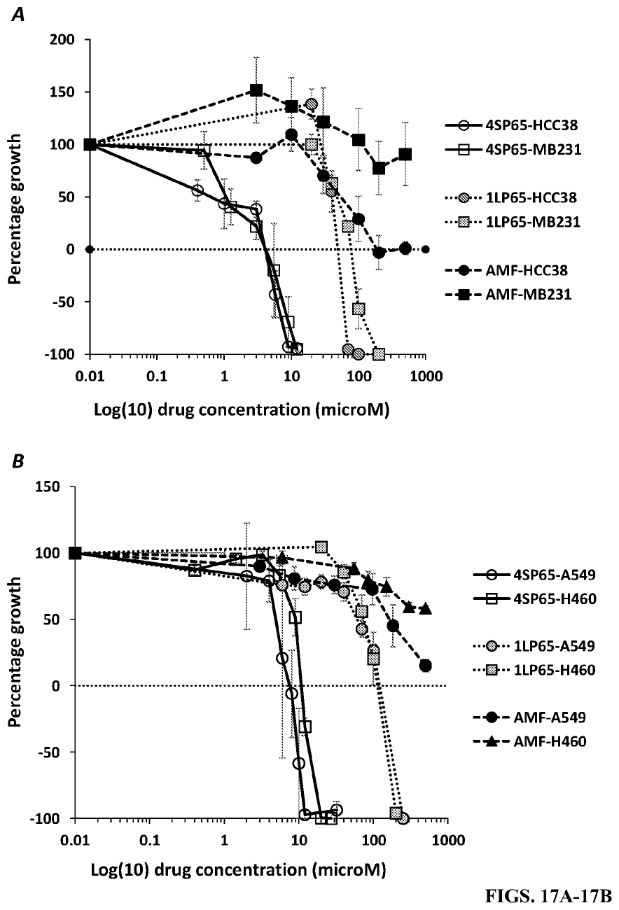

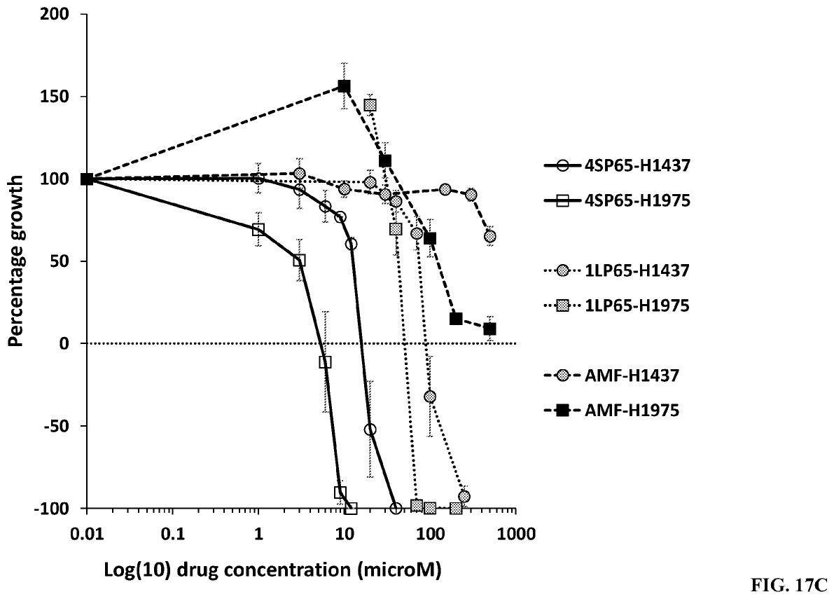

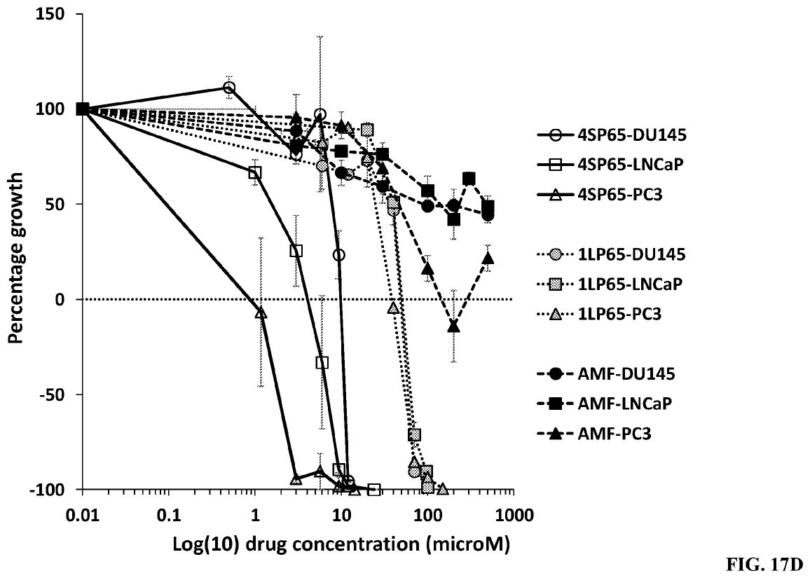

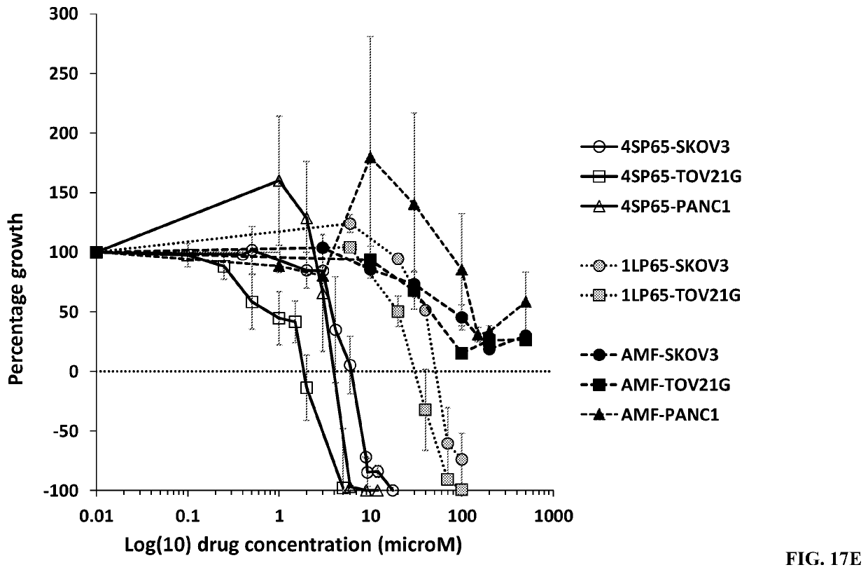

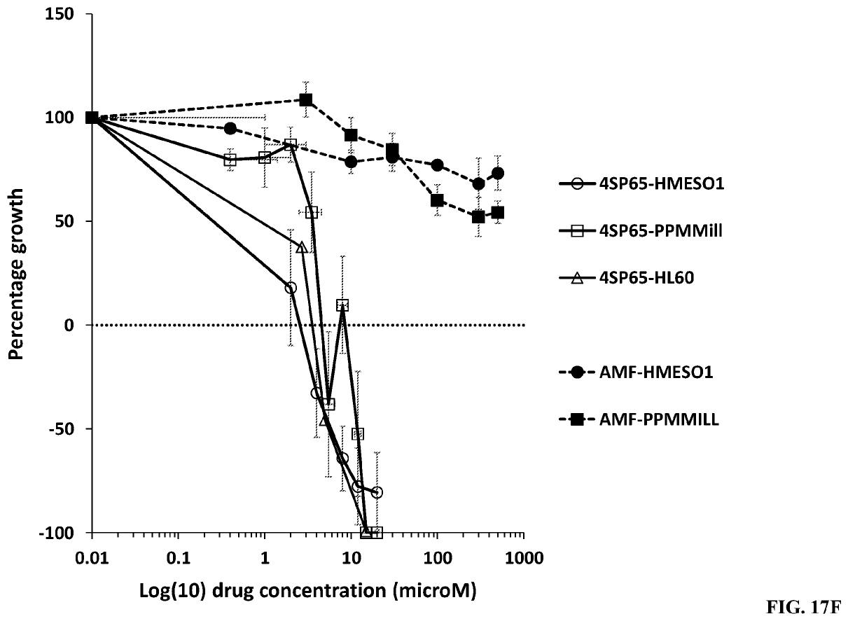

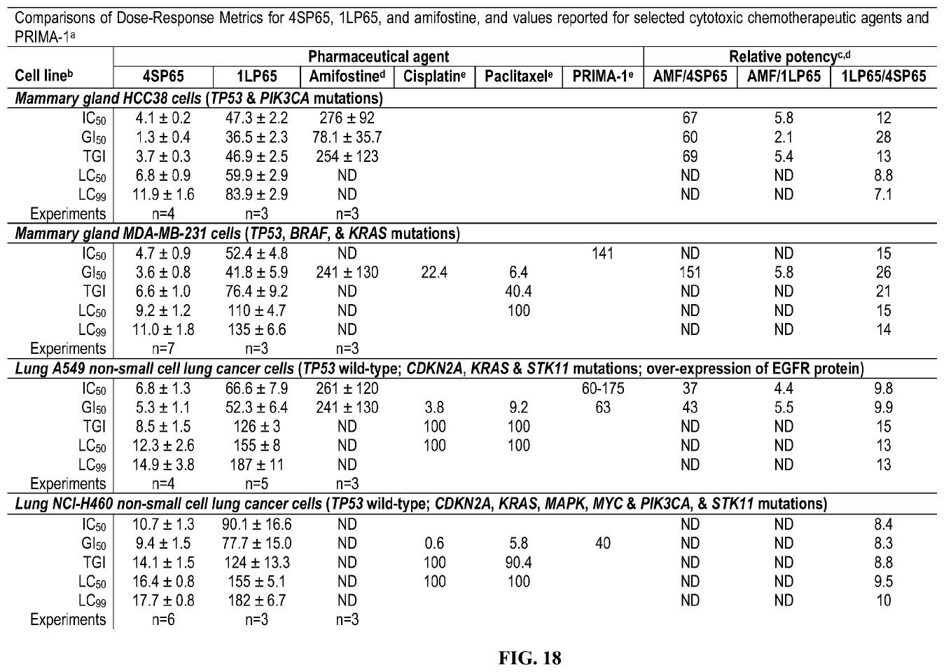

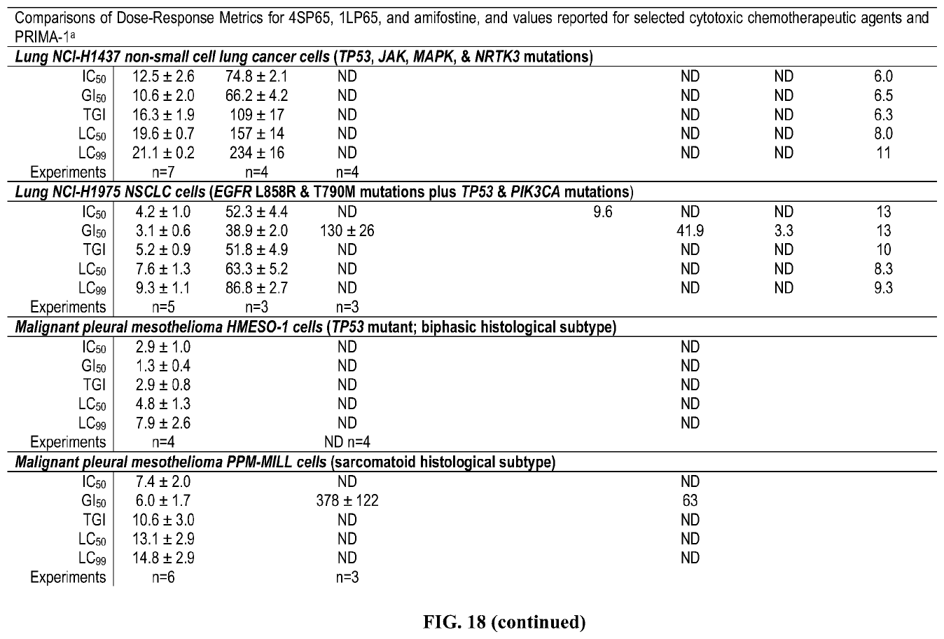

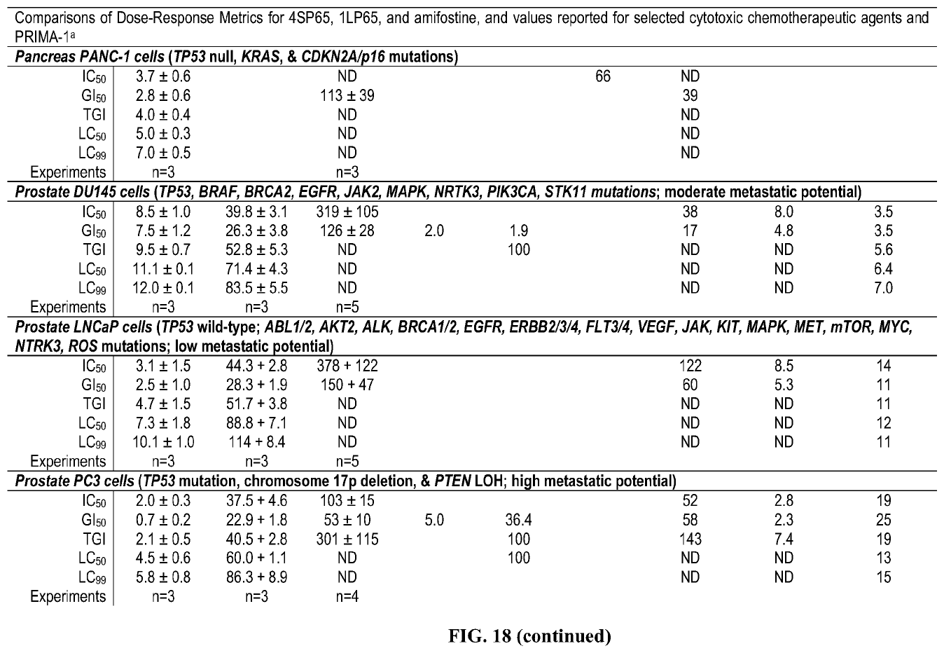

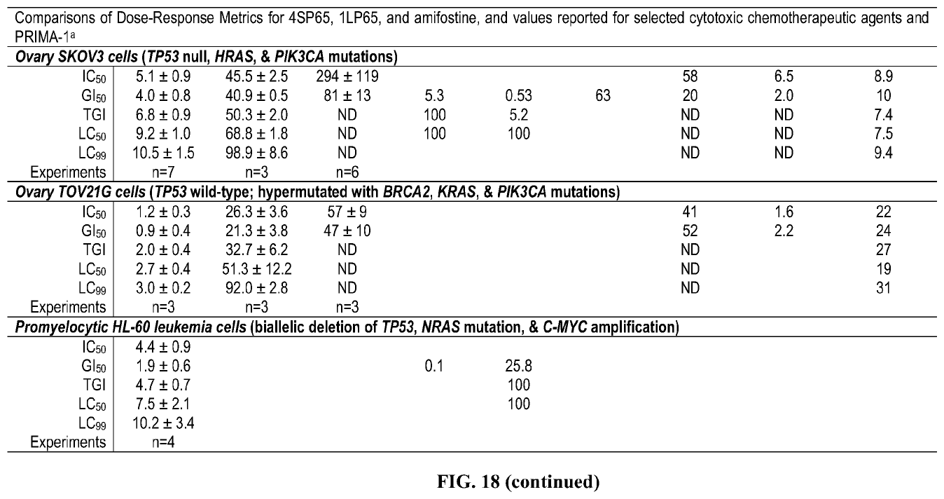

The BRG prodrug 4SP65 yielded broad-spectrum in vitro anticancer effects against cell lines representing two triple negative breast cancers, four non-small cell lung cancers, two malignant pleural mesotheliomas, one pancreatic cancer, three prostate cancers, a serous and a clear cell ovarian carcinoma, and promyelocytic leukemia. The results of cell proliferation assays demonstrated that, based on individual dose-response metrics listed in FIG. 18, single 48-hour exposures to 4SP65 had dose-dependent activities against all 15 cancer cell lines tested, yielding a consistent pattern where cytostatic effects transitioned to cytocidal effects as drug levels increased (FIGS. 17A-17F). The GI50 values show that cell growth above starting cell numbers was blocked by 50% at an average concentration of 4.1±0.8 μM 4SP65, ranging from 0.7 to 10.6 μM for individual cell lines. TGI values indicate that cell growth above starting cell numbers was completely blocked at an average concentration of 6.8±1.1 μM 4SP65 (range, 2.0-16.3 μM). LC₅₀ values demonstrate that starting cell numbers were reduced by 50% at an average concentration of 9.1±1.2 μM 4SP65 (range, 2.7 to 19.6 μM). LC₉₉ values reveal that an average concentration of 11.2=1.2 μM 4SP65 (range, 3.0-21.1 μM) induced near-complete cell death in all cancer cell lines, displaying a high degree of disease resolution at drug levels that likely can be achieved in patients as realistically required in a clinical setting (Liston and Davis, “Clinically Relevant Concentrations of Anticancer Drugs: A Guide for Nonclinical Studies,” Clin. Cancer Res. 23 (14): 3489-3498 (2017), which is hereby incorporated by reference in its entirety). Moreover, if the IC₅₀ of 76 for the most sensitive of the three NHMEC strains treated with 4SP65 was used to calculate a selectivity index, then the average values for HCC38 and MDA-MB-231 breast cancer cells were >19 and >16, respectively, indicating that 4SP65 is a safe anticancer agent.

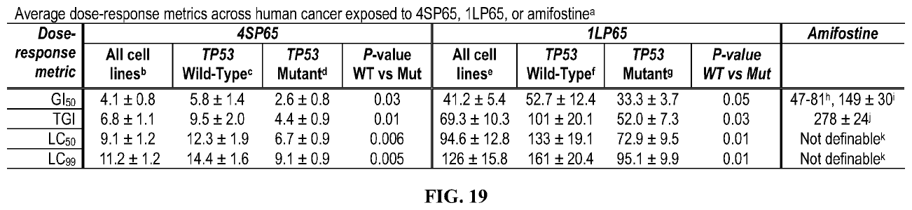

FIG. 18 includes a list of the genes with pathogenic mutations associated with each human cancer cell line treated with 4SP65. Among eight cancer cell lines with TP53 mutations (not including TP53-null cell lines), H1437 cells were much less responsive to the cytostatic/cytocidal effects of 4SP65 with significantly higher values observed for each dose-response metric compared to other TP53 mutant cancer cell lines (P-values of 2×10⁻⁷ for GI₅₀s to 6×10⁶ for LC₉₉s). One pilot study suggested that the low effectiveness of 4SP65 in TP53-mutant H1437 cells is a consequence of the restricted active transport of the drug. This phenomenon also was observed in H460 cells and was overcome when 4SP65 was combined with cisplatin, resulting in a dramatic increase in the efficacy of 4SP65 (36-fold at the GI₅₀ level) likely associated with the induction of a polyamine stress response by cisplatin. Among the five cell lines with wild-type TP53, TOV21G cells were much more responsive to the cytostatic/cytocidal effects of 4SP65 with significantly lower dose-response metrics compared to other TP53 wild-type cancer cell lines (P-values=0.035 for GI₅₀s to 0.001 for LC₉₉s). TOV21G cells have wild-type TP53 but exhibit a ‘hypermutator’ genotype that clearly sets them apart from other ovarian cancer cell lines including SKOV3 and high-grade serous ovarian cancer cell lines (Domcke et al., “Evaluating Cell Lines as Tumour Models by Comparison of Genomic Profiles,” Nat. Commun. 4:2126 (2013), which is hereby incorporated by reference in its entirety). If H1437 and TOV21G cells are excluded from analyses, comparisons of individual dose-response metrics for the remaining seven TP53 mutant cancer cell lines to corresponding values for the other four TP53 wild-type cancer cells lines show that the TP53-mutated cell lines are significantly more responsive than TP53 wild-type cell lines to a 48-h 4SP65 treatment, with the P-values progressing from 0.03 at GI₅₀ level cytostatic effects to 0.005 at LC₉₉ level cytolytic effects (FIG. 19).

Dose-response metric values for cisplatin and paclitaxel from the NCI COMPARE database allowed comparisons between the corresponding values for seven human cancer cell lines treated with 4SP65 (FIG. 18). In these cell lines, the cytostatic effects of 4SP65 generally overlapped with those of cisplatin and paclitaxel at the GI₅₀ level. For example, the GI₅₀ values ranged from 0.7 to 9.4 μM 4SP65 compared to a range of therapeutic levels of 0.1 to 5.3 μM cisplatin in all cell lines excluding the GI₅₀ of 22.4 μM cisplatin in MDA-MB-231 cells. In contrast, 4SP65 is 6- to 22-fold more efficacious than cisplatin or paclitaxel at the LC₅₀ level; the reported LC₅₀ concentrations of cisplatin and paclitaxel neither achievable nor safe in cancer patients (Rajkumar et al., “Cisplatin Concentrations in Long and Short Duration Infusion: Implications for the Optimal Time of Radiation Delivery,” J. Clin. Diagn. Res. 10 (7): XC01-XC04 (2016) and Stage et al., “Clinical Pharmacokinetics of Paclitaxel Monotherapy: An Updated Literature Review,” Clin. Pharmacokinet. 57 (1): 7-19 (2018), which are hereby incorporated by reference in their entirety).

FIG. 18 also shows further comparisons of GI₅₀ values for 4SP65 with those for PRIMA-1 or PRIMA-1met, which are anticancer drugs that reactivate mutant p53 protein (Bykov et al., “Mutant p53-Dependent Growth Suppression Distinguishes PRIMA-1 from known Anticancer Drugs: A Statistical Analysis of Information in the National Cancer Institute Database,” Carcinogenesis 23 (12): 2011-2018 (2002), which is hereby incorporated by reference in its entirety). Among the seven cancer cell lines available for comparisons, 4SP65 was more effective as a cytostatic agent than PRIMA-1/PRIMA-1met by 3- to 12-fold in TP53 wild-type cells, 2.3- to 30-fold in TP53 mutant cells, and 16-fold in TP53-null SKOV3 cells.

### Example 11-1LP65 has Broad-Spectrum In Vitro Anticancer Effectiveness

In vitro treatment with 1LP65, delivering only one WR1065/molecule intracellularly, also produced broad-spectrum anticancer effects (FIGS. 17A-17F; FIG. 18). Across 11 human cancer cell lines treated for 48 hours with 1LP65, the dose-response metric averages were 41.2±5.4 μM (range, 21.3-77.7) for GI₅₀s, 69.3±10.3 μM (range, 32.7-126) for TGIs, 94.6±12.8 μM (range, 51.3-157) for LC₅₀s, and 126±15.8 μM (range, 83.5-234) for LC₉₉s. As was the case with 4SP65, if H1437 and TOV21G cells were omitted from the analyses, comparisons of the dose-response metrics for the remaining six TP53 mutant cancer cell lines to the corresponding values for the other three TP53 wild-type cancer cell lines showed that the TP53-mutated cell lines were significantly more responsive than TP53 wild-type cell lines to 1LP65 treatment (FIG. 19).

FIG. 18 shows comparisons between dose-response metrics for 1LP65, cisplatin, and paclitaxel in the six human cancer cell lines treated for 48 hours. At the GI50 level, the doses of cisplatin that achieved cytostatic effects were ˜5- to 14-fold lower than those of 1LP65 in the four cell lines and 130-fold lower in H460 cells. MDA-MB-231 cells were the exception, where a GI₅₀ of 22.4 μM cisplatin was toxic in cancer patients (Rajkumar et al., “Cisplatin Concentrations in Long and Short Duration Infusion: Implications for the Optimal Time of Radiation Delivery,”. J. Clin. Diagn. Res. 10 (7): XC01-XC04 (2016), which is hereby incorporated by reference in its entirety) while a GI50 of 41.8 μM 1LP65 likely is safe and clinically achievable, desirable characteristics for anticancer agents (Liston and Davis, “Clinically Relevant Concentrations of Anticancer Drugs: A Guide for Nonclinical Studies,” Clin. Cancer Res. 23 (14): 3489-3498 (2017), which is hereby incorporated by reference in its entirety). Similarly, at the GI50 level, the cytostatic effects of paclitaxel were achieved at approximately 6- to 14-fold lower doses than 1LP65 in four cell lines and 77-fold lower in SKOV3 cells. PC3 cells were the exception, where a GI50 of 36.4 μM paclitaxel is highly toxic in vivo (Stage et al., “Clinical Pharmacokinetics of Paclitaxel Monotherapy: An Updated Literature Review,” Clin. Pharmacokinet. 57 (1): 7-19 (2018), which is hereby incorporated by reference in its entirety), while a GI₅₀ of 22.9 μM 1LP65 likely is safe and clinically achievable. However, the levels of cisplatin and paclitaxel required for (LC₅₀) cytolytic effects in these cancer cell lines occur at drug concentrations that are neither safe nor achievable in cancer patients (Rajkumar et al., “Cisplatin Concentrations in Long and Short Duration Infusion: Implications for the Optimal Time of Radiation Delivery,” J. Clin. Diagn. Res. 10 (7): XC01-XC04 (2016) and Stage et al., “Clinical Pharmacokinetics of Paclitaxel Monotherapy: An Updated Literature Review,” Clin. Pharmacokinet. 57 (1): 7-19 (2018), which are hereby incorporated by reference in their entirety). While in vitro studies indicate that 500 μM 1LP65 is not cytotoxic in NHMECs, pharmacokinetic studies need to be conducted to determine if concentrations of 51.3-157 μM 1LP65 that induce LC₅₀ level cytolytic effects across the 11 cancer cell lines tested can be achieved safely in vivo.

### Example 12—Amifostine has Limited Cytostatic and Lacks Cytolytic Effectiveness in Human Cancer Cell Lines Compared to 4SP65 and 1LP65

The potential growth inhibitory/cytolytic effects of amifostine in human cancer cell lines were ascertained to allow direct comparisons to the anticancer effectiveness of 4SP65 and 1LP65 (FIGS. 17A-17F, FIG. 18, and FIG. 19). Although plasma concentrations of 100 μM amifostine and exposures of over 3 hours are not readily achievable in humans or animals due to inherent drug delivery restrictions and dose-limiting toxicities (Grdina et al., “Amifostine: Mechanisms of Action Underlying Cytoprotection and Chemoprevention,” Drug Metabol Drug Interact. 16 (4): 237-279 (2000), which is hereby incorporated by reference in its entirety), for comparison purposes, cell lines were exposed for 48 hours to 0-500 μM drug. Among 14 human cancer cell lines, GI50 values of 47-81 μM amifostine were attained in four cell lines reported to exhibit moderate/high expression of alkaline phosphatase (Ravenni et al., “A Human Monoclonal Antibody Specific to Placental Alkaline Phosphatase, a Marker of Ovarian Cancer,” MAbs. 6 (1): 86-94 (2014) and Yao et al., “An ALP-Activatable and Mitochondria-Targeted Probe for Prostate Cancer-Specific Bimodal Imaging and Aggregation-Enhanced Photothermal Therapy,” Nanoscale. 11 (13): 6307-6314 (2019), which are hereby incorporated by reference in their entirety), GI₅₀ values between 113-378 μM were achieved in seven cell lines, and exposures up to 500 μM failed to induce growth inhibitory effects in three cell lines. Amifostine concentrations >100 μM induced TGI level effects in only two cell lines, HCC38 and PC3. Cytolytic effects were not observed in any cell line, even after exposures to 500 μM amifostine. At high concentrations of 200-500 μM amifostine, the dose-response curves were U-shaped for 6/14 cell lines, indicating that exposure to higher drug levels did not induce a greater effect. In the other cell lines, the dose-response curves flattened between 200-500 μM amifostine, so that increasing levels of growth inhibition could not be achieved. In contrast, dose-response curves for 4SP65 and 1LP65 did not display a U-shape at any concentration range in cancer cell lines, and did not flatten until >97% relative growth inhibition was achieved.

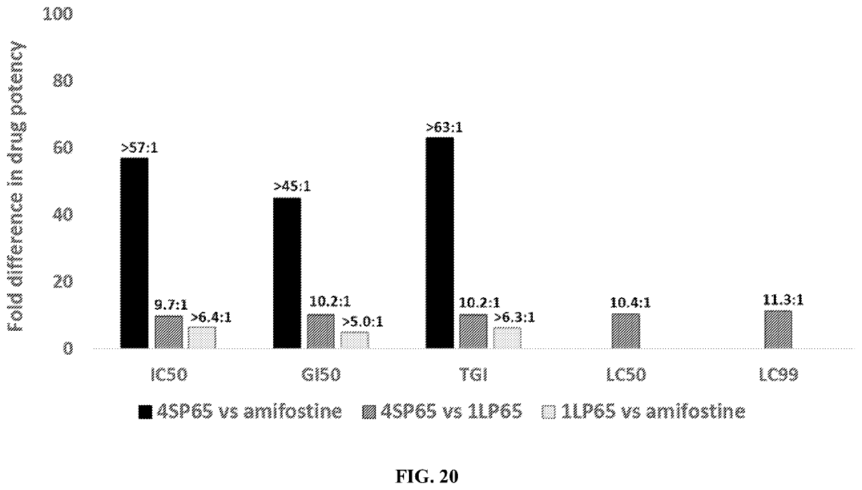

FIG. 20 shows the results of the estimates of the relative anticancer potency of 4SP65, 1LP65, and amifostine based on the averages of individual dose-response metrics for each compound across a panel of cancer cell lines. First, 4SP65 was 45- to 63-fold (averaging 55-fold) more potent than amifostine as a cytostatic agent, far exceeding the difference predicted solely by the ratio of four versus one WR1065/molecule in 4SP65 compared to amifostine. Second, 1LP65 was 5.0- to 6.4-fold (averaging 5.9-fold) more potent than amifostine as a cytostatic agent; however, only 1LP65 achieved cytolytic effects even though both drugs delivered a single WR1065 molecule. Third, 4SP65 was 9.7- to 11.3-fold (averaging 10.4-fold) more potent than 1LP65 depending on the dose-response metric, with the difference increasing as drug levels are increased to achieve LC₅₀ or LC₉₉ effects. Thus, the potency of 4SP65 was ˜2.5-fold greater than that predicted by the ratio of four WR1065s/molecule in 4SP65 versus one in 1LP65. If the analysis of the relative cytostatic potency between 4SP65 or 1LP65 versus amifostine is restricted to comparisons where amifostine achieved a GI₅₀ value at <100 μM drug in four cell lines (HCC38, PC3, SKOV3, and TOV21G), then the average differences in potency remain significant (P<0.007) with a 38-fold greater effect for 4SP65 (average GI50=1.7 μM) than amifostine (average GI50)=64.8 μM) and a 2.1-fold greater effect for 1LP65 (average GI50)=30.4 μM) than amifostine. Finally, the relative potency of 4SP65 and 1LP65 as cytocidal agents compared to amifostine is immeasurable because the latter drug did not induce cytolytic effects in any cancer cell line.

### Discussion of Examples 7-12

Based on the examples described herein, it is clear that 4SP65 and 1LP65 showed surprising therapeutic effectiveness against a range of human cancer cell lines that were TP53 wild-type, mutant, or null. 4SP65 and 1LP65 had significantly greater cytostatic activity than amifostine and showed dose-dependent cytolysis in all cell lines tested, while amifostine failed to induce cytolytic effects. 4SP65 also induced cytolytic effects in human cancer cell lines where cisplatin or paclitaxel did not achieve such effects at safe drug levels, and 4SP65 did so without cytotoxicity to normal cells. These results support the hypothesis that WR1065-mediated cytoprotection occurs in a cellular context of increased nuclear p53 protein along with normal-cell stress response function. 4SP65 and 1LP65 induced cell cycle-arrest in only normal cells, a recognized effect of p53 activation (Bitsue and Fekede, “The Effect of Amifostine, in Chemotherapy, Radiotherapy, as Potential Cytoprotectant and Immunomodulatory, in Cancer and Autoimmunity Treatment and Prevention,” International Journal of Scientific & Engineering Research 8 (9): 52-72 (2017) and Shen et al., “Binding of the Aminothiol WR-1065 to Transcription Factors Influences Cellular Response to Anticancer Drugs,” J. Pharmacol. Exp. Ther. 297 (3): 1067-1073 (2001), which are hereby incorporated by reference in their entirety), and did not induce cytotoxic effects.

The results presented herein show that WR1065 delivered via 4SP65 and 1LP65 resulted in cancer cell death. The cytolytic effects of 4SP65 and 1LP65 in the above cell lines are consistent with evidence that WR1065 increases nuclear p53 levels and extends p53 stability to antagonize the effects of mutant KRAS and activated NF-kB, resulting in cell death.

The findings reported herein also place aminothiol-conjugates of the present disclosure (e.g., 4SP65 and 1LP65) among a few anticancer agents that function to reactivate mutant p53 protein (Hu et al., “Targeting Mutant p53 for Cancer Therapy: Direct and Indirect Strategies,” J. Hematol. Oncol. 14 (1): 157 (2021), which is hereby incorporated by reference in its entirety), such as PRIMA-1/PRIMA-1met, which interact with key cysteine thiols via the active metabolite methylene quinuclidinone (Russo et al., “PRIMA-1 Cytotoxicity Correlates with Nucleolar Localization and Degradation of Mutant p53 in Breast Cancer Cells,” Biochem. Biophys. Res. Commun. 402 (2): 345-350 (2010), which is hereby incorporated by reference in its entirety).

In summary, the aminothiol-conjugates of the present disclosure offer a novel strategy for intracellular delivery of WR1065 or other bioactive aminothiols for effective and safe uses in new clinical areas, particularly in the treatment of cancer. Reports of amifostine/WR1065 showing cytoprotective effects in normal cells and concomitant anticancer effects in rare neoplasms (Dai et al., “A Potential Synergistic Anticancer Effect of Paclitaxel and Amifostine on Endometrial Cancer,” Cancer Res. 65 (20): 9517-9524 (2005) and Yuhas et al., “Treatment of Tumours with the Combination of WR-2721 and Cis-Dichlorodiammineplatinum (II) or Cyclophosphamide,” Br. J. Cancer 42 (4): 574-585 (1980), which are hereby incorporated by reference in their entirety) suggest that the superior anticancer effectiveness of aminothiol-conjugates described herein in vitro can be replicated in vivo.

### Materials and Methods for Examples 13-17

### Chemicals, Pharmaceuticals, and Reagents

4SP65 and 1LP65 were prepared as representative BRG prodrug as described above. Other pharmaceuticals/reagents including cisplatin (DDP) (Tocris Bioscience; catalog #2251/250), gefitinib (LC Laboratories; catalog #G44081G), erlotinib (Cayman Chemical; cat #10483250), and afatinib (Advanced Chemblocks, Inc.; catalog #G7208100MG), Trypan Blue solution, and CyQUANTR NF Cell Proliferation Assay Kits were obtained from ThermoFisher Scientific (Waltham, MA).

The aminothiol-conjugates of the present disclosure were developed by conjugation of the aminothiol via bioreducible disulfide bonds to discrete PEG-thiol scaffolds, yielding conjugates stable extracellularly with single/multiple active moieties released from the PEG scaffold in the cytosol of both normal and diseased cells non-enzymatically or enzymatically by essential thiol oxidation/reduction and thiol-disulfide exchange reaction components.

### Cell Lines and Culture Conditions

Human cancer cell lines including A549/ATCC, NCI-H460, NCI-H1975, and SKOV3, confirmed to be free of Mycoplasma, were purchased from ATCC (Manassas, VA). HMESO1, PPMMill, and TOV21G cells were verified by Dr. Brian Cunniff's laboratory to be Mycoplasma negative using LookOut® Mycoplasma PCR Detection Kit (Sigma-Aldrich) and to match previously annotated DNA fingerprints using analysis of STR profiles. Cell culture medium components from various vendors were obtained from/through ThermoFisher Scientific, and cell lines were cultured at 37° C. in a humidified incubator holding 5% CO₂ in 50:50 DMEM: F12 medium (mesothelioma and ovarian cancer cell lines), standard RPMI 1640 medium (A549 and H460 cells), or ATCC modification of RPMI 1640 medium (H1975 cells) with 10% (vol/vol) fetal bovine serum (FBS; Corning) and 100 U/mL Penicillin-Streptomycin (Corning).

### Cell Viability Assays

For testing drug efficacy of individual drugs or drug combinations, the CyQUANT® NF Cell Proliferation Assay was used according to manufacturer instructions for quantifying cell numbers with a Tecan Infinite 200 PRO plate reader (San Jose, CA) (i) to generate standard curves for cell numbers for each cell line, (ii) to measure starting cell numbers at the initiation of drug treatments, and (iii) to obtain fluorescence readings from cells in 96-well plates for comparisons to a standard curve for the relevant cell line for determining cell viability after drug treatments for 48 hours. Trypan Blue exclusion was applied as a complementary confirmation probe for cell death.

A series of in vitro experiments were conducted to assess the relative effectiveness of 4SP65 alone or binary pairs of 4SP65 with cisplatin or an EGFR-TKI against selected human cancer cell lines. First, drug combination studies were conducted to compare dose-response metrics for 4SP65 alone, cisplatin alone, or both drugs combined against six human cancer cell lines derived from three tissues. Then, the dose-response metrics for 4SP65 alone, a selected EGFR-TKI alone, or binary 4SP65-TKI pairs were evaluated in H1975 and A549 non-small cell lung cancer (NSCLC) cell lines. For short-term cell viability assays in these cancer cell lines, 2000-5000 cells/well were plated in 96-well dishes and incubated for ˜24 hours for cells to adhere and enter log phase growth. Then starting cell numbers were determined and remaining cells were treated with 0 to <50 μM 4SP65 alone, 0 to >15 μM cisplatin alone, 0 to 25 μM of an EGFR-TKI, and to 4SP65 combined with cisplatin or an EGFR-TKI at selected ratios to measure five dose-response metrics for drug effectiveness following a single 48-hour exposure.

### Data Analyses and Statistical Testing

The experimental design applied guidelines for comparisons between drugs used by the NCI (Monks et al., “Feasibility of a High-Flux Anticancer Drug Screen Using a Diverse Panel of Cultured Human Tumor Cell Lines,” J. Natl. Cancer Inst. 83 (11): 757-766 (1991), which is hereby incorporated by reference in its entirety) along with modifications for the Chou-Talalay method (Zhang et al., “Synergistic Combination of Microtubule Targeting Anticancer Fludelone with Cytoprotective Panaxytriol Derived from Panax ginseng Against MX-1 Cells In Vitro: Experimental Design and Data Analysis Using the Combination Index Method,” Am. J. Cancer Res. 6 (1): 97-104 (2015), which is hereby incorporated by reference in its entirety), while data analyses used the NCI methodology (Monks et al., “Feasibility of a High-Flux Anticancer Drug Screen Using a Diverse Panel of Cultured Human Tumor Cell Lines,” J. Natl. Cancer Inst. 83 (11): 757-766 (1991) and Hafner et al., “Growth Rate Inhibition Metrics Correct for Confounders in Measuring Sensitivity to Cancer Drugs,” Nat. Methods 13 (6): 521-527 (2016) which are hereby incorporated by reference in their entirety), together with those of Brooks and colleagues (Brooks et al., “Applicability of Drug Response Metrics for Cancer Studies Using Biomaterials,” Philos. Trans. R. Soc. Lond. B. Biol. Sci. 374 (1779): 20180226 (2019), which is hereby incorporated by reference in its entirety) to characterize concentration parameters that measure drug effectiveness following a single/paired antagonist cancer drug treatment (Tong, F. P. “Statistical Methods for Dose-Response Assays,” Thesis, University of California, Berkeley (2010), which is hereby incorporated by reference in its entirety). The inhibitory concentration 50% (IC₅₀) was calculated as the concentration of drug that reduces the growth of treated cells by 50% compared to control cells. To define the shape of the dose-response curves following exposure to a single/paired test agent, four additional metrics were calculated as described infra), including drug concentrations resulting in growth inhibition of 50% (GI50) relative to control cell numbers after subtracting out starting cell numbers, total growth inhibition (TGI) compared to starting cell numbers, and 50%/99% reduction in starting cell numbers (LC₅₀/LC₉₉) at the end of the drug treatment. Relationships between drug exposure levels and cell counts were calculated and then modeled using best fit regression curves and trend line formulas generated by Excel. Statistical comparisons of differences in individual dose-response metrics between individual/paired drug treatments and various cell lines were conducted using the Student's t-test, with P-values <0.05 considered significant.

Possible synergy between combination treatments with 4SP65-cisplatin or 4SP65-TKI drug pairs was further explored using SynergyFinder 2.0 (Ianeski et al., “SynergyFinder 2.0: Visual Analytics of Multi-Drug Combination Synergies,” Nucleic Acids Res. 48 (W1): W488-W493 (2020), which is hereby incorporated by reference in its entirety), a stand-alone web-application for interactive analysis that allows for inputting data from independent replicate experiments in order to calculate a 95% confidence interval for synergy scoring. Relative cell growth inhibition values for differing levels each drug alone and in combination were input into SynergyFinder to generated dose-response curves for each drug alone, to produce a dose-response matrix, to visualize the distribution of areas of differing degrees of synergism in a 2-dimension heatmap and 3-dimension volcano plot, and to calculate average and maximum synergy scores in selected human cancer cell lines. The expected drug combination responses were calculated based on the highest single agent (HSA) model (Yadav et al., “Searching for Drug Synergy in Complex Dose-Response Landscapes Using an Interaction Potency Model,” Comput. Struct. Biotechnol. J. 13:504-513 (2015), which is hereby incorporated by reference in its entirety). Synergy scores <10 means that the interaction between two drugs was likely to be antagonistic; scores from −10 to 10 means the interaction between two drugs was likely to be additive; and scores >10 means the interaction between two drugs was likely to be synergistic.

### Example 13—4SP65 and 1LP65 have Cytostatic and Cytocidal Effects in Immortalized Cell Lines

To determine potential cytostatic or cytocidal effects of BRG prodrugs in immortalized, non-tumorigenic cells, Vero, HEK-293t, LP9, and HPM3 cells were exposed to 4SP65 while only Vero cells also were treated with 1LP65. The results are summarized in Table 2. These data show that both cytostatic and cytocidal effects are induced by both BRG drugs (i.e., 4SP65 and 1LP65) at doses that likely can be achieved in vivo. In Vero cells exposed to 4SP65, the average drug concentration that induced GI₅₀ level cytostatic effects (i.e., 2.9±1.4 μM) was significantly different from the level of 4SP65 that caused the same level of growth inhibition in strain 3 of normal human mammary epithelial cells (NHMECs) (i.e., GI₅₀=41.4±6.2; P=0.007). GI₅₀ values could not be calculated or predicted in Strain-1 and Strain-2 of NHMECs treated with 4SP65 because these strains were more resistant to 4SP65 and GI₅₀ level cytostatic effects were not obtained, and thus, could not be calculated. Comparisons across other dose-response metrics could not be made because such levels of effects were not induced in NHMECs exposed to 4SP65. In Vero cells exposed to 1LP65, drug concentrations that induced GI₅₀ level cytostatic effects were not significantly different from those that induced the same level of inhibition in NHMECs. Since cytostatic and cytolytic effects at the TGI and LC_{50/99} levels were induced in Vero cells exposed to 1LP65 but not in NHMECs, there is a significant difference in the effects of 1LP65 in Vero cells compared to NHMECs, but p values based upon t-test evaluations cannot be defined due to lack of numerical data.

TABLE 2
Average dose-response metrics for four immortalized,
non-tumorigenic cell lines of differing cellular origins.

| Dose-response | 4SP65 | | | | 1LP65 |
|---|---|---|---|---|---|
| metric | VERO | HEK-293t | LP9 | HPM3 | VERO |
| GI₅₀ | 2.9 ± 1.4 | 15.8 | 9.2 | ~10.0 | 46.6 ± 2.6 |
| TGI | 6.4 ± 2.6 | 58.8 | 13.5 | | 82.8 ± 2.9 |
| LC₅₀ | 11.6 ± 6.3 | 60.6 | 19.0 | | 129.1 ± 3.4 |
| LC₉₉ | 14.0 ± 9.9 | 62.4 | ND | | 183.4 ± 6.2 |

### Example 14—4SP65 Enhances Anticancer Effectiveness and Overcomes Drug Resistance in Combination with Cisplatin

A series of experiments were conducted to define the degree to which co-treatment with aminothiol-conjugates of Formula (I) enhances the anticancer effectiveness and overcomes drug resistance to cisplatin, as a representative cytotoxic chemotherapeutic, in relevant panels of human cancer cell lines.

A series of experiments were conducted to assess the relative drug efficacy of 4SP65 and cisplatin as single agents or combined against two NSCLC, two pleural mesothelioma, and two ovarian cancer cell lines. For these experiments, a human cancer cell line was considered to be resistant to cisplatin when the amount of drug required to achieve cytolytic effects in cell culture was ≥15 μM, which exceeds reported peak plasma levels (Cₘₐₓ) of free drug averaging from 3.8±1.7 μM to 7.5±6.8 μM cisplatin during routine 1 hour IV infusions of differing sets of cancer patients (Rajkumar et al., “Cisplatin Concentrations in Long and Short Duration Infusion: Implications for the Optimal Time of Radiation Delivery,” J. Clin. Diagn. Res. 10 (7): XC01-XC04 (2016) and Gerina-Berzina et al., “Determination of Cisplatin in Human Blood Plasma and Urine Using Liquid Chromatography-Mass Spectrometry for Oncological Patients with a Variety of Fatty Tissue Mass for Prediction of Toxicity,” Exp. Oncol. 39 (2): 124-030 (2017), which are hereby incorporated by reference in their entirety). 4SP65 was deemed to overcome resistance to cisplatin when the two drugs combined resulted in a significant fold-increase in the level of cytocidal effects for cisplatin at concentrations below 15 μM, and the efficacy of 4SP65 was increased as well.

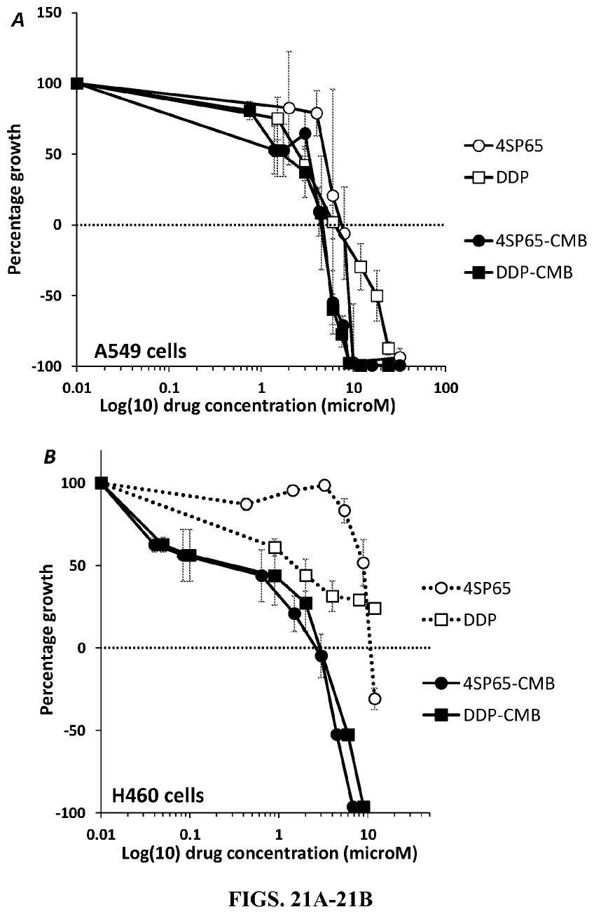

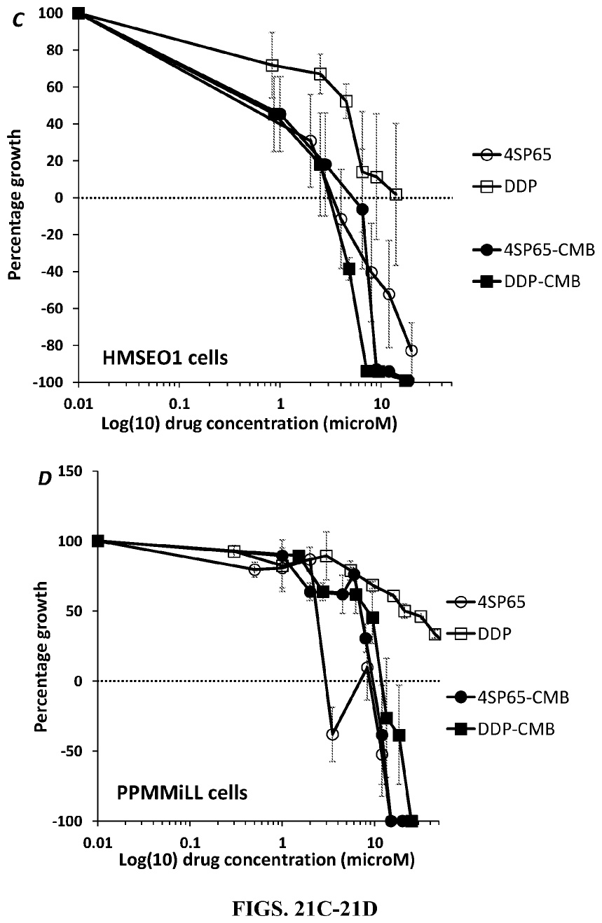

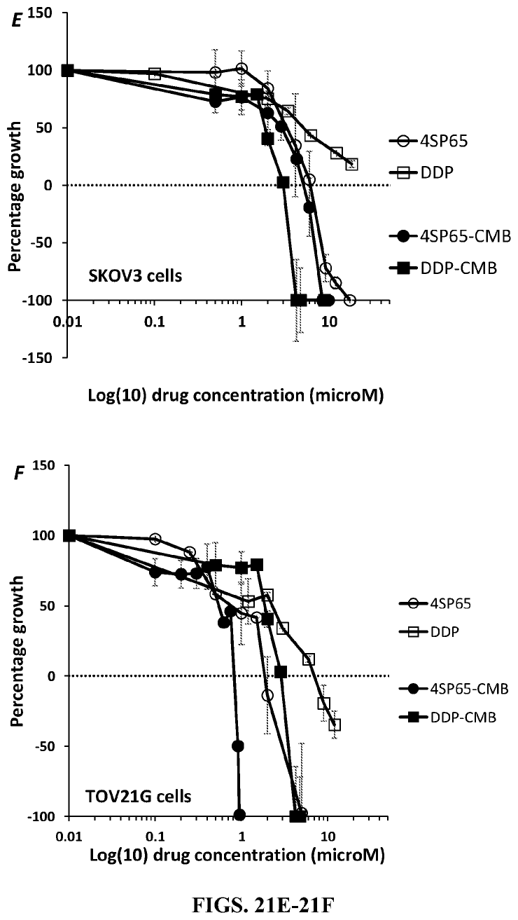

For each cancer cell line, FIGS. 21A-21F show the dose-response curves for cell growth after 48-hour treatments with each drug alone or combined, while Table 3 lists the corresponding dose-response metrics, the fold-increase in efficacy for each drug in the combination (with P-values indicated where increases are significant), and the synergy scores for the combination. For H460, HMESO-1, PPMMill, and SKOV3 cells, cisplatin alone induced only cytostatic effects, consistent with slowing of progressive disease. In contrast, 4SP65-cisplatin combined resulted in cytolytic effects, consistent with tumor size reduction, at plasma-equivalent levels of cisplatin, with significant gains of both cytostatic and cytolytic activities by 1.7- to 6.9-fold for cisplatin in all four cancer cell lines. Co-treatment with 4SP65-cisplatin also resulted in significant fold increases in the cytostatic and cytolytic activities by 1.3- to 36-fold in H460 and SKOV3 cells, but the modest gains of effect for 4SP65 (1.1- to 1.3-fold) in HMESO-1 and PPMMill cells were not significant. In A549 cells and TOV21G cells, treatment with cisplatin alone achieved an LC₅₀, but not an LC₉₉, at <15 μM drug. Combined 4SP65-cisplatin treatment resulted in significant gains of both cytostatic and cytolytic activities by 1.9- to 2.4-fold for 4SP65 and 1.7- to 3.5-fold for cisplatin in A549 cells to achieve an LC₉₉ at 8 μM 4SP65 plus 7 μM cisplatin. In TOV21G cells, co-treatment with 4SP65-cisplatin also yielded significant gains in total growth inhibition and cytolytic effects by 2.4- to 3.2-fold for 4SP65 and 1.3- to 2.9-fold for cisplatin, reducing the LC₉₉ value of 3.0 μM for 4SP65 alone and 17.2 μM for cisplatin alone to 0.95 μM 4SP65 plus 5.9 μM cisplatin when combined.

TABLE 3
Dose response metrics for 4SP65 alone, cisplatin alone, and binary
combinations of 4SP65-cisplatin in drug-exposed human cancer cell lines^{a}
Experiments using 4SP65 and/or cisplatin (DDP)

| | | | | | Fold increase in efficacy | |
|---|---|---|---|---|---|---|
| Cell line | 4SP65 | 4SP65-c | DDP | DDP-c | 4SP65/4SP65-c | DDP/DDP-c |

A549 NSCLC cells (n = 4 experiments)

| | | | | | | |
|---|---|---|---|---|---|---|
| IC₅₀ | 6.8 ± 1.3 | 3.1 ± 0.5 | 4.7 ± 0.8 | 2.8 ± 0.7 | 2.2* | 1.7* |
| GI₅₀ | 5.4 ± 2.5 | 2.3 ± 0.5 | 3.5 ± 0.7 | 2.0 ± 0.6 | 2.4* | 2.3* |
| TGI | 8.3 ± 1.5 | 4.3 ± 0.5 | 8.2 ± 1.0 | 3.8 ± 0.7 | 1.9* | 1.8** |
| LC₅₀ | 12.1 ± 2.7 | 6.5 ± 0.6 | 13.6 ± 2.2 | 5.7 ± 0.8 | 1.9* | 2.0** |
| LC₉₉ | 14.9 ± 3.8 | 8.0 ± 0.6 | 24.4 ± 2.6 | 7.0 ± 0.8 | 1.9* | 3.5*** |

^{b}(Average HSA synergy score on scale of 60 = 23.6; Most synergistic area score = 29.8)
NCI-H460 NSCLC cells (n = 4 experiments)

| IC₅₀ | 13.3 ± 1.0 | 0.67 ± 0.04 | 4.0 ± 0.1 | 0.88 ± 0.53 | 20** | 4.6*** | |
|---|---|---|---|---|---|---|---|
| GI₅₀ | 12.3 ± 1.1 | 0.34 ± 0.07 | 3.1 ± 0.1 | 0.45 ± 0.09 | 36** | 6.9*** | |
| TGI | 16.9 ± 0.6 | 2.3 ± 0.3 | ND (>15) | 2.6 ± 0.9 | 7.4^{c} | >5.8 | GoE |
| LC₅₀ | 17.6 ± 0.5 | 3.3 ± 0.2 | ND (>15) | 4.4 ± 0.3 | 5.3^{c} | >3.4 | GoE |
| LC₉₉ | 18.2 ± 0.4 | 4.2 ± 0.3 | ND (>15) | 5.6 ± 0.4 | 4.3^{c} | >2.7 | GoE |

^{b}(Average HSA synergy score = 36.4; Most synergistic area score = 57.5)
HMESO-1 malignant mesothelioma cells (n = 3 experiments)

| IC₅₀ | 4.7 ± 0.2 | 4.4 ± 0.7 | 8.6 ± 1.1 | 3.0 ± 0.3 | 1.1 (NS) | 2.9* | |
|---|---|---|---|---|---|---|---|
| GI₅₀ | 2.0 ± 0.3 | 1.3 ± 0.3 | 3.0 ± 0.4 | 0.8 ± 0.2 | 1.5 (NS) | 3.9* | |
| TGI | 4.3 ± 0.2 | 3.4 ± 0.2 | 6.7 ± 0.7 | 2.6 ± 0.3 | 1.3 (NS) | 2.6** | |
| LC₅₀ | 7.0 ± 0.1 | 7.0 ± 1.0 | ND (>15) | 4.8 ± 0.3 | 1.0 (NS) | >3.1 | GoE |
| LC₉₉ | 12.4 ± 0.1 | 10.9 ± 0.6 | ND (>15) | 8.9 ± 0.4 | 1.1 (NS) | >1.7 | GoE |

^{b}(Average HSA synergy score = 13.1; Most synergistic area score = 36.1)
PPM-Mill malignant mesothelioma cells (n = 4 experiments)

| IC₅₀ | 10.0 ± 1.9 | 7.7 ± 1.5 | 40.5 ± 8.1 | 11.6 ± 2.3 | 1.3 (NS) | 3.5** | |
|---|---|---|---|---|---|---|---|
| GI₅₀ | 8.4 ± 1.5 | 6.3 ± 1.4 | 27.2 ± 3.4 | 9.5 ± 2.1 | 1.3 (NS) | 5.5** | |
| TGI | 14.1 ± 3.3 | 10.8 ± 1.8 | ND (>100) | 16.3 ± 2.7 | 1.3 (NS) | >6.1 | GoE |
| LC₅₀ | 16.2 ± 3.2 | 12.9 ± 1.3 | ND (>100) | 19.3 ± 1.9 | 1.3 (NS) | >5.2 | GoE |
| LC₉₉ | 17.6 ± 3.5 | 13.9 ± 1.2 | ND (>100) | 20.9 ± 1.8 | 1.3 (NS) | >4.8 | GoE |

^{b}(Average HSA synergy score = 5.8; Most synergistic area score = 20.1)
SKOV3 ovarian cancer cells (n = 4 experiments)

| IC₅₀ | 5.2 ± 1.0 | 2.9 ± 0.6 | 6.7 ± 0.6 | 2.6 ± 0.3 | 1.8** | 3.2** | |
|---|---|---|---|---|---|---|---|
| GI₅₀ | 4.3 ± 1.0 | 2.7 ± 0.6 | 5.3 ± 0.4 | 2.3 ± 0.2 | 1.5** | 1.7** | |
| TGI | 7.3 ± 0.8 | 4.9 ± 0.6 | ND (>21) | 4.8 ± 0.9 | 1.5* | >4.4 | (GoE) |
| LC₅₀ | 9.7 ± 0.5 | 7.4 ± 0.3 | ND (>21) | 8.4 ± 2.8 | 1.3** | >2.5 | (GoE) |
| LC₉₉ | 10.0 ± 0.8 | 7.7 ± 0.1 | ND (>21) | 12.9 ± 3.8 | 1.3* | >1.6 | (GoE) |

^{b}(Average HSA synergy score = 18.2; Most synergistic area score = 42.1)
TOV21G ovarian cancer cells (n = 3 experiments)

| | | | | | | | |
|---|---|---|---|---|---|---|---|
| IC₅₀ | 1.2 ± 0.3 | 0.62 ± 0.01 | 3.0 ± 0.3 | 3.8 ± 0.1 | 1.9 (NS) | 0.8 | (NS) |
| GI₅₀ | 0.93 ± 0.37 | 0.41 ± 0.13 | 2.2 ± 0.4 | 2.5 ± 0.8 | 2.3 (NS) | 0.9 | (NS) |

| | | | | | | |
|---|---|---|---|---|---|---|
| TGI | 2.0 ± 0.4 | 0.85 ± 0.01 | 6.7 ± 0.3 | 5.2 ± 0.1 | 2.4* | 1.3* |
| LC₅₀ | 2.7 ± 0.1 | 0.91 ± 0.01 | 13.2 ± 2.2 | 5.6 ± 0.1 | 3.0*** | 2.4* |
| LC₉₉ | 3.0 ± 0.2 | 0.95 ± 0.02 | 17.2 ± 5.2 | 5.9 ± 0.1 | 3.2*** | 2.9* |

^{b}(Average HSA synergy score = 18.2; Most synergistic area score = 26.4)
^{a}Cell lines were plated in 96-well microtiter dishes, incubated for~24 h prior, and then treated over a
dose range for 48 hours. Calculation of values for inhibitory concentration 50% (IC₅₀), growth
inhibitor concentration 50% (GI₅₀), total growth inhibitory concentration (TGI), lethal concentration
50% (LC₅₀) and 99% (LC₉₉) was done as set forth by the NCI (Monks et al., “Feasibility of a High-
Flux Anticancer Drug Screen Using a Diverse Panel of Cultured Human Tumor Cell Lines,” J. Natl.
Cancer Inst. 83(11): 757-766 (1991) and Hafner et al., “Growth Rate Inhibition Metrics Correct for
Confounders in Measuring Sensitivity to Cancer Drugs,” Nat. Methods 13(6): 521-527 (2016), which
are hereby incorporated by reference in their entirety). All units are μM, with means and standard
errors. Abbreviations: 4SP65-c and DDP-c refer to the amounts of each drug in combination at
each dose-response metric. At each dose response metric, the amount of 4SP65 and of cisplatin
(DDP) is as indicated in the table. The total drug concentration at each dose response metric is the
sum of the 4SP-c concentration and the DDP-c concentration. For more detailed description of
methods, see ‘how the combination studies were done’ in the notes. ND, not definable, with
following number in parentheses indicating the highest concentration of drug tested in μM for a
given cancer cell line; GoE, gain of effect; NS, non-significant.
^{b}Synergy score data obtained via SynergyFinder (Ianeski et al., “SynergyFinder 2.0: Visual Analytics
of Multi-Drug Combination Synergies,” Nucleic Acids Res. 48(W1): W488-W493 (2020), which is
hereby incorporated by reference in its entirety); scores <−10 likely antagonistic, −10 to +10 likely
additive, and >10 likely synergistic.
*P < 0.05,
**P ≤ 0.01,
***P ≤ 0.001

### Example 15—Visual Analytics (Synergy Reports) of Combination Synergies 4SP65 Plus Cisplatin

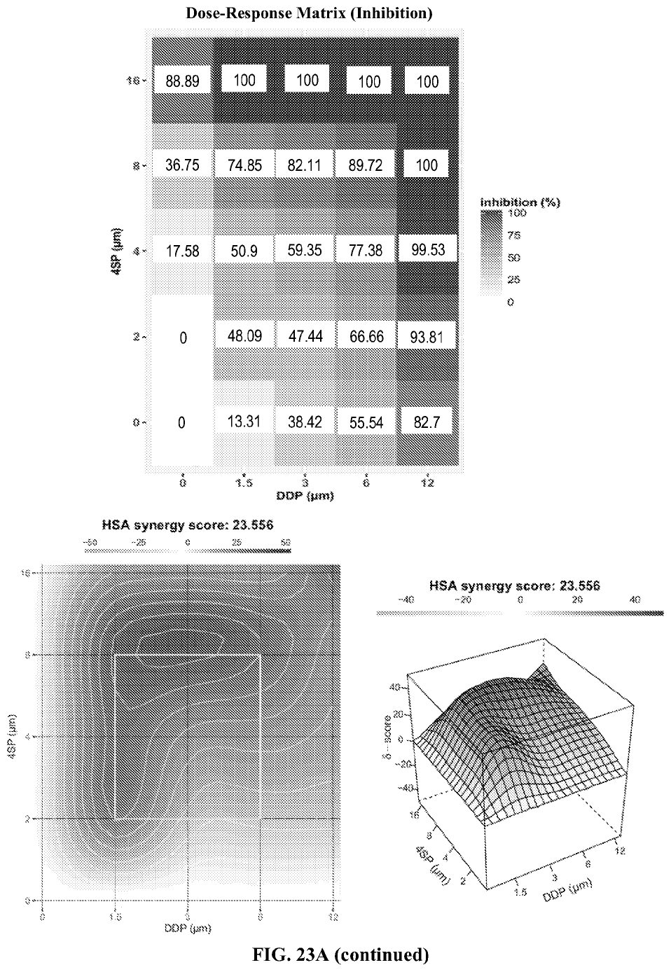

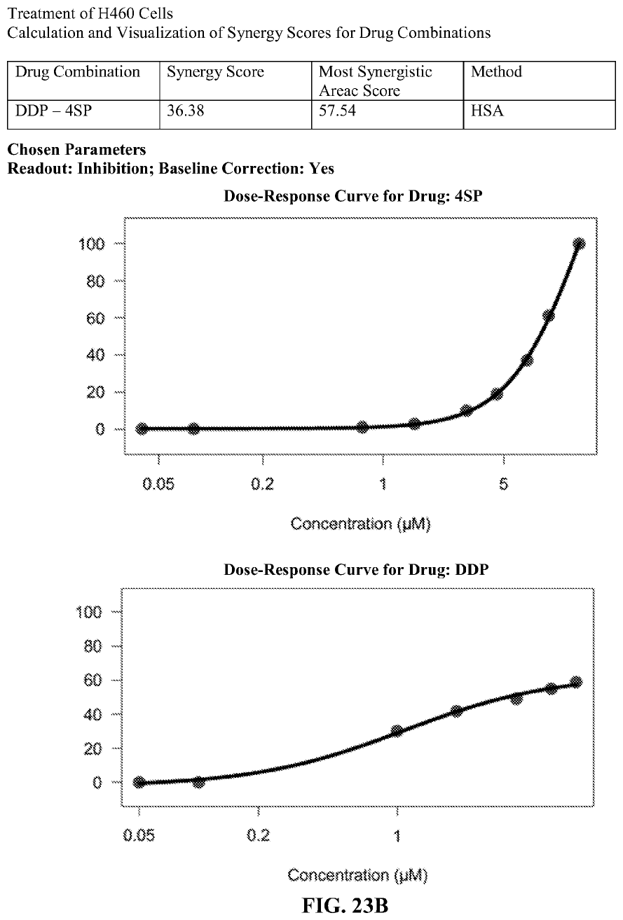

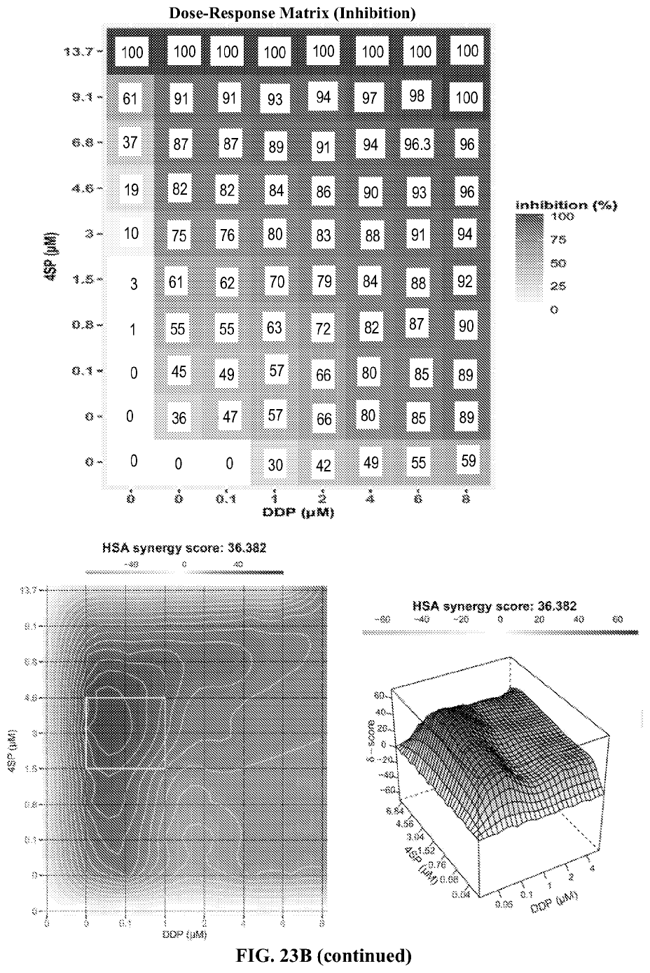

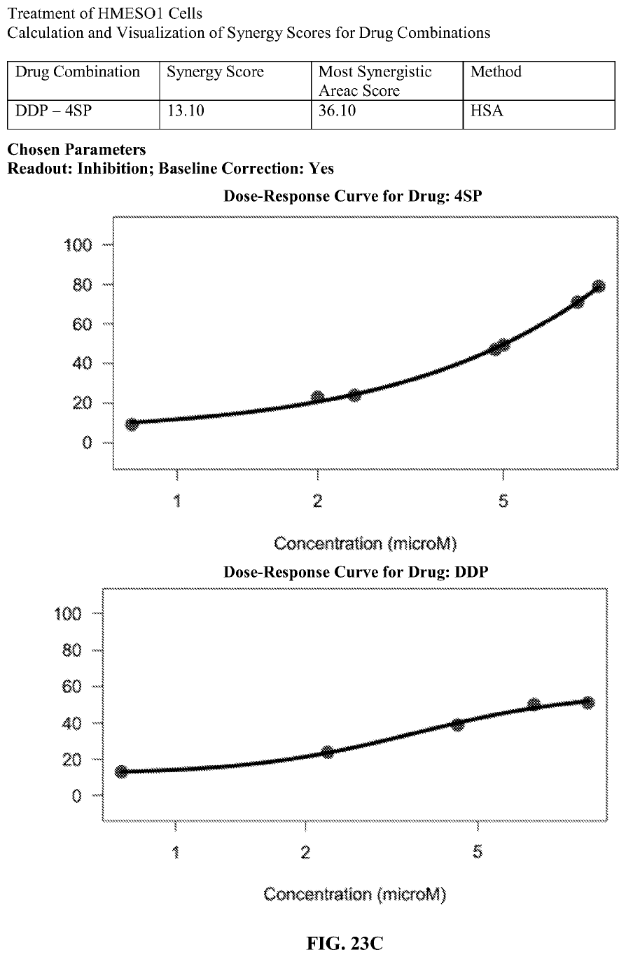

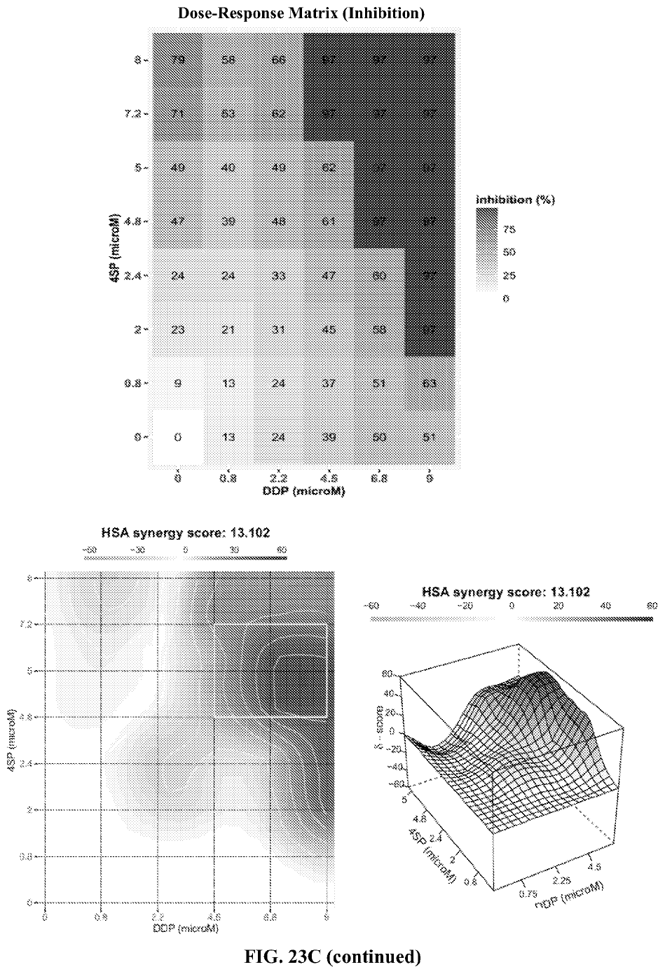

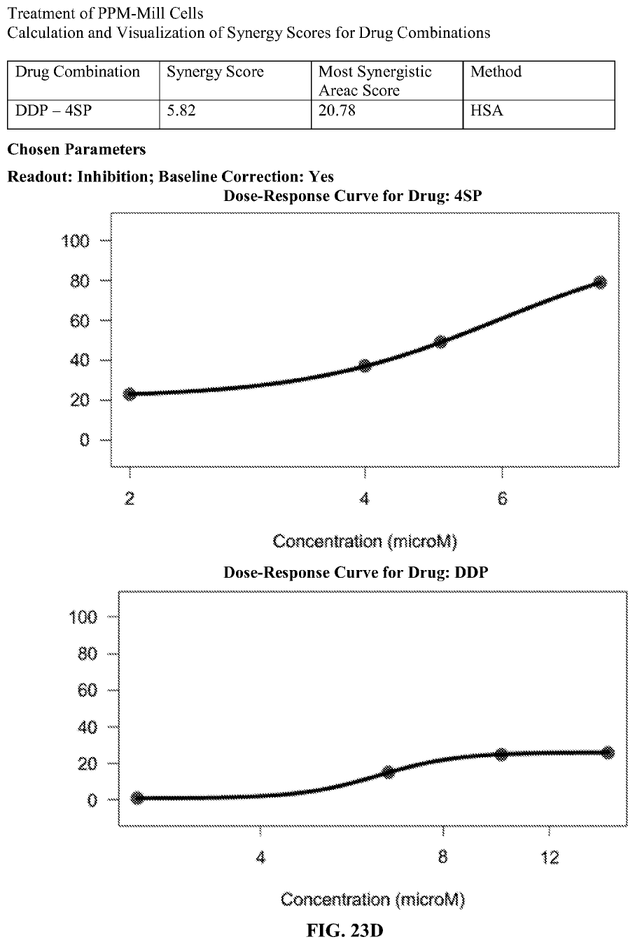

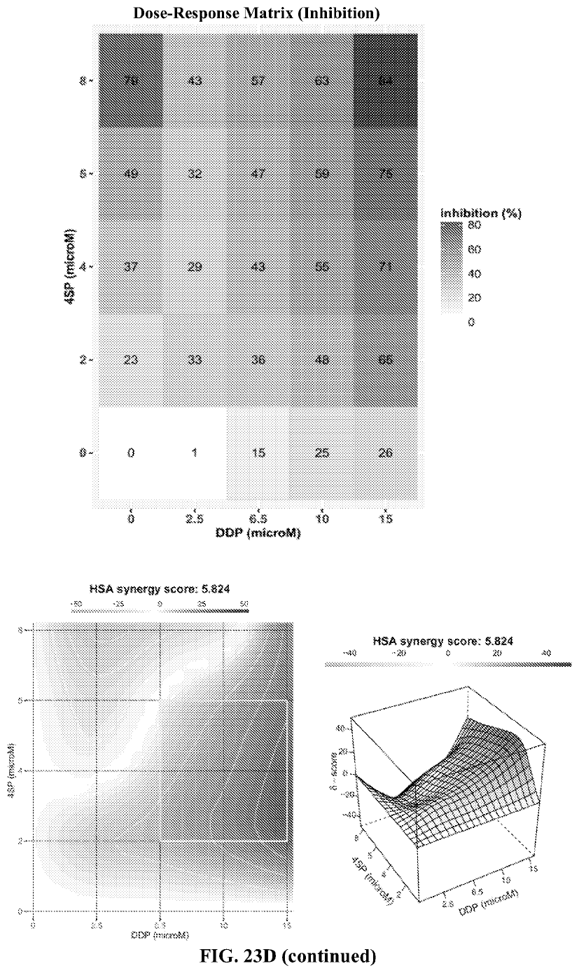

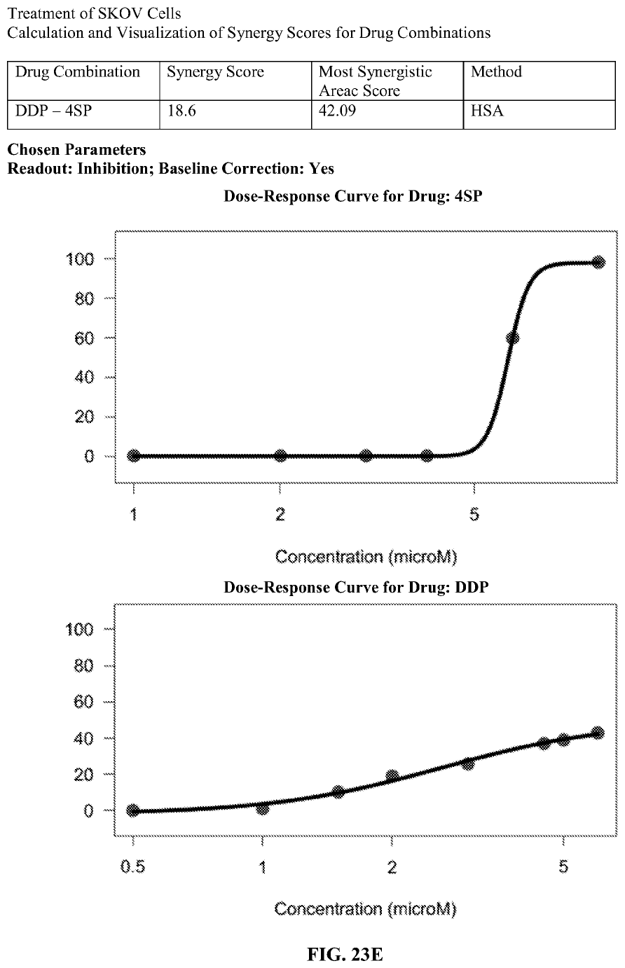

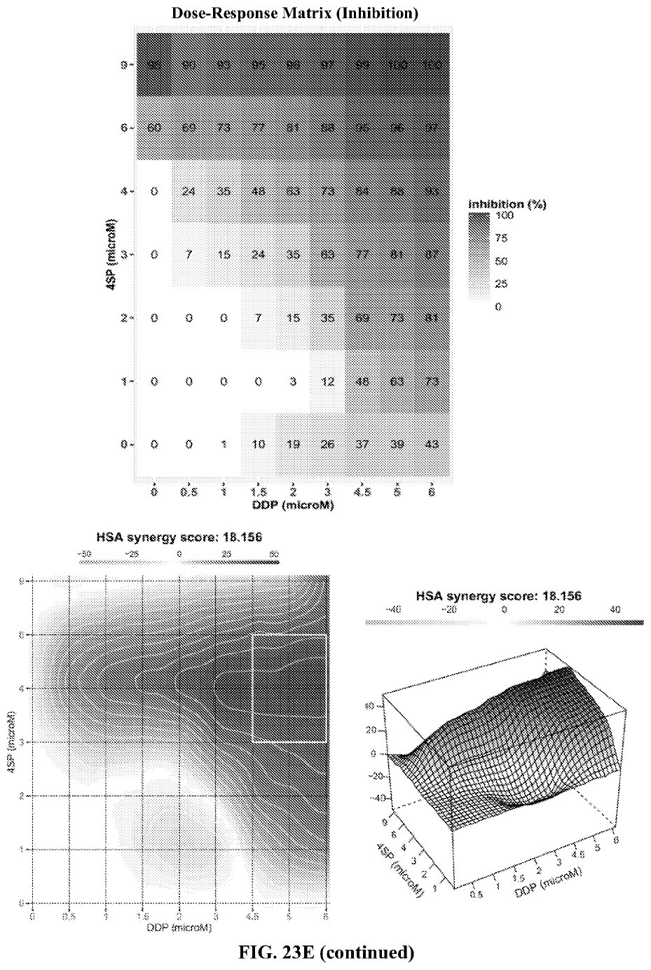

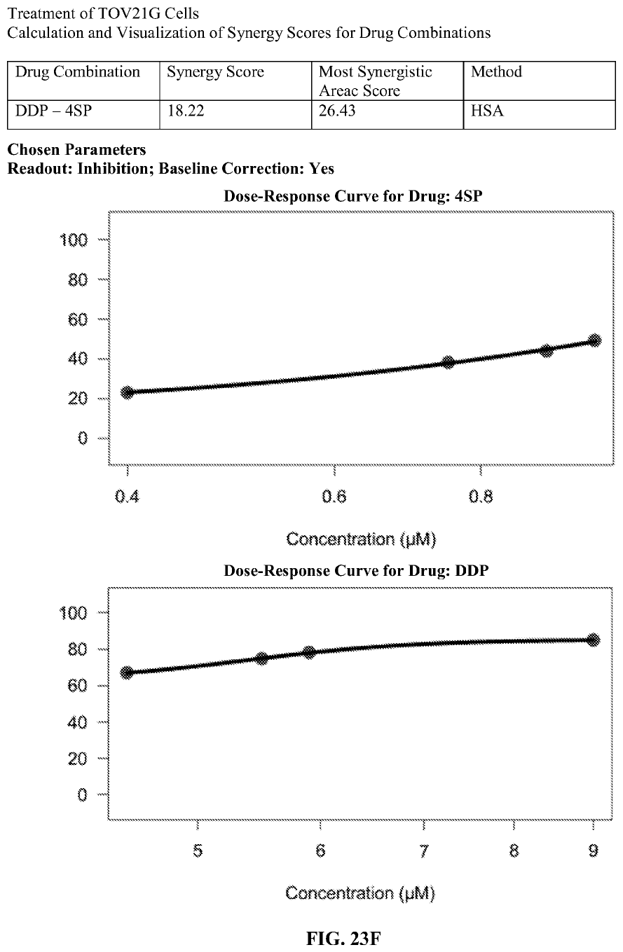

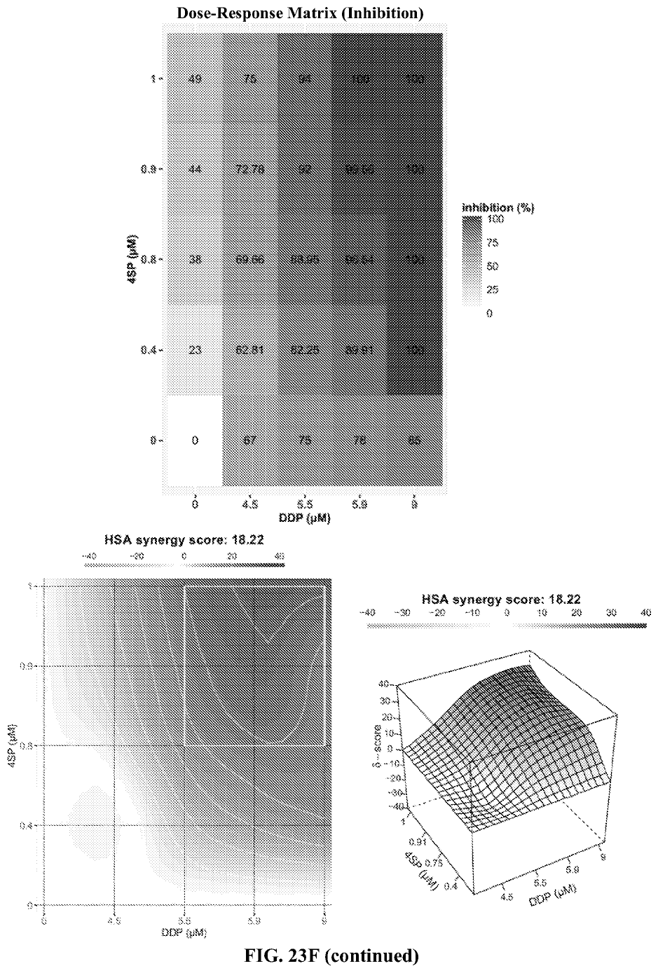

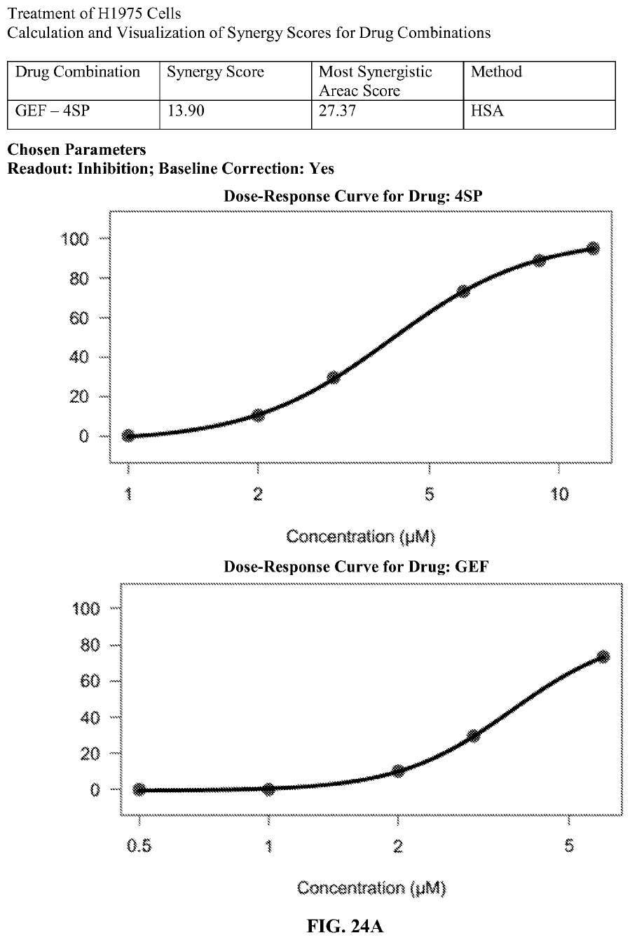

The nature of the combined cytostatic/cytocidal effects of 4SP65-cisplatin was further surveyed via synergy reports where the upper limits for cisplatin were set at 15 μM to focus on responses at in vitro treatment levels corresponding to reported peak plasma levels of free drug. Higher single agent (HSA) synergy scores <10 mean that the interaction between two drugs is likely to be antagonistic; scores from −10 to 10 mean the interaction between two drugs is likely to be additive; and scores >10 means the interaction between two drugs is likely to be synergistic. The highest average HSA synergy score of 36.37 on a scale of 60 was observed in H460 cells, where the potentiation of 4SP65-cisplatin combined was most prominent in the low-dose region of both drugs as shown in the heat map and in a ridge of high synergy score in the volcano plot of 45 to 55 at concentrations of ≤1 μM cisplatin combined with 1.5 to 4.6 μM 4SP65 (FIG. 23B). A similar pattern with an average synergy score of 20 was observed in A549 cells with, for example, treatment with 8 μM cisplatin and 6 μM 4SP65 yielding 90% reduction in starting cell numbers (FIG. 23A) while 6 μM cisplatin alone only produced 56% reduction (Table 3). Average synergy scores of 18.16 and 18.22, respectively, for 4SP65-cisplatin treated SKOV3 and TOV21G cells were associated with near complete to complete cell killing in each cell line (FIGS. 23E-23F), while corresponding levels of cisplatin alone was not cytolytic in SKOV3 cells and only reached an LC₅₀ at 13.2 μM cisplatin in TOV21G cells (Table 3). 4SP65-cisplatin combined was least effective against HMESO-1 and PPM-Mill cells, with weak potentiation or additive effects driven primarily by significant increases in the cytolytic effects of cisplatin in the combination not achieved by plasma-equivalent levels of cisplatin alone (Table 3, FIGS. 23C-23D).

### Example 16—4SP65 Enhances Effectiveness and Overcomes Drug Resistance in Combination with EGFR TKIs in NSCLC Cell Lines Resistant to EGFR-Selective TKIs

A series of experiments were conducted to define the degree to which co-treatment with aminothiol-conjugates of Formula (I) enhances the anticancer effectiveness and overcomes drug resistance to gefitinib, as a representative small molecule targeting epidermal growth factor receptor (EGFR) mutations, in relevant panels of human cancer cell lines.

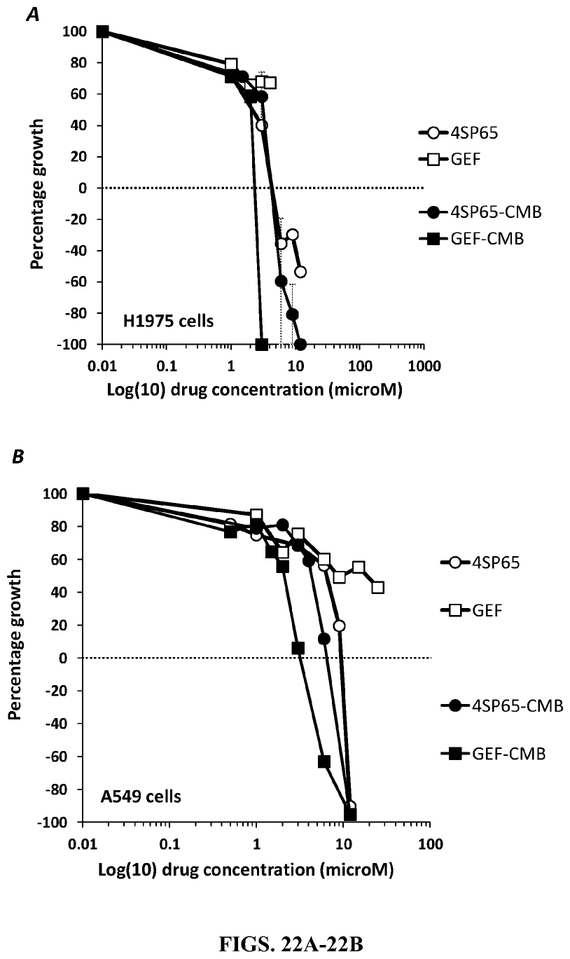

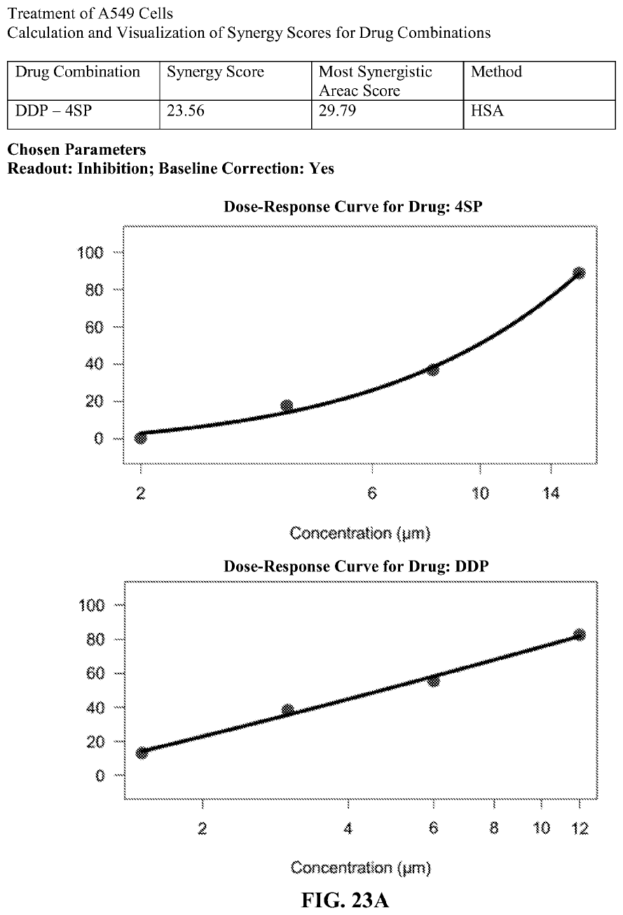

A limited set of experiments was conducted to determine if 4SP65 enhances the activity of and overcomes resistance to the EGFR-selective tyrosine kinase inhibitor (TKI), gefitinib, in H1975 cells that carry the L858R mutation sensitive to gefitinib activity plus the T790M mutation that confers resistance to first-generation TKIs (Chen and Riess, “Advances in Targeting Acquired Resistance Mechanisms to Epidermal Growth Factor Receptor Tyrosine Kinase Inhibitors,” J. Thorac. Dis. 12 (5): 2859-2876 (2020) and de Wit, “Mutation and Drug-Specific Intracellular Accumulation of EGFR Predict Clinical Responses to Tyrosine Kinase Inhibitors,” EBioMedicine 56:102796 (2020), which are hereby incorporated by reference in their entirety). For comparison, the growth inhibitory effects of gefitinib with and without 4SP65 were investigated in A549 cells, which overexpress wild-type EGFR but are considered a representative cell line for innate EGFR-TKI resistance (Dong et al., “The Long Non-Coding RNA, GAS5, Enhances Gefitinib-Induced Cell Death in Innate EGFR Tyrosine Kinase Inhibitor-Resistant Lung Adenocarcinoma Cells with Wide-Type EGFR via Downregulation of the IGF-1R Expression,” J. Hematol. Oncol. 8:43 (2015), which is hereby incorporated by reference in its entirety). For these experiments, a human cancer cell line was considered to be resistant to gefitinib when the amount of drug required to achieve cytolytic effects in cell culture was >6.0 μM gefitinib. This upper limit was based upon reports that, in cancer patients receiving 250 mg gefitinib daily, Cₘₐₓ plasma concentrations are 0.5-1 μM or more (Cohen et al., “United States Food and Drug Administration Drug Approval Summary: Gefitinib (ZD1839; Iressa) Tablets,” Clin. Cancer Res. 10 (4): 1212-1218 (2004), which is hereby incorporated by reference in its entirety) but ‘trough levels’ (i.e., the concentration of drug in the blood immediately before the next dose is administered) range from 0.257-4.50 μM and 0.282-6.55 μM after 3 and 8 days of treatment, respectively, due to the long elimination half-life of 48 hours for gefitinib (Nakamura et al., “Pharmacokinetics of gefitinib Predicts Antitumor Activity for Advanced Non-Small Cell Lung Cancer,” J. Thorac. Oncol. 5 (9): 1404-1409 (2010), which is hereby incorporated by reference in its entirety). For each cancer cell line, FIGS. 22A-22B show the dose-response curves for cell growth after 48-hour treatment with each drug alone or 4SP65-gefitinib combined, while Table 4 lists the corresponding dose-response metrics, the fold-increase in efficacy for each drug in the combination (with P-values indicated where increases are significant), and the synergy scores for the combination.

TABLE 4
Dose response metrics for 4SP65 alone, gefitinib alone, and 4SP65-
gefitinib combined in drug-exposed human cancer cell lines^{a}

| | | | | | Fold increase in efficacy | |
|---|---|---|---|---|---|---|
| Cell line | 4SP65 | 4SP65-c | GEF | GEF-c | 4SP65/4SP65-c | GEF/GEF-c |

H1975 (n = 4 experiments)

| | | | | | | |
|---|---|---|---|---|---|---|
| IC₅₀ | 3.5 ± 0.9 | 3.3 ± 0.6 | ND (>25) | 1.0 ± 0.5 | 1.1 (NS) | >25 (GoE) |
| GI₅₀ | 3.2 ± 1.1 | 2.5 ± 0.8 | 10.8 ± 3.4 | 0.75 ± 0.34 | 1.3 (NS) | 14.0** |
| TGI | 5.5 ± 1.4 | 4.6 ± 1.3 | ND (>25) | 1.4 ± 0.6 | 1.2 (NS) | >18 (GoE) |
| LC₅₀ | 7.2 ± 1.4 | 7.6 ± 1.4 | ND (>25) | 2.3 ± 1.0 | 1.0 (NS) | >11 (GoE) |
| LC₉₉ | 8.9 ± 1.6 | 9.1 ± 1.7 | ND (>25) | 2.8 ± 2.2 | 1.0 (NS) | >8.9 (GoE) |

^{b}(Average HSA synergy score = 13.9; Most synergistic area score = 27.4)
A549 (n = 5 experiments)

| | | | | | | |
|---|---|---|---|---|---|---|
| IC₅₀ | 7.4 ± 0.7 | 5.3 ± 0.8 | 23.3 ± 5.7 | 2.6 ± 0.5 | 1.4** | 9.0*** |
| GI₅₀ | 6.0 ± 0.6 | 4.1 ± 0.7 | 17.5 ± 4.4 | 2.0 ± 0.5 | 1.5** | 8.8** |
| TGI | 11.3 ± 1.5 | 7.8 ± 0.9 | ND (>25) | 3.9 ± 0.8 | 1.5** | >6.4 (GoE) |
| LC₅₀ | 11.4 ± 2.7 | 10.9 ± 0.5 | ND (>25) | 5.4 ± 1.1 | 1.1 (NS) | >4.6 (GoE) |
| LC₉₉ | 12.3 ± 3.8 | 12.5 ± 0.8 | ND (>25) | 6.2 ± 1.4 | 1.0 (NS) | >4.0 (GoE) |

^{b}(Average HSA synergy score = 14.7; Most synergistic area score = 20.5)
^{a}Cell lines were plated in 96-well microtiter dishes, incubated for approximately 24 hours prior, and then
treated over a dose range for 48 hours. Calculation of values for inhibitory concentration 50% (IC₅₀),
growth inhibitor concentration 50% (GI₅₀), total growth inhibitory concentration (TGI), lethal
concentration 50% (LC₅₀) and 99% (LC₉₉) was done as set forth by the NCI (Monks et al., “Feasibility of
a High-Flux Anticancer Drug Screen using a Diverse Panel of Cultured Human Tumor Cell Lines,” J.
Natl. Cancer Inst. 83(11): 757-66 (1991) and Hafner et al., “Growth Rate Inhibition Metrics Correct for
Confounders in Measuring Sensitivity to Cancer Drugs,” Nat. Methods 13(6): 521-527 (2016), which are
hereby incorporated by reference in their entirety). All units are μM, with means and standard
deviations. Abbreviations: 4SP65-c and DDP-c refer to the amounts of each drug in combination at
each dose-response metric. The total amount of each drug administered to obtain each dose response
metric is the sum of the concentration of 4SP65-c and GEF-c. (see ‘how the combination experiments
were done’ in notes for more details. ND, not definable, with following number in parentheses indicating
the highest concentration of drug tested in μM for a given cancer cell line; GoE, gain of effect; NS, non-
significant.
^{b}Synergy score data obtained via SynergyFinder (Ianeski et al., “SynergyFinder 2.0: Visual Analytics of
Multi-Drug Combination Synergies,” Nucleic Acids Res. 48(W1): W488-W493 (2020), which is hereby
incorporated by reference in its entirety).
*P < 0.05,
**P ≤ 0.01,
***P ≤ 0.001

In H1975 cells, exposure for 48 hours to gefitinib alone at up to 25 μM drug failed to exhibit total growth inhibition or cytolytic effects. In contrast, co-exposure to 4SP65-gefinitib triggered significant potentiation of both the cytostatic and cytocidal effects of gefitinib to achieve a GI₅₀ in the nanomolar range and a LC₉₉ at <3 μM gefitinib in the combination (FIG. 22A). The increase in the efficacy of gefitinib in the combination was significant for all dose-response metrics, as exemplified by a 14-fold increase at the GI₅₀ level (Table 4); however, while co-treatment with gefitinib resulted in small increases in the efficacy of 4SP65 at the IC50, GI50, and TGI levels, it had no impact at the LC_{50/99} levels, with the result that there was no significant increase in 4SP65 efficacy for this combination (p>0.05 for all dose metrics).

In A549 cells, exposure to gefitinib alone at up to 25 μM did not induce total growth inhibition or cytolytic effects, and the drug concentration that induced a GI₅₀ level effect occurred at drug concentrations roughly three-fold greater than the highest trough concentrations (FIG. 22B). In contrast, co-treatment of A549 cells with 4SP65 resulted in gefitinib having a significant therapeutic contribution at clinically relevant levels ranging from 2.0 μM gefitinib at the GI₅₀ level to 5.4 μM gefitinib in the drug-combination (Table 4). The amplification in the efficacy of gefitinib in the combination was sizable for all dose-response metrics, as exampled by approximately 9-fold increases at the IC₅₀ and GI₅₀ levels. Co-exposure to gefitinib also yielded significant increases in the growth inhibitory effects but not the cytolytic effects of 4SP65 in the combination in A549 cells, as demonstrated by the finding that 4SP65 efficacy in combination did not change at the LC50/99 effect levels (Table 4). The data support the conclusion that 4SP65 works synergistically with gefitinib to induce cell growth inhibition, but cell killing is governed largely by the effects of 4SP65 alone.

### Example 17—Visual Analytics (Synergy Reports) of Combination Synergies of 4SP65 Plus an EGFR-TKI

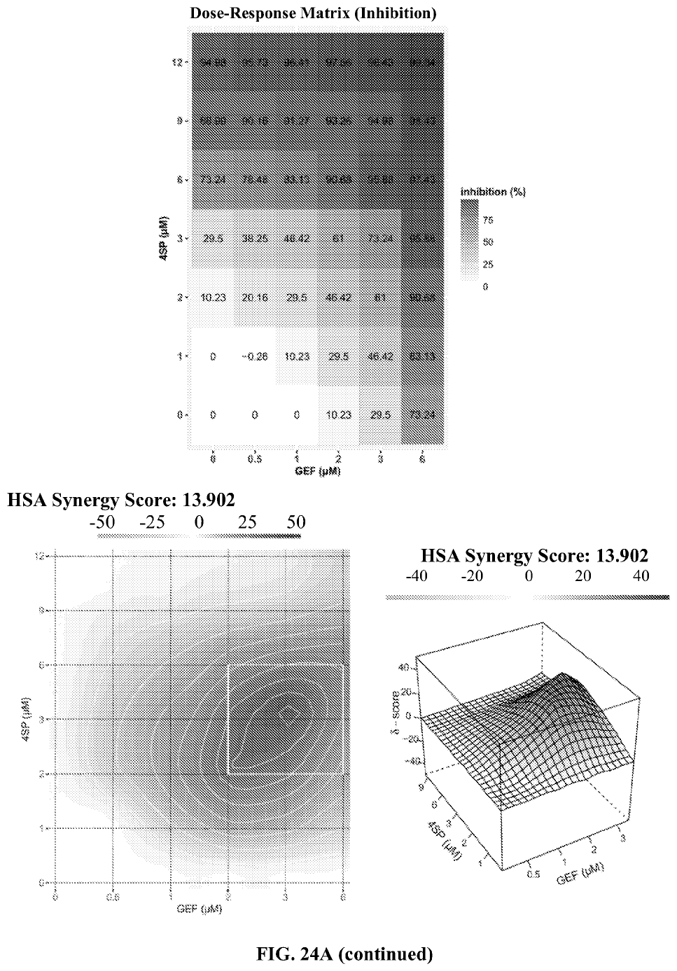

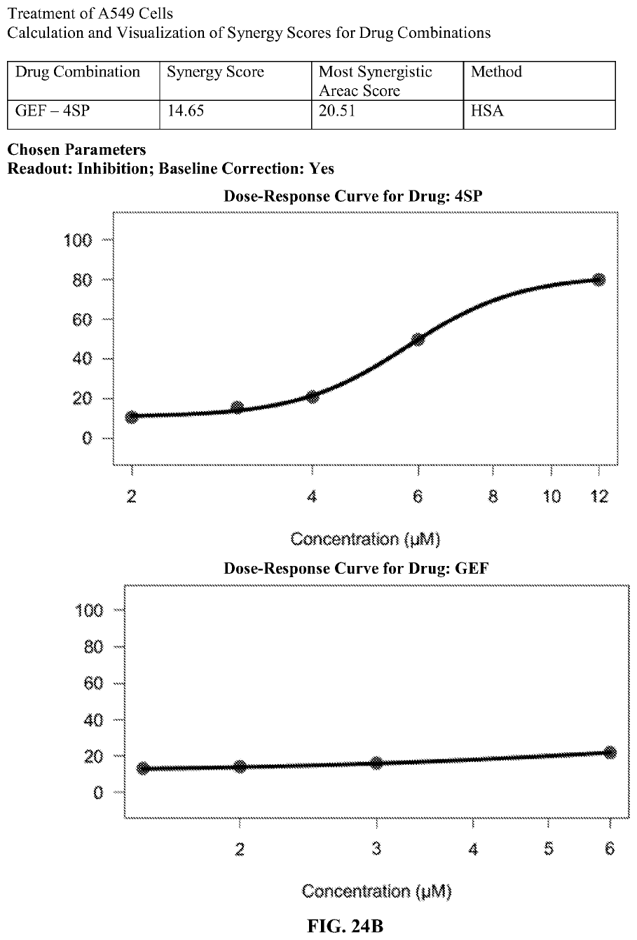

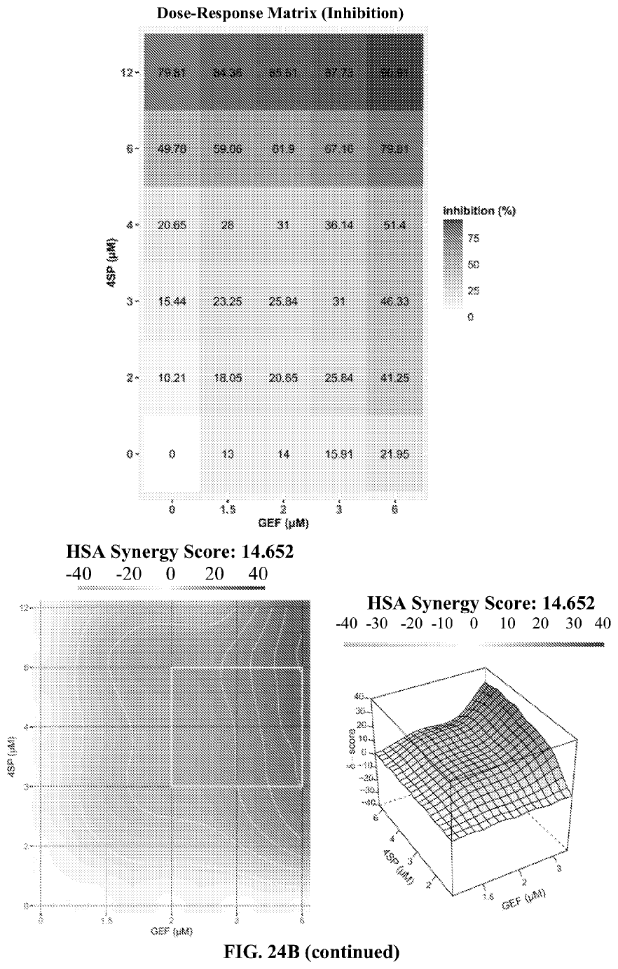

The nature of the combined cytostatic/cytocidal effects of 4SP65 plus gefitinib was further surveyed via synergy reports where the upper limits for cisplatin were set at 6 μM to focus on responses at in vitro treatment levels corresponding to reported peak plasma trough levels of free drug at 4.50 μM and 6.55 μM after 3 and 8 days of treatment in patients with NSCLC (Nakamura et al., “Pharmacokinetics of gefitinib Predicts Antitumor Activity for Advanced Non-Small Cell Lung Cancer,” J. Thorac. Oncol. 5 (9): 1404-1409 (2010), which is hereby incorporated by reference in its entirety). In H1975 cells, the dose-response matrix in the synergy report shows that 48-hour treatments with combinations of 0.5 μM gefitinib plus 9.0 μM 4SP65, 2.0 μM gefitinib plus 6.0 μM 4SP65, or 6.0 μM gefitinib plus 2.0 μM 4SP65 induced an approximately 90% reduction in starting cell numbers, while the heat map and volcano plot demonstrate that the average synergy score of approximately 13.90 was driven by the most synergistic area score of 27.3 occurring between 1 and 3 μM of both gefitinib and 4SP65 (FIG. 24A). However, as noted above, a 48-hour treatment with 25 μM gefitinib alone failed to induce total growth inhibition or cytolytic effects due to the T790M mutation that confers resistance in H1975 cells.

The synergy report for A549 cells shows that in spite of a basically flat dose-response curve for gefitinib alone, co-treatment with 4SP65-gefitinib triggered both cytostatic and cytolytic effects at biologically-relevant concentrations of both drugs, achieving ˜59% reduction in starting cell numbers at 1.5 μM gefitinib plus 6 μM 4SP65 and ˜91% reduction in starting cell numbers at 6 μM gefitinib plus 12 μM 4SP65 (FIG. 24B). The dose-response matrix in the synergy report shows that 48-hour co-treatment with 4SP65-gefitinib resulted in ˜60 to 67% inhibition of starting cell numbers as the concentration of gefitinib was increased from 1.5 to 3 μM in combination with 6 μM 4SP65 (FIG. 24B). The heat map and volcano plot further demonstrate that the average synergy score of 14.65 was driven by the most synergistic area score of 20.51 occurring between 2 and 6 μM gefitinib and 3 and 6 μM 4SP65.

### Discussion of Examples 13-17

Examples 13-17 investigate the degree to which the anticancer effects of 4SP65 and 1LP65 can be extended to drug combinations and effects upon drug resistance. The results of initial studies demonstrating the broad-spectrum anticancer activity of 4SP65 and 1LP65 were extended to show efficacy against representative immortalized cell lines, synergy when used in combination with a representative cytotoxic chemotherapeutic (cisplatin) or a EGFR-TKI (gefitinib), and reversal of drug resistance for these two anticancer agents. The combination of 4SP65 with either cisplatin or gefitinib resulted in increased cytostatic effectiveness for both drugs, and reduced the amount of each drug needed to achieve this level of effects to levels that can be achieved in vivo. In addition, in cell lines where cisplatin was unable to induce cytolytic effects when used alone, when used in combination with 4SP65 LC₉₉ levels effects were achieved at doses of cisplatin that can be obtained in patients. These results were confirmed by both NCI methodology-based evaluations and by evaluations performed using Synergyfinder 2.0.

The results presented herein support the postulate that the anticancer effects, drug synergism, and drug resistance reversal effects of the aminothiol-conjugates of the present disclosure (e.g., 4SP65 and 1LP65) result from their metabolism and the nature of their active ingredients. These considerations also suggest explanations for differences in BRG agent effects between normal cells and cancer cells, and indicate reasons for differences between BRG prodrugs and amifostine.

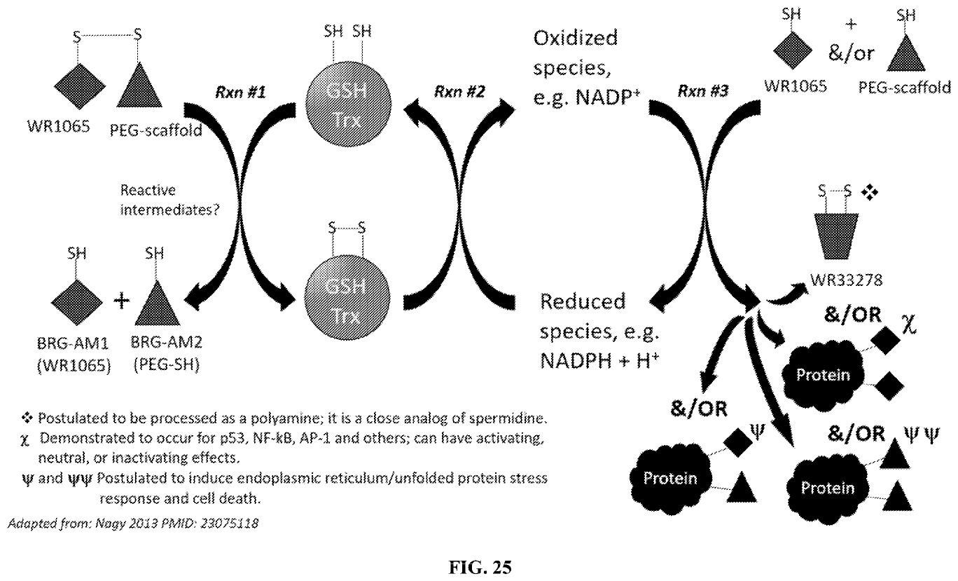

The aminothiol-conjugates of the present disclosure (e.g., 4SP65 and 1LP65) are disulfides that require reduction by glutathione or thioredoxin for the two constituent molecules (WR1065 and PEG-SH scaffold) to be released. FIG. 25 shows the metabolism of a BRG prodrug (e.g., 4SP65 and 1LP65), which occurs through a sequence of kinetically-favored reactions (Nagy, P., “Kinetics and Mechanisms of Thiol-Disulfide Exchange Covering Direct Substitution and Thiol Oxidation-Mediated Pathways,” Antioxid Redox Signal 18 (13): 1623-1641 (2013), which is hereby incorporated by reference in its entirety). After an aminothiol-conjugate of the present disclosure (e.g., 4SP65 and 1LP65) enters the cytoplasm, either glutathione or thioredoxin reduces the BRG disulfide bond (Rxn #1) to yield reduced WR1065 and PEG-SH scaffold. In normal cells with a reducing cytosolic redox balance, evidence of formation of free radical intermediates during this reaction has not been reported, but formation of thioredoxin anion has been reported (Nagy, P., “Kinetics and Mechanisms of Thiol-Disulfide Exchange Covering Direct Substitution and Thiol Oxidation-Mediated Pathways,” Antioxid Redox Signal 18 (13): 1623-1641 (2013), which is hereby incorporated by reference in its entirety). Conversely, in cancer cells where the cellular environment is shifted towards oxidizing conditions, there is evidence that reactive species such as thiyl free radicals can be formed (Nagy, P., “Kinetics and Mechanisms of Thiol-Disulfide Exchange Covering Direct Substitution and Thiol Oxidation-Mediated Pathways,” Antioxid. Redox Signal 18 (13): 1623-1641 (2013), which is hereby incorporated by reference in its entirety), which can have adverse effects in neoplastic cells. Oxidized glutathione or thioredoxin is reduced (Rxn #2) by NADPH and hydrogen, generating oxidized NADP⁺. Evidence for reduction of NADP⁺ by BRG active ingredients (Rxn #3) comes from experiments using Alamar Blue (resazurin), which fluoresces when reduced by NADPH but not when oxidized (Davis et al., “Diaphorase Coupling Protocols for Red-Shifting Dehydrogenase Assays” Assay Drug Dev Technol. 2016 April; 14 (3): 207-12 (2016), which is hereby incorporated by reference in its entirety). For example, in H1437 NSCLC cells exposed to 4SP65 in the presence of resazurin, the cellular reducing capacity was maintained at near 100% of control cells until cell numbers had been reduced to approximately 50% of controls. The reduction of NADP⁺ to NADPH (Rxn #3) results in oxidized WR1065 and/or PEG-scaffold. It is not known whether a thiolate anion or a thiyl free radical is formed as an intermediate. Evidence suggests that in normal cells the formation of such intermediates is low, while in cancer cells the probability is increased under oxidative environment; these phenomena depend upon the nature of the reacting thiol along with the cell context, i.e., reducing versus oxidizing conditions (Romero et al. “The reactivity of thiols and disulfides with different redox states of myoglobin. Redox and addition reactions and formation of thiyl radical intermediates” J Biol Chem 267 (3): 1680-1688 (1992), which is hereby incorporated by reference in its entirety).

Oxidizing conditions also can result in altered redox states of protein cysteines. FIG. 25 shows known and postulated products of reaction #3. WR33278 is one product, which also is formed after exposure to amifostine. The other products include disulfide bond formation between WR1065 with protein cysteines; similar binding is postulated for oxidized PEG-SH scaffold. While some proteins are known to be bound by WR1065, it is probable that binding of both WR1065 and the PEG scaffold can occur.

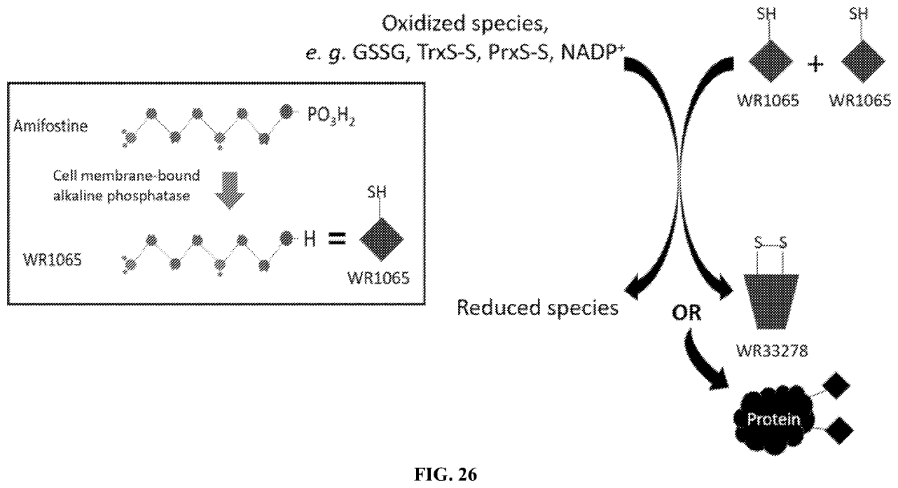

The metabolism of amifostine differs in significant ways from that of the aminothiol-conjugates of the present disclosure (e.g., 4SP65 and 1LP65). FIG. 26 shows a metabolic scheme for amifostine and reported reaction products for WR1065, a potent, reductant nucleophile. Under ideal conditions amifostine is converted to WR1065 by membrane-bound alkaline phosphatase. This hydrolysis reaction does not perturb the redox balance. WR1065 is reported to donate hydrogen ions and, thus, can reduce oxidized species such as oxidized glutathione (GSSG), thioredoxin (with an intramolecular disulfide bond), peroxiredoxin (with an intramolecular disulfide bond), and NADP″ (the oxidized form). Other oxidized molecules also can be reduced by WR1065. The net effect is to increase the reducing capacity of cells; under stress condition this WR1065-mediated effect increases the cellular ability to neutralize electrophilic species. WR1065 also is reported to bind to a selected subset of proteins, including both wild-type and mutant p53 protein, components of nuclear factor kappa B (NF-kB) pathways and subcomponents of the AP-1 transcription factor. Binding sites are postulated to be at electrophilic oxidized cysteine residues. Evidence from WR1065 studies supports the conclusions that the homodimer WR33278 (composed of two WR1065 molecules) is stable, and has effects similar to polyamines (WR33278 is a close analog of the polyamine spermidine). WR1065 bound to proteins is reported to be stable for up to 60 hours (Pluquet et al., “Activation of p53 by the Cytoprotective Aminothiol WR1065: DNA-Damage-Independent Pathway and Redox-Dependent Modulation of p53 DNA-Binding Activity,” Biochem. Pharmacol. 65 (7): 1129-1137 (2003), which is hereby incorporated by reference in its entirety). The cytoprotective effects of amifostine can be attributed to these processes.

Taken together, these considerations support that WR1065 delivered as an aminothiol-conjugate of the present disclosure (e.g., 4SP65 and 1LP65) has a different profile of reactions and/or binding sites in normal cells versus cancer cells, and the protein binding profile also differs from that of WR1065 delivered as amifostine. These distinctions can explain differences in effects between drugs. In addition, there is a potential for the PEG-SH scaffold to also bind proteins and/or form disulfides, which can perturb protein processing due to their bulkiness. Altered protein processing can result in an endoplasmic reticulum/unfolded protein stress response and subsequent cell death. The ability of WR1065 and other p53 protein binding anticancer agents to induce this type of stress response has been reported (Bykov et al., “Mutant p53-Dependent Growth Suppression Distinguishes PRIMA-1 from known Anticancer Drugs: A Statistical Analysis of Information in the National Cancer Institute Database,” Carcinogenesis 23 (12): 2011-2018 (2002); Pluquet et al., “Activation of p53 by the Cytoprotective Aminothiol WR1065: DNA-Damage-Independent Pathway and Redox-Dependent Modulation of p53 DNA-Binding Activity,” Biochem. Pharmacol. 65 (7): 1129-1137 (2003); Dedieu et al. “The cytoprotective drug amifostine modifies both expression and activity of the pro-angiogenic factor VEGF-A” BMC Med 8:19 (2010), which are hereby incorporated by reference in its entirety).

The above considerations provide an explanation for the synergistic and drug resistance reversal effects of the aminothiol-conjugates of the present disclosure (e.g., 4SP65 and 1LP65). By perturbing the redox balance of cancer cells and adding to proteotoxic stress by binding to proteins, aminothiol-conjugates of the present disclosure (e.g., 4SP65 and 1LP65) compromise two cellular systems or machines, needed by cancer cells for survival (Dobbelstein et al. “Targeting tumour-supportive cellular machineries in anticancer drug development” Nat Rev Drug Discov 13 (3): 179-196 (2014), which is hereby incorporated by reference in its entirety). When combined with cisplatin, which compromises DNA machinery as well as adding to redox and proteotoxic stress, the compensatory/adaptive capability of cancer cells is overloaded, resulting in cell death. Similar effects occur with gefitinib, where targeting of the EGFR-TKI signaling system adds to redox and proteotoxic stress to induce cell death.

Although certain embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of increasing sensitivity of a cell to treatment with an anti-neoplastic drug or antimicrobial drug said method comprising:
selecting a neoplastic cell, a microbial cell, or a cell infected with a microbe; and
administering to the cell an aminothiol-conjugate of Formula (I): 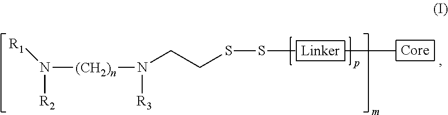
wherein 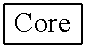
is optional and, if present, is an atom, a molecule, or a macromolecule; 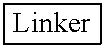
is a linker group, wherein the linker group is a polymer, a section of a polymer, an arm of a polymer, an arm of a copolymer, or a branch of a dendrimer;
wherein each 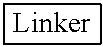
can be the same or different;
R₁, R₂, and R₃ are independently selected from hydrogen and C₁₋₆ alkyl;
m is 1 to 100,000;
n is 1 to 10; and
p is independently 0 to 2,500,
or a pharmaceutically acceptable salt, hydrate, polymorph, or solvate thereof in an amount effective to increase sensitivity of the cell to treatment with the anti-neoplastic drug or antimicrobial drug.

2. The method according to claim 1, wherein the cell is a neoplastic cell.

3. The method according to claim 2, wherein the neoplastic cell is selected from the group consisting of a non-small lung cancer cell, breast cancer cell, ovarian cancer cell, cervical cancer cell, colon cancer cell, lung cancer cell, skin cancer cell (malignant melanoma cell), lymphoreticular tumor cell, lung cancer cell, prostate cancer cell, colorectal cancer cell, melanoma cell, bladder cancer cell, lymphoma cell, Hodgkin lymphoma cell, non-Hodgkin lymphoma cell, endometrial cancer cell, leukemia cell, kidney cancer cell, pancreatic cancer cell, thyroid cancer cell, liver cancer cell, sarcoma cell, a cell from a myelodysplastic condition, a cell from an undifferentiated tumor, and combinations thereof.

4. The method according to any one of claims 1-3, wherein the anti-neoplastic drug is a protein kinase inhibitor.

5. The method according to any one of claims 1-3, wherein the anti-neoplastic drug is selected from the group consisting of cisplatin, erlotinib, gefitinib, afatinib, osimertinib, abemaciclib, carboplatin, oxaliplatin, nedaplatin, and lobaplatin or derivatives thereof.

6. The method according to any one of claim 1-3 or 5, wherein the anti-neoplastic drug is cisplatin.

7. The method according to any one of claims 1-5, wherein the anti-neoplastic drug is gefitinib.

8. The method according to claim 1, wherein the antimicrobial drug is selected from the group consisting of an antibiotic drug, an antiviral drug, an antifungal drug, and antiparasitic drug, and an antibacterial drug.

9. The method according to claim 1, wherein the antimicrobial drug is selected from the group consisting of (i) eukaryotic and prokaryotic protein kinase inhibitors, (ii) nucleoside analogs, (iii) bacterial cell wall or cell envelope breakdown agents, (iv) antifungal drugs, (v) antiparasitic drugs, (vi) antiviral drugs, or derivatives thereof.

10. The method according to claim 1, wherein the antimicrobial drug is an antiviral drug selected from the group consisting of (i) eukaryotic and prokaryotic protein kinase inhibitors and (ii) nucleoside analogs or derivatives thereof.

11. The method according to any one of claims 1-10, further comprising: administering to the neoplastic cell, the microbial cell, or the cell infected with a microbe the anti-neoplastic drug or antimicrobial drug together with or after said administering the aminothiol-conjugate of Formula (I).

12. The method according to any one of claims 1-11, wherein the method is carried out in vitro.

13. The method according to any one of claims 1-11, wherein the method is carried out in vivo.

14. The method according to any one of claims 1-13, wherein the aminothiol-conjugate of Formula (I) has the following structure: 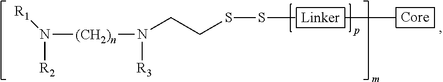
wherein 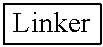
is polyethylene glycol (PEG) or a repeating unit of PEG; 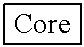
is optional and, if present, is a polymer core or 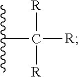
m is 1, 2, 3, 4, 5, 6, 7, or 8;
R is independently selected from hydrogen and C₁₋₆ alkyl;
R₁, R₂, and R₃ are H;
n is 3; and
each p is independently 1 to 2,500.

15. The method according to any one of claims 1-14, wherein the aminothiol-conjugate of Formula (I) has the structure of:

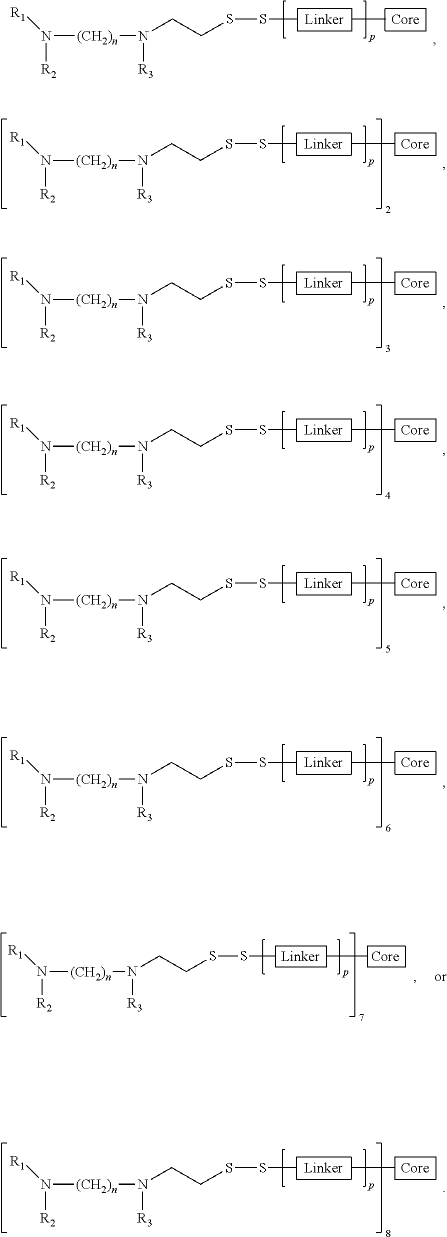

16. The method according to any one of claims 1-15, wherein the aminothiol-conjugate of Formula (I) has the following structure: 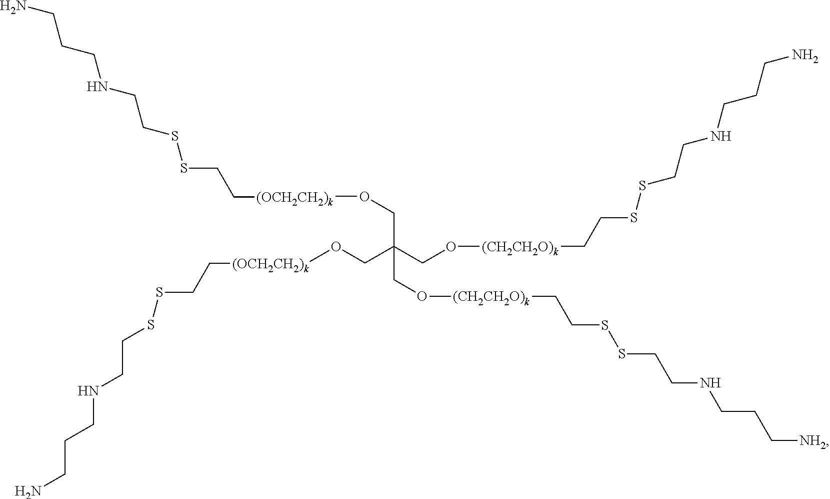
wherein k is independently 1 to 2,500, or a pharmaceutically acceptable salt thereof.

17. The method according to any one of claims 1-15,
wherein the aminothiol-conjugate of Formula (I) has the following structure: 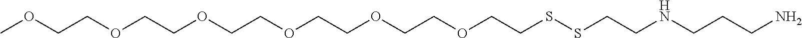 [SMILES: COCCOCCOCCOCCOCCOCCSSCCNCCCN]
or a pharmaceutically acceptable salt thereof.

18. A method of treating a neoplastic condition in a subject in need thereof, said method comprising:
administering to said subject a combination therapy comprising:
(i) an aminothiol-conjugate of Formula (I): 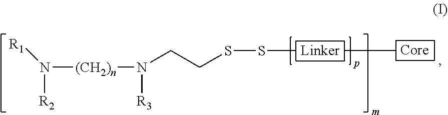
wherein 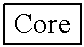
is optional and, if present, is an atom, a molecule, or a macromolecule; 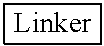
is a linker group, wherein the linker group is a polymer, a section of a polymer, an arm of a polymer, an arm of a copolymer, or a branch of a dendrimer;
wherein each 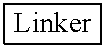
can be the same or different;
R₁, R₂, and R₃ are independently selected from hydrogen and C₁₋₆ alkyl;
m is 1 to 100,000;
n is 1 to 10; and
p is independently 0 to 2,500,
or a pharmaceutically acceptable salt, hydrate, polymorph, or solvate thereof, and
(ii) an anti-neoplastic drug, wherein the combination therapy is administered in an amount effective to treat the neoplastic condition in the subject.

19. The method according to claim 18, wherein the anti-neoplastic drug is a protein kinase inhibitor.

20. The method according to claim 18, wherein the anti-neoplastic drug is selected from the group consisting of cisplatin, erlotinib, gefitinib, afatinib, osimertinib, tetraplatin, ipraplatin, abemaciclib, carboplatin, oxaliplatin, nedaplatin, lobaplatin, and/or derivatives of any thereof.

21. The method according to claim 18 or claim 20, wherein the anti-neoplastic drug is cisplatin.

22. The method according to any one of claims 18-20, wherein the anti-neoplastic drug is gefitinib.

23. The method according to any one of claims 18-22, wherein the neoplastic condition is selected from the group consisting of lung cancer, breast cancer, ovarian cancer, cervical cancer, colon cancer, skin cancer, lymphoreticular tumors, prostate cancer, colorectal cancer, bladder cancer, lymphoma, Hodgkin lymphoma, non-Hodgkin lymphoma, endometrial cancer, leukemia, kidney cancer, pancreatic cancer, thyroid cancer, liver cancer, sarcomas, myelodysplastic condition, undifferentiated tumors, and combinations thereof.

24. The method according to any one of claims 18-23, wherein the subject receives a radiation therapy, a chemotherapy, or a combination thereof and the combination therapy is administered under conditions effective to reduce or decrease an adverse or undesirable side-effect of the radiation therapy, the chemotherapy, or the combination thereof.

25. The method according to any one of claims 18-24, wherein the subject is a mammal.

26. The method according to claim 25, wherein the mammal is a human.

27. The method according to any one of claims 18-26, wherein the neoplastic condition being treated is resistant to a treatment with the anti-neoplastic drug.

28. The method according to any one of claims 18-27, wherein the aminothiol-conjugate of Formula (I) has the following structure: 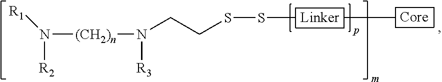
wherein 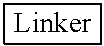
is polyethylene glycol (PEG) or a repeating unit of PEG; 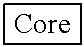
is optional and, if present, is a polymer core or 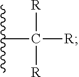
m is 1, 2, 3, 4, 5, 6, 7, or 8;
R is independently selected from hydrogen and C₁₋₆ alkyl;
R₁, R₂, and R₃ are H;
n is 3; and
each p is independently 1 to 2,500.

29. The method according to any one of claims 18-28, wherein the aminothiol-conjugate of Formula (I) has the structure of:

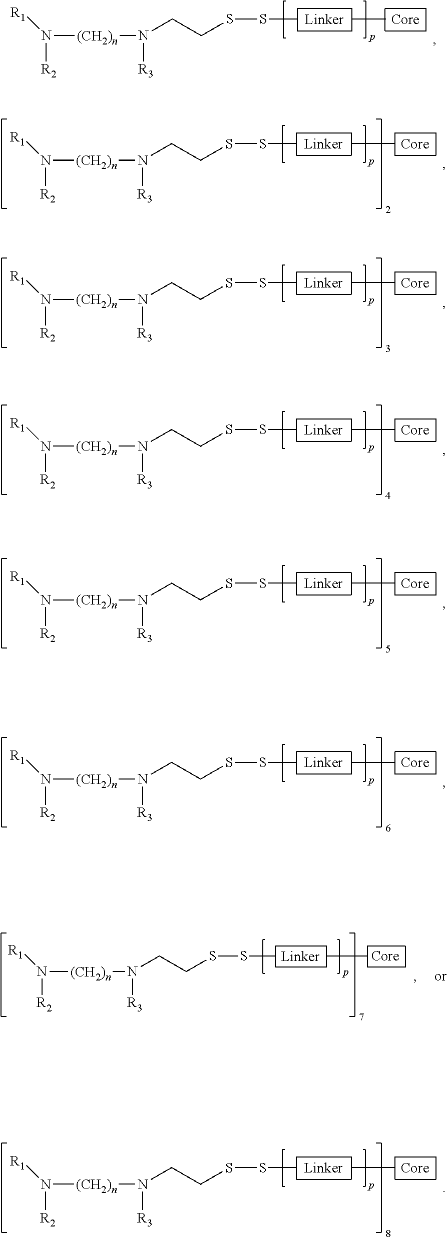

30. The method according to any one of claims 18-29, wherein the aminothiol-conjugate of Formula (I) has the following structure: 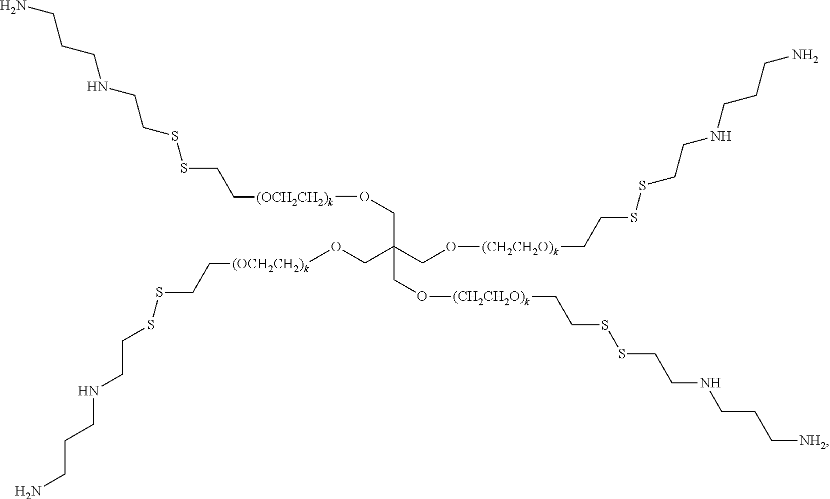
wherein k is independently 1 to 2,500, or a pharmaceutically acceptable salt thereof.

31. The method according to any one of claims 18-29,
wherein the aminothiol-conjugate of Formula (I) has the following structure: 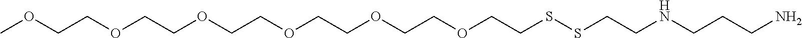 [SMILES: COCCOCCOCCOCCOCCOCCSSCCNCCCN]
or a pharmaceutically acceptable salt thereof.

32. A combination therapeutic comprising:
(i) an aminothiol-conjugate of Formula (I): 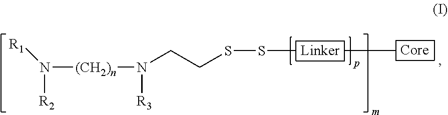
wherein 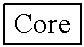
is optional and, if present, is an atom, a molecule, or a macromolecule; 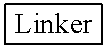
is a linker group, wherein the linker group is a polymer, a section of a polymer, an arm of a polymer, an arm of a copolymer, or a branch of a dendrimer;
wherein each 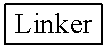
can be the same or different;
R₁, R₂, and R₃ are independently selected from hydrogen and C₁₋₆ alkyl;
m is 1 to 100,000;
n is 1 to 10; and
p is independently 0 to 2,500,
or a pharmaceutically acceptable salt, hydrate, polymorph, or solvate thereof, and
(ii) an anti-neoplastic drug.

33. The combination therapeutic according to claim 32, wherein the anti-neoplastic drug is a protein kinase inhibitor.

34. The combination therapeutic according to claim 32, wherein the anti-neoplastic drug is selected from the group consisting of cisplatin, erlotinib, gefitinib, afatinib, osimertinib, abemaciclib, tetraplatin, ipraplatin, abemaciclib, carboplatin, oxaliplatin, nedaplatin, lobaplatin, and/or derivatives thereof.

35. The combination therapeutic according to claim 32 or claim 34, wherein the anti-neoplastic drug is cisplatin.

36. The combination therapeutic according to any one of claims 32-34, wherein the anti-neoplastic drug is gefitinib.

37. The combination therapeutic according to any one of claims 32-36, wherein the aminothiol-conjugate of Formula (I) and the anti-neoplastic drug are formulated together in a single pharmaceutical composition.

38. The combination therapeutic according to any one of claims 32-36, wherein the aminothiol-conjugate of Formula (I) and the anti-neoplastic drug are formulated as separate pharmaceutical compositions.

39. The combination therapeutic according to any one of claims 32-38, wherein a dosage unit of the combination therapeutic comprises about 0.001 mg to about 1,000 mg of the aminothiol-conjugate of Formula (I).

40. The combination therapeutic according to claim 39, wherein a dosage unit of the combination therapeutic comprises about 0.01 mg to about 500 mg of the aminothiol-conjugate of Formula (I).

41. The combination therapeutic according to claim 39, wherein a dosage unit of the combination therapeutic comprises about 0.001 mg to about 500 mg of the aminothiol-conjugate of Formula (I).

42. The combination therapeutic according to any one of claims 32-41, wherein a dosage unit of the combination therapeutic comprises about 0.001 mg to about 1,000 mg of the anti-neoplastic drug.

43. The combination therapeutic according to any one of claims 32-42, wherein the aminothiol-conjugate of Formula (I) has the following structure: 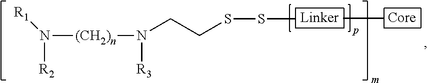
wherein 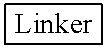
is polyethylene glycol (PEG) or a repeating unit of PEG; 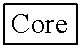
is optional and, if present, is a polymer core or 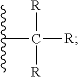
m is 1, 2, 3, 4, 5, 6, 7, or 8;
R is independently selected from hydrogen and C₁₋₆ alkyl;
R₁, R₂, and R; are H;
n is 3; and
each p is independently 1 to 2,500.

44. The combination therapeutic according to any one of claims 32-43, wherein the aminothiol-conjugate of Formula (I) has the structure of:

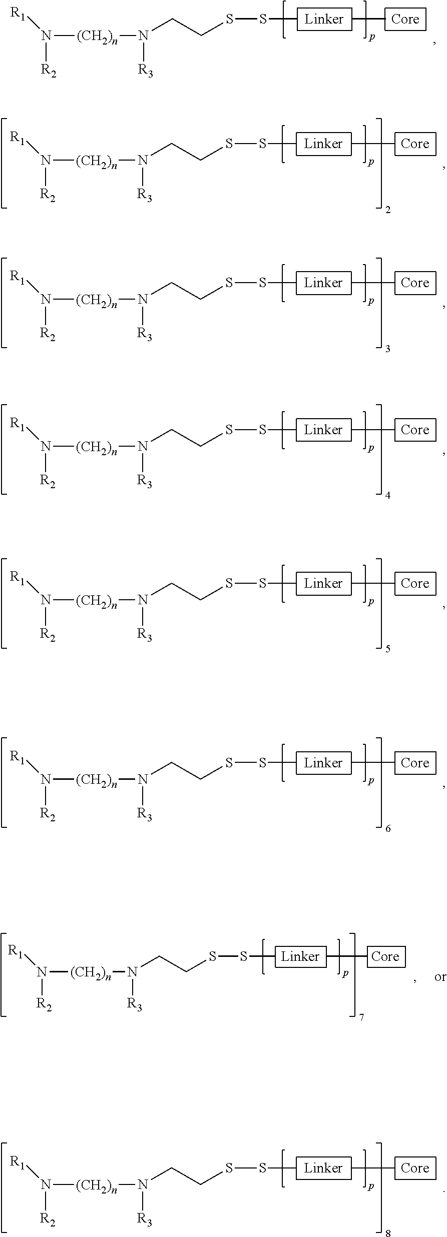

45. The combination therapeutic according to any one of claims 32-44, wherein the aminothiol-conjugate of Formula (I) has the following structure: 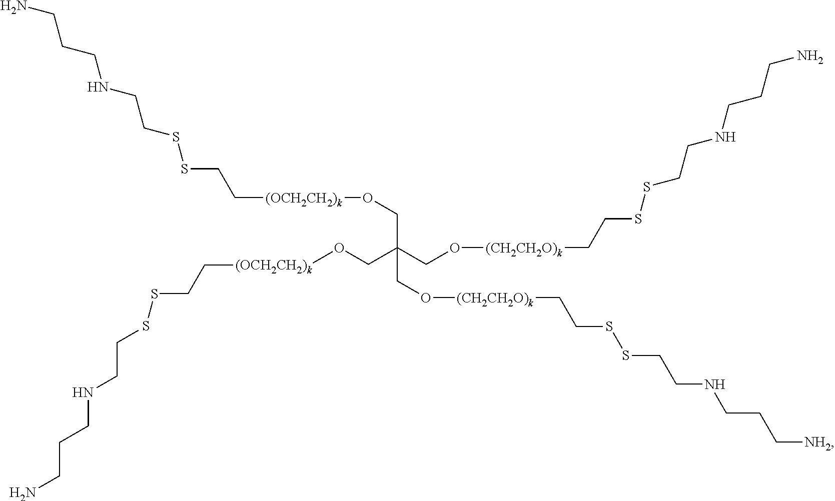
wherein k is independently 1 to 2,500, or a pharmaceutically acceptable salt thereof.

46. The combination therapeutic according to any one of claims 32-44, wherein the aminothiol-conjugate of Formula (I) has the following structure: 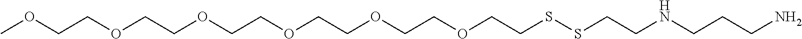 [SMILES: COCCOCCOCCOCCOCCOCCSSCCNCCCN]
or a pharmaceutically acceptable salt thereof.

47. A method of treating a subject in need of antimicrobial treatment, said method comprising:
administering to said subject a combination therapy comprising: (i) an aminothiol-conjugate of Formula (I): 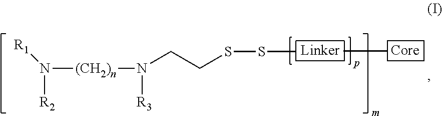
wherein 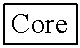
is optional and, if present, is an atom, a molecule, or a macromolecule; 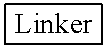
is a linker group, wherein the linker group is a polymer, a section of a polymer, an arm of a polymer, an arm of a copolymer, or a branch of a dendrimer;
wherein each 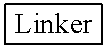
can be the same or different;
R₁, R₂, and R₃ are independently selected from hydrogen and C₁₋₆ alkyl;
m is 1 to 100,000;
n is 1 to 10; and
p is independently 0 to 2,500,
or a pharmaceutically acceptable salt, hydrate, polymorph, or solvate thereof, and (ii) an antimicrobial drug, wherein the combination therapy is administered in an amount effective to treat a microbial condition in the subject.

48. The method according to claim 47, wherein the an antimicrobial drug is selected from the group consisting of an antibiotic drug, an antiviral drug, an antifungal drug, an antiparasitic drug, or a derivative thereof.

49. The method according to claim 47, wherein the antimicrobial drug is selected from the group consisting of (i) eukaryotic and prokaryotic protein kinase inhibitors, (ii) nucleoside analogs, (iii) bacterial cell wall or cell envelope breakdown agents, (iv) antifungal drugs, (v) antiparasitic drugs, (vi) antiviral drugs, or derivatives thereof.

50. The method according to claim 47, wherein the antimicrobial drug is an antiviral drug selected from the group consisting of (i) eukaryotic and prokaryotic protein kinase inhibitors and (ii) nucleoside analogs or derivatives thereof.

51. The method according to any one of claims 47-50, wherein the subject is a mammal.

52. The method according to claim 51, wherein the mammal is a human.

53. The method according to any one of claims 47-52, wherein the subject has a condition resistant to a treatment with the antimicrobial drug.

54. The method according to claim 53, wherein the condition is a bacterial infection, a parasitic infection, a fungal infection, or a viral infection.

55. The method according to claim 53, wherein the condition is resistant to treatment with an antiviral drug.

56. The method according to any one of claims 47-48, wherein the subject is in need of antiviral treatment for HIV.

57. The method according to any one of claims 40-55, wherein the subject is infected with a virus that encodes a reverse transcriptase, orthomyxovirus, RNA viruses, or DNA viruses.

58. The method according to claim 57, wherein the subject is infected with an influenza virus type selected from the group consisting of H1N1, H5N1, and H3N2.

59. The method according to claim 57, wherein the subject is infected with an adenovirus species selected from the group consisting of B, C, and E.

60. The method according to any one of claims 47-55, wherein the subject is not infected with HIV.

61. The method according to any one of claims 47-60, wherein the aminothiol-conjugate of Formula (I) has the following structure: 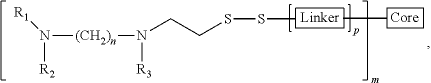
wherein 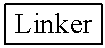
is a polyethylene glycol (PEG) or a repeating unit of PEG; 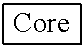
is optional and, if present, is a polymer core or 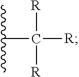
m is 1, 2, 3, 4, 5, 6, 7, or 8;
R is independently selected from hydrogen and C₁₋₆ alkyl;
R₁, R₂, and R₅ are H;
n is 3; and
each p is independently 1 to 2,500.

62. The method according to any one of claims 47-61, wherein the aminothiol-conjugate of Formula (I) has the structure of:

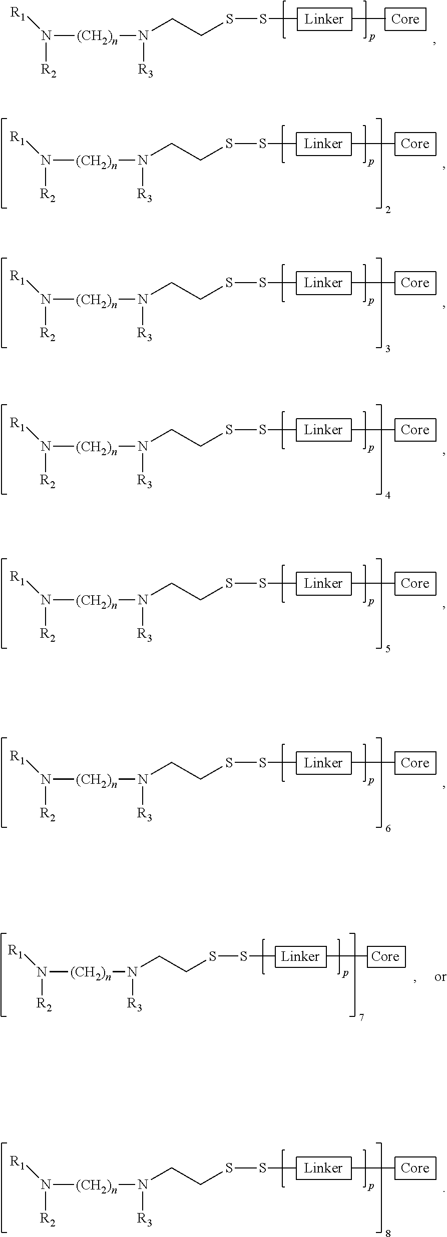

63. The method according to any one of claims 47-62, wherein the aminothiol-conjugate of Formula (I) has the following structure: 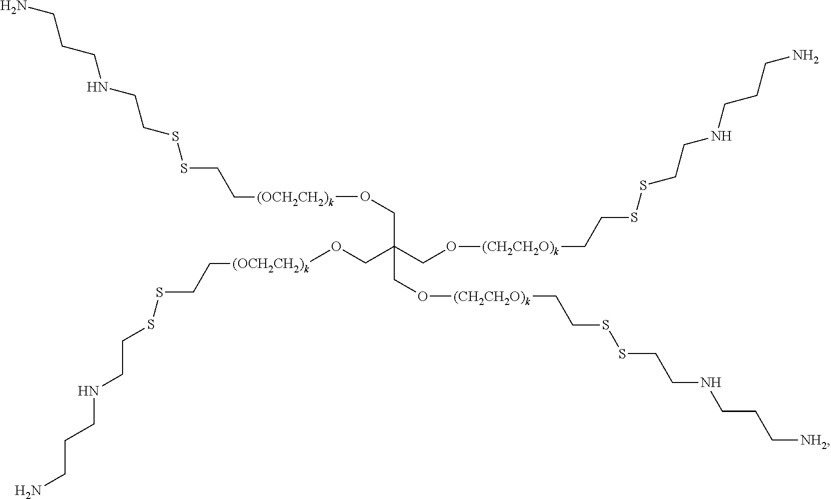
wherein k is independently 1 to 2,500, or a pharmaceutically acceptable salt thereof.

64. The method according to any one of claims 47-62,
wherein the aminothiol-conjugate of Formula (I) has the following structure: 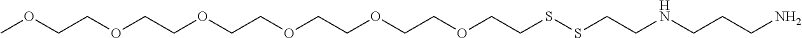 [SMILES: COCCOCCOCCOCCOCCOCCSSCCNCCCN]
or a pharmaceutically acceptable salt thereof.

65. A combination therapeutic comprising:
(i) an aminothiol-conjugate of Formula (I): 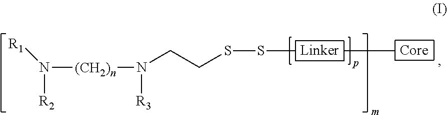
wherein 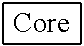
is optional and, if present, is an atom, a molecule, or a macromolecule; 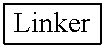
is a linker group, where the linker group is a polymer, a section of a polymer, an arm of a polymer, an arm of a copolymer, or a branch of a dendrimer;
wherein each 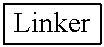
can be the same or different;
R₁, R₂, and R₃ are independently selected from hydrogen and C₁₋₆ alkyl;
m is 1 to 100,000;
n is 1 to 10; and
p is independently 0 to 2,500,
or a pharmaceutically acceptable salt, hydrate, polymorph, or solvate thereof, and
(ii) an antimicrobial drug.

66. The combination therapeutic according to claim 65, wherein the antimicrobial drug is selected from the group consisting of an antibiotic drug, an antiviral drug, an antifungal drug, an antiparasitic drug, or a derivative thereof.

67. The combination therapeutic according to claim 65, wherein the antimicrobial drug is selected from the group consisting of (i) eukaryotic and prokaryotic protein kinase inhibitors, (ii) nucleoside analogs, (iii) bacterial cell wall or cell envelope breakdown agents, (iv) antifungal drugs, (v) antiparasitic drugs, (vi) antiviral drugs, or derivatives thereof.

68. The combination therapeutic according to claim 65, wherein the antimicrobial drug is an antiviral selected from the group consisting of (i) eukaryotic and prokaryotic protein kinase inhibitors and (ii) nucleoside analogs or derivatives thereof.

69. The combination therapeutic according to any one of claims 65-68, wherein the aminothiol-conjugate of Formula (I) and the antimicrobial drug are formulated together in a single pharmaceutical composition.

70. The combination therapeutic according to any one of claims 65-68, wherein the aminothiol-conjugate of Formula (I) and the antimicrobial are formulated as separate pharmaceutical compositions.

71. The combination therapeutic according to any one of claims 65-70, wherein a dosage unit of the combination therapeutic comprises about 0.001 mg to about 1,000 mg of the aminothiol-conjugate of Formula (I).

72. The combination therapeutic according to claim 71, wherein a dosage unit of the combination therapeutic comprises about 0.01 mg to about 500 mg of the aminothiol-conjugate of Formula (I).

73. The combination therapeutic according to claim 71, wherein a dosage unit of the combination therapeutic comprises about 0.001 mg to about 500 mg of the aminothiol-conjugate of Formula (I).

74. The combination therapeutic according to any one of claims 65-73, wherein a dosage unit of the combination therapeutic comprises about 0.001 mg to about 1,000 mg of the antimicrobial drug.

75. The combination therapeutic according to any one of claims 65-74 for administration to a mammal.

76. The combination therapeutic according to claim 75, wherein the mammal is a human.

77. The combination therapeutic according to any one of claims 65-76, wherein the aminothiol-conjugate of Formula (I) has the following structure: 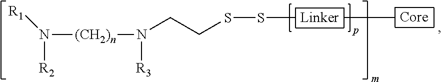
wherein 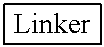
is polyethylene glycol (PEG) or a repeating unit of PEG; 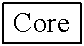
is optional and, if present, is a polymer core or 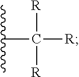
m is 1, 2, 3, 4, 5, 6, 7, or 8;
R is independently selected from hydrogen and C₁₋₆ alkyl;
R₁, R₂, and R₃ are H;
n is 3; and
each p is independently 1 to 2,500.

78. The combination therapeutic according to any one of claims 65-77, wherein the aminothiol-conjugate of Formula (I) has the structure of:

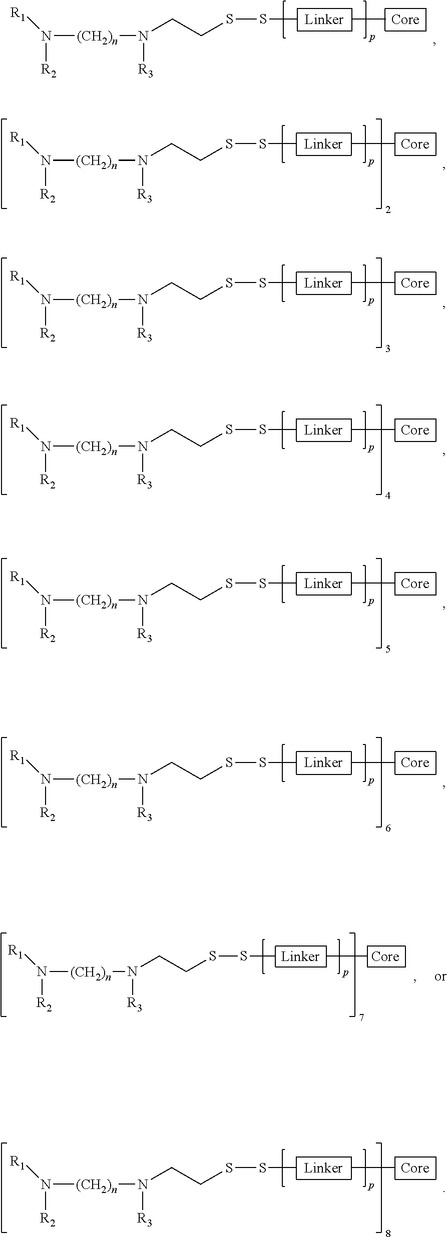

79. The combination therapeutic according to any one of claims 65-78, wherein the aminothiol-conjugate of Formula (I) has the following structure: 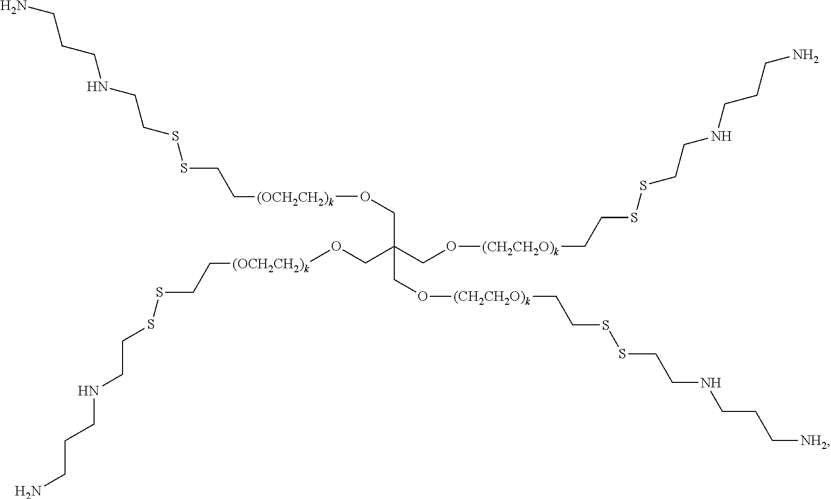
wherein k is independently 1 to 2,500, or a pharmaceutically acceptable salt thereof.

80. The combination therapeutic according to any one of claims 65-78,
wherein the aminothiol-conjugate of Formula (I) has the following structure: 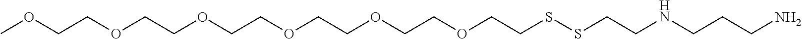 [SMILES: COCCOCCOCCOCCOCCOCCSSCCNCCCN]
or a pharmaceutically acceptable salt thereof.

81. The method according to any one of claim 1-31 or 47-64 or the combination therapeutic of any one of claim 32-46 or 65-80, wherein the linker is a PEG having an average molecular weight in the range from about 0.05 kDa to about 1 kDa.

82. The method according to any one of claim 1-31 or 47-64 or the combination therapeutic of any one of claim 32-46 or 65-80, wherein the aminothiol-conjugate of Formula (I) has an average molecular weight in the range from about 0.25 kDa to about 100 kDa.

83. The method according to any one of claims 1-31 or 47-64 or the combination therapeutic of any one of claim 32-46 or 65-80, wherein the aminothiol-conjugate of Formula (I) has an average molecular weight in the range from about 0.1 kDa to about 1 kDa.

84. The method according to any one of claim 1-15, 18-29, or 47-62 or the combination therapeutic of any one of claim 32-44 or 65-78, wherein the aminothiol-conjugate of Formula (I) has the following structure: 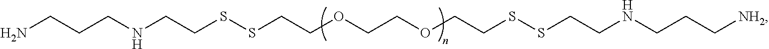
wherein n is independently 1 to 2,500.

85. The method according to any one of claim 1-16, 18-30, or 47-63 or the combination therapeutic of any one of claim 32-45 or 65-79, wherein the aminothiol-conjugate of Formula (I) is a multi-armed PEG-containing molecule.

86. The method according to any one of claim 1-16, 18-30, or 47-63 or the combination therapeutic of any one of claim 32-45 or 65-79, wherein the aminothiol-conjugate of Formula (I) is an asymmetric multi-armed PEG-containing molecule.
